(19) United States
(12) Patent Application Publication
(10) Pub. No.: US 2016/0133847 A1
(43) Pub. Date: May 12, 2016

(54) AROMATIC AMINE DERIVATIVE AND ORGANIC ELECTROLUMINESCENT ELEMENT

(71) Applicant: IDEMITSU KOSAN CO., LTD., Chiyoda-ku, Tokyo (JP)

(72) Inventors: Takahiro Fujiyama, Kisarazu-shi (JP); Tomoki Kato, Ichihara-shi (JP); Masakazu Funahashi, Chiba-shi (JP); Yoshiaki Takahashi, Chiba-shi (JP)

(73) Assignee: IDEMITSU KOSAN CO., LTD., Chiyoda-ku, Tokyo (JP)

(21) Appl. No.: 14/899,037

(22) PCT Filed: Jun. 19, 2014
(86) PCT No.: PCT/JP2014/003312

§ 371 Date: Dec. 16, 2015

(30) Foreign Application Priority Data
Jun. 19, 2013 (JP) 2013-128789

(51) Int. Cl. H01L 51/00; C07C 211/54; C07D 307/91; C07C 211/61

(52) U.S. Cl. CPC H01L 51/0059; C07C 211/61; H01L 51/006; C07C 211/54; C07D 307/91; H01L 51/0061; H01L 51/5064

(57) ABSTRACT

An object of the invention is to provide an organic EL device which operates at a reduced driving voltage and has a prolonged lifetime. The aromatic amine derivative of the invention is represented by formula (1):

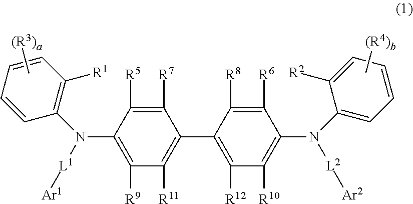

wherein Ar¹ and Ar² each represent an aryl group or a heteroaryl group; L¹ and L² each represent a single bond, an arylene group or a heteroarylene group; R¹ to R⁴ each represent an alkyl group, an aryl group, a heteroaryl group, an alkoxy group, an aralkyl group, an aryloxy group, an arylthio group, an alkoxycarbonyl group, a halogen atom, a cyano group, a nitro group, a silyl group, a hydroxyl group, or a carboxyl group; a and b each represent an integer of 0 to 4; and R⁵ to R¹² each represent a hydrogen atom, an alkyl group, an aryl group, a heteroaryl group or a silyl group; provided that at least one selected from R⁵ and R⁷ is not a hydrogen atom, wherein when R⁵ is not a hydrogen atom, R⁶ is not a hydrogen atom, and when R⁷ is not a hydrogen atom, R⁸ is not a hydrogen atom.

### TECHNICAL FIELD

The present invention relates to aromatic amine derivatives and organic electroluminescence (EL) devices employing the aromatic amine derivatives.

### BACKGROUND ART

In recent years, research and development has been actively directed to functional materials of organic compound. Recently, the development of an organic electroluminescence device (organic EL device) employing an organic compound as a material for constituting a light emitting device is made actively.

An organic electroluminescence device has a structure wherein a thin film comprising a light emitting organic compound is interposed between an anode and a cathode. The electrons and the holes each injected into the thin film recombine to generate excitons. Since an organic EL device of laminate type capable of driving at low voltage has been reported by C. W. Tang et al. of Eastman Kodak Company (Non-Patent Literature 1), many studies have been made on an organic EL device which comprises an organic material. Tang et al. used tris(8-quinolinolato)aluminum in a light emitting layer and a triphenyldiamine derivative in a hole transport layer. Advantages of the laminate structure are that the efficiency of hole injection into a light emitting layer can be increased; the efficiency of forming excitons by recombination can be increased by blocking electrons injected from a cathode; and excitons formed can be confined in a light emitting layer. As the structure of organic EL devices, a two-layered structure having a hole transporting (injecting) layer and an electron transporting light emitting layer and a three-layered structure having a hole transporting (injecting) layer, a light emitting layer and an electron transporting (injecting) layer are well known. To increase the efficiency of recombination of injected holes and electrons in laminated devices, the device structure and the process for forming the device have been studied.

Generally, when an organic EL device is driven or stored in an environment of high temperature, there occur drawbacks, such as a change in the luminescent color, a decrease in current efficiency, an increase in driving voltage, and a decrease in a lifetime of light emission. To prevent these drawbacks, it has been necessary to increase the glass transition temperature (Tg) of a hole transporting material. Therefore, it is necessary to introduce many aromatic groups into a molecule of hole transporting material (for example, an aromatic diamine derivative of Patent Literature 1 and a fused aromatic ring diamine derivative of Patent Literature 2), and a compound generally having 8 to 12 benzene rings are preferably used.

However, a hole transporting material having a large number of aromatic groups in its molecule is liable to be crystallized during the thin film-forming process for the production of organic EL device. Therefore, the outlet of a crucible for use in vapor deposition is clogged and the defects caused on a thin film due to the crystallization reduce the yields of organic EL device. In addition, since a compound having a large number of aromatic groups in its molecule generally has a high glass transition temperature (Tg), but has a high sublimation temperature, the compound may be decomposed during the vapor deposition and the deposited film may be nonuniform, thereby shortening the lifetime of organic EL device.

In addition, a tetraaryldiamine derivative has been discloses. For example, the tetraaryldiamine derivatives disclosed in Patent Literatures 3 and 4 have only a para- or meta-substituted biphenyl group, and the exemplary compounds and the compounds used in the working examples disclosed therein include no ortho-substituted derivative. Patent Literature 5 discloses a heat-stable asymmetric compound having a high glass transition temperature. However, no diamine compound is described in the exemplary compounds and the working examples. In addition, these reports are completely silent about the effect of reducing the driving voltage.

Although an organic EL device with a long lifetime has been reported, the improvement in the lifetime and the reduction in the driving voltage are not necessarily sufficient. Thus, an organic EL device with sufficient performance cannot be obtained by merely increasing the molecular weight of the material. Therefore, it has been strongly demanded to develop an organic EL device with improved performance.

### CITATION LIST

### Patent Literature

Patent Literature 1: U.S. Pat. No. 4,720,432
Patent Literature 2: U.S. Pat. No. 5,061,569
Patent Literature 3: JP 8-48656A
Patent Literature 4: JP 2008-247904A
Patent Literature 5: JP 2002-53533A

### Non Patent Literature

Non-Patent Literature 1: C. W. Tang and S. A. Vanslyke, Applied Physics Letters, vol. 51, p 913, 1987

### SUMMARY OF INVENTION

### Technical Problem

The present invention has been made to solve the above technical problem and an object of the invention is to provide an aromatic amine derivative with a specific structure which reduces a driving voltage and improves a lifetime when used in an organic EL device as a hole transporting material.

### Solution to Problem

As a result of extensive research for achieving the above object, the inventors have found that the above technical problem is solved by using an aromatic amine derivative having a specific structure represented by formula (1) in an organic EL device, particularly as a hole transporting material. The invention is based on this finding.

The diamine compound has a high glass transition temperature because the free rotation of the groups around the amine nitrogen atom is inhibited. In addition, the decomposition hardly occurs during vapor deposition because of its low molecular weight. Further, an organic EL device employing the diamine compound operates at a reduced driving voltage and has a prolonged lifetime. Thus, it has been found that the diamine compound has a remarkable effect on prolonging the lifetime.

First, the present invention relates to the following aromatic amine derivative:

(1) an aromatic amine derivative represented by formula (1):

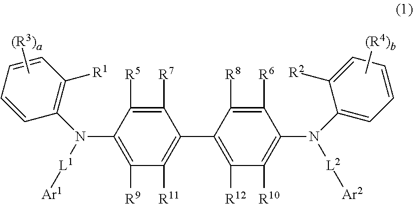

wherein:

Ar¹ and Ar² each independently represent a substituted or unsubstituted aryl group having 6 to 50 ring carbon atoms or a substituted or unsubstituted heteroaryl group having 3 to 50 ring atoms;

L¹ and L² each independently represent a single bond, a substituted or unsubstituted arylene group having 6 to 50 ring carbon atoms, or a substituted or unsubstituted heteroarylene group having 3 to 50 ring atoms;

R¹ to R⁴ each independently represents a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;

a and b each represent an integer of 0 to 4; and

R⁵ to R¹² each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group;

provided that at least one selected from R⁵ and R⁷ is not a hydrogen atom, wherein when R⁵ is not a hydrogen atom, R⁶ is not a hydrogen atom, and when R⁷ is not a hydrogen atom, R⁸ is not a hydrogen atom;

(2) the aromatic amine derivative of item 1, wherein the aromatic amine derivative is represented by formula (2):

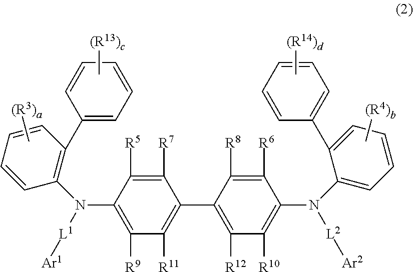

wherein:

R¹³ and R¹⁴ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;

c and d each represent an integer of 0 to 5, and when c and d each represent 2, groups R¹³ or groups R¹⁴ may be bonded to each other to form a ring, provided that R¹³ together with its adjacent aromatic ring and R¹⁴ together with its adjacent aromatic ring do not form a dibenzofuran ring, a dibenzothiophene ring or a fluorene ring; and

Ar¹, Ar², L¹, L², R³ to R¹², a, and b are as defined in formula (1); (3) the aromatic amine derivative of item 2, wherein the aromatic amine derivative is represented by formula (3):

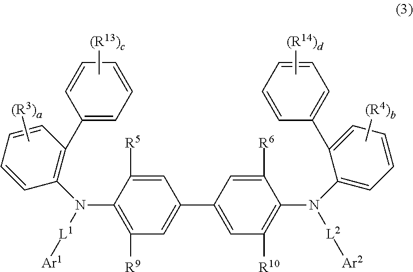

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², L¹, L², R³, R⁴, R⁹, R¹⁰, R¹³, R⁴, a, b, c, and d are as defined in formula (2);

(4) the aromatic amine derivative of item 2, wherein the aromatic amine derivative is represented by formula (4):

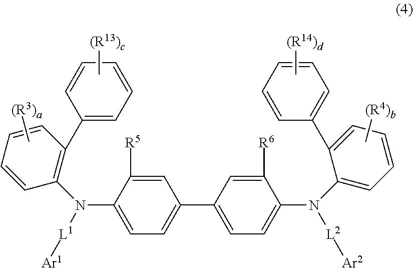

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², L¹, L², R³, R⁴, R¹³, R¹⁴, a, b, c, and d are as defined in formula (2);

(5) the aromatic amine derivative of item 2, wherein the aromatic amine derivative is represented by formula (5):

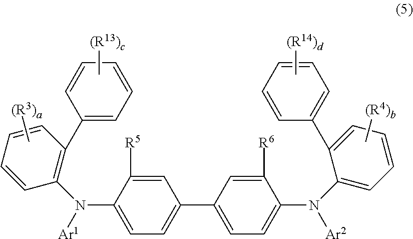

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², R³, R⁴, R¹³, R¹⁴, a, b, c, and d are as defined in formula (2);

(6) the aromatic amine derivative of item 2, wherein the aromatic amine derivative is represented by formula (6):

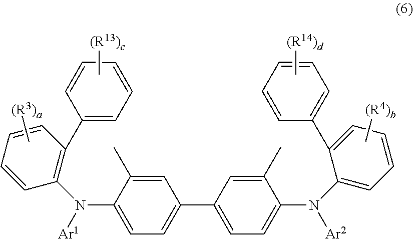

wherein Ar¹, Ar², R³, R⁴, R¹³, R¹⁴, a, b, c, and d are as defined in formula (2);
(7) the aromatic amine derivative of item 2, wherein the aromatic amine derivative is represented by formula (7):

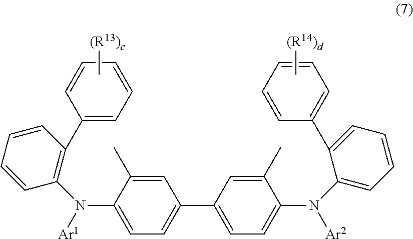

wherein Ar¹, Ar², R¹³, R¹⁴, c, and d are as defined in formula (2);
(8) the aromatic amine derivative of item 1, wherein the aromatic amine derivative is represented by formula (8):

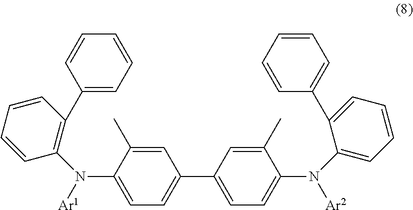

wherein Ar¹ and Ar² are as defined in formula (1);
(9) the aromatic amine derivative of item 3, wherein Ar¹ and Ar² each represent a substituted or unsubstituted aryl group having 10 to 50 ring carbon atoms;
(10) the aromatic amine derivative of item 2, wherein the aromatic amine derivative is represented by formula (26):

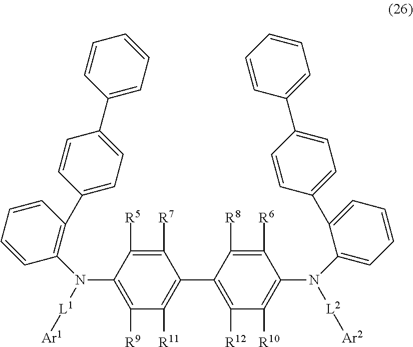

wherein Ar¹, Ar², L¹, L², and R⁵ to R¹² are as defined in formula (2);
(11) the aromatic amine derivative of item 10, wherein R⁵ is not a hydrogen atom and R⁶ is not a hydrogen atom;
(12) the aromatic amine derivative of item 1, wherein the aromatic amine derivative is represented by formula (17):

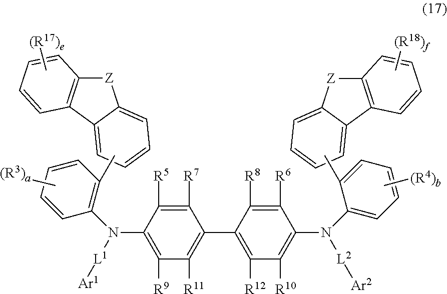

wherein:

Z represents —O—, —S—, or —CR¹⁹R²⁰—, wherein R¹⁹ and R²⁰ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group;

R¹⁷ and R¹⁸ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;

e and f each independently represent an integer of 0 to 4; and

Ar¹, Ar², L¹, L², R³ to R¹², a, and b are as defined in formula (1);

(13) the aromatic amine derivative of item 12, wherein the aromatic amine derivative is represented by formula (18):

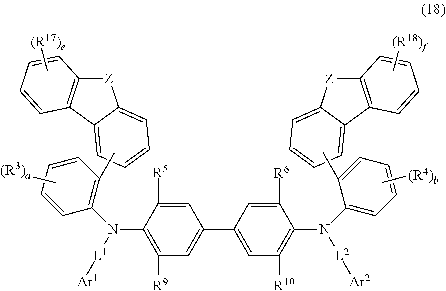

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², L¹, L², R³, R⁴, R⁹, R¹⁰, R¹⁷, R¹⁸, a, b, e, f, and Z are as defined in formula (17);

(14) the aromatic amine derivative of item 12, wherein the aromatic amine derivative is represented by formula (19):

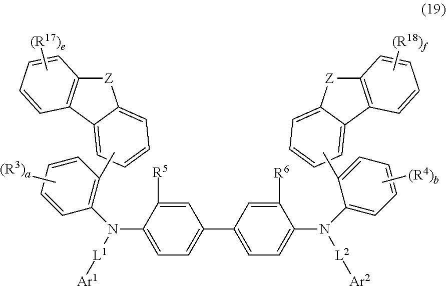

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², L¹, L², R³, R⁴, R¹⁷, R¹⁸, a, b, e, f and Z are as defined in formula (17);

(15) the aromatic amine derivative of item 12, wherein the aromatic amine derivative is represented by formula (20):

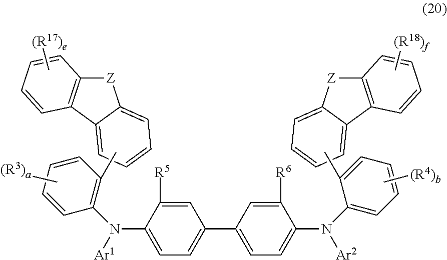

wherein:

R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and

Ar¹, Ar², R³, R⁴, R¹⁷, R¹⁸, a, b, e, f, and Z are as defined in formula (17);

(16) the aromatic amine derivative of item 12, wherein the aromatic amine derivative is represented by formula (21):

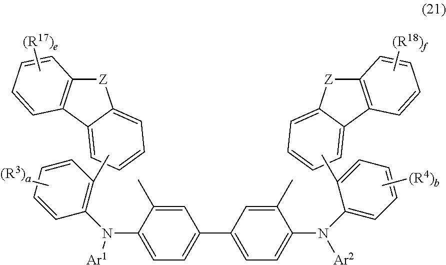

wherein Ar¹, Ar², R³, R⁴, R¹⁷, R¹⁸, a, b, e, f, and Z are as defined in formula (17);
(17) the aromatic amine derivative of item 12, wherein the aromatic amine derivative is represented by formula (22):

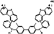

wherein Ar¹, Ar², R¹⁷, R¹⁸, e, f, and Z are as defined in formula (17);
(18) the aromatic amine derivative of item 1, wherein the aromatic amine derivative is represented by any one of formulae (23) to (25):

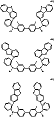

wherein Ar¹, Ar² and Z are as defined in formula (17);
(19) the aromatic amine derivative of any one of items 1 to 4 and 10 to 14, wherein L¹ and L² in any of formulae (17) to (19) and (26) each independently represent a single bond or a divalent group represented by formula (27):

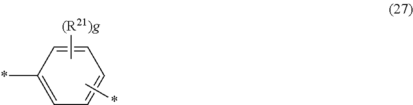

wherein:

each R²¹ independently represents a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group; and

g represents an integer of 0 to 4;

(20) the aromatic amine derivative of any one of items 1 to 18, wherein Ar¹ and Ar² in any of formulae (1) to (8) and (17) to (26) are each independently represented by any one of formulae (9) to (16):

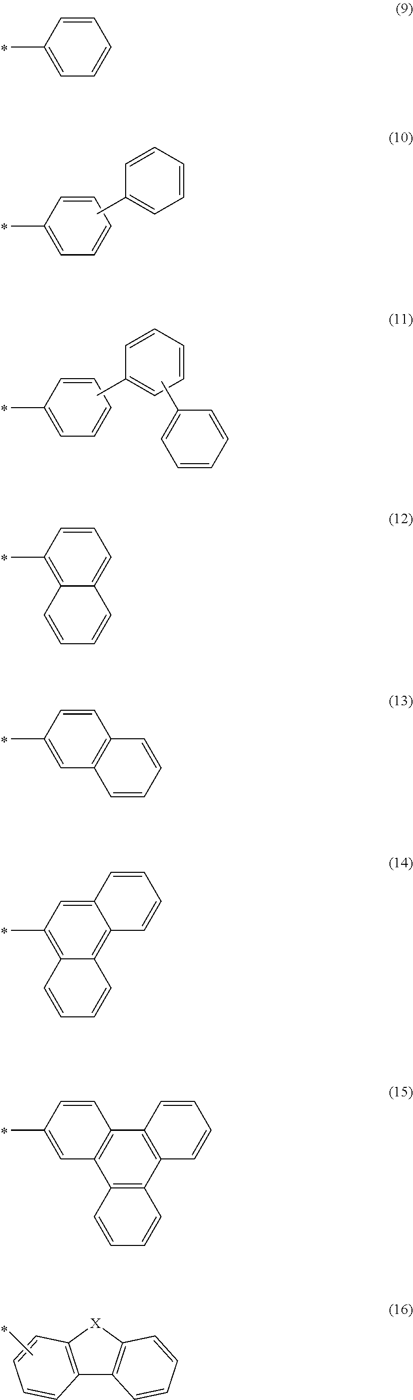

wherein:

X represents —O—, —S—, or —CR¹⁵R¹⁶—; and

R¹⁵ and R¹⁶ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group;

(21) a material for organic electroluminescence devices, which comprises the aromatic amine derivative of any one of items 1 to 20;
(22) a hole transporting material for organic electroluminescence devices, which comprises the aromatic amine derivative of any one of items 1 to 20;
(23) a hole transporting material for organic electroluminescence devices, which comprises a hole transporting layer adjacent to an acceptor layer and the hole transporting layer comprises the aromatic amine derivative of any one of items 1 to 20; and
(24) an organic electroluminescence device, which comprises an organic thin film layer between an anode and a cathode opposite to the anode and at least one layer of the organic thin film layer comprises the aromatic amine derivative of any one of items 1 to 20.

### Advantageous Effects of Invention

The present invention provides an aromatic amine derivative which reduces the driving voltage and improves the lifetime of an organic EL device.

### DESCRIPTION OF EMBODIMENTS

The aromatic amine derivative of the invention is represented by formula (1).

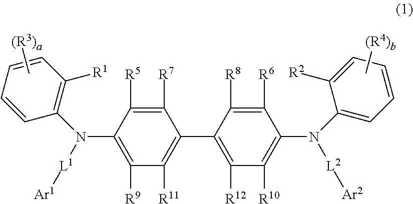

In formula (1), Ar¹ and Ar² each independently represent a substituted or unsubstituted aryl group having 6 to 50 ring carbon atoms or a substituted or unsubstituted heteroaryl group having 3 to 50 ring atoms.

In formula (1), L¹ and L² each independently represents a single bond, a substituted or unsubstituted arylene group having 6 to 50 ring carbon atoms, or a substituted or unsubstituted heteroarylene group having 3 to 50 ring atoms, with a single bond and a substituted or unsubstituted arylene group having 6 to 50 ring carbon atoms being preferred.

In formula (1), R¹ to R⁴ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, an amino group having a substituted or unsubstituted aryl substituent having 5 to 50 ring carbon atoms, a halogen atom, a cyano group, a nitro group, a silyl group, a hydroxyl group, or a carboxyl group.

Preferably, R¹ and R² each independently represent a substituted or unsubstituted aryl group having 6 to 50 ring carbon atoms, a substituted or unsubstituted heteroaryl group having 5 to 50 ring atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, or an amino group having a substituted or unsubstituted aryl substituent having 5 to 50 ring carbon atoms, with a substituted or unsubstituted aryl group having 6 to 50 ring carbon atoms and a substituted or unsubstituted heteroaryl group having 5 to 50 ring atoms being more preferred. The substituted or unsubstituted aryl group having 6 to 50 ring carbon atoms and the substituted or unsubstituted heteroaryl group having 5 to 50 ring atoms are preferably a substituted or unsubstituted aryl group having 6 to 20 ring carbon atoms and a substituted or unsubstituted heteroaryl group having 5 to 20 ring atoms, respectively. These groups are preferred because they are bulky and, as described below, steric hindrance easily occurs between these groups and the groups on the nitrogen atom.

In formula (1), a and b are each an integer of 0 to 4 and preferably 0 or 1.

In formula (1), R⁵ to R¹² each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group. The hole transporting ability is enhanced by introducing a group which increases the electron density of a conjugated portion participating in the hole transport. Therefore, as a group other than a hydrogen atom, a substituted or unsubstituted alkyl group having 1 to 50 carbon atoms and a substituted or unsubstituted alkoxy group having 1 to 50 carbon atoms are preferred, and a substituted or unsubstituted alkyl group having 1 to 50 carbon atoms is more preferred, because these groups are electron donating. The substituted or unsubstituted alkyl group having 1 to 50 carbon atoms is preferably a substituted or unsubstituted alkyl group having 1 to 30 carbon atoms and more preferably a substituted or unsubstituted alkyl group having 1 to 10 carbon atoms.

At least one selected from R⁵ and R⁷ is not a hydrogen atom. When R⁵ is not a hydrogen atom, R⁶ is not a hydrogen atom, and when R⁷ is not a hydrogen atom, R⁸ is not a hydrogen atom. Preferably, neither R⁵ nor R⁶ is a hydrogen atom.

In formula (1), two groups -L₁-Ar₁ and —C₆H₄R¹(R³)ₐ on one nitrogen atom and two groups -L₂-Ar₂ and —C₆H₄R²(R⁴)_{b} on the other nitrogen atom may be the same or different, respectively, and preferably different in view of obtaining the effect of the invention efficiently.

The aromatic amine derivative of the invention is preferably represented by formula (2).

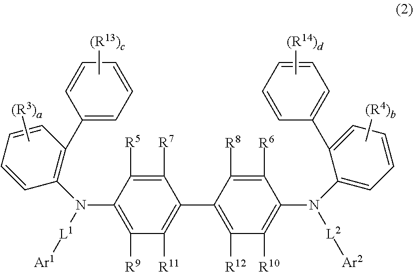

In formula (2), R¹³ and R¹⁴ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group, with a substituted or unsubstituted alkyl group having 1 to 50 carbon atoms, a substituted or unsubstituted aryl group having 6 to 50 ring carbon atoms, and a substituted or unsubstituted heteroaryl group having 5 to 50 ring atoms being preferred.

In formula (2), c and d each represent an integer of 0 to 5 and preferably an integer of 0 to 2. When c and d each represent 2, the groups R¹³ or the groups R¹⁴ may be bonded to each other to form a ring, such as a substituted or unsubstituted, saturated or unsaturated, five- or six-membered ring structure. Examples of the five- or six-membered ring structure include a cycloalkane having 4 to 12 carbon atoms, such as cyclopentane, cyclohexane, adamantane, and norbornane; a cycloalkene having 4 to 12 carbon atoms, such as cyclopentene and cyclohexene; a cycloalkadiene having 6 to 12 carbon atoms, such as cyclopentadiene and cyclohexadiene; and an aromatic ring having 6 to 50 carbon atoms, such as benzene, naphthalene, phenanthrene, anthracene, pyrene, chrysene, and acenaphthylene, with an aromatic ring having 6 to 50 carbon atoms being preferred and an aromatic ring having 6 to 10 carbon atoms being more preferred. Provided that R¹³ and R¹⁴ in formula (2) each are not bonded to an adjacent aromatic ring to form a dibenzofuran ring, a dibenzothiophene ring or a fluorene ring.

In formula (2), Ar¹, Ar², L¹, L², R³ to R¹², a, and b are as defined in formula (1).

The aromatic amine derivative of the invention is preferably represented by any of formulae (3) to (8) and (26).

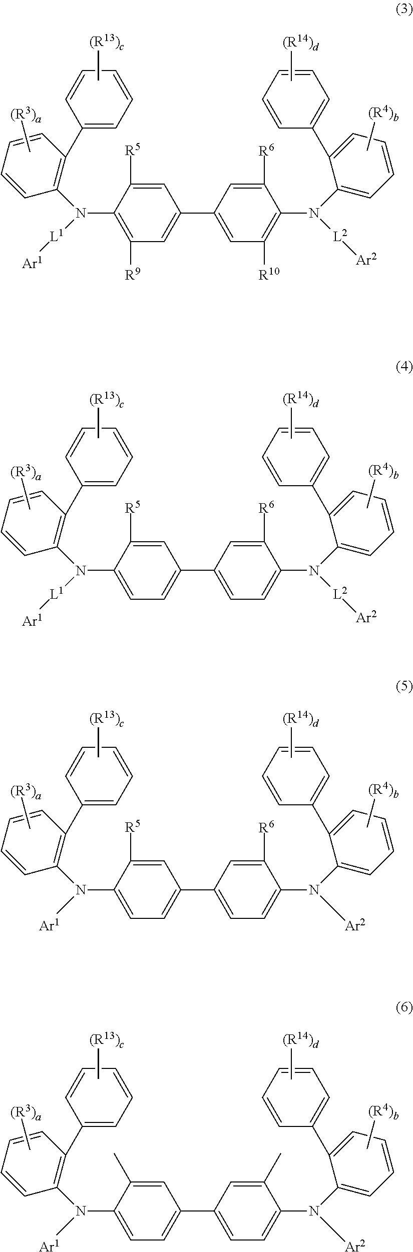

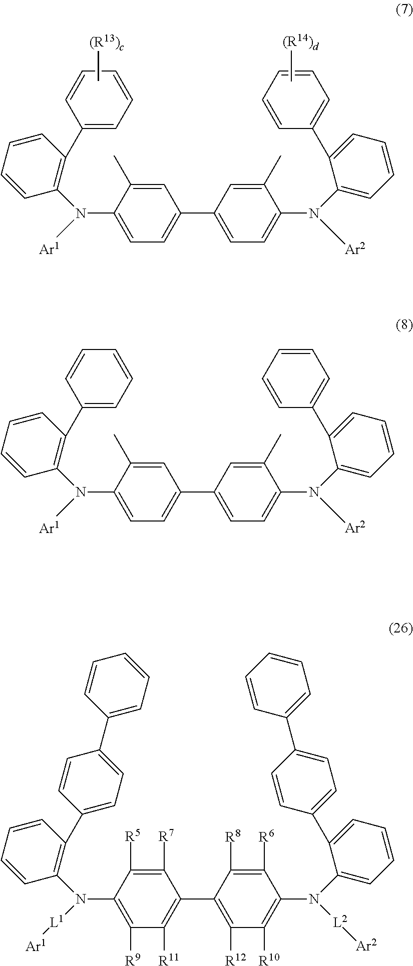

In formulae (3) to (8) and (26), R⁵, R⁶, Ar¹, Ar², L¹, L², R³, R⁴, R⁹, R¹⁰, R¹³, R¹⁴, a, b, c, and d are as defined in formula (1) or (2).

In formula (26), Ar¹, Ar², L¹, L², and R⁵ to R¹⁰ are as defined in formula (1). At least one selected from R⁵ and R⁷ is not a hydrogen atom. When R⁵ is not a hydrogen atom, R⁶ is not a hydrogen atom, and when R⁷ is not a hydrogen atom, R⁸ is not a hydrogen atom. Preferably, neither R⁵ nor R⁶ is a hydrogen atom.

In addition to formula (2), the aromatic amine derivative of the invention is also preferably represented by formula (17).

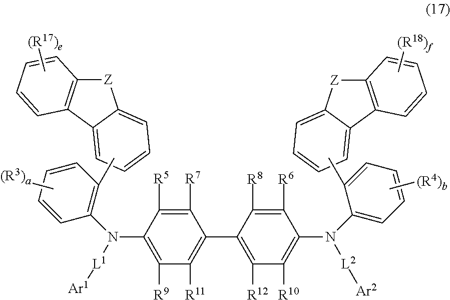

In formula (17), Z represents —O—, —S—, or —CR¹⁹R²⁰— and preferably —O— or —CR¹⁹R²⁰—, wherein R¹⁹ and R²⁰ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group, preferably a hydrogen atom, a substituted or unsubstituted alkyl group having 1 to 50 carbon atoms, or a substituted or unsubstituted aryl group having 6 to 50 ring carbon atoms, and more preferably a hydrogen atom, a substituted or unsubstituted alkyl group having 1 to 20 carbon atoms or a substituted or unsubstituted aryl group having 6 to 20 ring carbon atoms.

In formula (17), R¹⁷ and R¹⁸ each independently represents a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group, preferably a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a substituted or unsubstituted alkoxy group having 1 to 50 carbon atoms.

In formula (17), e and f each represent an integer of 0 to 4, preferably 0.

In formula (17), Ar¹, Ar², L¹, L², R³ to R¹², a, and b are as defined in formula (1).

The aromatic amine derivative of the invention is preferably represented by any of formulae (18) to (25).

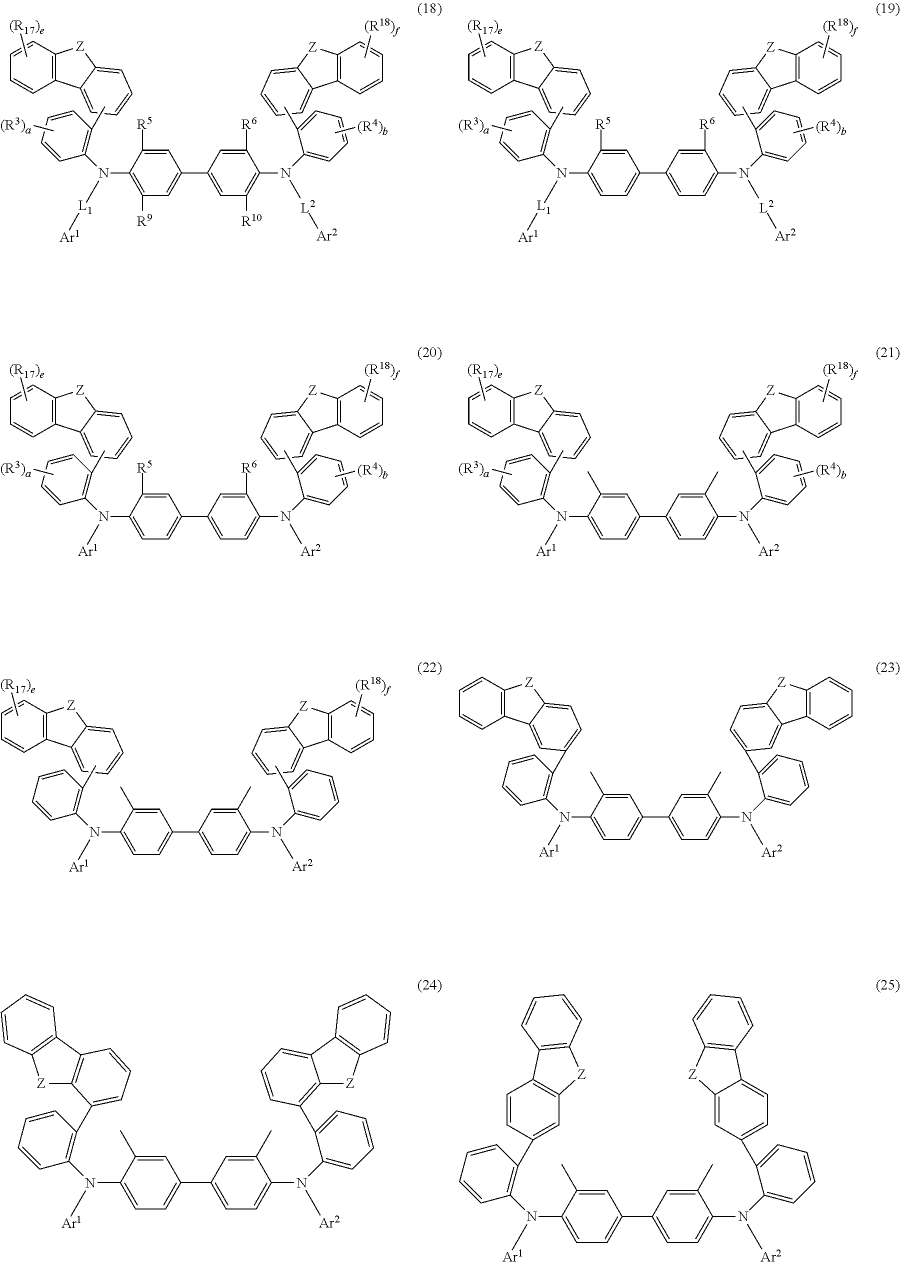

In formulae (1) to (4), (17) to (19), and (26), L¹ and L² each independently represent a single bond, a substituted or unsubstituted arylene group having 6 to 50 ring carbon atoms, or a substituted or unsubstituted heteroarylene group having 3 to 50 ring atoms, preferably a single bond or a substituted or unsubstituted arylene group having 6 to 50 ring carbon atoms. The arylene group is preferably a group represented by formula (27) below and more preferably a phenylene group. Preferred examples of the heteroarylene group include a dibenzofuranylene group and a dibenzothiophenylene group.

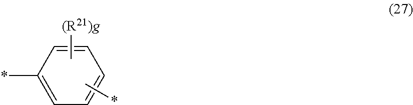

In formula (27), each R²¹ independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group, and preferably a substituted or unsubstituted alkyl group having 1 to 50 carbon atoms. The subscript g represents an integer of 0 to 4, preferably 0 or 1.

The aryl group having 6 to 50 ring carbon atoms for R¹ to R¹⁴, R¹⁷ to R¹⁸, and R²¹ in formulae (1) to (8) and (17) to (27) is preferably an aryl group having 6 to 30 ring carbon atoms and more preferably an aryl group having 6 to 20 ring carbon atoms. Examples thereof include a phenyl group, a biphenylyl group, a terphenylyl group, a biphenylenyl group, a naphthyl group, an acenaphthylenyl group, an anthryl group, a benzanthryl group, an aceanthryl group, a phenanthryl group, a benzophenanthryl group, a triphenylenyl group, a phenalenyl group, a fluorenyl group, a 9,9-dimethylfluorenyl group, a 9,9-diphenylfluorenyl group, a pentacenyl group, a picenyl group, a pentaphenyl group, a pyrenyl group, a chrysenyl group, a benzochrysenyl group, a s-indacenyl group, an as-indacenyl group, a fluoranthenyl group, and a perylenyl group.

The aryl group for R¹ to R¹⁴, R¹⁷ to R¹⁸, and R²¹ is more preferably a phenyl group, a biphenylyl group, a terphenylyl group, a naphthyl group, a fluorenyl group, a phenanthryl group, or a triphenylenyl group.

The heteroaryl group having 5 to 50 ring carbon atoms for R¹ to R¹⁴, R¹⁷ to R¹⁸, and R²¹ in formulae (1) to (8) and (17) to (27) is preferably a heteroaryl group having 5 to 30 ring atoms and more preferably a heteroaryl group having 5 to 20 ring atoms. Examples thereof include a pyrrolyl group, a furyl group, a thienyl group, a pyridyl group, a pyridazinyl group, a pyrimidyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, an indolyl group, an isoindolyl group, a benzofuranyl group, an isobenzofuranyl group, a dibenzofuranyl group, a benzothienyl group, a dibenzothienyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an imidazolyl group, a benzisoxazolyl group, a benzisothiazolyl group, a phenanthridinyl group, an acridinyl group, a phenanthrolinyl group, a phenazinyl group, a phenothiazinyl group, a phenoxazinyl group, and a xanthenyl group.

The heteroaryl group for R¹ to R¹⁴, R¹⁷ to R¹⁸, and R²¹ is more preferably a furyl group, a thienyl group, a benzofuranyl group, a benzothienyl group, a dibenzofuranyl group, or a dibenzothienyl group.

The alkyl group having 1 to 50 carbon atoms for R¹ to R¹⁴, R¹⁷ to R¹⁸, and R²¹ in formulae (1) to (8) and (17) to (27) is preferably an alkyl group having 1 to 30 carbon atoms and more preferably an alkyl group having 1 to 20 carbon atoms. Examples thereof include a methyl group, an ethyl group, a n-propyl group, an isopropyl group, a n-butyl group, a s-butyl group, an isobutyl group, a t-butyl group, a n-pentyl group, a n-hexyl group, a n-heptyl group, a n-octyl group, a n-nonyl group, a n-decyl group, and a neopentyl group, with a methyl group, an ethyl group, a n-propyl group, an isopropyl group, a n-butyl group, a s-butyl group, an isobutyl group, and a t-butyl group being preferred.

The alkoxy group having 1 to 50 carbon atoms for R¹ to R¹⁴, R¹⁷ to R¹⁸, and R²¹ in formulae (1) to (8) and (17) to (27) is represented by —OY, wherein Y is an alkyl group selected from those mentioned above.

The aralkyl group having 6 to 50 carbon atoms for R¹ to R¹⁴, R¹⁷ to R¹⁸, and R²¹ in formulae (1) to (8) and (17) to (27) is preferably an aralkyl group having 6 to 30 carbon atoms and more preferably an aralkyl group having 6 to 20 carbon atoms. Examples thereof include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, a 1-pyrrolylmethyl group, a 2-(1-pyrrolyl)ethyl group, a p-methylbenzyl group, a m-methylbenzyl group, an o-methylbenzyl group, a p-chlorobenzyl group, a m-chlorobenzyl group, an o-chlorobenzyl group, a p-bromobenzyl group, a m-bromobenzyl group, an o-bromobenzyl group, a p-iodobenzyl group, a m-iodobenzyl group, an o-iodobenzyl group, a p-hydroxybenzyl group, a m-hydroxybenzyl group, an o-hydroxybenzyl group, a p-aminobenzyl group, a m-aminobenzyl group, an o-aminobenzyl group, a p-nitrobenzyl group, a m-nitrobenzyl group, an o-nitrobenzyl group, a p-cyanobenzyl group, a m-cyanobenzyl group, an o-cyanobenzyl group, a 1-hydroxy-2-phenylisopropyl group, and a 1-chloro-2-phenylisopropyl group.

The aryloxy group having 5 to 50 ring carbon atoms for R¹ to R¹⁴, R¹⁷ to R¹⁸, and R²¹ in formulae (1) to (8) and (17) to (27) is represented by —OY′, wherein Y is an aryl group selected from those mentioned above.

The arylthio group having 5 to 50 ring carbon atoms for R¹ to R¹⁴, R¹⁷ to R¹⁸, and R²¹ in formulae (1) to (8) and (17) to (27) is represented by —SY′, wherein Y′ is an aryl group selected from those mentioned above.

The alkoxycarbonyl group having 2 to 50 carbon atoms for R¹ to R¹⁴, R¹⁷ to R¹⁸, and R²¹ in formulae (1) to (8) and (17) to (27) is represented by —COOY, wherein Y is an alkyl group selected from those mentioned above.

The aryl substituent in the amino group having an aryl substituent having 5 to 50 ring carbon atoms for R¹ to R¹⁴, R¹⁷ to R¹⁸, and R²¹ in formulae (1) to (8) and (17) to (27) is selected from the aryl groups mentioned above.

Examples of the halogen atom for R¹ to R¹⁴, R¹⁷ to R¹⁸, and R²¹ in formulae (1) to (8) and (17) to (27) include a fluorine atom, a chlorine atom, a bromine atom, and an iodine atom.

The aryl group, the heteroaryl group, the alkyl group, the alkoxy group, the aralkyl group, the aryloxy group, the arylthio group, the alkoxycarbonyl group, and the amino group having an aryl substituent may have a substituent. Preferred examples of the substituent include an alkyl group having 1 to 6 carbon atoms, such as an ethyl group, a methyl group, an isopropyl group, a n-propyl group, a s-butyl group, a t-butyl group, a pentyl group, a hexyl group, a cyclopentyl group, and a cyclohexyl group; an alkoxy group having 1 to 6 carbon atoms, such as an ethoxy group, a methoxy group, an isopropoxy group, a n-propoxy group, a s-butoxy group, t-butoxy group, a pentoxy group, hexyloxy group, a cyclopentoxy group, and a cyclohexyloxy group; an aryl group having 5 to 40 ring carbon atoms, preferably an aryl group having 6 to 12 ring carbon atoms; a heteroaryl group having 5 to 20 ring carbon atoms; an amino group having an aryl substituent having 5 to 40 ring carbon atoms; an ester group having an aryl group having 5 to 40 ring carbon atoms; an ester group having an alkyl group having 1 to 6 carbon atoms; a cyano group; a nitro group; and a halogen atom, such as a chlorine atom, a bromine atom, and an iodine atom.

R¹ to R¹² in formulae (1) to (8) and (17) to (26) each may be independently bonded to an adjacent aromatic ring to form a substituted or unsubstituted, saturated or unsaturated, five- or six-membered ring structure. Examples of the five- or six-membered ring structure include a cycloalkane having 4 to 12 carbon atoms, such as cyclopentane, cyclohexane, adamantane, and norbornane; a cycloalkene having 4 to 12 carbon atoms, such as cyclopentene and cyclohexene; a cycloalkadiene having 6 to 12 carbon atoms, such as cyclopentadiene and cyclohexadiene; and an aromatic ring having 6 to 50 carbon atoms, such as benzene, naphthalene, phenanthrene, anthracene, pyrene, chrysene, and acenaphthylene.

In formula (1) to (8) and (17) to (26), a and b each independently represent an integer of 0 to 4, and c and d each independently represent an integer of 0 to 5.

In formula (1) to (8) and (17) to (26), Ar¹ and Ar² each independently represent a substituted or unsubstituted aryl group having 6 to 50 ring carbon atoms or a substituted or unsubstituted heteroaryl group having 5 to 50 ring atoms. Example of the aryl group is selected from those described above with respect to R¹ to R¹⁴. Example of the heteroaryl group is selected from those described above with respect to R¹ to R¹⁴ and R¹⁷ to R¹⁸.

The aryl group having 6 to 50 ring carbon atoms for Ar¹ and Ar² is preferably an aryl group having 6 to 31 ring carbon atoms and more preferably an aryl group having 6 to 20 ring carbon atoms. When the aryl group is a fused ring group, the aryl group has preferably three or less fused rings, for example, as in a naphthyl group. The heteroaryl group having 5 to 50 ring atoms for Ar¹ and Ar² is preferably a heteroaryl group having 5 to 30 ring atoms and more preferably a heteroaryl group having 5 to 20 ring atoms. Preferred examples of the heteroaryl group having 5 to 50 ring atoms include a furyl group, a thienyl group, a benzofuranyl group, a benzothienyl group, a dibenzofuranyl group, and a dibenzothienyl group.

Preferred examples of Ar¹ and Ar² in formulae (1) to (8) and (17) to (26) include the following groups.

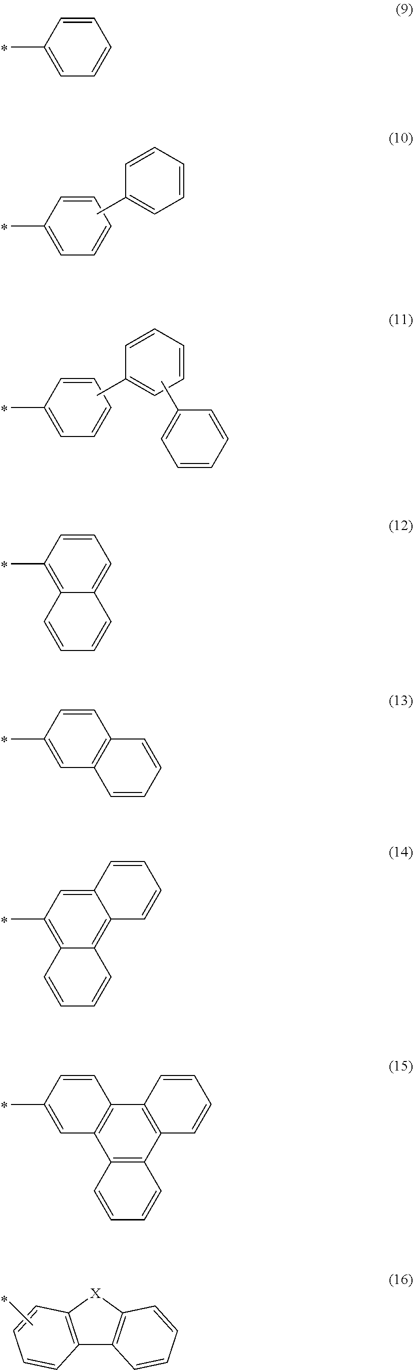

In formula 16, X represents —O—, —S—, or —CR¹⁵R¹⁶—, wherein R¹⁵ and R¹⁶ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group.

When c=0 or d=0 in formula (2), the aryl group having 6 to 50 ring carbon atoms for Ar¹ and Ar² is preferably an aryl group having 10 to 50 ring carbon atoms and more preferably an aryl group having 10 to 30 ring carbon atoms, because the glass transition temperature is maintained high. Examples of the aryl group having 10 to 50 ring carbon atoms include those represented by formulae (10) to (16) and more preferably those represented by formulae (11) to (16).

Examples of the aromatic amine derivative of the invention are shown below, although not limited thereto.

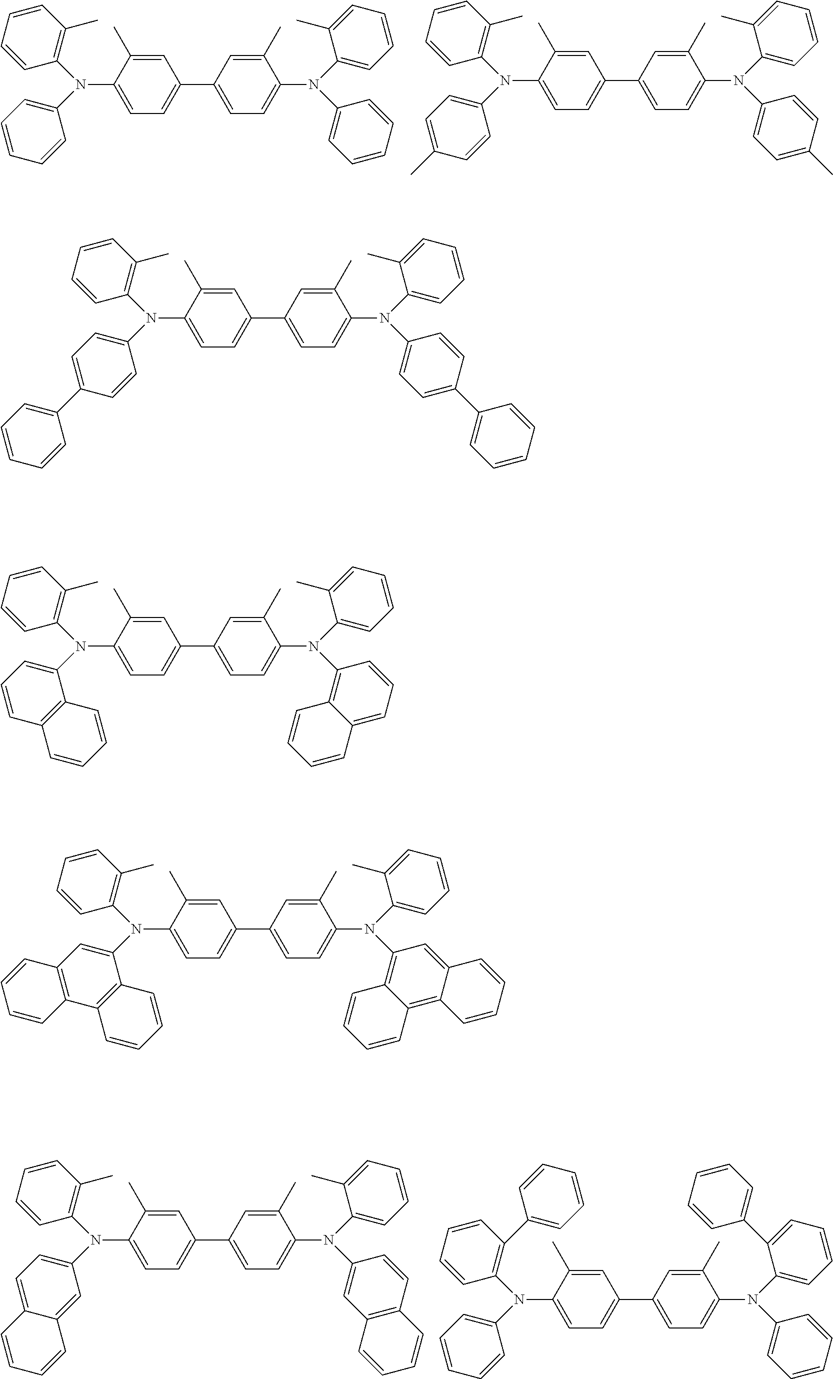

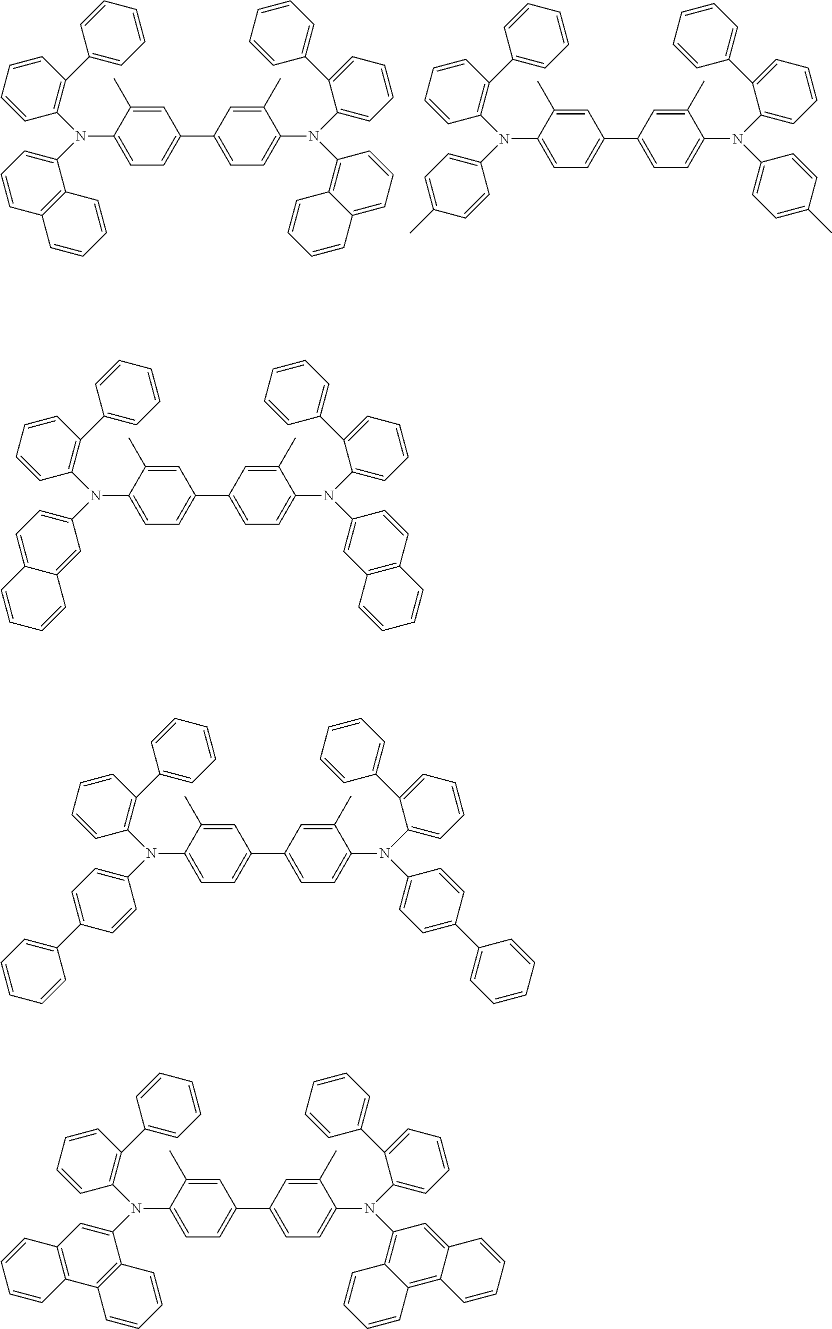

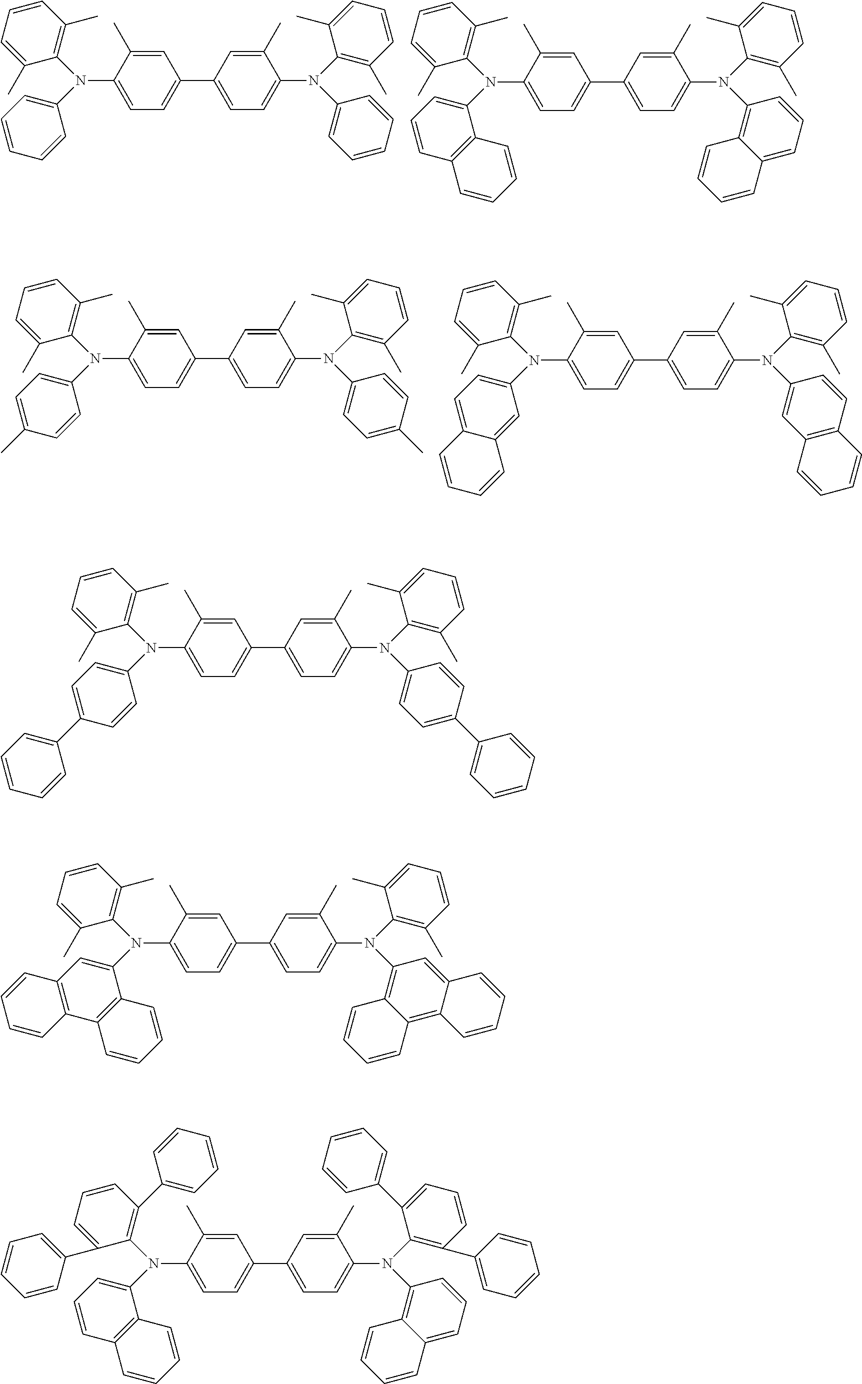

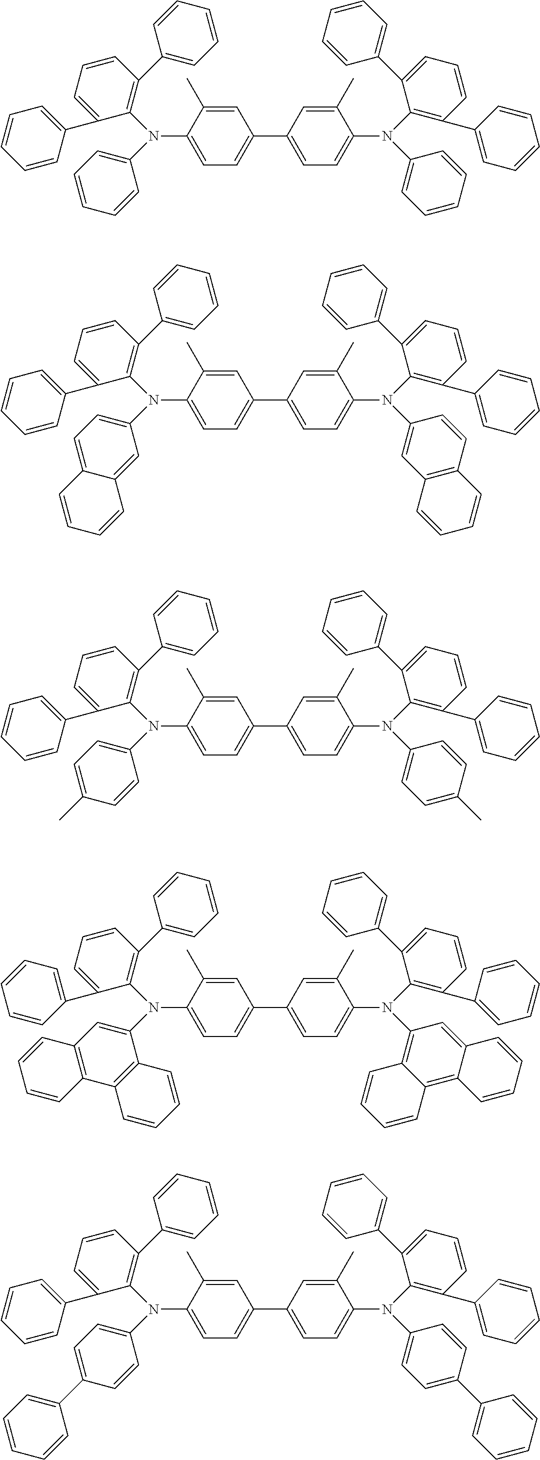

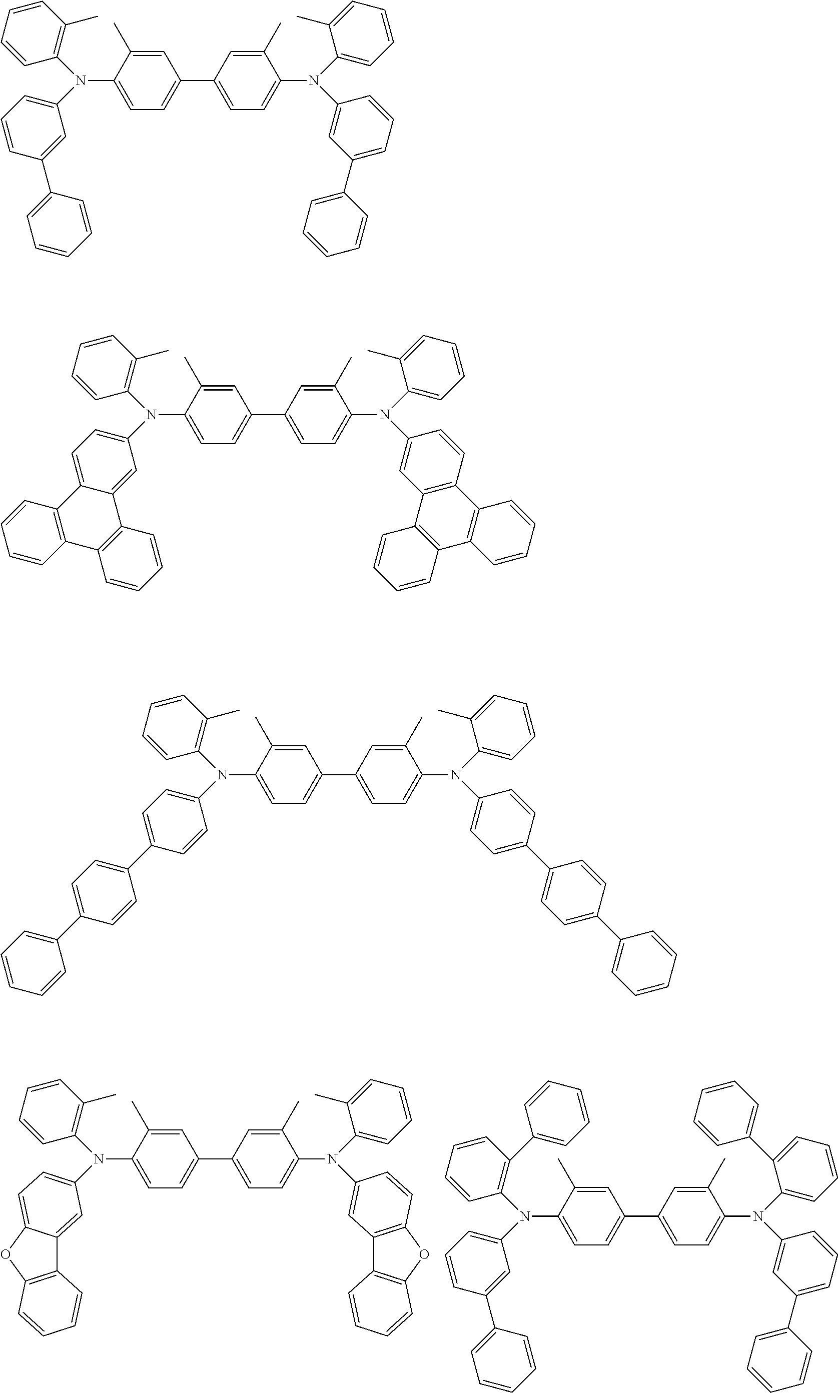

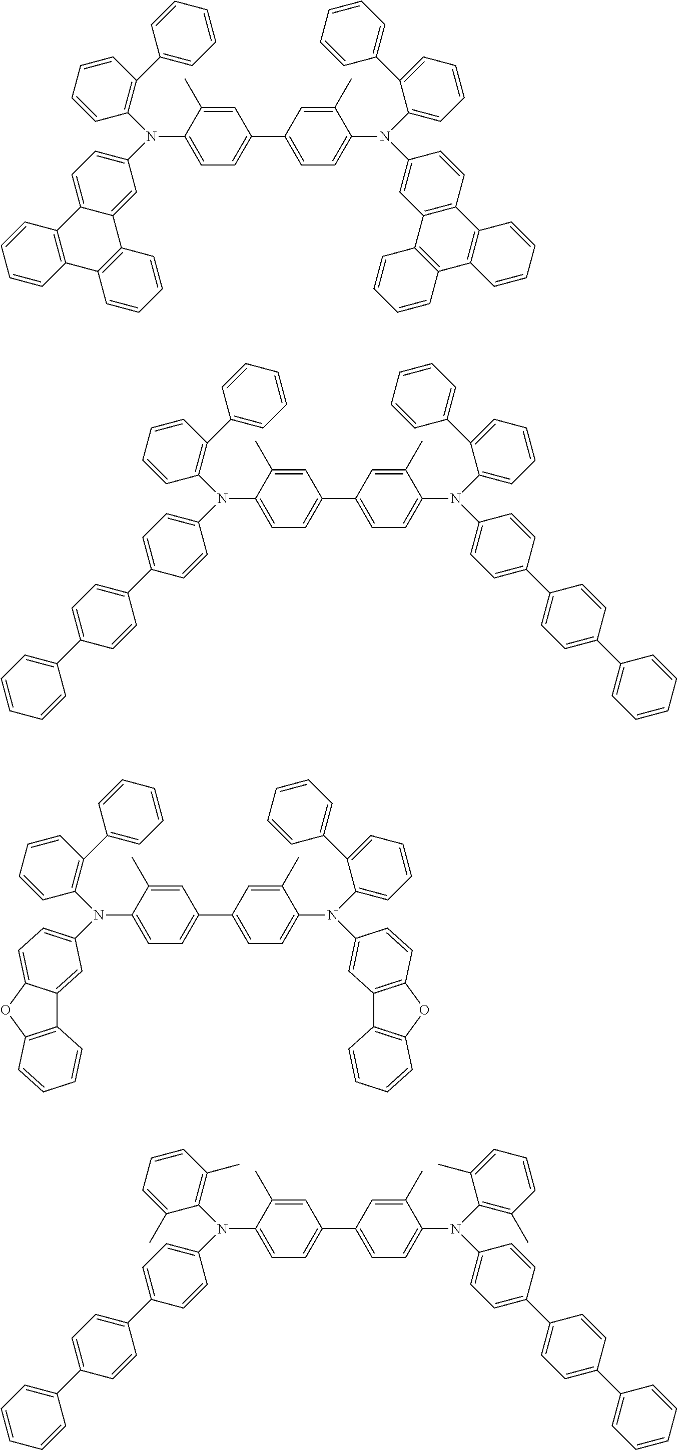 [SMILES: Cc1cc(-c2ccc(N(c3ccc4c5ccccc5c5ccccc5c4c3)c3ccccc3-c3ccccc3)c(C)c2)ccc1N(c1ccc2c3ccccc3c3ccccc3c2c1)c1ccccc1-c1ccccc1 ; Cc1cc(-c2ccc(N(c3ccc(-c4ccc(-c5ccccc5)cc4)cc3)c3ccccc3-c3ccccc3)c(C)c2)ccc1N(c1ccc(-c2ccc(-c3ccccc3)cc2)cc1)c1ccccc1-c1ccccc1 ; Cc1cc(-c2ccc(N(c3ccc4oc5ccccc5c4c3)c3ccccc3-c3ccccc3)c(C)c2)ccc1N(c1ccc2oc3ccccc3c2c1)c1ccccc1-c1ccccc1 ; Cc1cc(-c2ccc(N(c3ccc(-c4ccc(-c5ccccc5)cc4)cc3)c3c(C)cccc3C)c(C)c2)ccc1N(c1ccc(-c2ccc(-c3ccccc3)cc2)cc1)c1c(C)cccc1C]

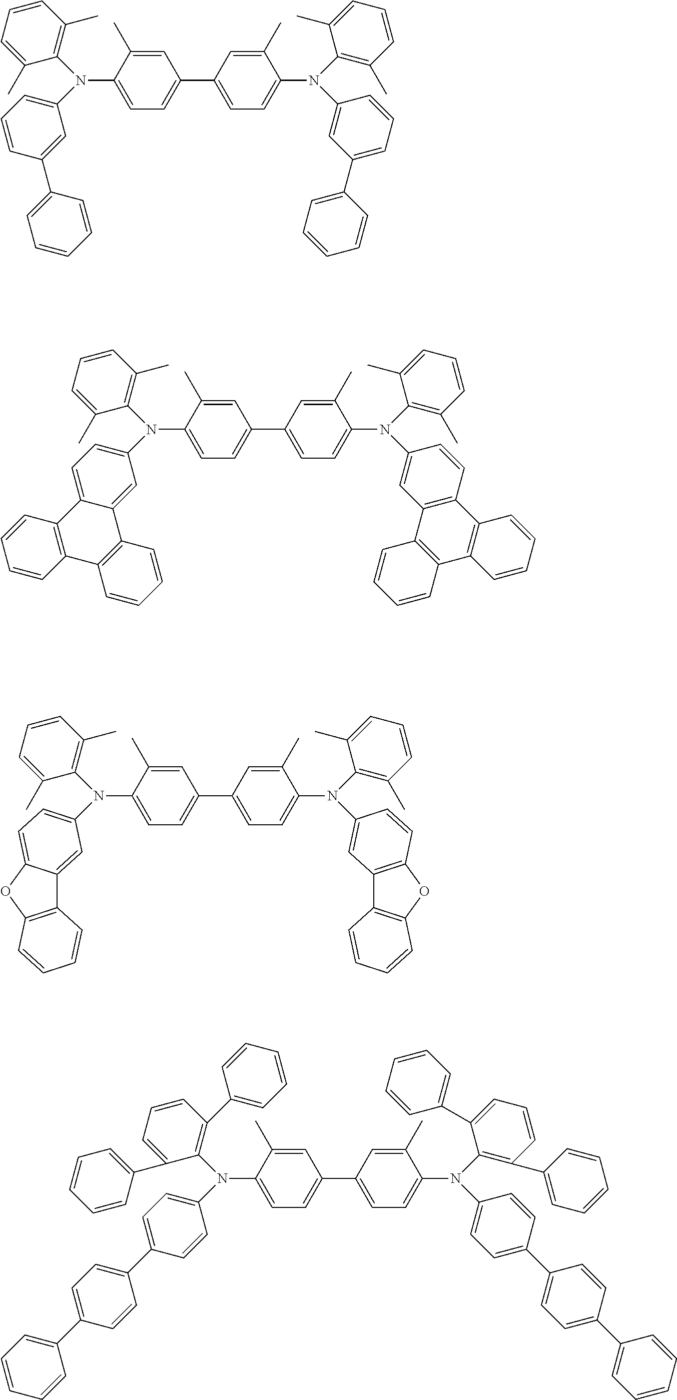 [SMILES: Cc1cc(-c2ccc(N(c3cccc(-c4ccccc4)c3)c3c(C)cccc3C)c(C)c2)ccc1N(c1cccc(-c2ccccc2)c1)c1c(C)cccc1C ; Cc1cc(-c2ccc(N(c3ccc4c5ccccc5c5ccccc5c4c3)c3c(C)cccc3C)c(C)c2)ccc1N(c1ccc2c3ccccc3c3ccccc3c2c1)c1c(C)cccc1C ; Cc1cc(-c2ccc(N(c3ccc4oc5ccccc5c4c3)c3c(C)cccc3C)c(C)c2)ccc1N(c1ccc2oc3ccccc3c2c1)c1c(C)cccc1C ; Cc1cc(-c2ccc(N(c3ccc(-c4ccc(-c5ccccc5)cc4)cc3)c3c(-c4ccccc4)cccc3-c3ccccc3)c(C)c2)ccc1N(c1ccc(-c2ccc(-c3ccccc3)cc2)cc1)c1c(-c2ccccc2)cccc1-c1ccccc1]

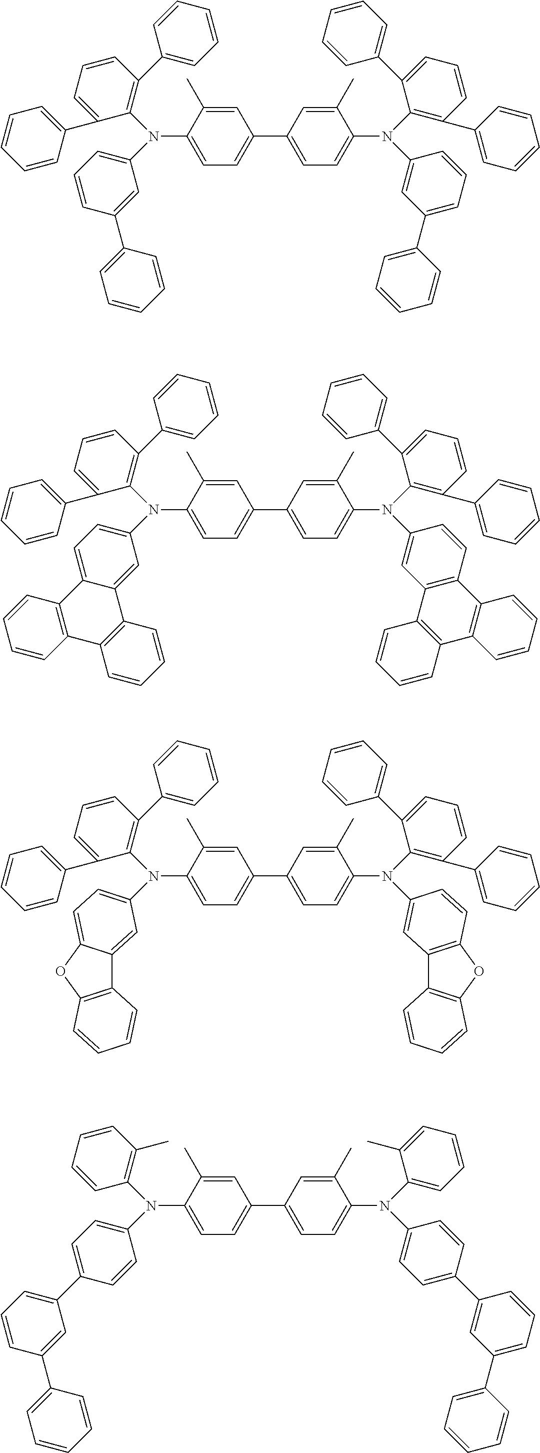

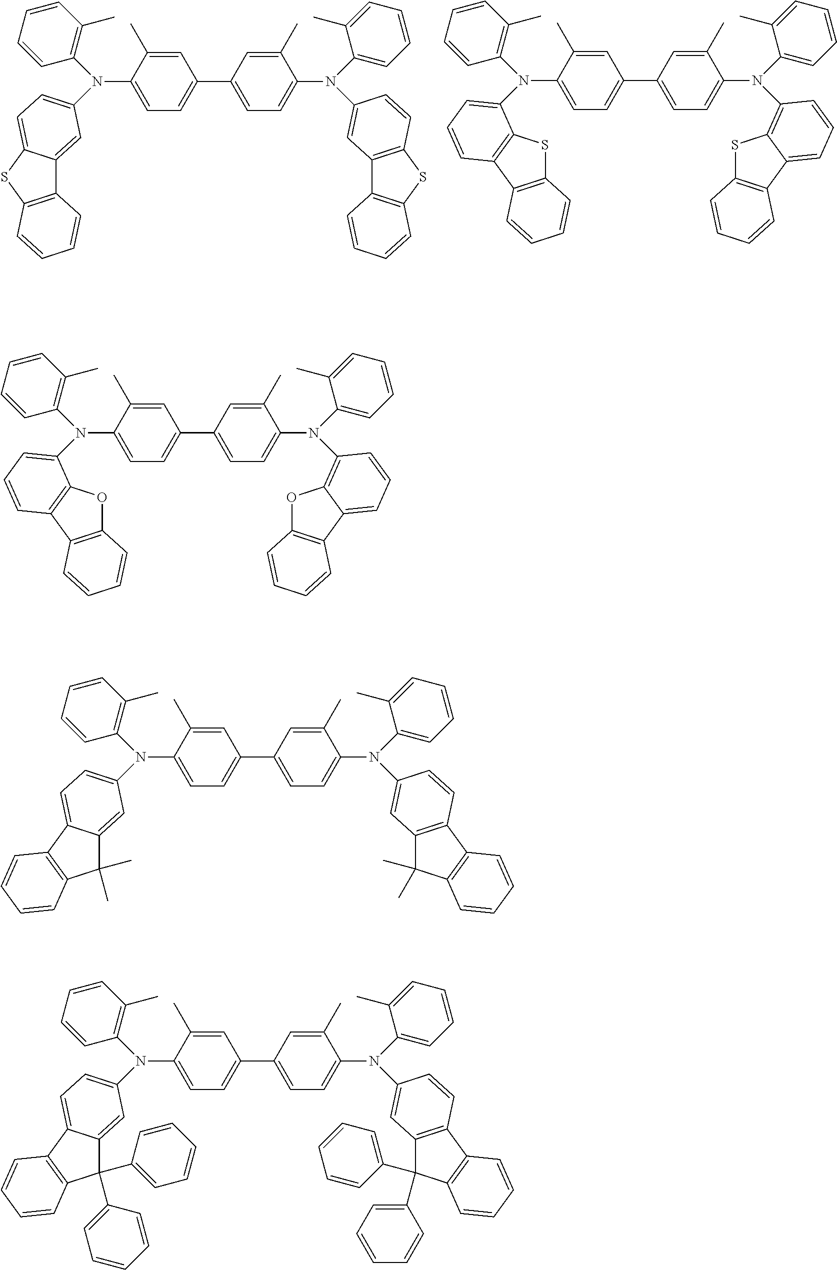

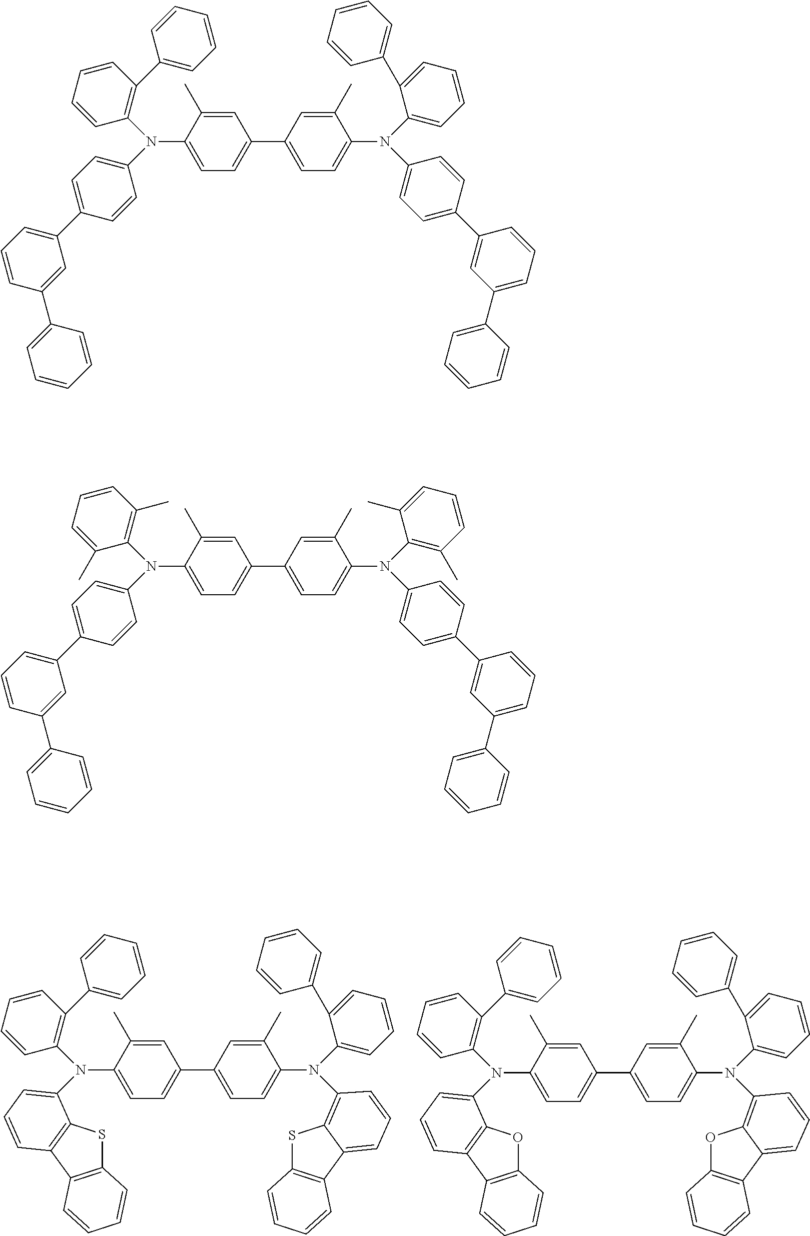

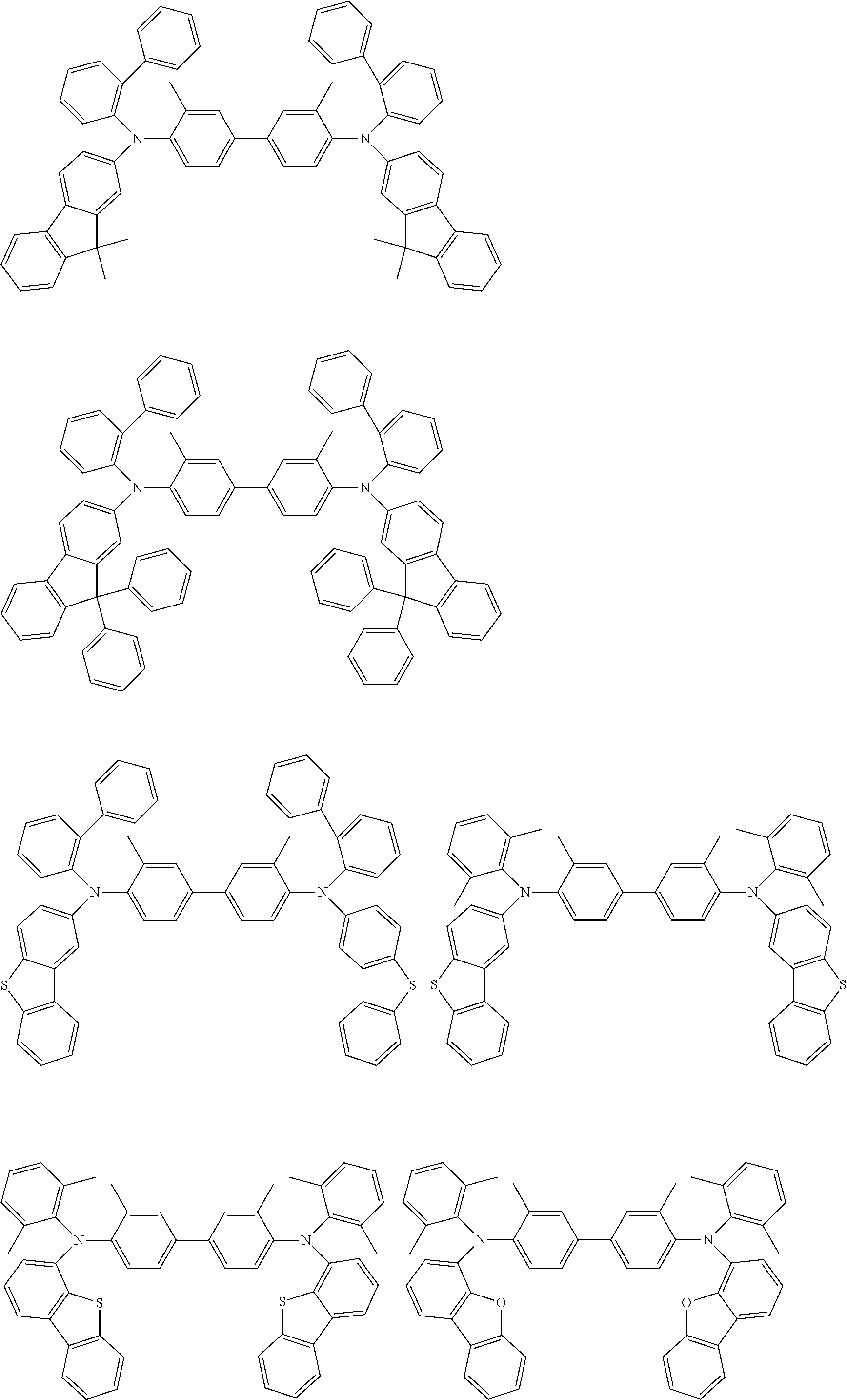

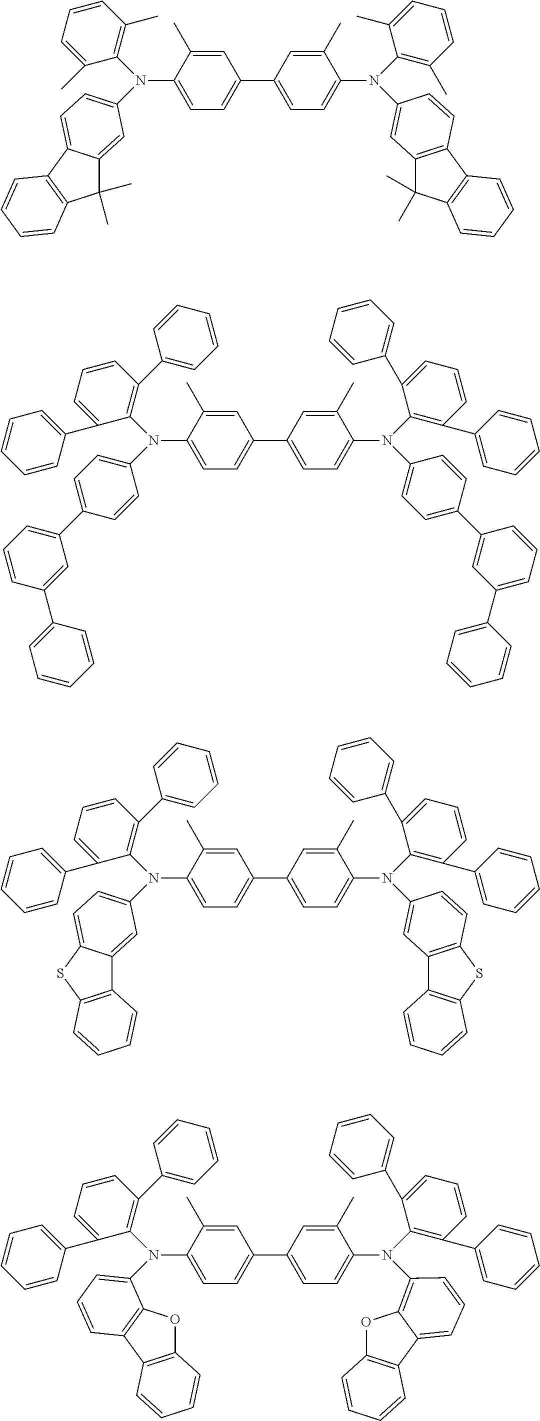

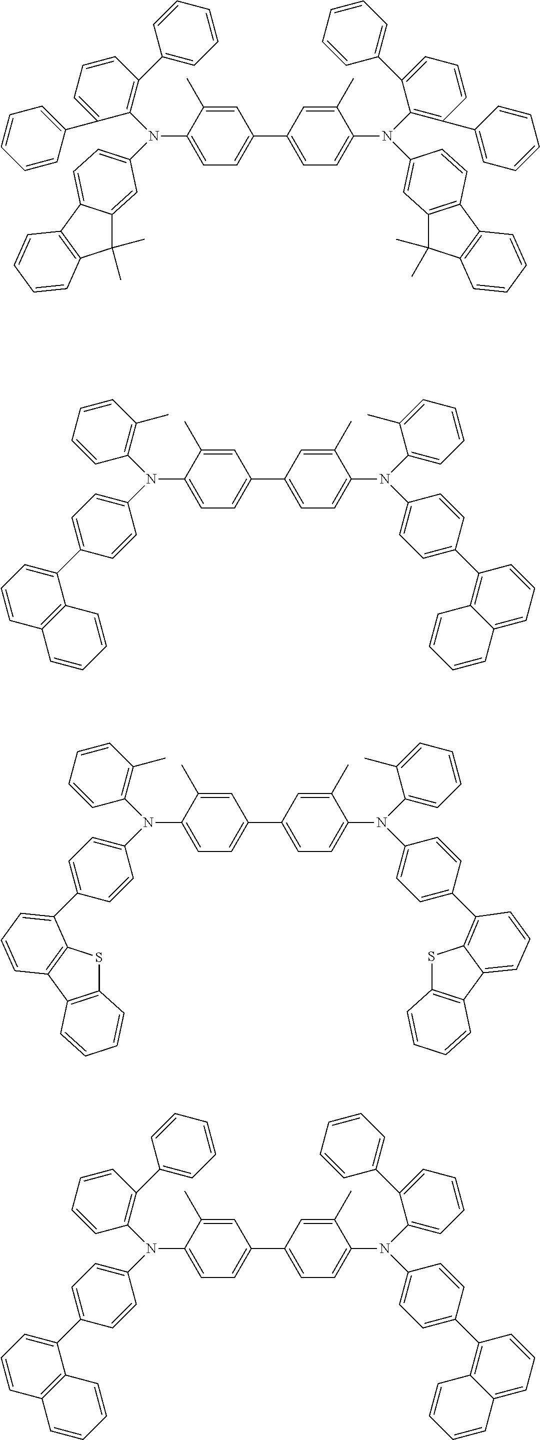 [SMILES: Cc1cc(-c2ccc(N(c3ccc4c(c3)C(C)(C)c3ccccc3-4)c3c(-c4ccccc4)cccc3-c3ccccc3)c(C)c2)ccc1N(c1ccc2c(c1)C(C)(C)c1ccccc1-2)c1c(-c2ccccc2)cccc1-c1ccccc1 ; Cc1ccccc1N(c1ccc(-c2cccc3ccccc23)cc1)c1ccc(-c2ccc(N(c3ccc(-c4cccc5ccccc45)cc3)c3ccccc3C)c(C)c2)cc1C ; Cc1ccccc1N(c1ccc(-c2cccc3c2sc2ccccc23)cc1)c1ccc(-c2ccc(N(c3ccc(-c4cccc5c4sc4ccccc45)cc3)c3ccccc3C)c(C)c2)cc1C ; Cc1cc(-c2ccc(N(c3ccc(-c4cccc5ccccc45)cc3)c3ccccc3-c3ccccc3)c(C)c2)ccc1N(c1ccc(-c2cccc3ccccc23)cc1)c1ccccc1-c1ccccc1]

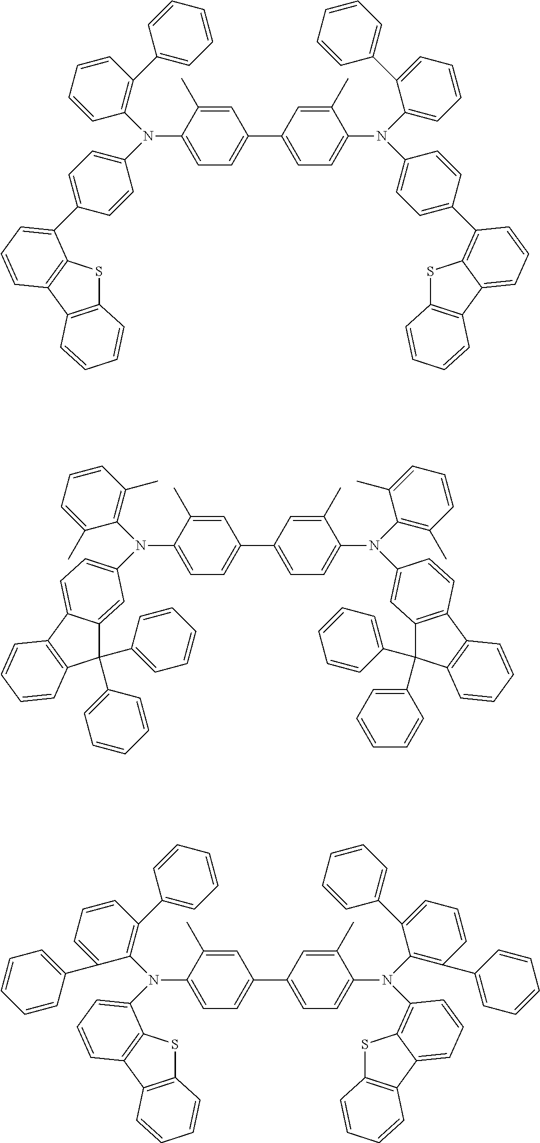 [SMILES: Cc1cc(-c2ccc(N(c3ccc(-c4cccc5c4sc4ccccc45)cc3)c3ccccc3-c3ccccc3)c(C)c2)ccc1N(c1ccc(-c2cccc3c2sc2ccccc23)cc1)c1ccccc1-c1ccccc1 ; Cc1cc(-c2ccc(N(c3ccc4c(c3)C(c3ccccc3)(c3ccccc3)c3ccccc3-4)c3c(C)cccc3C)c(C)c2)ccc1N(c1ccc2c(c1)C(c1ccccc1)(c1ccccc1)c1ccccc1-2)c1c(C)cccc1C ; Cc1cc(-c2ccc(N(c3c(-c4ccccc4)cccc3-c3ccccc3)c3cccc4c3sc3ccccc34)c(C)c2)ccc1N(c1c(-c2ccccc2)cccc1-c1ccccc1)c1cccc2c1sc1ccccc12]

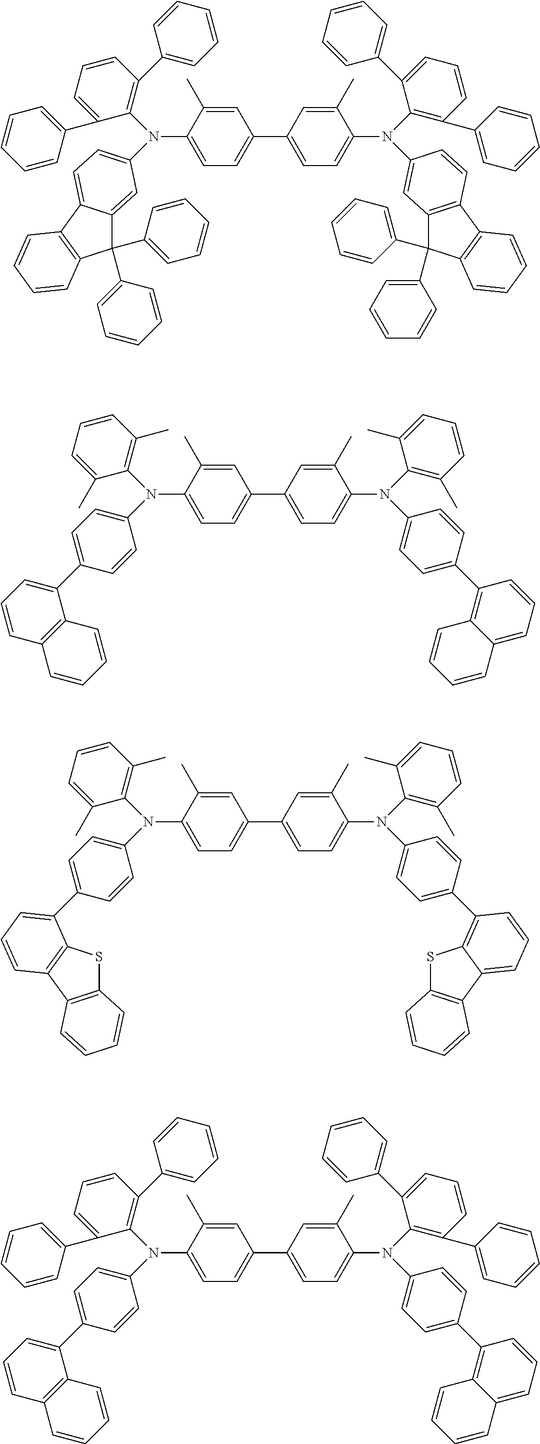

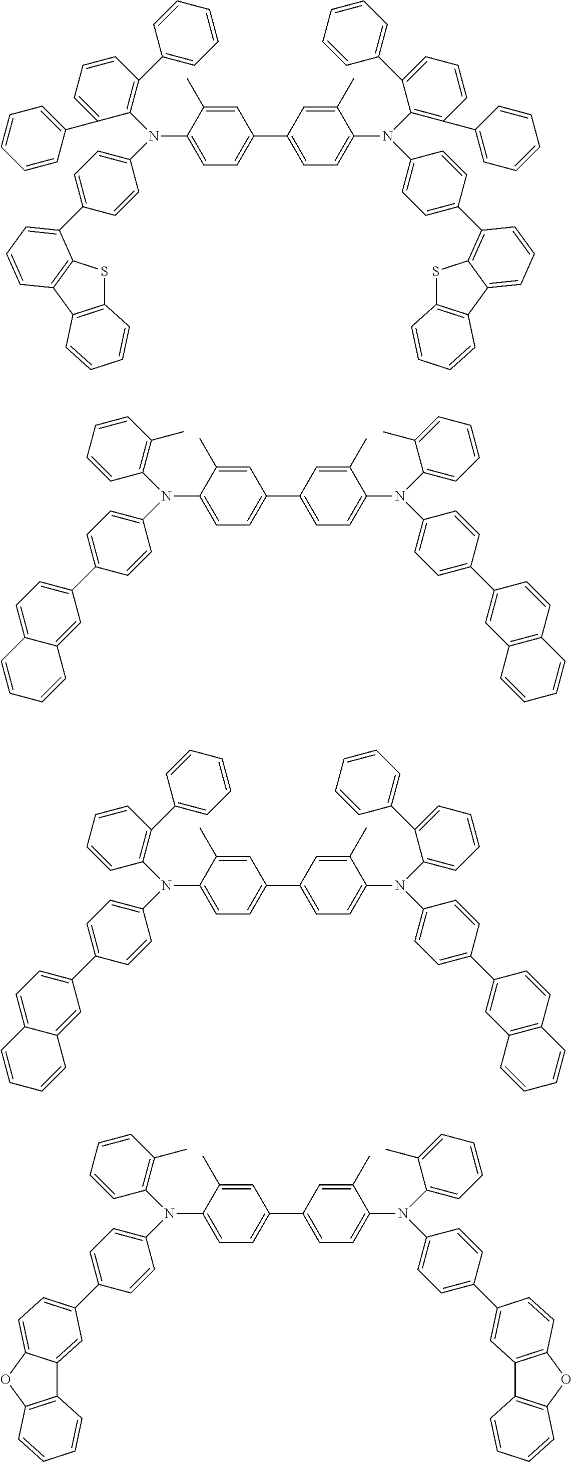 [SMILES: Cc1cc(-c2ccc(N(c3ccc(-c4cccc5c4sc4ccccc45)cc3)c3c(-c4ccccc4)cccc3-c3ccccc3)c(C)c2)ccc1N(c1ccc(-c2cccc3c2sc2ccccc23)cc1)c1c(-c2ccccc2)cccc1-c1ccccc1 ; Cc1ccccc1N(c1ccc(-c2ccc3ccccc3c2)cc1)c1ccc(-c2ccc(N(c3ccc(-c4ccc5ccccc5c4)cc3)c3ccccc3C)c(C)c2)cc1C ; Cc1cc(-c2ccc(N(c3ccc(-c4ccc5ccccc5c4)cc3)c3ccccc3-c3ccccc3)c(C)c2)ccc1N(c1ccc(-c2ccc3ccccc3c2)cc1)c1ccccc1-c1ccccc1 ; Cc1ccccc1N(c1ccc(-c2ccc3oc4ccccc4c3c2)cc1)c1ccc(-c2ccc(N(c3ccc(-c4ccc5oc6ccccc6c5c4)cc3)c3ccccc3C)c(C)c2)cc1C]

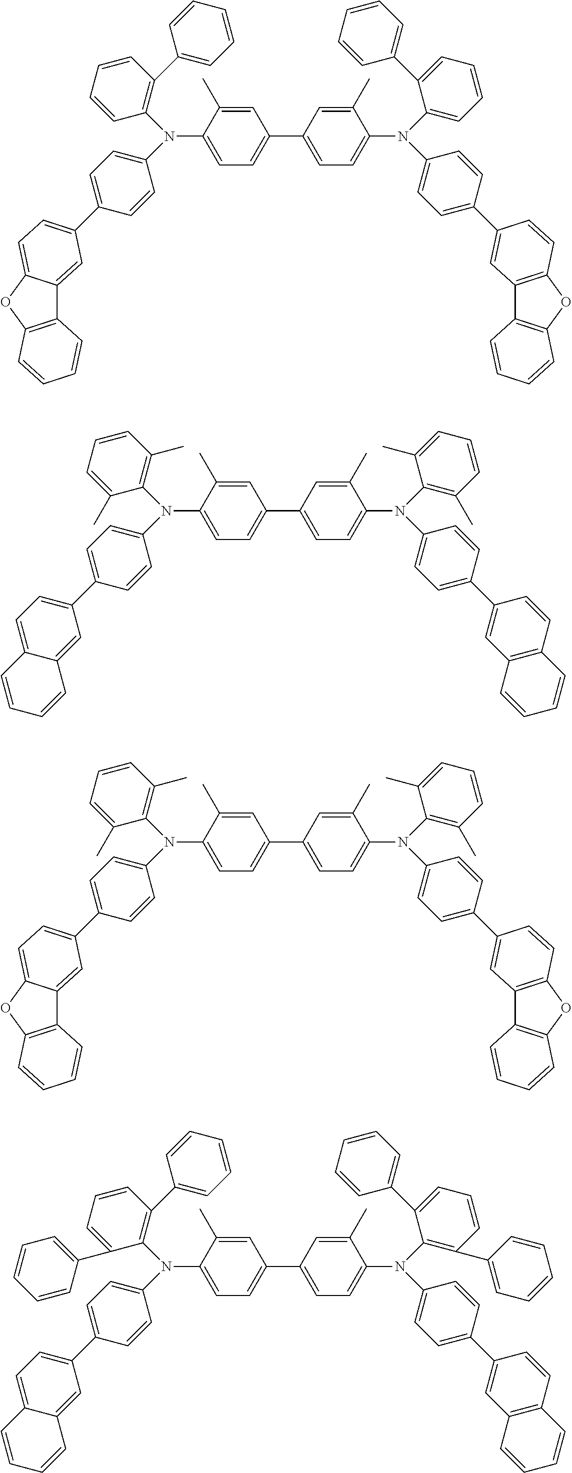 [SMILES: Cc1cc(-c2ccc(N(c3ccc(-c4ccc5oc6ccccc6c5c4)cc3)c3ccccc3-c3ccccc3)c(C)c2)ccc1N(c1ccc(-c2ccc3oc4ccccc4c3c2)cc1)c1ccccc1-c1ccccc1 ; Cc1cc(-c2ccc(N(c3ccc(-c4ccc5ccccc5c4)cc3)c3c(C)cccc3C)c(C)c2)ccc1N(c1ccc(-c2ccc3ccccc3c2)cc1)c1c(C)cccc1C ; Cc1cc(-c2ccc(N(c3ccc(-c4ccc5oc6ccccc6c5c4)cc3)c3c(C)cccc3C)c(C)c2)ccc1N(c1ccc(-c2ccc3oc4ccccc4c3c2)cc1)c1c(C)cccc1C ; Cc1cc(-c2ccc(N(c3ccc(-c4ccc5ccccc5c4)cc3)c3c(-c4ccccc4)cccc3-c3ccccc3)c(C)c2)ccc1N(c1ccc(-c2ccc3ccccc3c2)cc1)c1c(-c2ccccc2)cccc1-c1ccccc1]

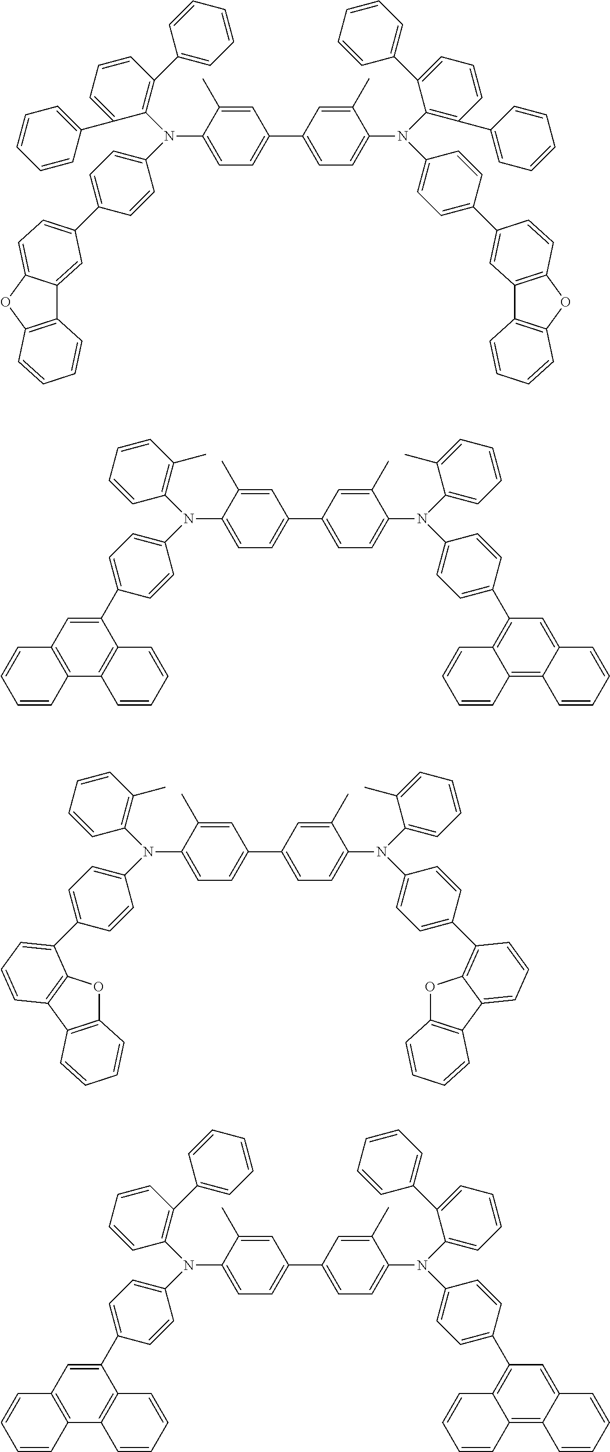

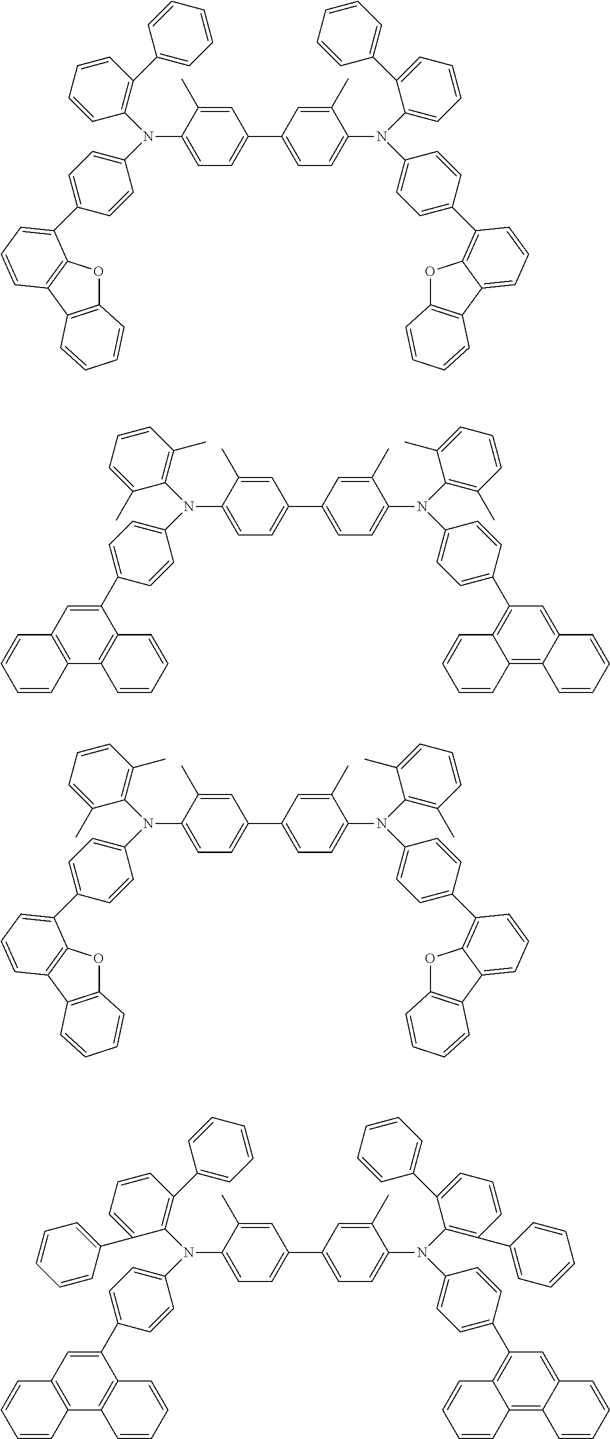

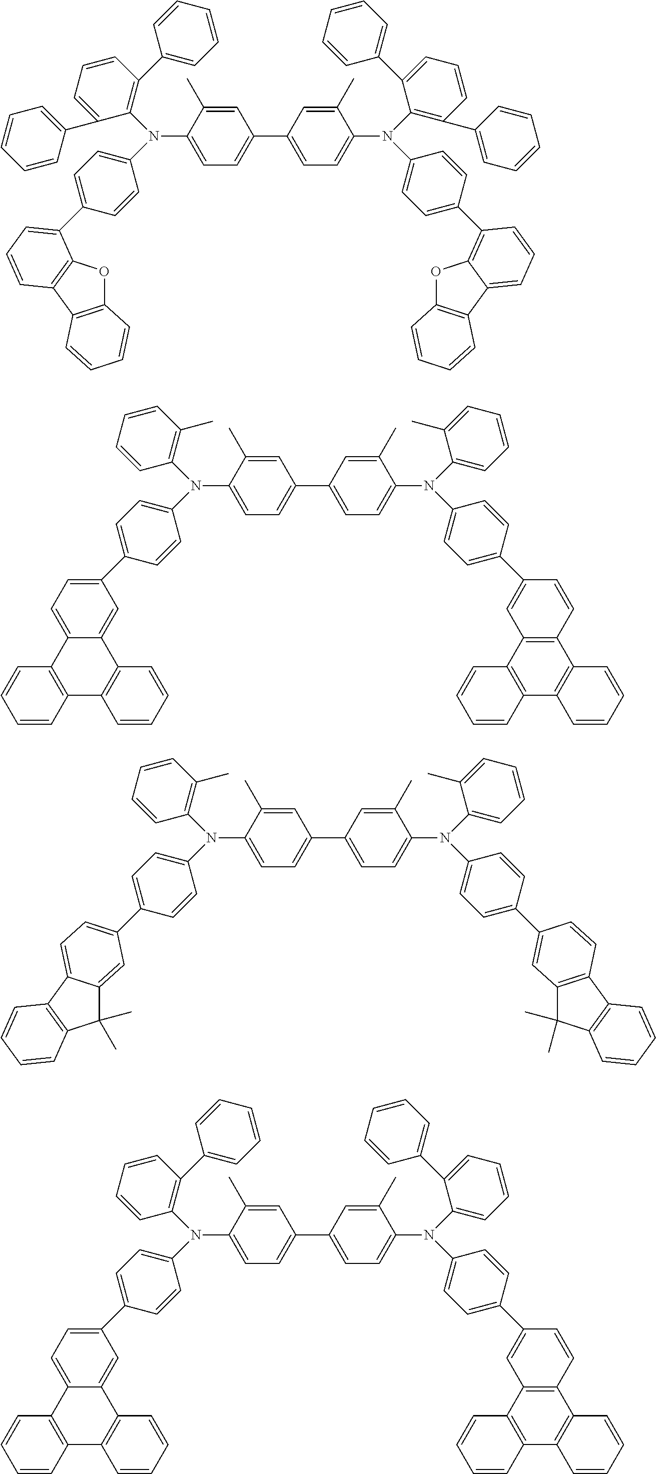

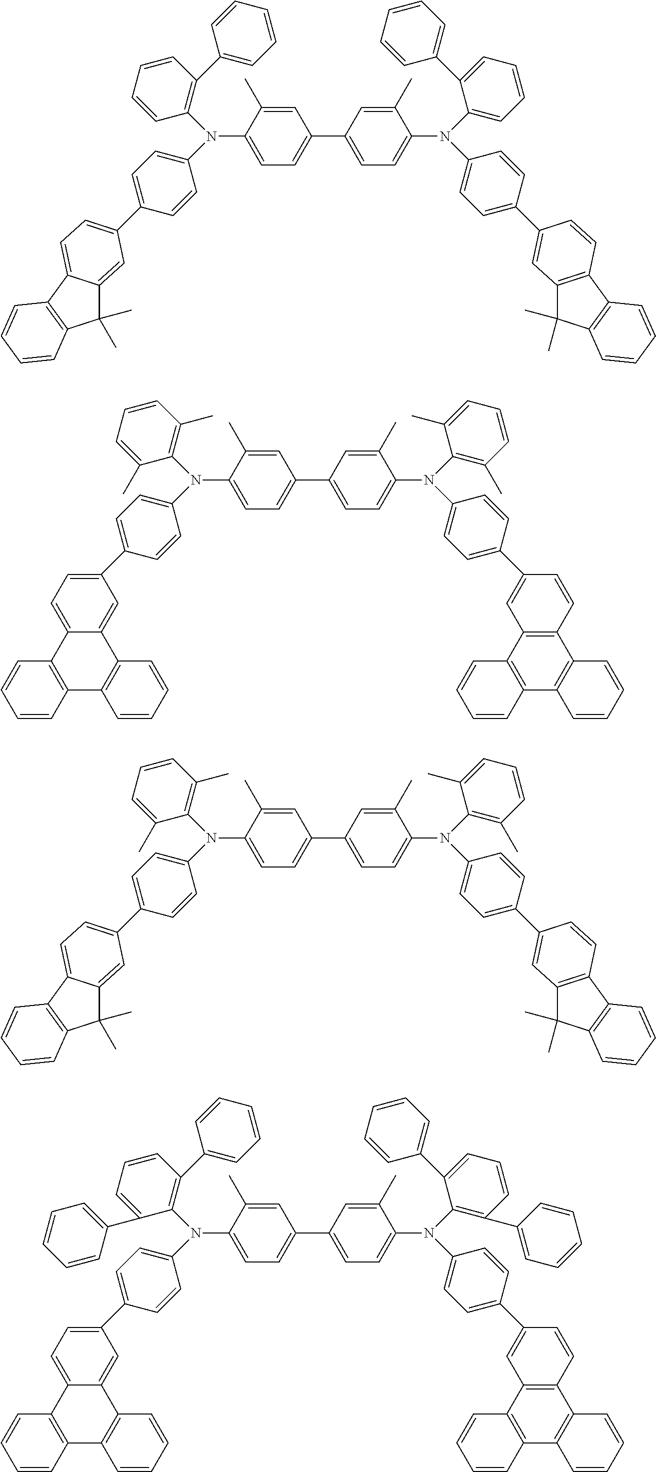

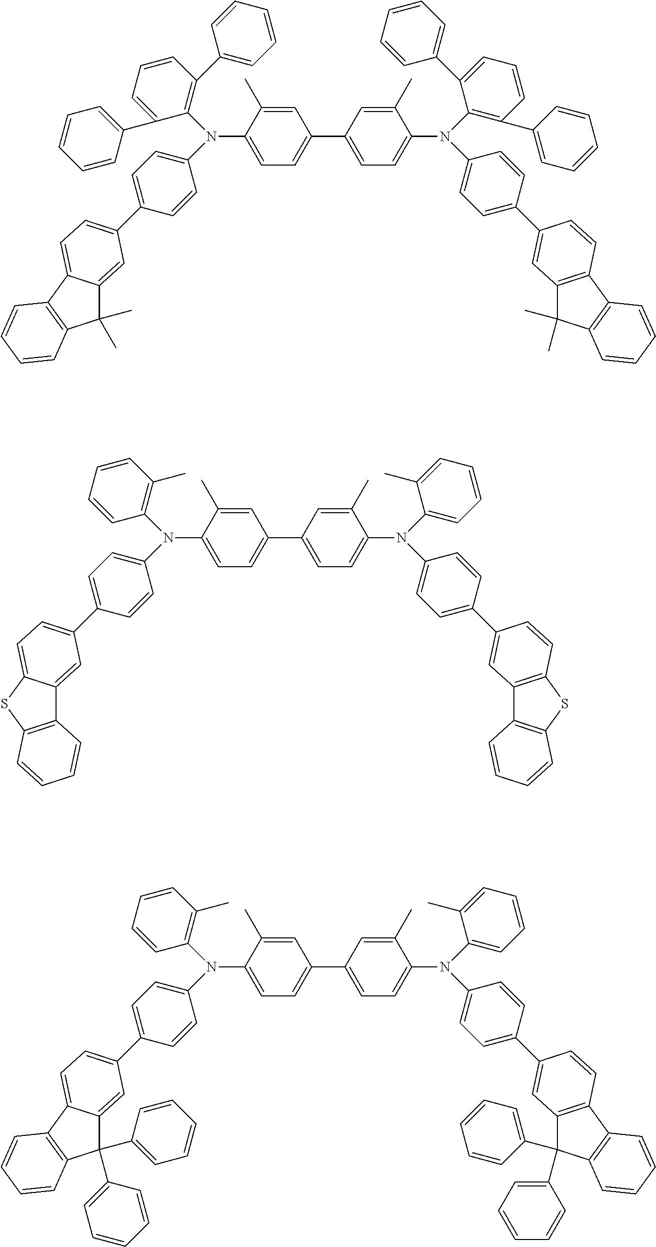 [SMILES: Cc1cc(-c2ccc(N(c3ccc(-c4ccc5c(c4)C(C)(C)c4ccccc4-5)cc3)c3c(-c4ccccc4)cccc3-c3ccccc3)c(C)c2)ccc1N(c1ccc(-c2ccc3c(c2)C(C)(C)c2ccccc2-3)cc1)c1c(-c2ccccc2)cccc1-c1ccccc1 ; Cc1ccccc1N(c1ccc(-c2ccc3sc4ccccc4c3c2)cc1)c1ccc(-c2ccc(N(c3ccc(-c4ccc5sc6ccccc6c5c4)cc3)c3ccccc3C)c(C)c2)cc1C ; Cc1ccccc1N(c1ccc(-c2ccc3c(c2)C(c2ccccc2)(c2ccccc2)c2ccccc2-3)cc1)c1ccc(-c2ccc(N(c3ccc(-c4ccc5c(c4)C(c4ccccc4)(c4ccccc4)c4ccccc4-5)cc3)c3ccccc3C)c(C)c2)cc1C]

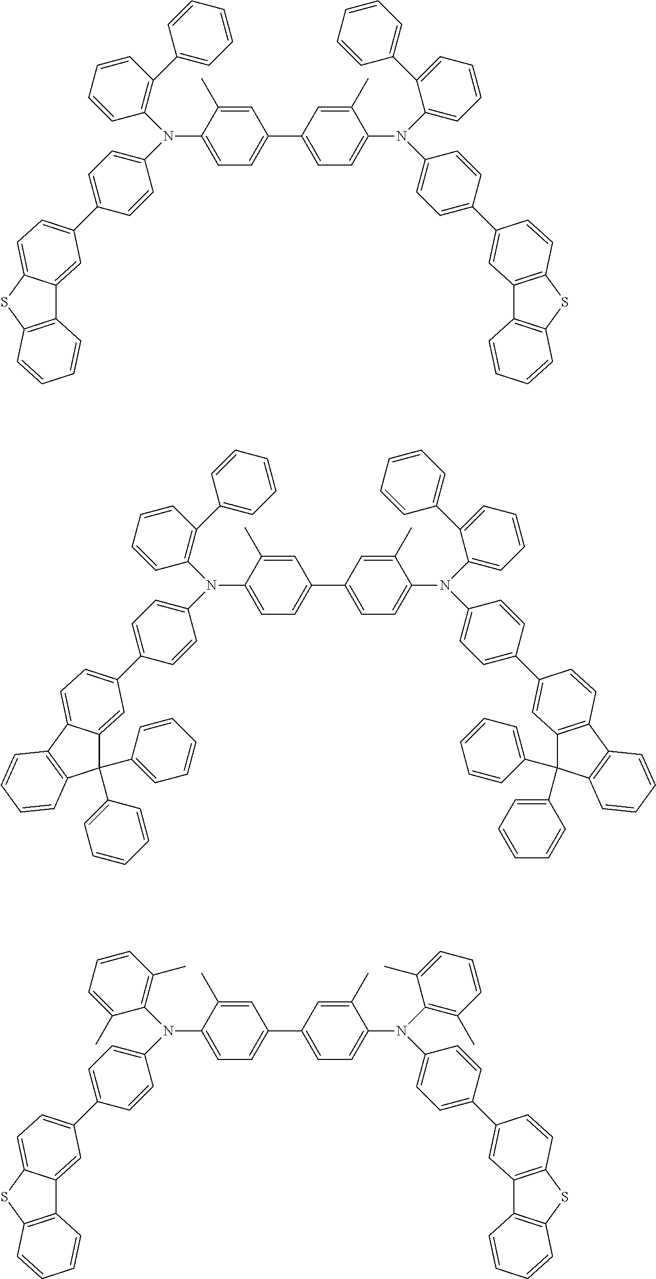 [SMILES: Cc1cc(-c2ccc(N(c3ccc(-c4ccc5sc6ccccc6c5c4)cc3)c3ccccc3-c3ccccc3)c(C)c2)ccc1N(c1ccc(-c2ccc3sc4ccccc4c3c2)cc1)c1ccccc1-c1ccccc1 ; Cc1cc(-c2ccc(N(c3ccc(-c4ccc5c(c4)C(c4ccccc4)(c4ccccc4)c4ccccc4-5)cc3)c3ccccc3-c3ccccc3)c(C)c2)ccc1N(c1ccc(-c2ccc3c(c2)C(c2ccccc2)(c2ccccc2)c2ccccc2-3)cc1)c1ccccc1-c1ccccc1 ; Cc1cc(-c2ccc(N(c3ccc(-c4ccc5sc6ccccc6c5c4)cc3)c3c(C)cccc3C)c(C)c2)ccc1N(c1ccc(-c2ccc3sc4ccccc4c3c2)cc1)c1c(C)cccc1C]

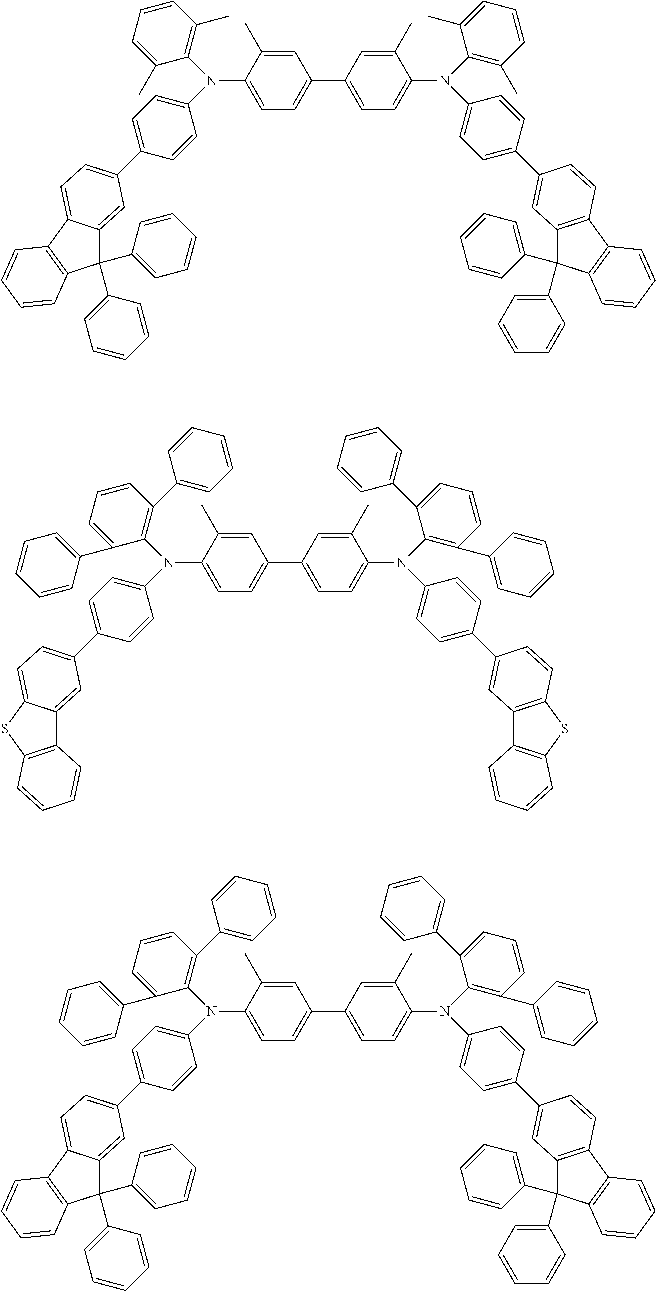

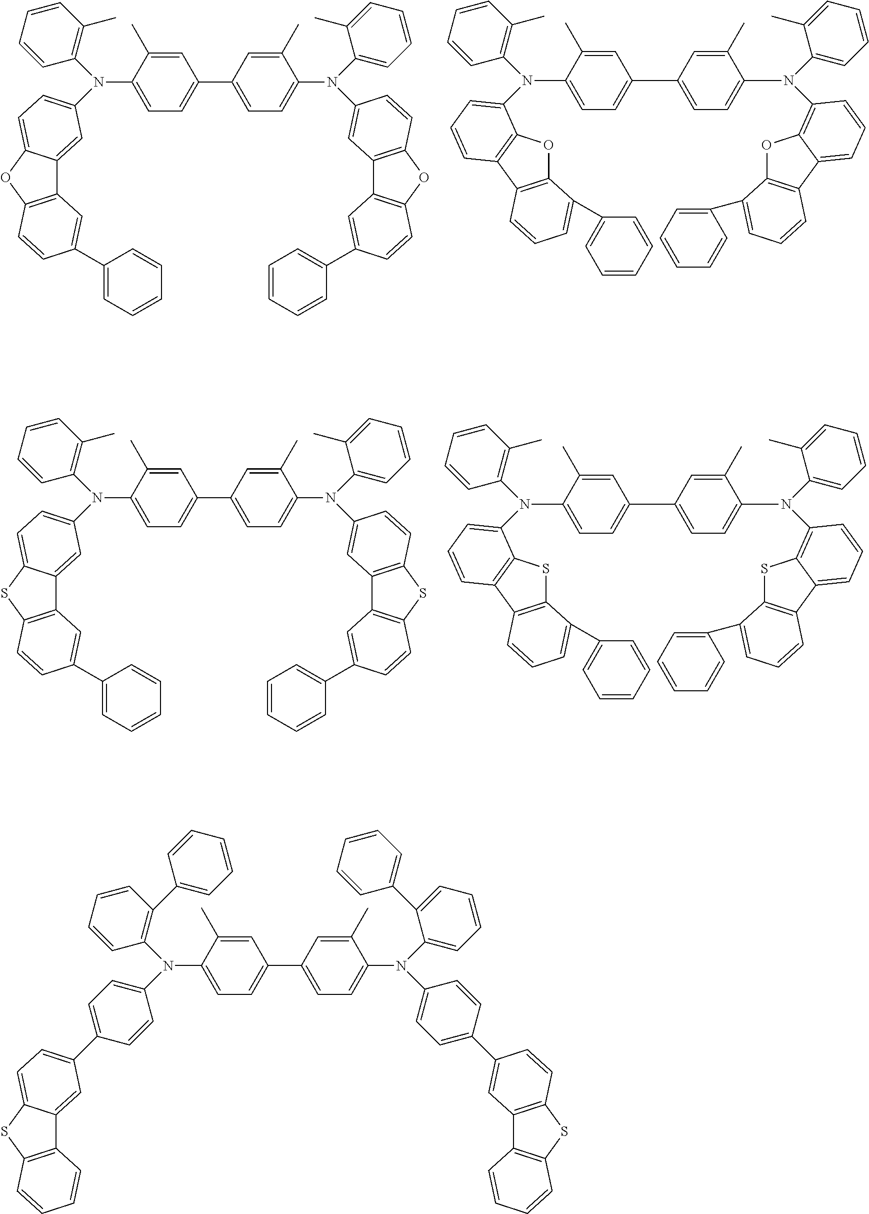

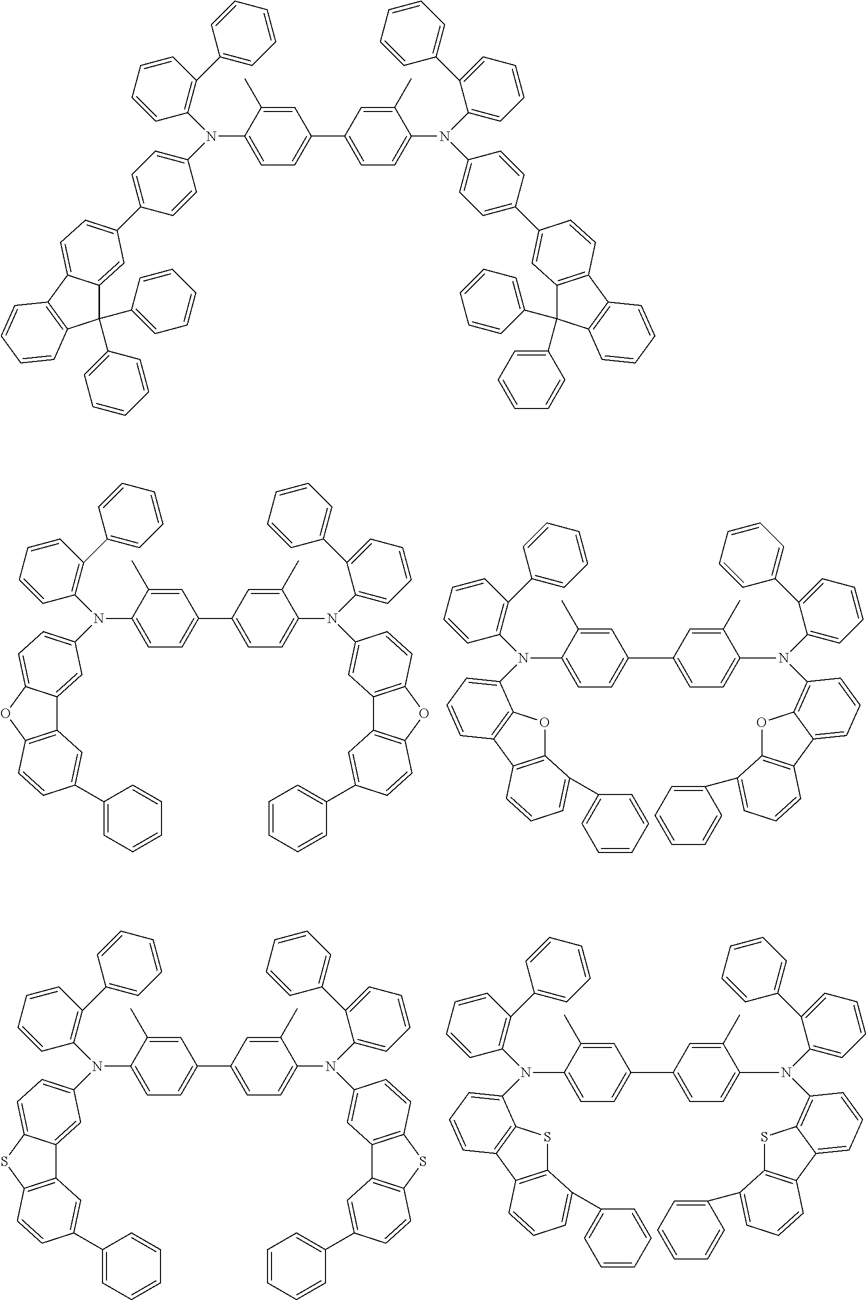

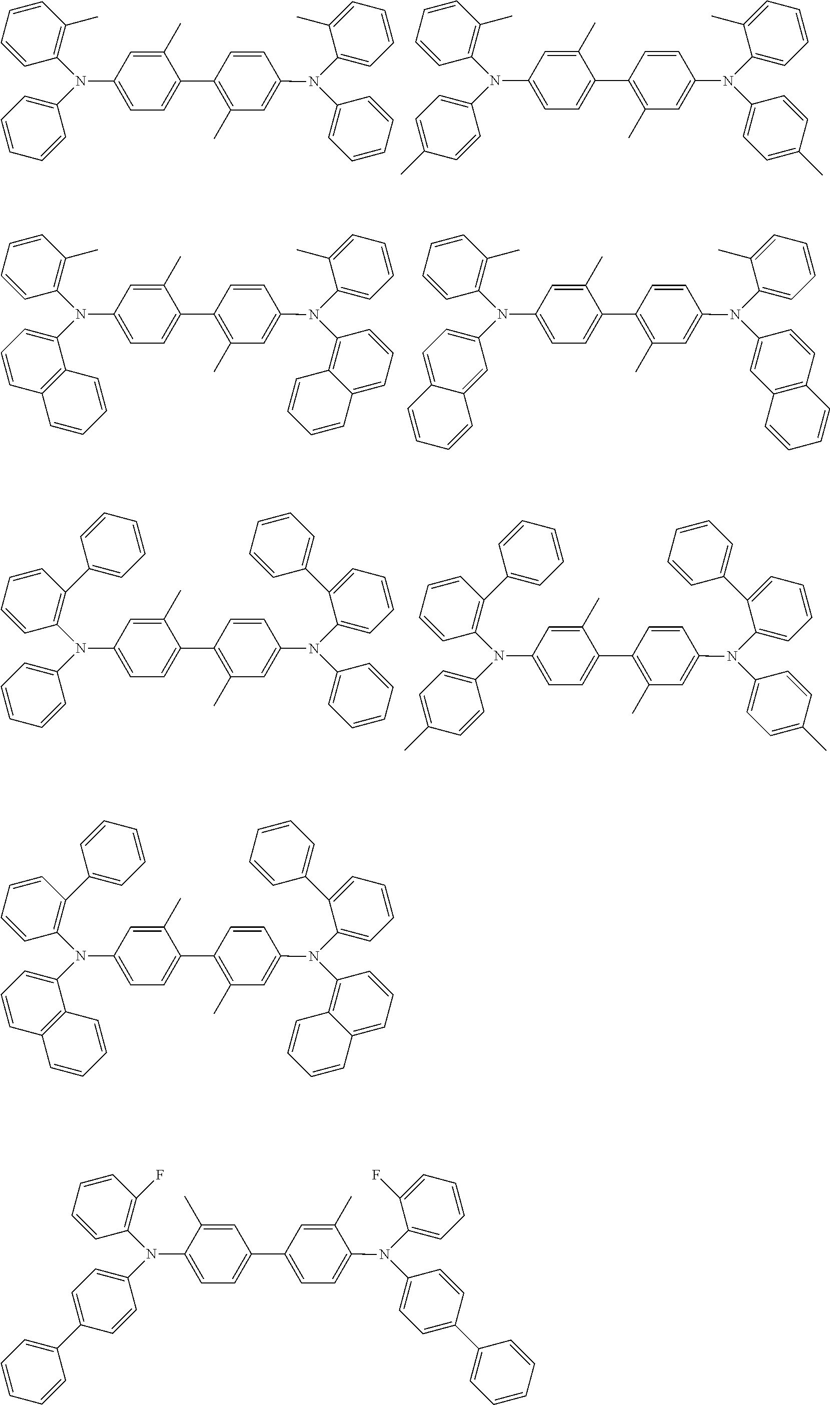

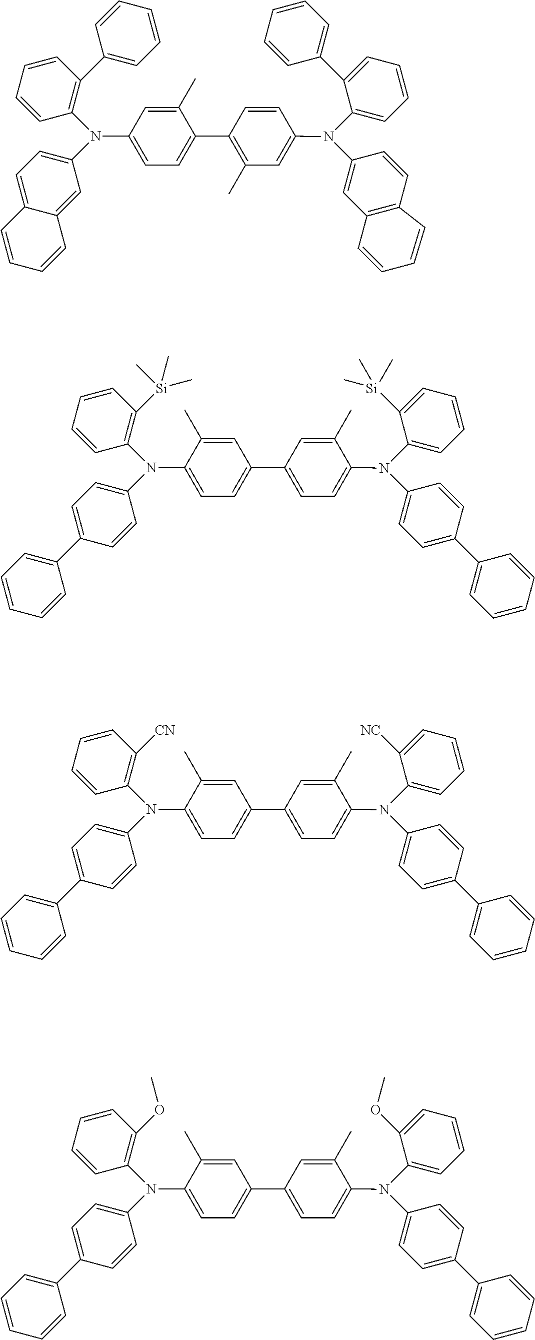 [SMILES: Cc1cc(N(c2ccc3ccccc3c2)c2ccccc2-c2ccccc2)ccc1-c1ccc(N(c2ccc3ccccc3c2)c2ccccc2-c2ccccc2)cc1C ; Cc1cc(-c2ccc(N(c3ccc(-c4ccccc4)cc3)c3ccccc3[Si](C)(C)C)c(C)c2)ccc1N(c1ccc(-c2ccccc2)cc1)c1ccccc1[Si](C)(C)C ; Cc1cc(-c2ccc(N(c3ccc(-c4ccccc4)cc3)c3ccccc3C#N)c(C)c2)ccc1N(c1ccc(-c2ccccc2)cc1)c1ccccc1C#N ; COc1ccccc1N(c1ccc(-c2ccccc2)cc1)c1ccc(-c2ccc(N(c3ccc(-c4ccccc4)cc3)c3ccccc3OC)c(C)c2)cc1C]

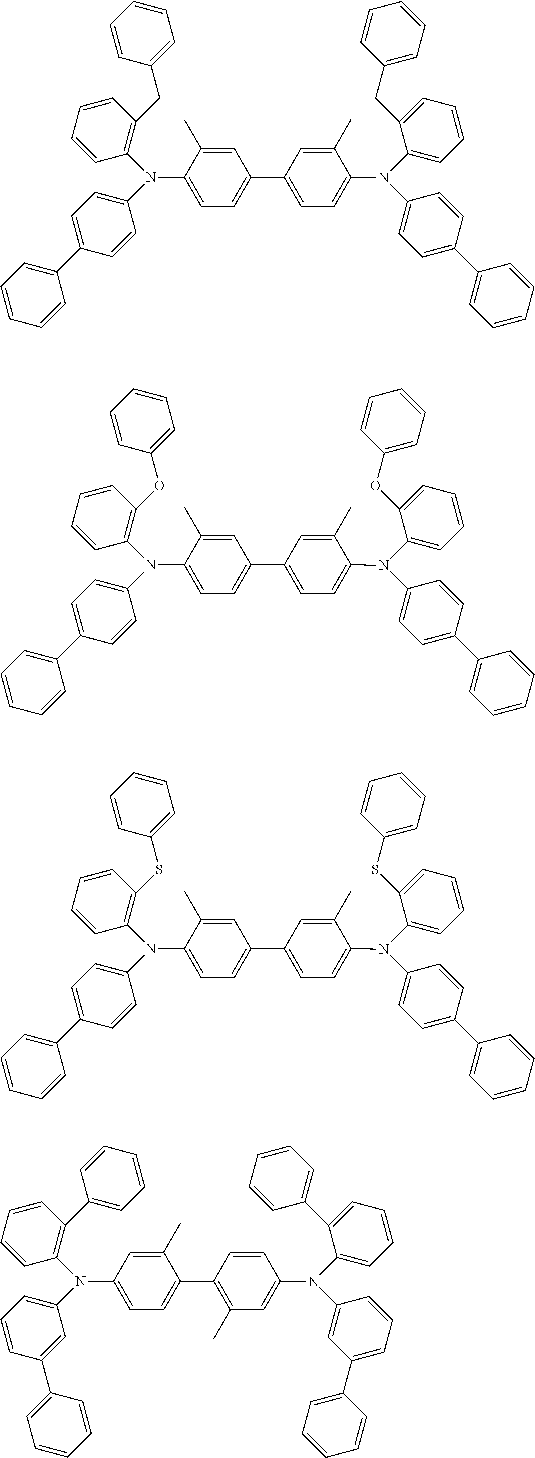 [SMILES: Cc1cc(-c2ccc(N(c3ccc(-c4ccccc4)cc3)c3ccccc3Cc3ccccc3)c(C)c2)ccc1N(c1ccc(-c2ccccc2)cc1)c1ccccc1Cc1ccccc1 ; Cc1cc(-c2ccc(N(c3ccc(-c4ccccc4)cc3)c3ccccc3Oc3ccccc3)c(C)c2)ccc1N(c1ccc(-c2ccccc2)cc1)c1ccccc1Oc1ccccc1 ; Cc1cc(-c2ccc(N(c3ccc(-c4ccccc4)cc3)c3ccccc3Sc3ccccc3)c(C)c2)ccc1N(c1ccc(-c2ccccc2)cc1)c1ccccc1Sc1ccccc1 ; Cc1cc(N(c2cccc(-c3ccccc3)c2)c2ccccc2-c2ccccc2)ccc1-c1ccc(N(c2cccc(-c3ccccc3)c2)c2ccccc2-c2ccccc2)cc1C]

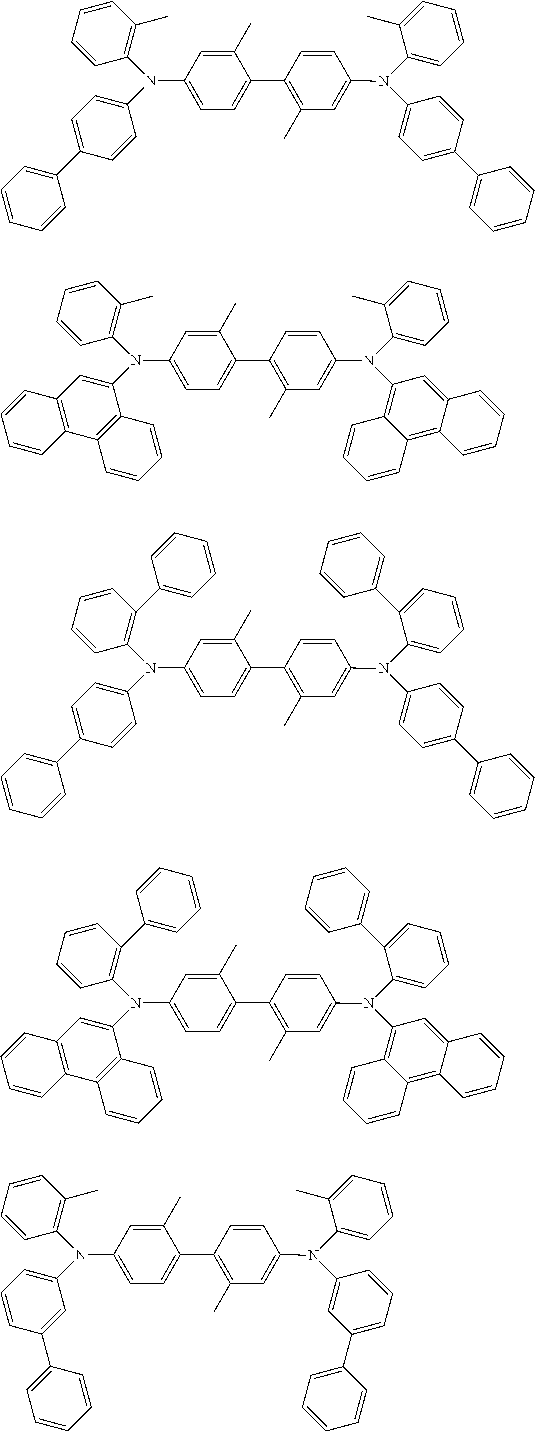 [SMILES: Cc1cc(N(c2ccc(-c3ccccc3)cc2)c2ccccc2C)ccc1-c1ccc(N(c2ccc(-c3ccccc3)cc2)c2ccccc2C)cc1C ; Cc1cc(N(c2ccccc2C)c2cc3ccccc3c3ccccc23)ccc1-c1ccc(N(c2ccccc2C)c2cc3ccccc3c3ccccc23)cc1C ; Cc1cc(N(c2ccc(-c3ccccc3)cc2)c2ccccc2-c2ccccc2)ccc1-c1ccc(N(c2ccc(-c3ccccc3)cc2)c2ccccc2-c2ccccc2)cc1C ; Cc1cc(N(c2ccccc2-c2ccccc2)c2cc3ccccc3c3ccccc23)ccc1-c1ccc(N(c2ccccc2-c2ccccc2)c2cc3ccccc3c3ccccc23)cc1C ; Cc1cc(N(c2cccc(-c3ccccc3)c2)c2ccccc2C)ccc1-c1ccc(N(c2cccc(-c3ccccc3)c2)c2ccccc2C)cc1C]

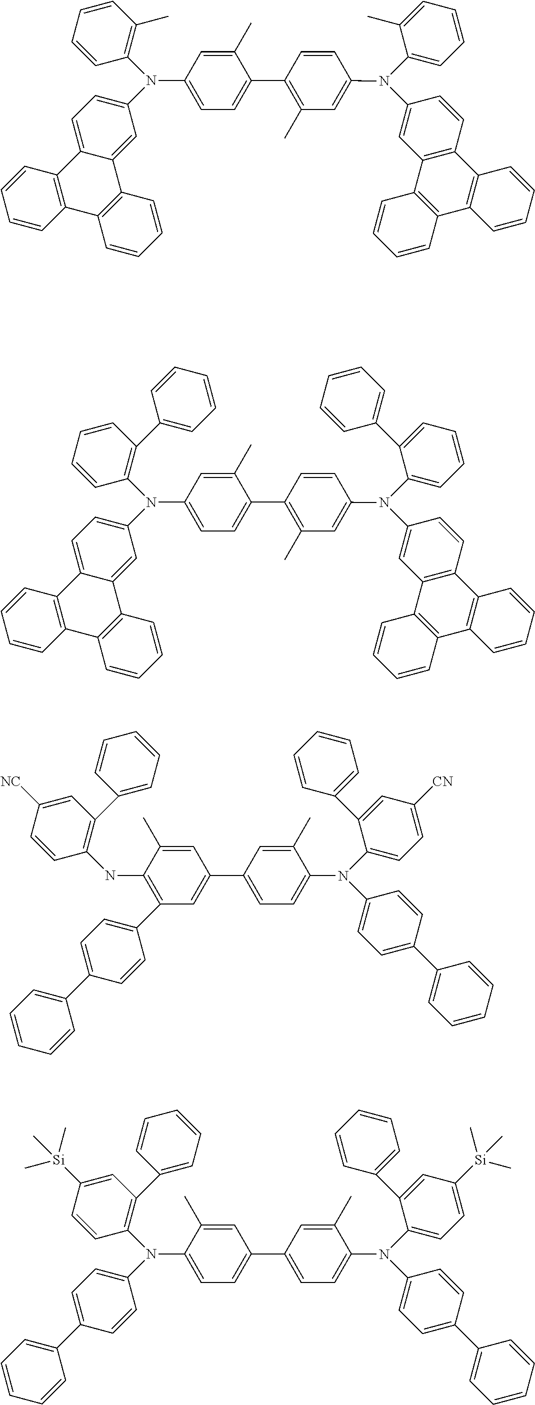 [SMILES: Cc1cc(N(c2ccc3c4ccccc4c4ccccc4c3c2)c2ccccc2C)ccc1-c1ccc(N(c2ccc3c4ccccc4c4ccccc4c3c2)c2ccccc2C)cc1C ; Cc1cc(N(c2ccc3c4ccccc4c4ccccc4c3c2)c2ccccc2-c2ccccc2)ccc1-c1ccc(N(c2ccc3c4ccccc4c4ccccc4c3c2)c2ccccc2-c2ccccc2)cc1C ; Cc1cc(-c2cc(C)c(Nc3ccc(C#N)cc3-c3ccccc3)c(-c3ccc(-c4ccccc4)cc3)c2)ccc1N(c1ccc(-c2ccccc2)cc1)c1ccc(C#N)cc1-c1ccccc1 ; Cc1cc(-c2ccc(N(c3ccc(-c4ccccc4)cc3)c3ccc([Si](C)(C)C)cc3-c3ccccc3)c(C)c2)ccc1N(c1ccc(-c2ccccc2)cc1)c1ccc([Si](C)(C)C)cc1-c1ccccc1]

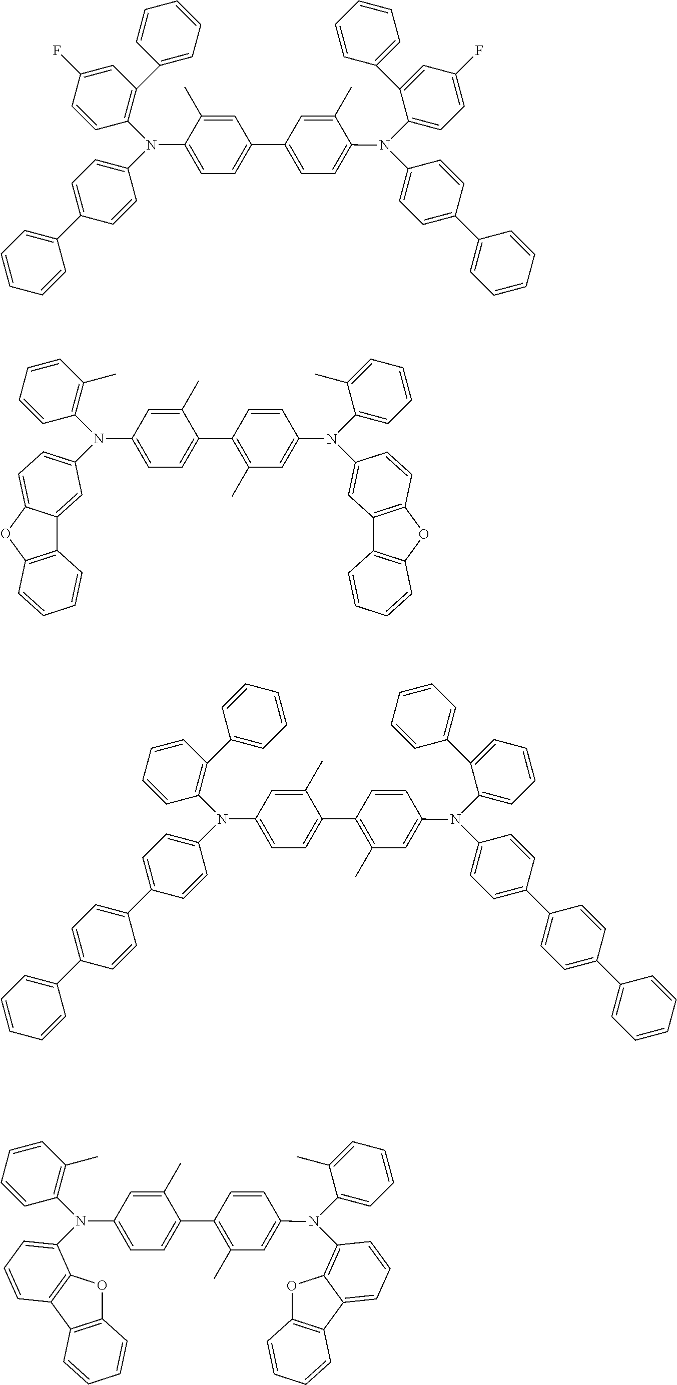 [SMILES: Cc1cc(-c2ccc(N(c3ccc(-c4ccccc4)cc3)c3ccc(F)cc3-c3ccccc3)c(C)c2)ccc1N(c1ccc(-c2ccccc2)cc1)c1ccc(F)cc1-c1ccccc1 ; Cc1cc(N(c2ccc3oc4ccccc4c3c2)c2ccccc2C)ccc1-c1ccc(N(c2ccc3oc4ccccc4c3c2)c2ccccc2C)cc1C ; Cc1cc(N(c2ccc(-c3ccc(-c4ccccc4)cc3)cc2)c2ccccc2-c2ccccc2)ccc1-c1ccc(N(c2ccc(-c3ccc(-c4ccccc4)cc3)cc2)c2ccccc2-c2ccccc2)cc1C ; Cc1cc(N(c2ccccc2C)c2cccc3c2oc2ccccc23)ccc1-c1ccc(N(c2ccccc2C)c2cccc3c2oc2ccccc23)cc1C]

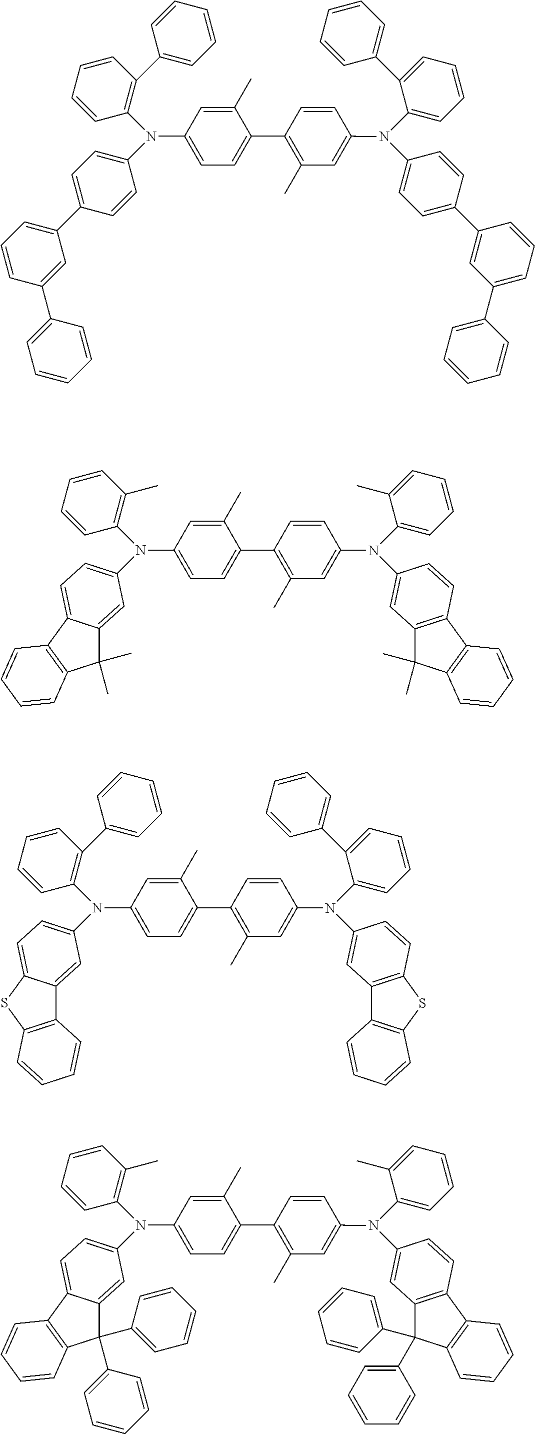 [SMILES: Cc1cc(N(c2ccc(-c3cccc(-c4ccccc4)c3)cc2)c2ccccc2-c2ccccc2)ccc1-c1ccc(N(c2ccc(-c3cccc(-c4ccccc4)c3)cc2)c2ccccc2-c2ccccc2)cc1C ; Cc1cc(N(c2ccc3c(c2)C(C)(C)c2ccccc2-3)c2ccccc2C)ccc1-c1ccc(N(c2ccc3c(c2)C(C)(C)c2ccccc2-3)c2ccccc2C)cc1C ; Cc1cc(N(c2ccc3sc4ccccc4c3c2)c2ccccc2-c2ccccc2)ccc1-c1ccc(N(c2ccc3sc4ccccc4c3c2)c2ccccc2-c2ccccc2)cc1C ; Cc1cc(N(c2ccc3c(c2)C(c2ccccc2)(c2ccccc2)c2ccccc2-3)c2ccccc2C)ccc1-c1ccc(N(c2ccc3c(c2)C(c2ccccc2)(c2ccccc2)c2ccccc2-3)c2ccccc2C)cc1C]

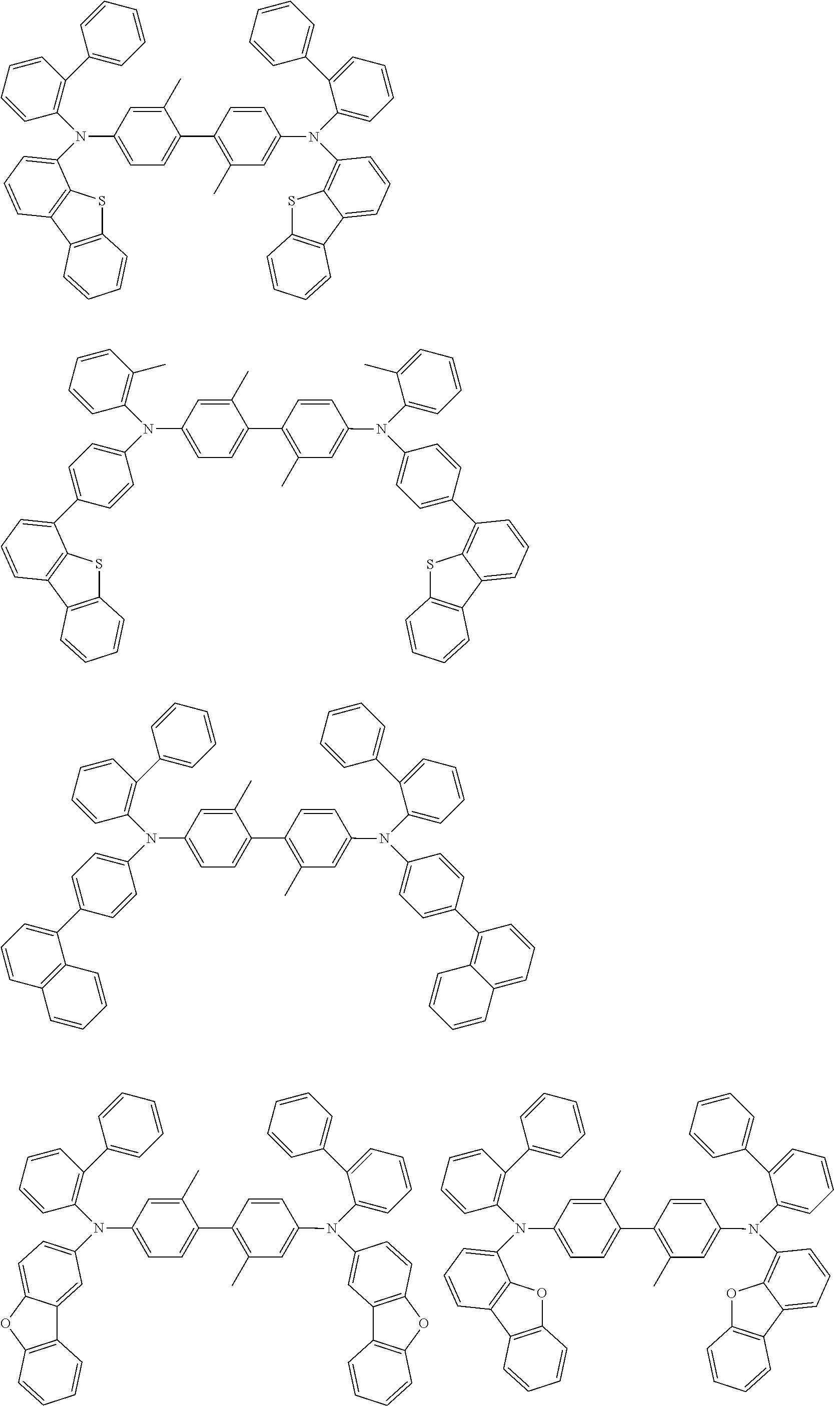

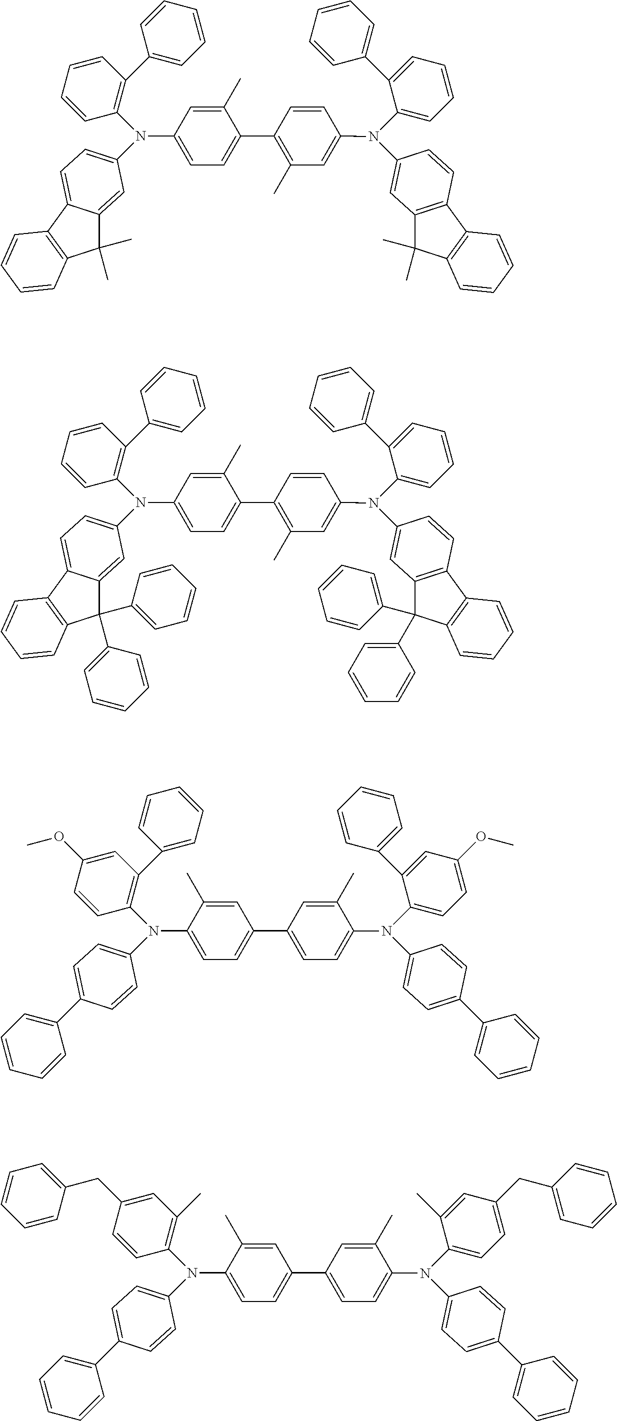 [SMILES: Cc1cc(N(c2ccc3c(c2)C(C)(C)c2ccccc2-3)c2ccccc2-c2ccccc2)ccc1-c1ccc(N(c2ccc3c(c2)C(C)(C)c2ccccc2-3)c2ccccc2-c2ccccc2)cc1C ; Cc1cc(N(c2ccc3c(c2)C(c2ccccc2)(c2ccccc2)c2ccccc2-3)c2ccccc2-c2ccccc2)ccc1-c1ccc(N(c2ccc3c(c2)C(c2ccccc2)(c2ccccc2)c2ccccc2-3)c2ccccc2-c2ccccc2)cc1C ; COc1ccc(N(c2ccc(-c3ccccc3)cc2)c2ccc(-c3ccc(N(c4ccc(-c5ccccc5)cc4)c4ccc(OC)cc4-c4ccccc4)c(C)c3)cc2C)c(-c2ccccc2)c1 ; Cc1cc(Cc2ccccc2)ccc1N(c1ccc(-c2ccccc2)cc1)c1ccc(-c2ccc(N(c3ccc(-c4ccccc4)cc3)c3ccc(Cc4ccccc4)cc3C)c(C)c2)cc1C]

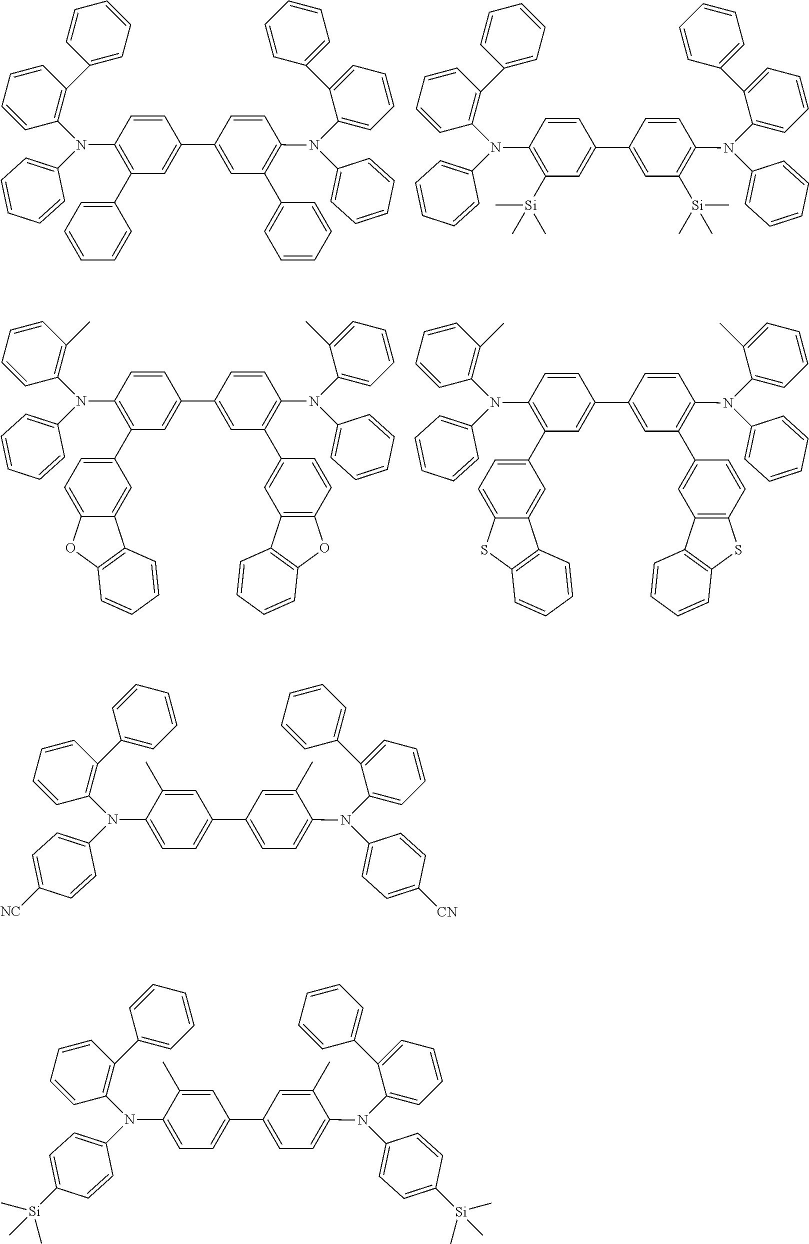

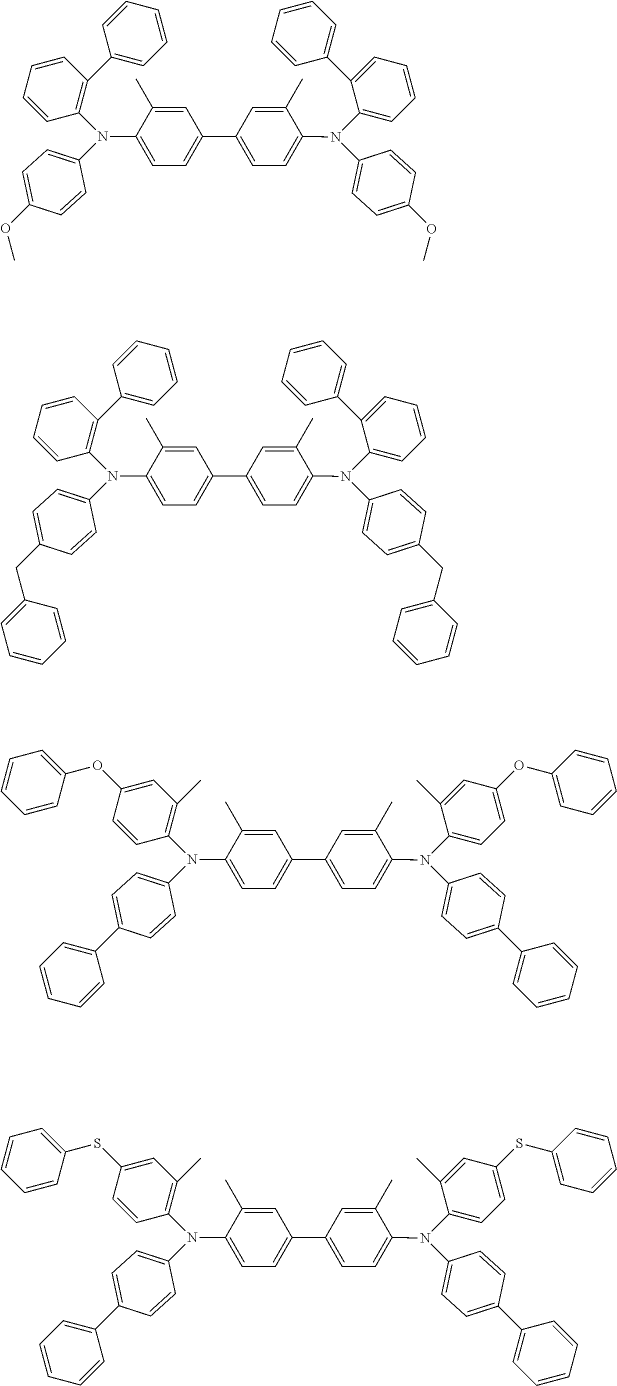 [SMILES: COc1ccc(N(c2ccc(-c3ccc(N(c4ccc(OC)cc4)c4ccccc4-c4ccccc4)c(C)c3)cc2C)c2ccccc2-c2ccccc2)cc1 ; Cc1cc(-c2ccc(N(c3ccc(Cc4ccccc4)cc3)c3ccccc3-c3ccccc3)c(C)c2)ccc1N(c1ccc(Cc2ccccc2)cc1)c1ccccc1-c1ccccc1 ; Cc1cc(Oc2ccccc2)ccc1N(c1ccc(-c2ccccc2)cc1)c1ccc(-c2ccc(N(c3ccc(-c4ccccc4)cc3)c3ccc(Oc4ccccc4)cc3C)c(C)c2)cc1C ; Cc1cc(Sc2ccccc2)ccc1N(c1ccc(-c2ccccc2)cc1)c1ccc(-c2ccc(N(c3ccc(-c4ccccc4)cc3)c3ccc(Sc4ccccc4)cc3C)c(C)c2)cc1C]

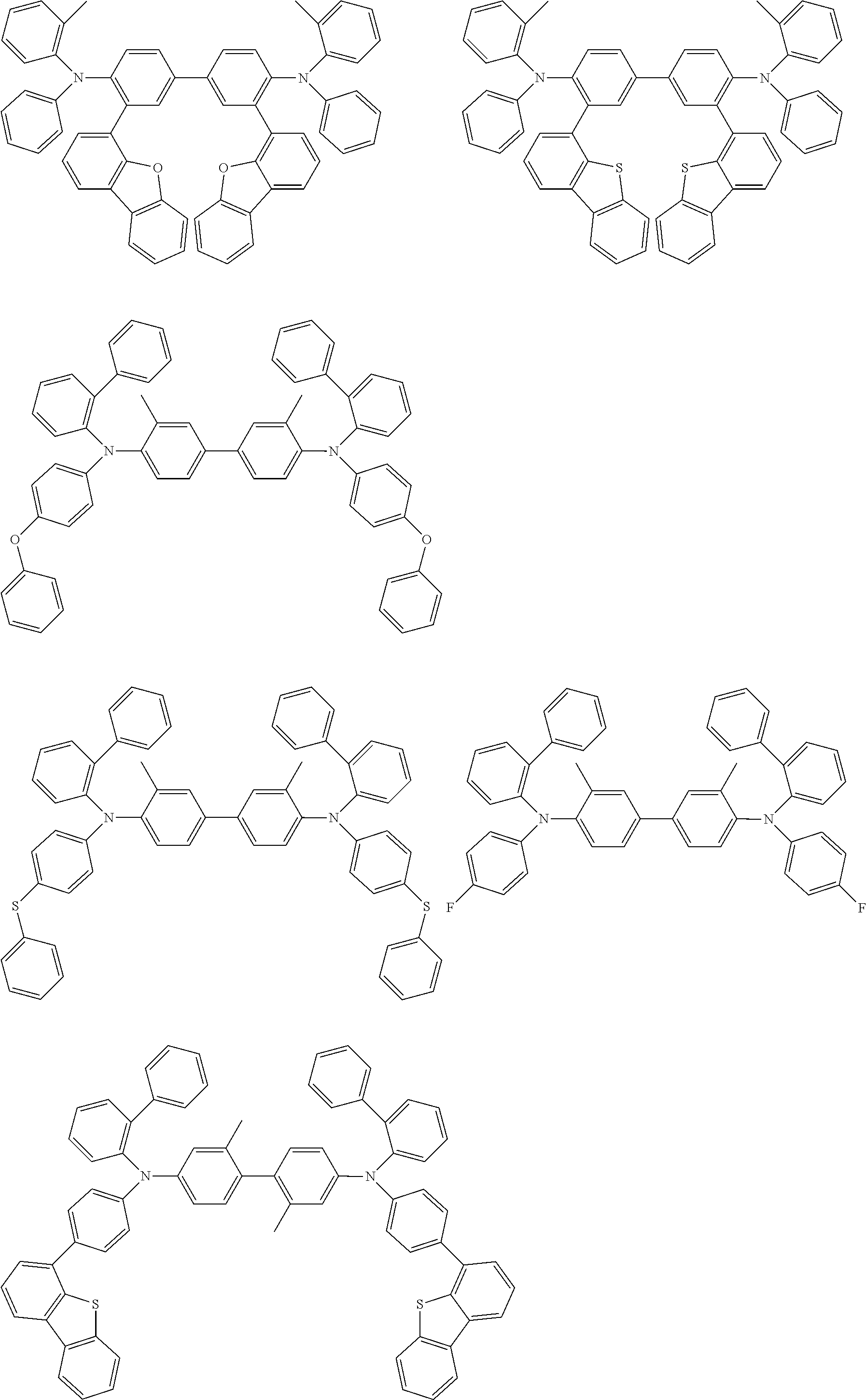

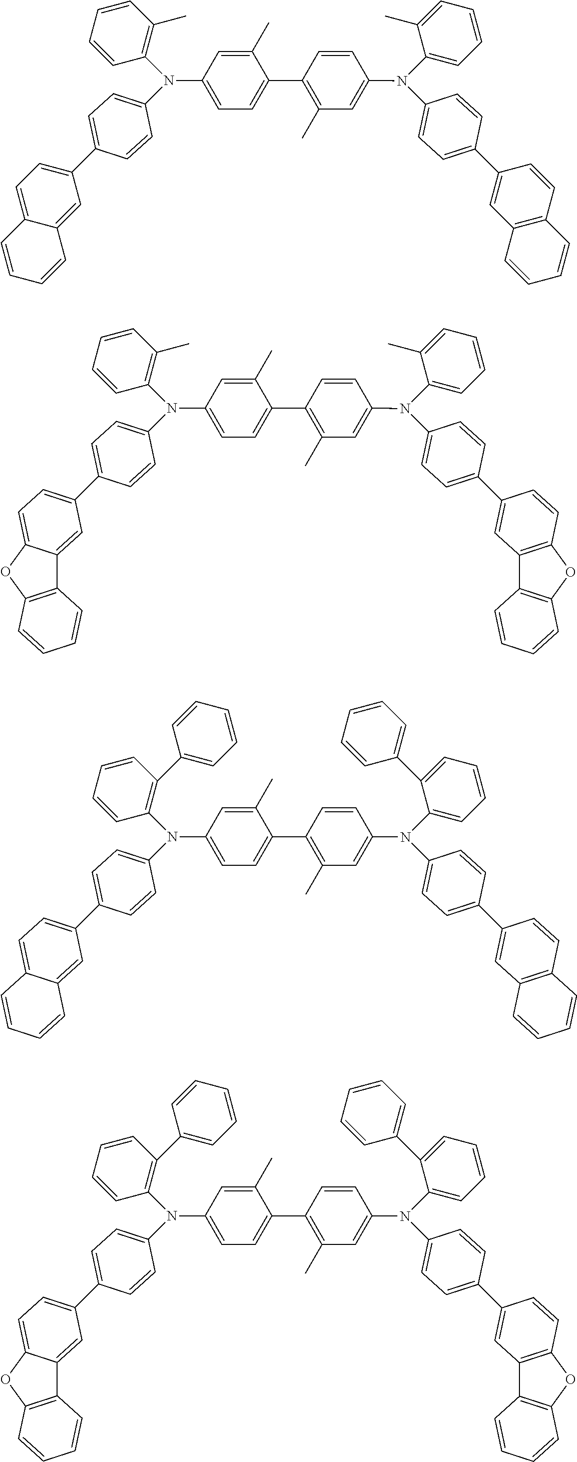 [SMILES: Cc1cc(N(c2ccc(-c3ccc4ccccc4c3)cc2)c2ccccc2C)ccc1-c1ccc(N(c2ccc(-c3ccc4ccccc4c3)cc2)c2ccccc2C)cc1C ; Cc1cc(N(c2ccc(-c3ccc4oc5ccccc5c4c3)cc2)c2ccccc2C)ccc1-c1ccc(N(c2ccc(-c3ccc4oc5ccccc5c4c3)cc2)c2ccccc2C)cc1C ; Cc1cc(N(c2ccc(-c3ccc4ccccc4c3)cc2)c2ccccc2-c2ccccc2)ccc1-c1ccc(N(c2ccc(-c3ccc4ccccc4c3)cc2)c2ccccc2-c2ccccc2)cc1C ; Cc1cc(N(c2ccc(-c3ccc4oc5ccccc5c4c3)cc2)c2ccccc2-c2ccccc2)ccc1-c1ccc(N(c2ccc(-c3ccc4oc5ccccc5c4c3)cc2)c2ccccc2-c2ccccc2)cc1C]

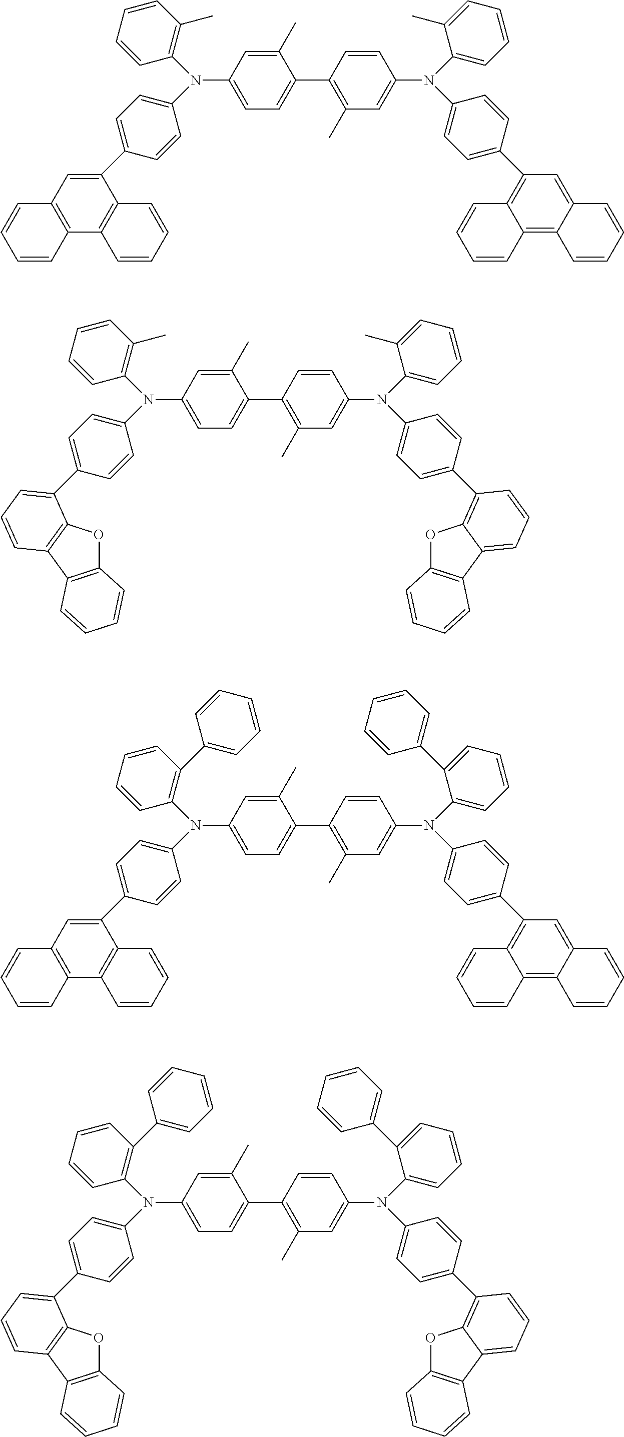 [SMILES: Cc1cc(N(c2ccc(-c3cc4ccccc4c4ccccc34)cc2)c2ccccc2C)ccc1-c1ccc(N(c2ccc(-c3cc4ccccc4c4ccccc34)cc2)c2ccccc2C)cc1C ; Cc1cc(N(c2ccc(-c3cccc4c3oc3ccccc34)cc2)c2ccccc2C)ccc1-c1ccc(N(c2ccc(-c3cccc4c3oc3ccccc34)cc2)c2ccccc2C)cc1C ; Cc1cc(N(c2ccc(-c3cc4ccccc4c4ccccc34)cc2)c2ccccc2-c2ccccc2)ccc1-c1ccc(N(c2ccc(-c3cc4ccccc4c4ccccc34)cc2)c2ccccc2-c2ccccc2)cc1C ; Cc1cc(N(c2ccc(-c3cccc4c3oc3ccccc34)cc2)c2ccccc2-c2ccccc2)ccc1-c1ccc(N(c2ccc(-c3cccc4c3oc3ccccc34)cc2)c2ccccc2-c2ccccc2)cc1C]

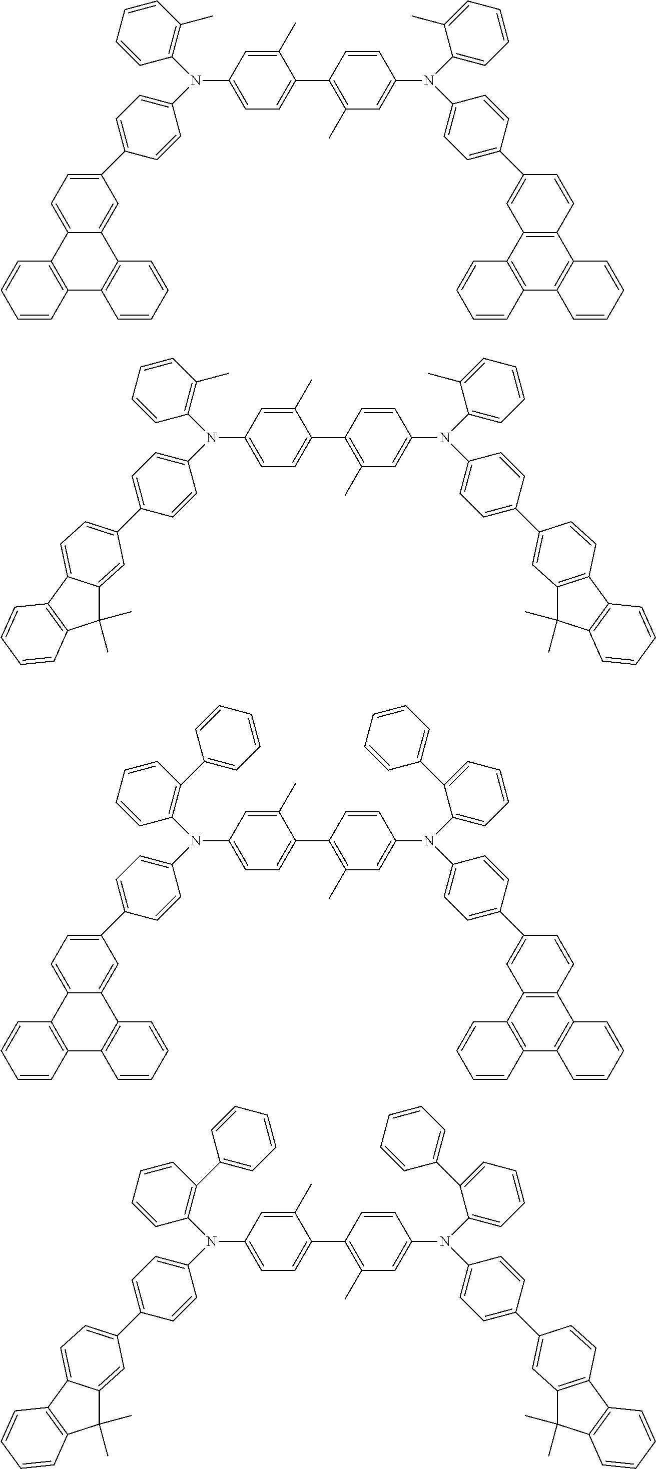

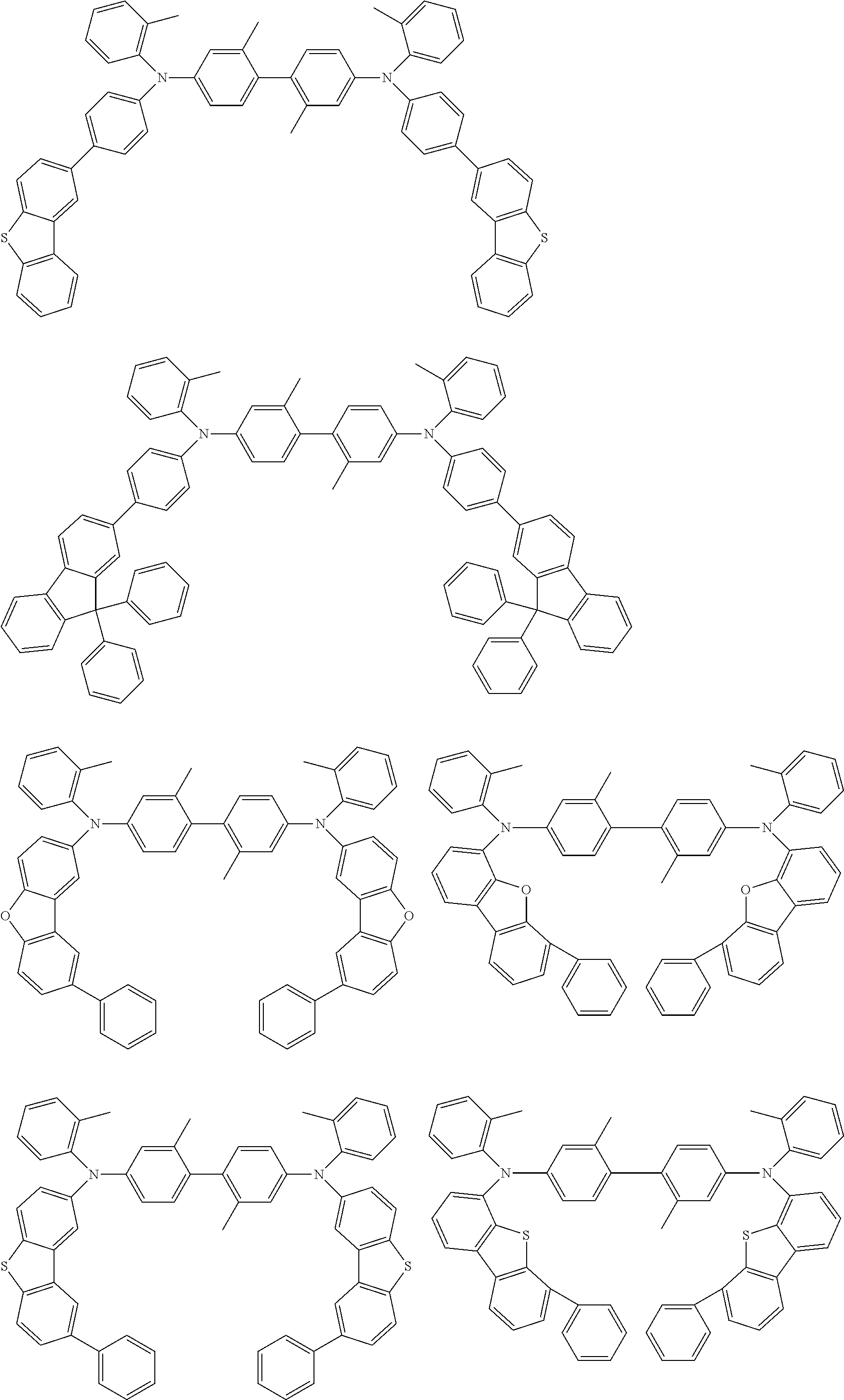

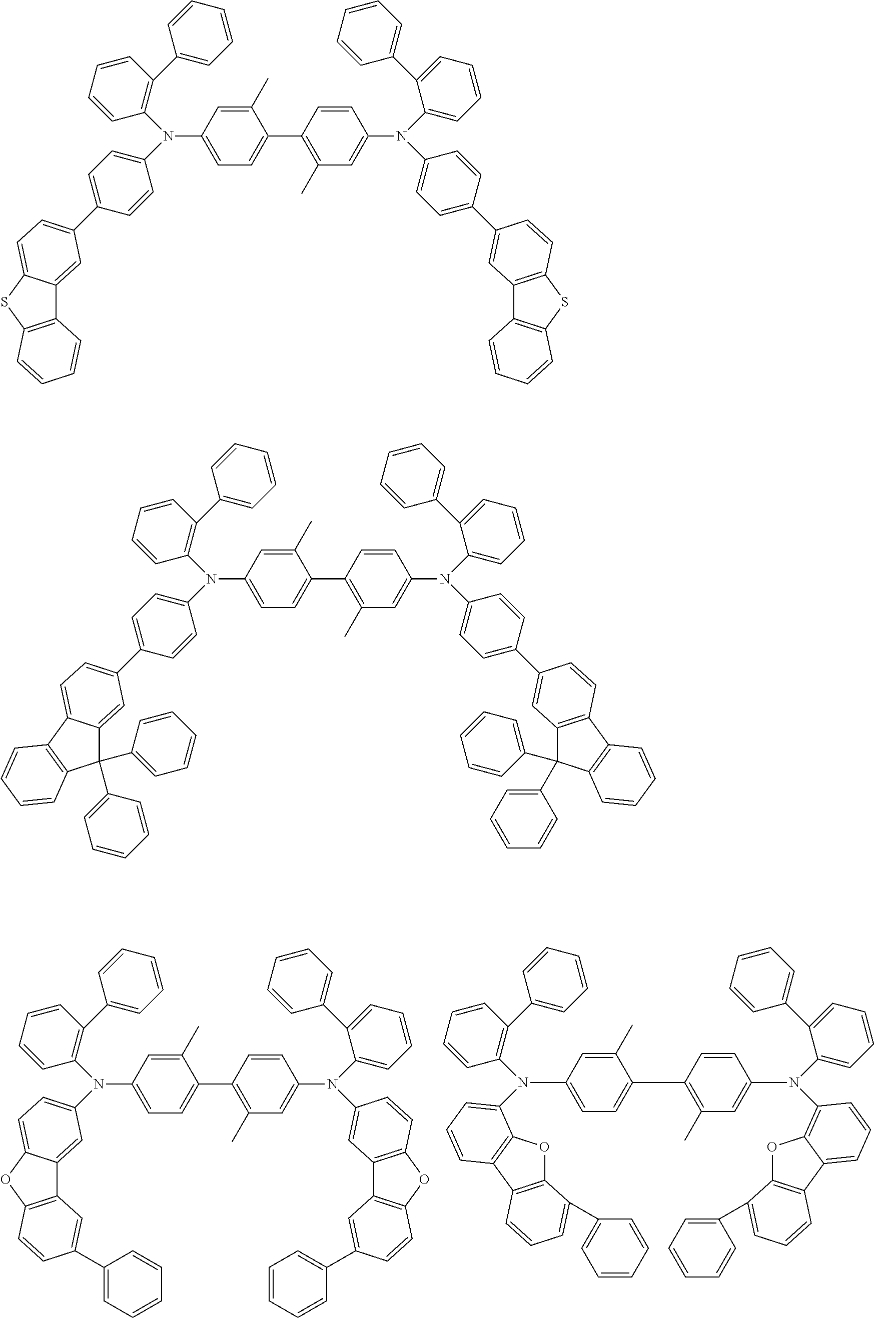

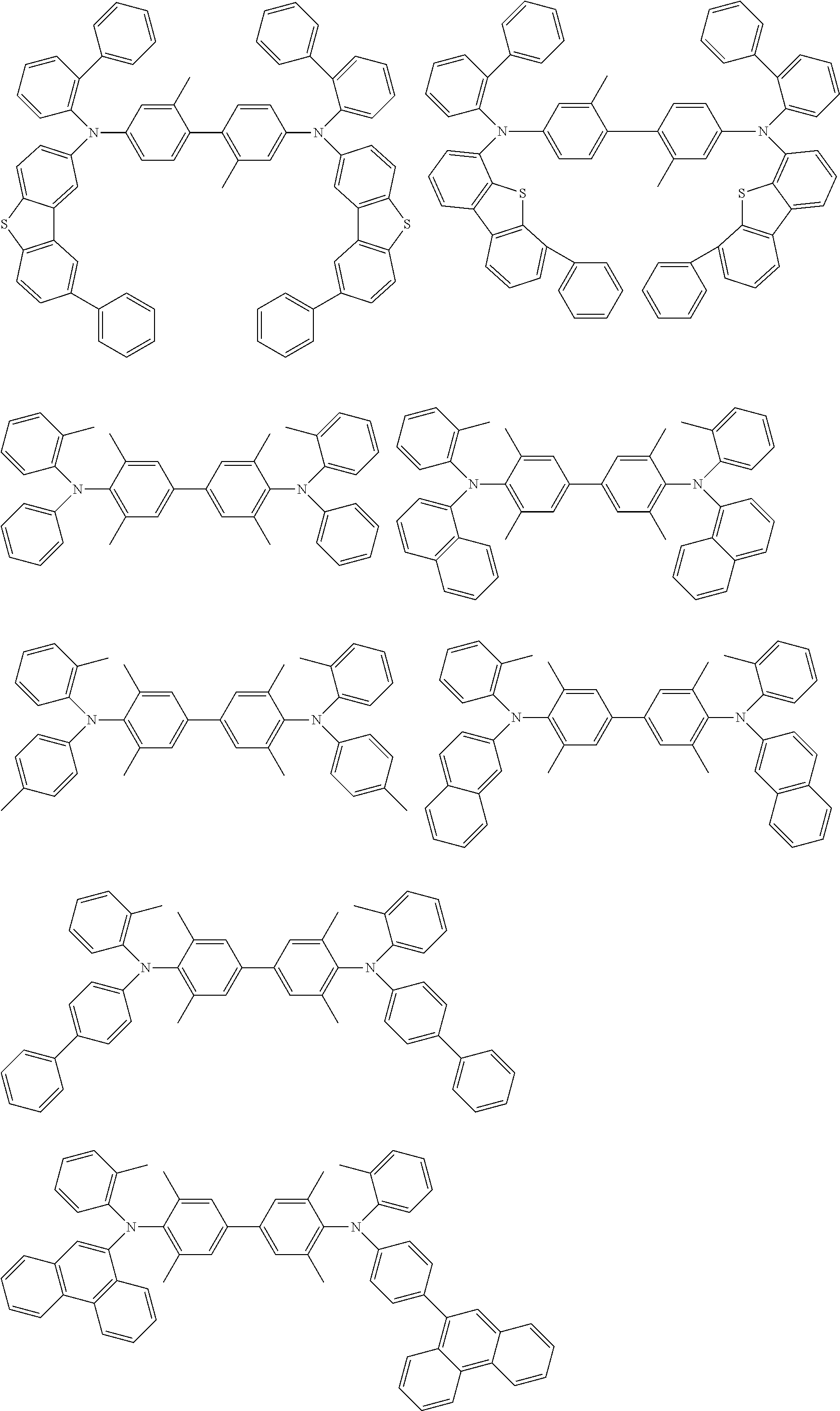

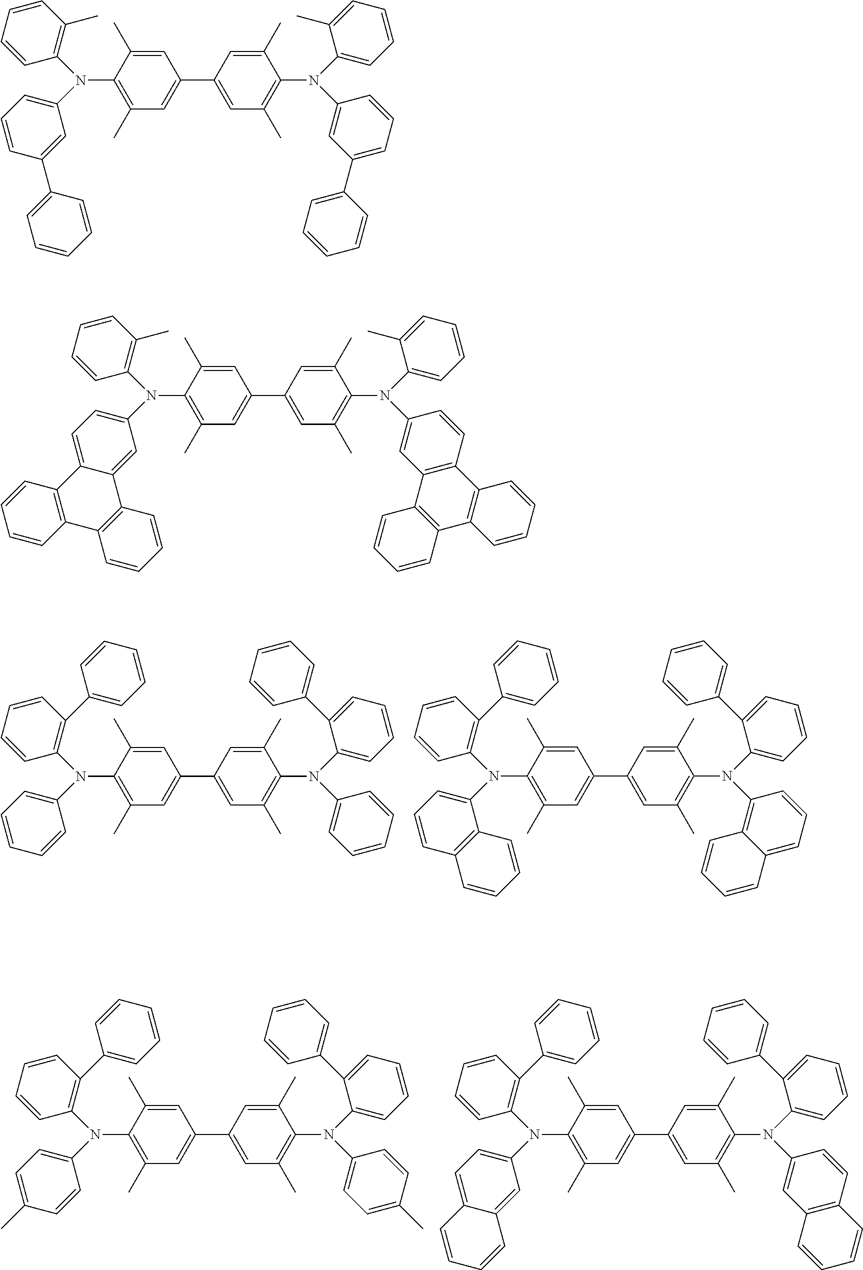

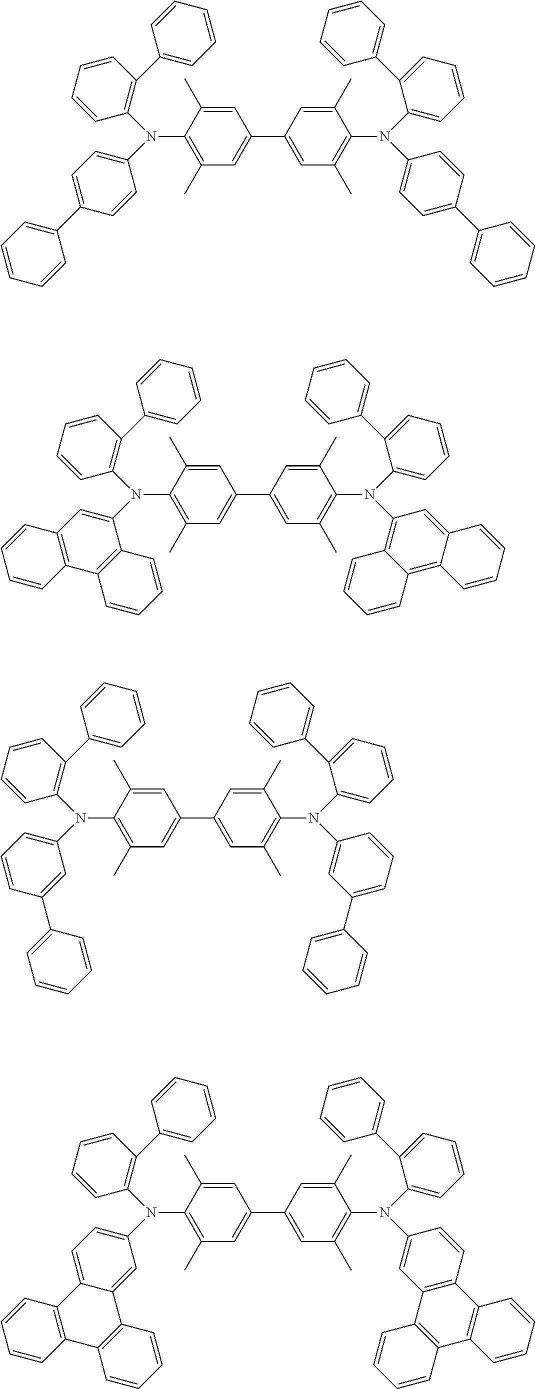 [SMILES: Cc1cc(-c2cc(C)c(N(c3ccc(-c4ccccc4)cc3)c3ccccc3-c3ccccc3)c(C)c2)cc(C)c1N(c1ccc(-c2ccccc2)cc1)c1ccccc1-c1ccccc1 ; Cc1cc(-c2cc(C)c(N(c3ccccc3-c3ccccc3)c3cc4ccccc4c4ccccc34)c(C)c2)cc(C)c1N(c1ccccc1-c1ccccc1)c1cc2ccccc2c2ccccc12 ; Cc1cc(-c2cc(C)c(N(c3cccc(-c4ccccc4)c3)c3ccccc3-c3ccccc3)c(C)c2)cc(C)c1N(c1cccc(-c2ccccc2)c1)c1ccccc1-c1ccccc1 ; Cc1cc(-c2cc(C)c(N(c3ccc4c5ccccc5c5ccccc5c4c3)c3ccccc3-c3ccccc3)c(C)c2)cc(C)c1N(c1ccc2c3ccccc3c3ccccc3c2c1)c1ccccc1-c1ccccc1]

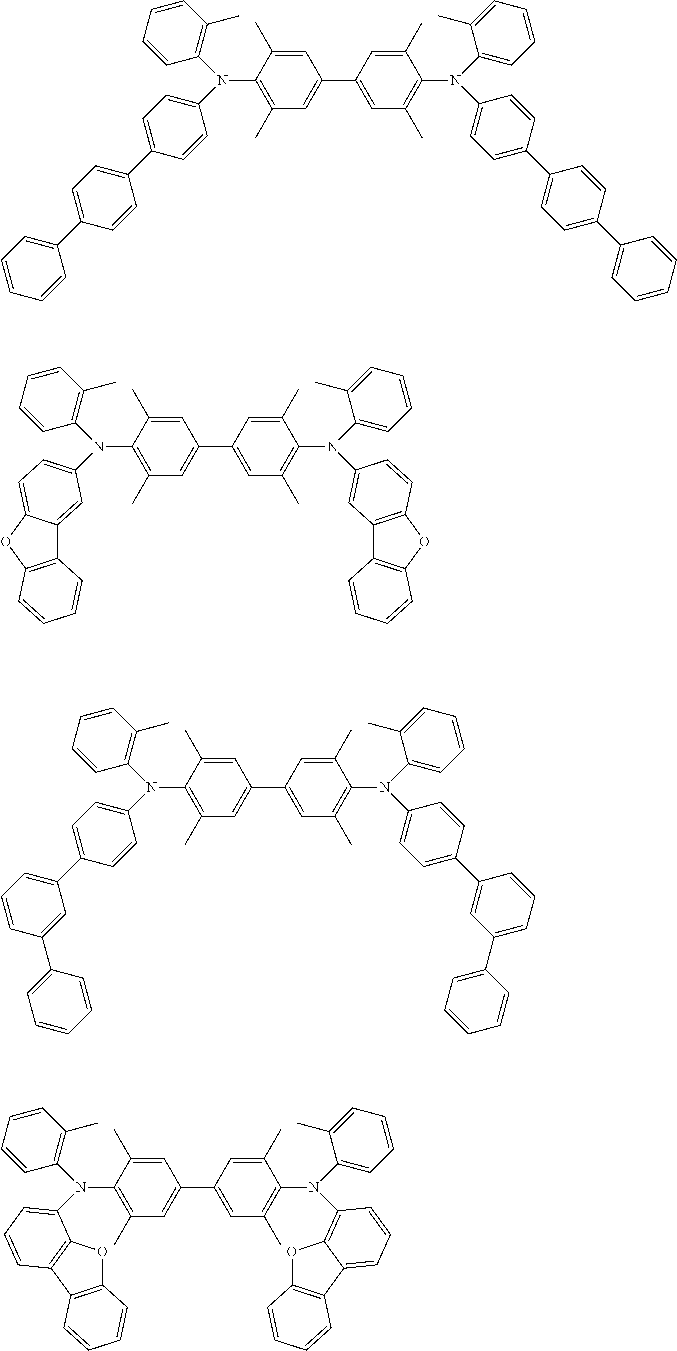 [SMILES: Cc1ccccc1N(c1ccc(-c2ccc(-c3ccccc3)cc2)cc1)c1c(C)cc(-c2cc(C)c(N(c3ccc(-c4ccc(-c5ccccc5)cc4)cc3)c3ccccc3C)c(C)c2)cc1C ; Cc1ccccc1N(c1ccc2oc3ccccc3c2c1)c1c(C)cc(-c2cc(C)c(N(c3ccc4oc5ccccc5c4c3)c3ccccc3C)c(C)c2)cc1C ; Cc1ccccc1N(c1ccc(-c2cccc(-c3ccccc3)c2)cc1)c1c(C)cc(-c2cc(C)c(N(c3ccc(-c4cccc(-c5ccccc5)c4)cc3)c3ccccc3C)c(C)c2)cc1C ; Cc1ccccc1N(c1c(C)cc(-c2cc(C)c(N(c3ccccc3C)c3cccc4c3oc3ccccc34)c(C)c2)cc1C)c1cccc2c1oc1ccccc12]

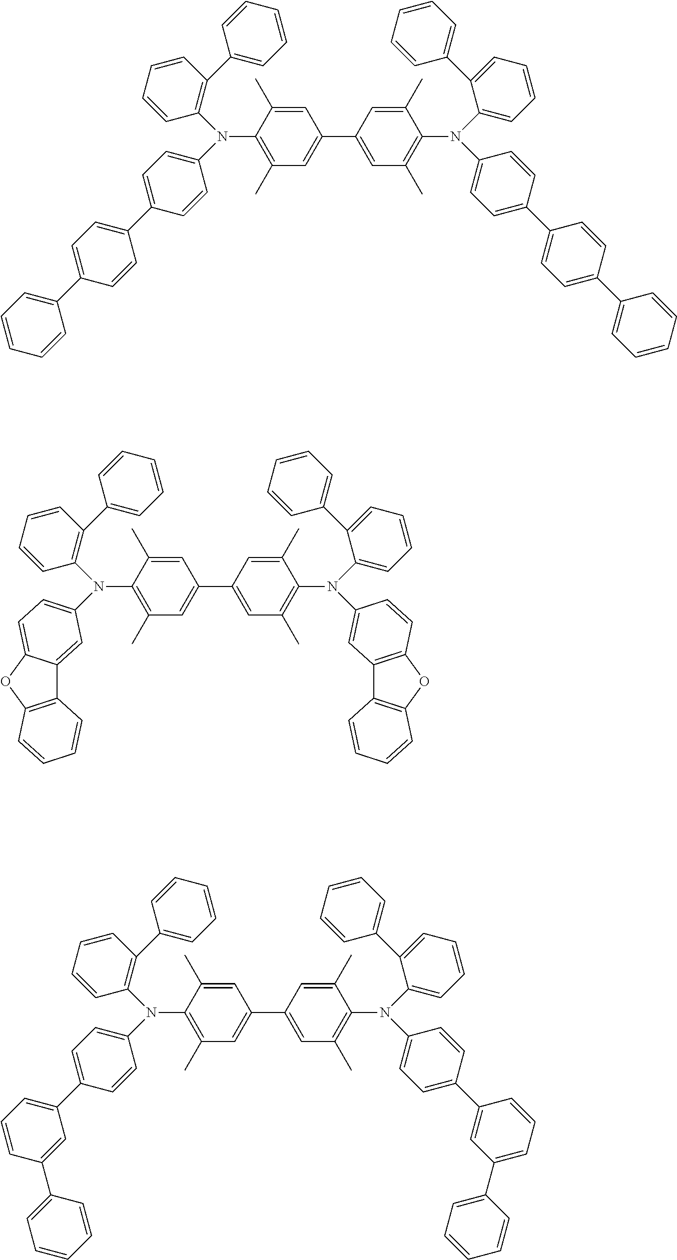 [SMILES: Cc1cc(-c2cc(C)c(N(c3ccc(-c4ccc(-c5ccccc5)cc4)cc3)c3ccccc3-c3ccccc3)c(C)c2)cc(C)c1N(c1ccc(-c2ccc(-c3ccccc3)cc2)cc1)c1ccccc1-c1ccccc1 ; Cc1cc(-c2cc(C)c(N(c3ccc4oc5ccccc5c4c3)c3ccccc3-c3ccccc3)c(C)c2)cc(C)c1N(c1ccc2oc3ccccc3c2c1)c1ccccc1-c1ccccc1 ; Cc1cc(-c2cc(C)c(N(c3ccc(-c4cccc(-c5ccccc5)c4)cc3)c3ccccc3-c3ccccc3)c(C)c2)cc(C)c1N(c1ccc(-c2cccc(-c3ccccc3)c2)cc1)c1ccccc1-c1ccccc1]

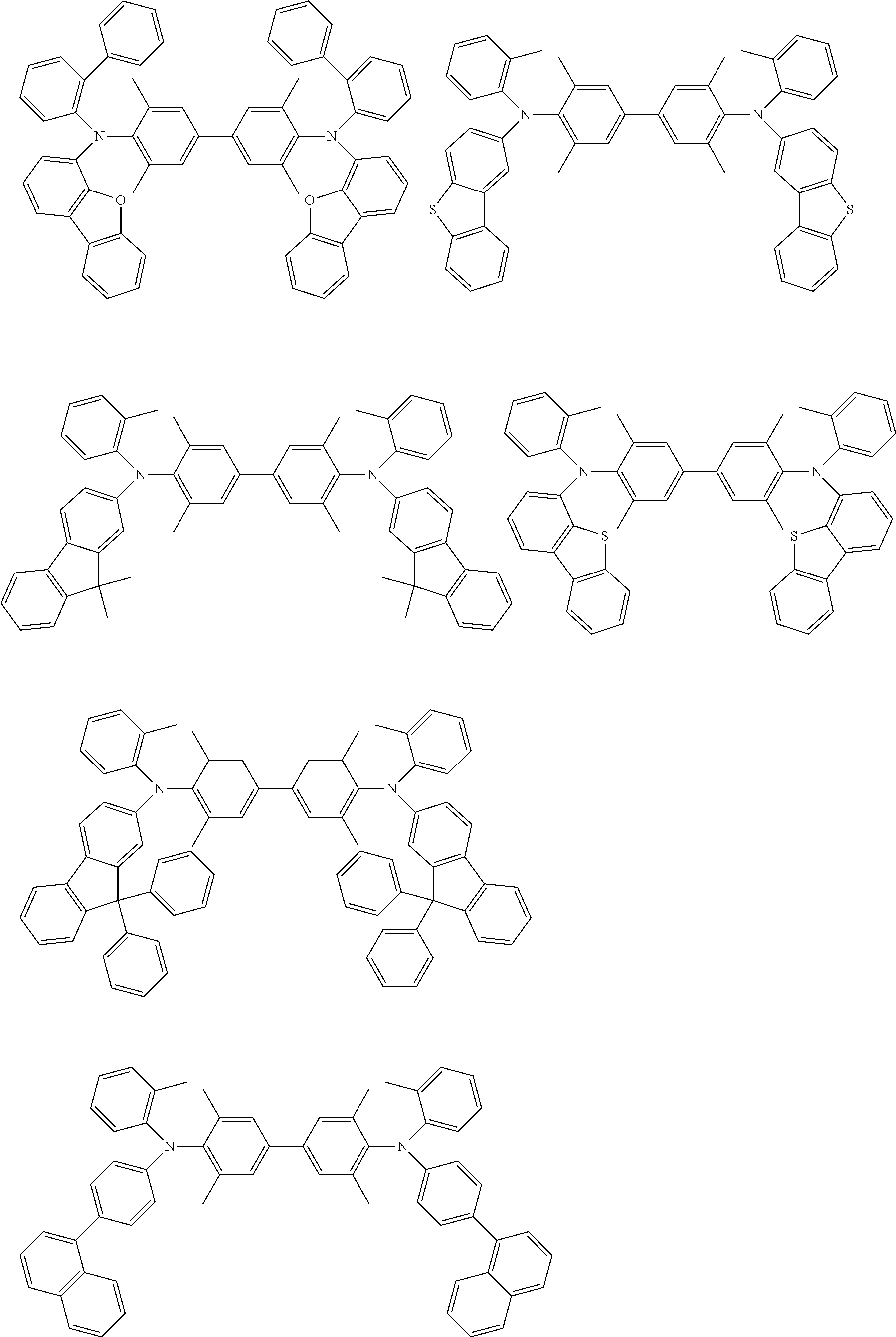

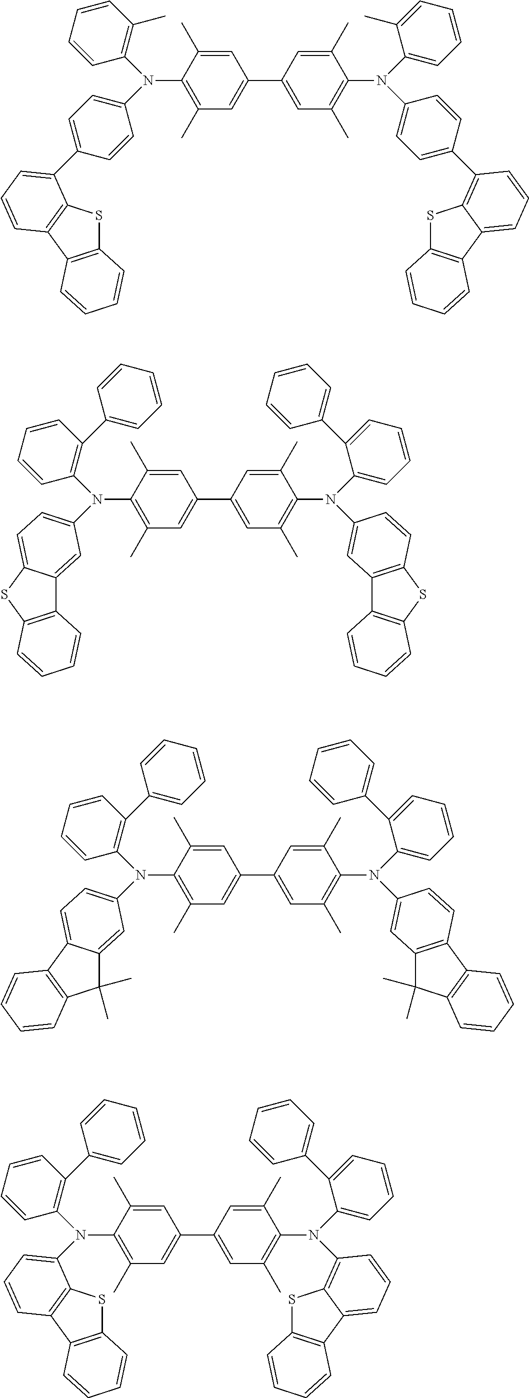 [SMILES: Cc1ccccc1N(c1ccc(-c2cccc3c2sc2ccccc23)cc1)c1c(C)cc(-c2cc(C)c(N(c3ccc(-c4cccc5c4sc4ccccc45)cc3)c3ccccc3C)c(C)c2)cc1C ; Cc1cc(-c2cc(C)c(N(c3ccc4sc5ccccc5c4c3)c3ccccc3-c3ccccc3)c(C)c2)cc(C)c1N(c1ccc2sc3ccccc3c2c1)c1ccccc1-c1ccccc1 ; Cc1cc(-c2cc(C)c(N(c3ccc4c(c3)C(C)(C)c3ccccc3-4)c3ccccc3-c3ccccc3)c(C)c2)cc(C)c1N(c1ccc2c(c1)C(C)(C)c1ccccc1-2)c1ccccc1-c1ccccc1 ; Cc1cc(-c2cc(C)c(N(c3ccccc3-c3ccccc3)c3cccc4c3sc3ccccc34)c(C)c2)cc(C)c1N(c1ccccc1-c1ccccc1)c1cccc2c1sc1ccccc12]

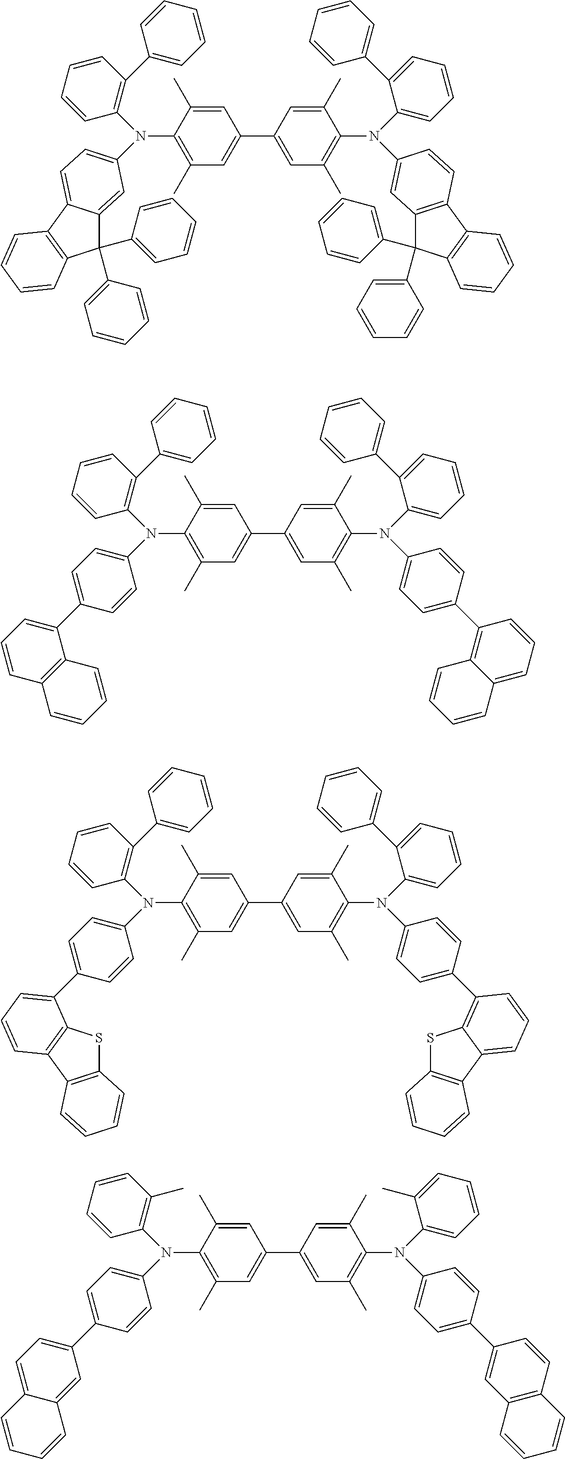

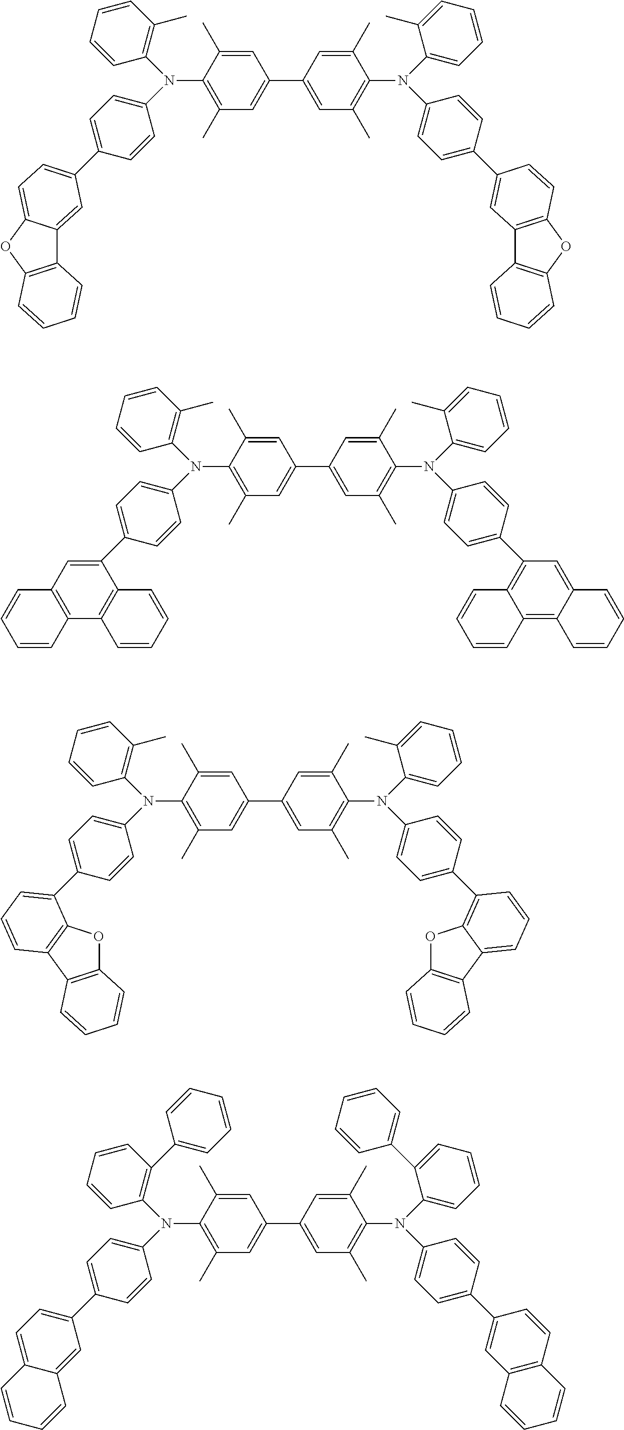 [SMILES: Cc1ccccc1N(c1ccc(-c2ccc3oc4ccccc4c3c2)cc1)c1c(C)cc(-c2cc(C)c(N(c3ccc(-c4ccc5oc6ccccc6c5c4)cc3)c3ccccc3C)c(C)c2)cc1C ; Cc1ccccc1N(c1ccc(-c2cc3ccccc3c3ccccc23)cc1)c1c(C)cc(-c2cc(C)c(N(c3ccc(-c4cc5ccccc5c5ccccc45)cc3)c3ccccc3C)c(C)c2)cc1C ; Cc1ccccc1N(c1ccc(-c2cccc3c2oc2ccccc23)cc1)c1c(C)cc(-c2cc(C)c(N(c3ccc(-c4cccc5c4oc4ccccc45)cc3)c3ccccc3C)c(C)c2)cc1C ; Cc1cc(-c2cc(C)c(N(c3ccc(-c4ccc5ccccc5c4)cc3)c3ccccc3-c3ccccc3)c(C)c2)cc(C)c1N(c1ccc(-c2ccc3ccccc3c2)cc1)c1ccccc1-c1ccccc1]

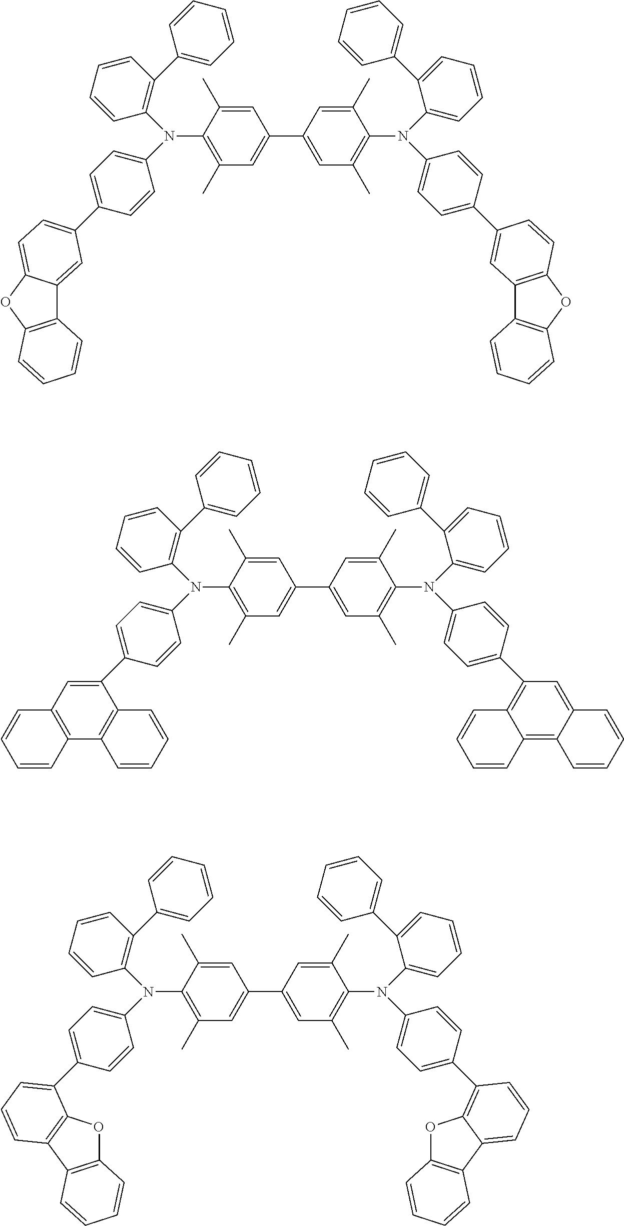 [SMILES: Cc1cc(-c2cc(C)c(N(c3ccc(-c4ccc5oc6ccccc6c5c4)cc3)c3ccccc3-c3ccccc3)c(C)c2)cc(C)c1N(c1ccc(-c2ccc3oc4ccccc4c3c2)cc1)c1ccccc1-c1ccccc1 ; Cc1cc(-c2cc(C)c(N(c3ccc(-c4cc5ccccc5c5ccccc45)cc3)c3ccccc3-c3ccccc3)c(C)c2)cc(C)c1N(c1ccc(-c2cc3ccccc3c3ccccc23)cc1)c1ccccc1-c1ccccc1 ; Cc1cc(-c2cc(C)c(N(c3ccc(-c4cccc5c4oc4ccccc45)cc3)c3ccccc3-c3ccccc3)c(C)c2)cc(C)c1N(c1ccc(-c2cccc3c2oc2ccccc23)cc1)c1ccccc1-c1ccccc1]

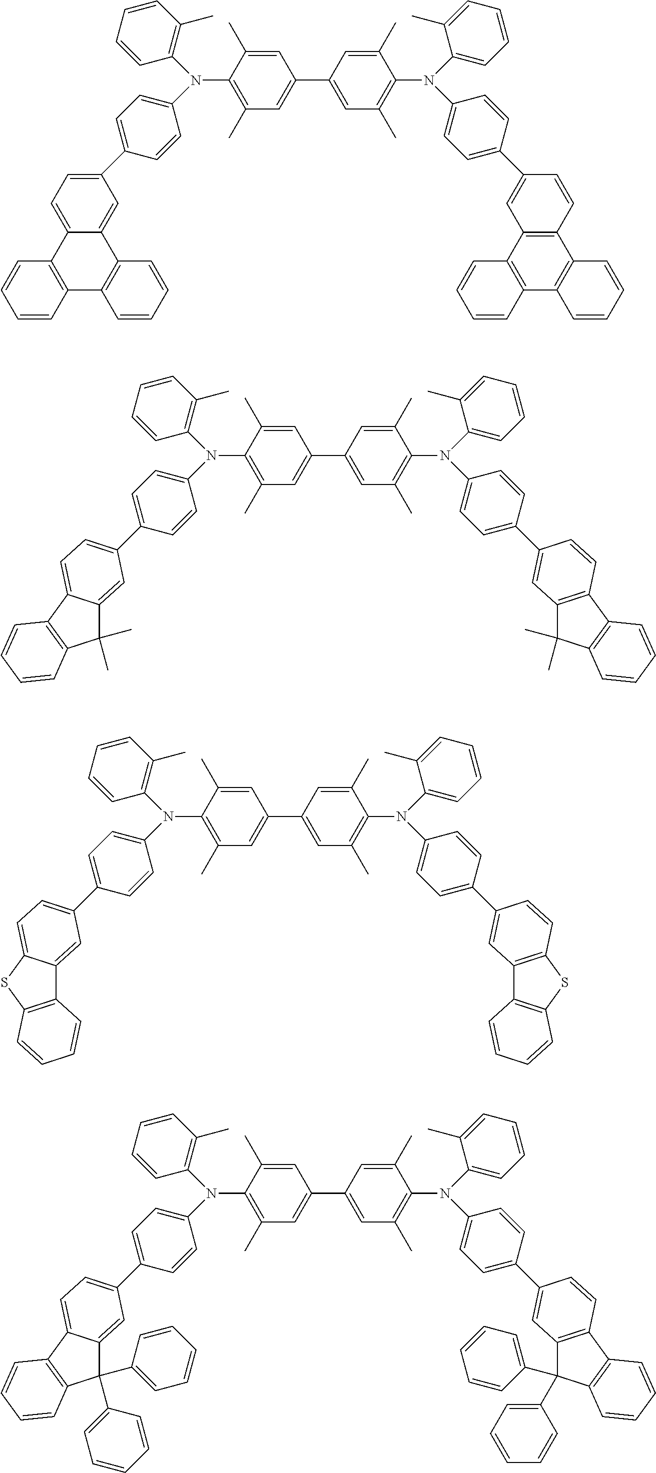

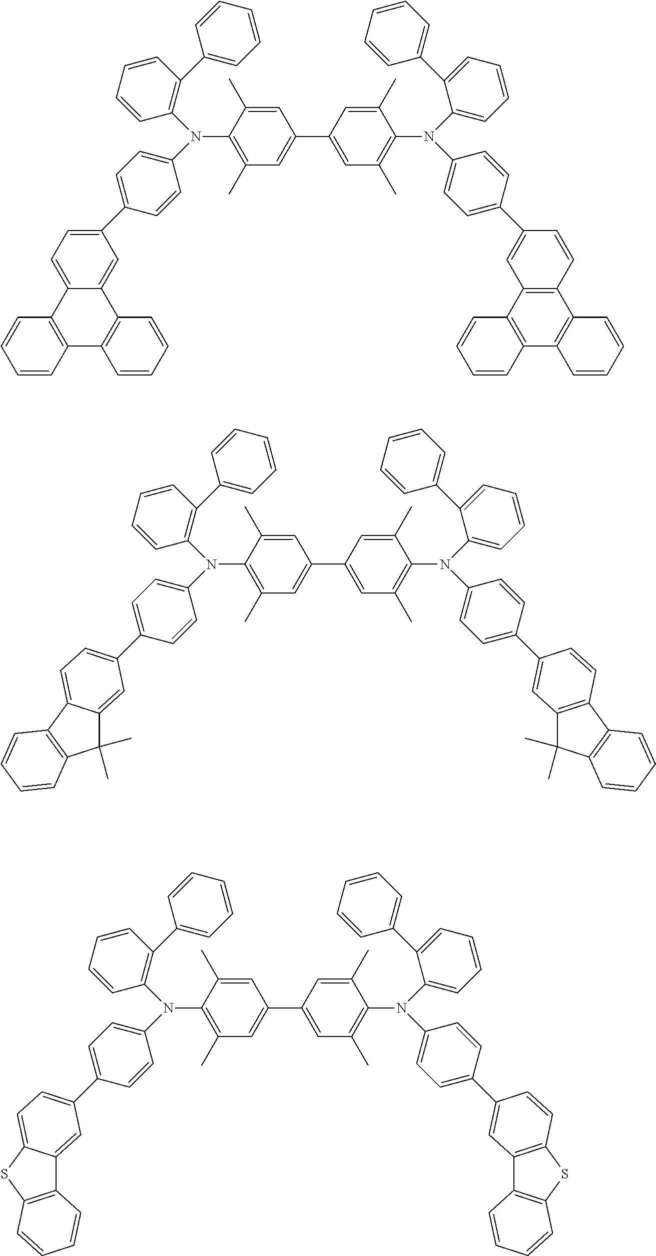 [SMILES: Cc1cc(-c2cc(C)c(N(c3ccc(-c4ccc5c6ccccc6c6ccccc6c5c4)cc3)c3ccccc3-c3ccccc3)c(C)c2)cc(C)c1N(c1ccc(-c2ccc3c4ccccc4c4ccccc4c3c2)cc1)c1ccccc1-c1ccccc1 ; Cc1cc(-c2cc(C)c(N(c3ccc(-c4ccc5c(c4)C(C)(C)c4ccccc4-5)cc3)c3ccccc3-c3ccccc3)c(C)c2)cc(C)c1N(c1ccc(-c2ccc3c(c2)C(C)(C)c2ccccc2-3)cc1)c1ccccc1-c1ccccc1 ; Cc1cc(-c2cc(C)c(N(c3ccc(-c4ccc5sc6ccccc6c5c4)cc3)c3ccccc3-c3ccccc3)c(C)c2)cc(C)c1N(c1ccc(-c2ccc3sc4ccccc4c3c2)cc1)c1ccccc1-c1ccccc1]

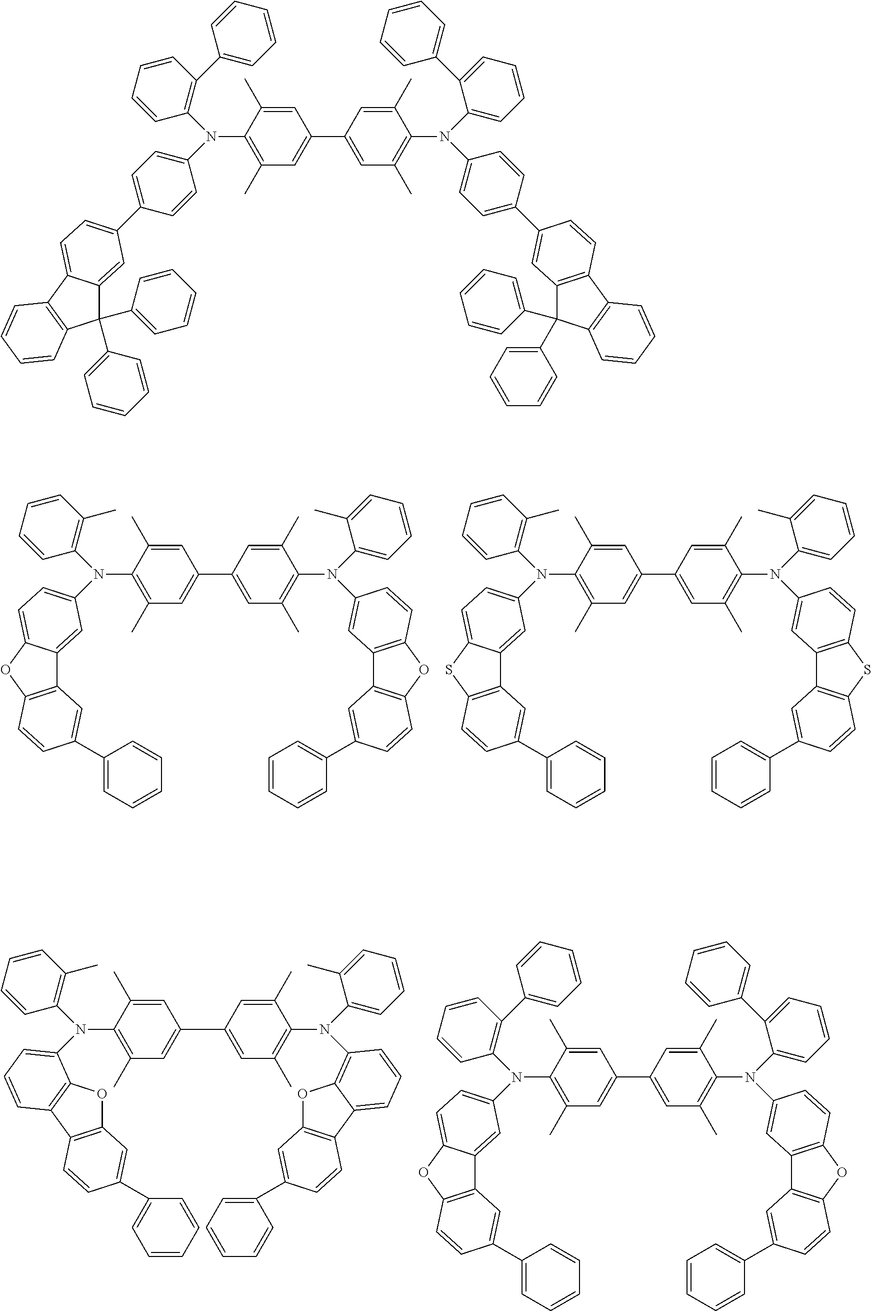

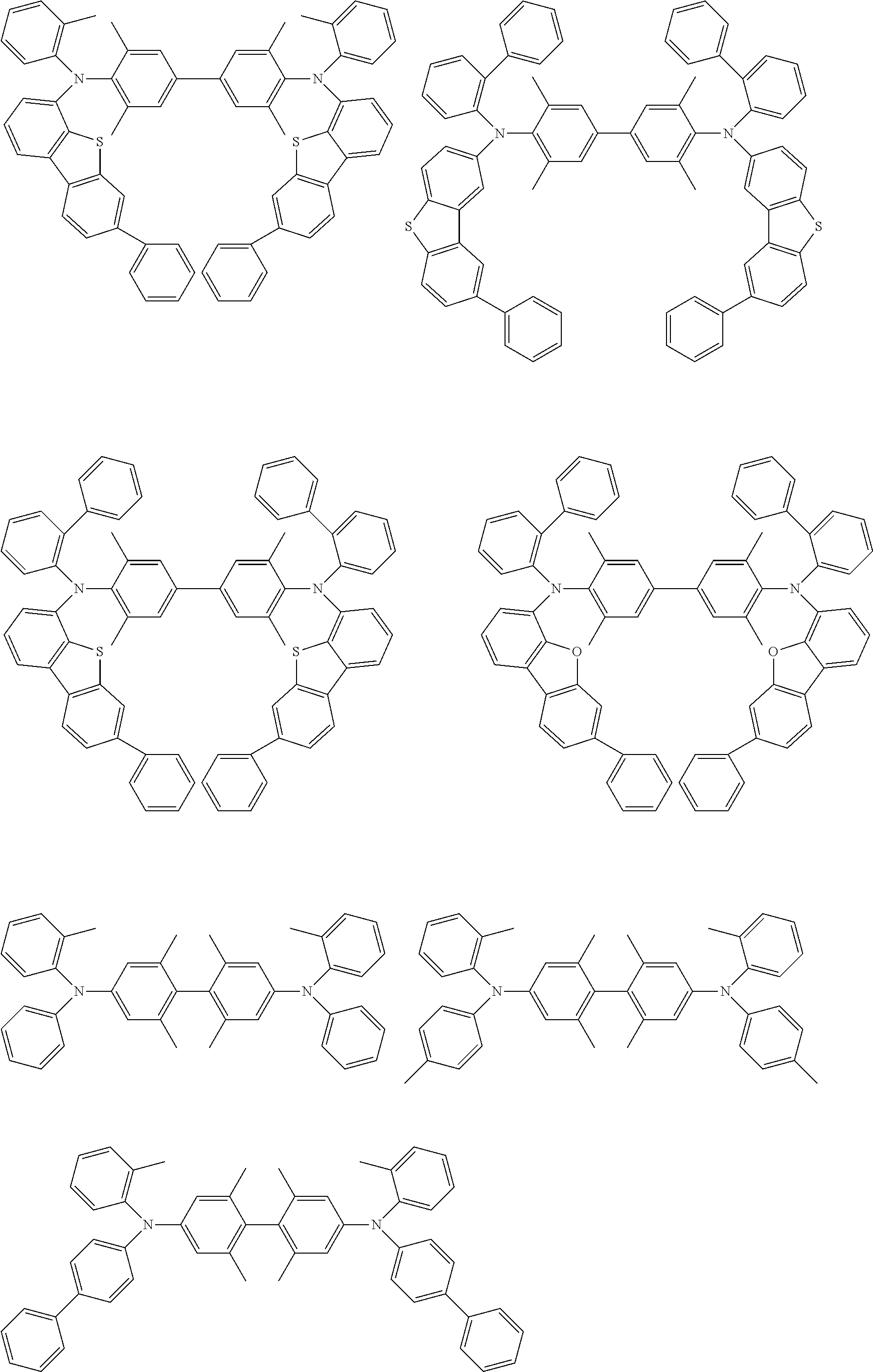

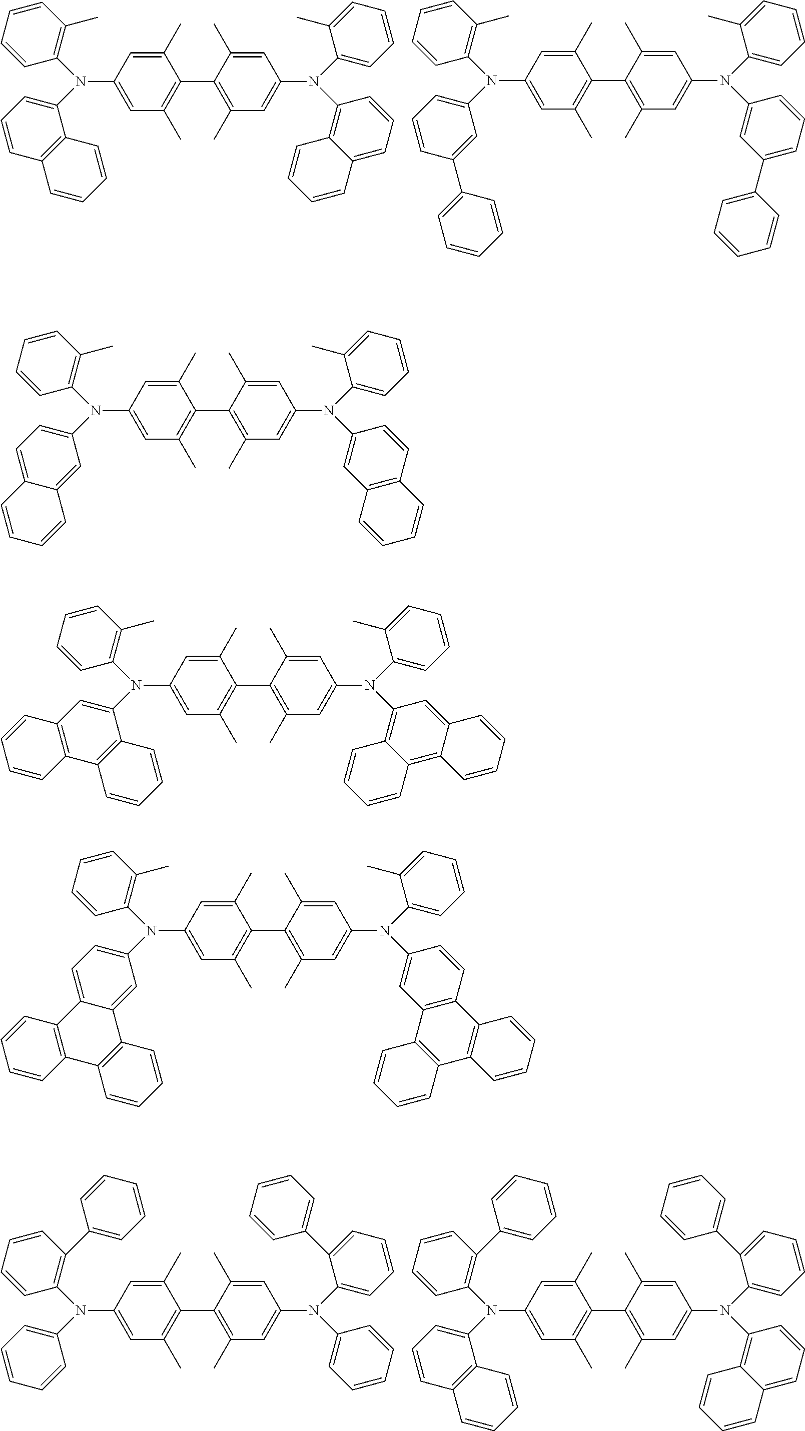

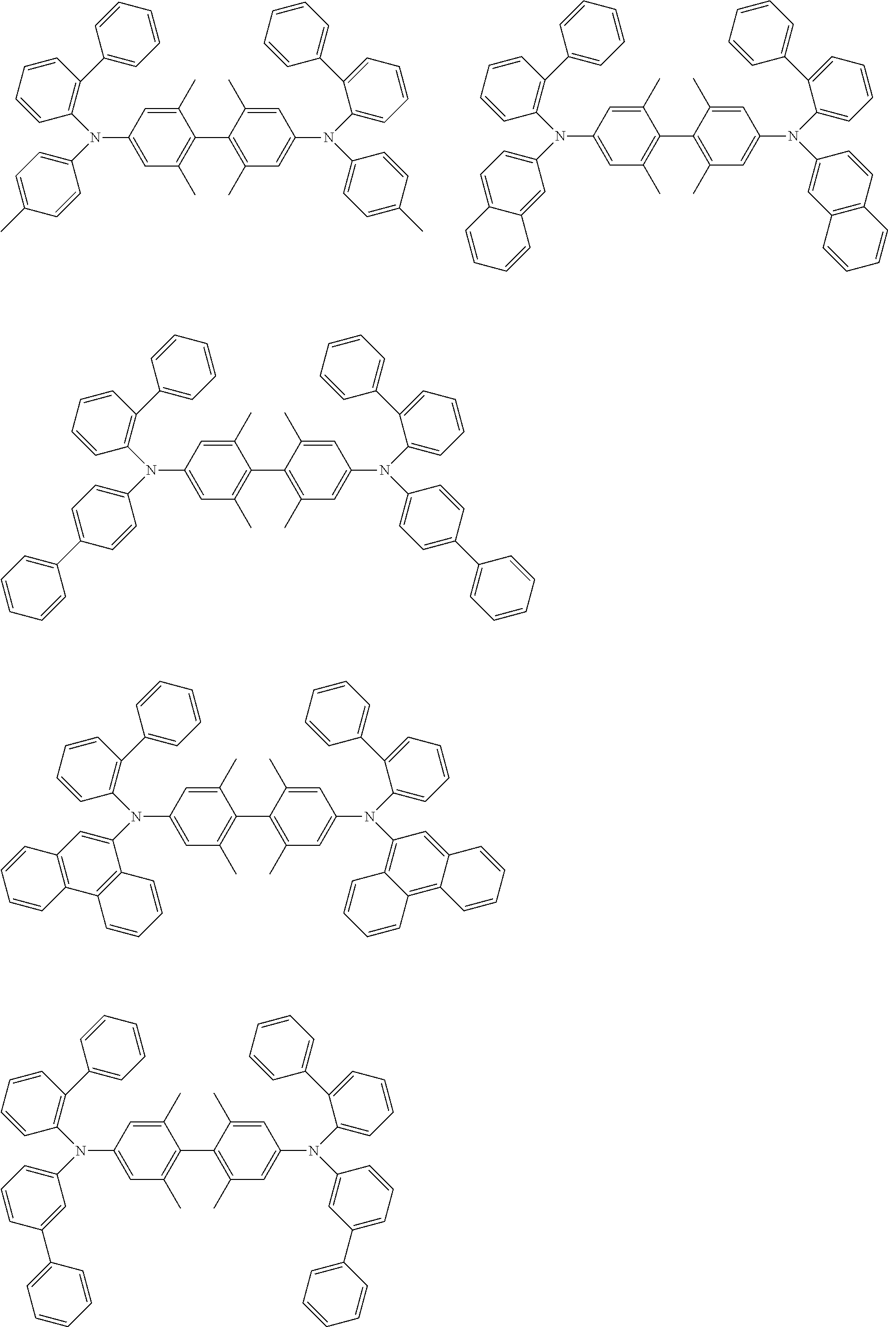

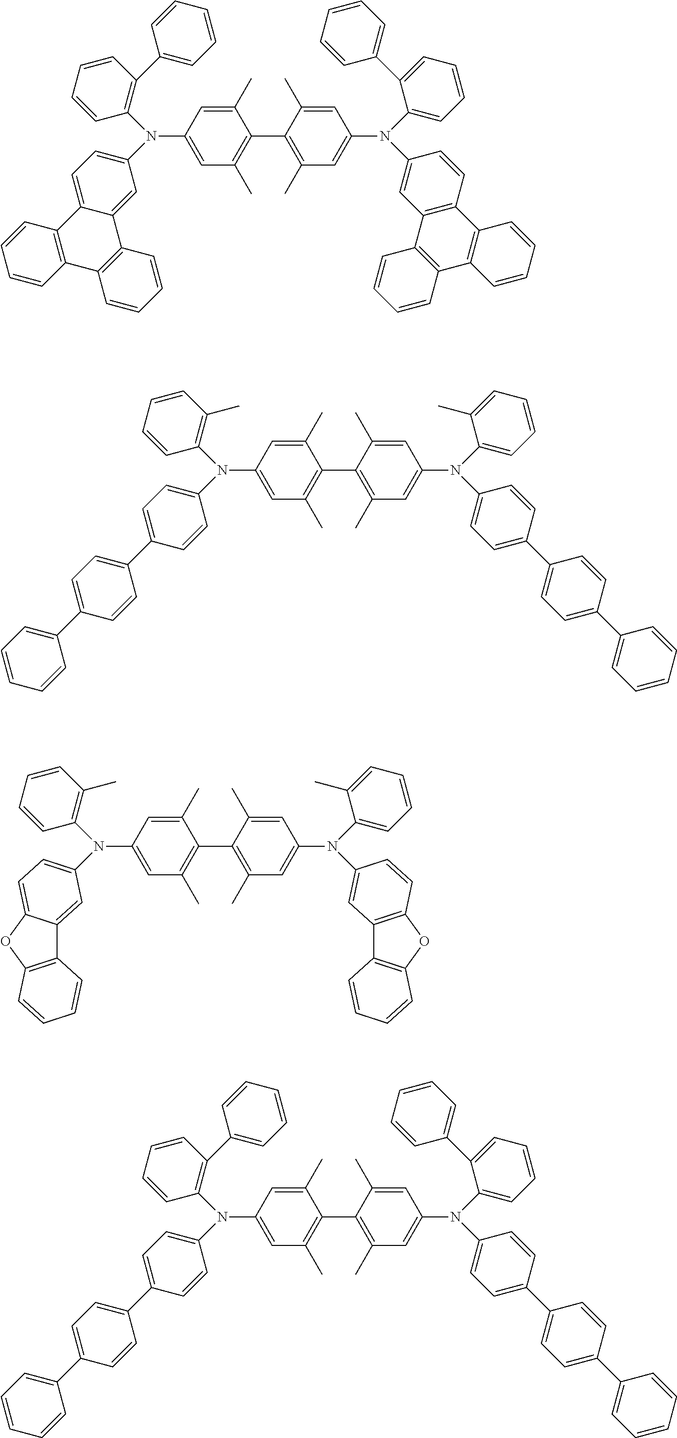 [SMILES: Cc1cc(N(c2ccc3c4ccccc4c4ccccc4c3c2)c2ccccc2-c2ccccc2)cc(C)c1-c1c(C)cc(N(c2ccc3c4ccccc4c4ccccc4c3c2)c2ccccc2-c2ccccc2)cc1C ; Cc1ccccc1N(c1ccc(-c2ccc(-c3ccccc3)cc2)cc1)c1cc(C)c(-c2c(C)cc(N(c3ccc(-c4ccc(-c5ccccc5)cc4)cc3)c3ccccc3C)cc2C)c(C)c1 ; Cc1ccccc1N(c1cc(C)c(-c2c(C)cc(N(c3ccc4oc5ccccc5c4c3)c3ccccc3C)cc2C)c(C)c1)c1ccc2oc3ccccc3c2c1 ; Cc1cc(N(c2ccc(-c3ccc(-c4ccccc4)cc3)cc2)c2ccccc2-c2ccccc2)cc(C)c1-c1c(C)cc(N(c2ccc(-c3ccc(-c4ccccc4)cc3)cc2)c2ccccc2-c2ccccc2)cc1C]

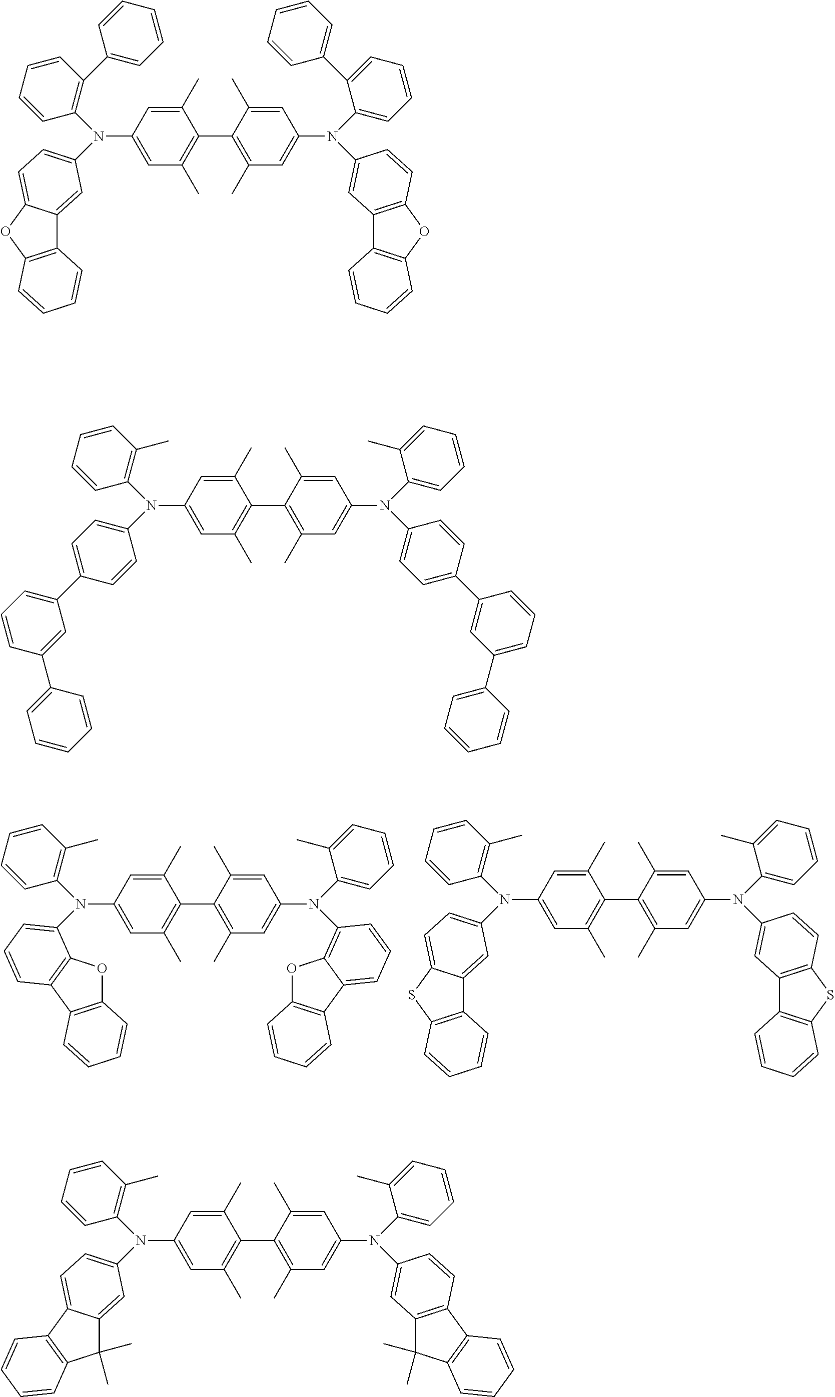

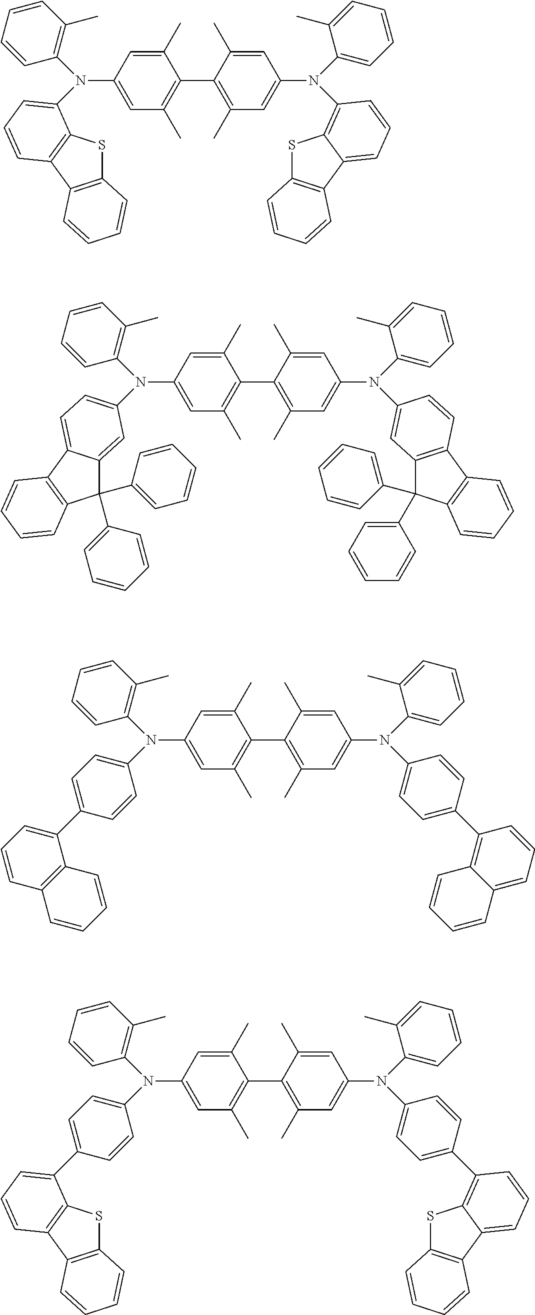 [SMILES: Cc1ccccc1N(c1cc(C)c(-c2c(C)cc(N(c3ccccc3C)c3cccc4c3sc3ccccc34)cc2C)c(C)c1)c1cccc2c1sc1ccccc12 ; Cc1ccccc1N(c1cc(C)c(-c2c(C)cc(N(c3ccc4c(c3)C(c3ccccc3)(c3ccccc3)c3ccccc3-4)c3ccccc3C)cc2C)c(C)c1)c1ccc2c(c1)C(c1ccccc1)(c1ccccc1)c1ccccc1-2 ; Cc1ccccc1N(c1ccc(-c2cccc3ccccc23)cc1)c1cc(C)c(-c2c(C)cc(N(c3ccc(-c4cccc5ccccc45)cc3)c3ccccc3C)cc2C)c(C)c1 ; Cc1ccccc1N(c1ccc(-c2cccc3c2sc2ccccc23)cc1)c1cc(C)c(-c2c(C)cc(N(c3ccc(-c4cccc5c4sc4ccccc45)cc3)c3ccccc3C)cc2C)c(C)c1]

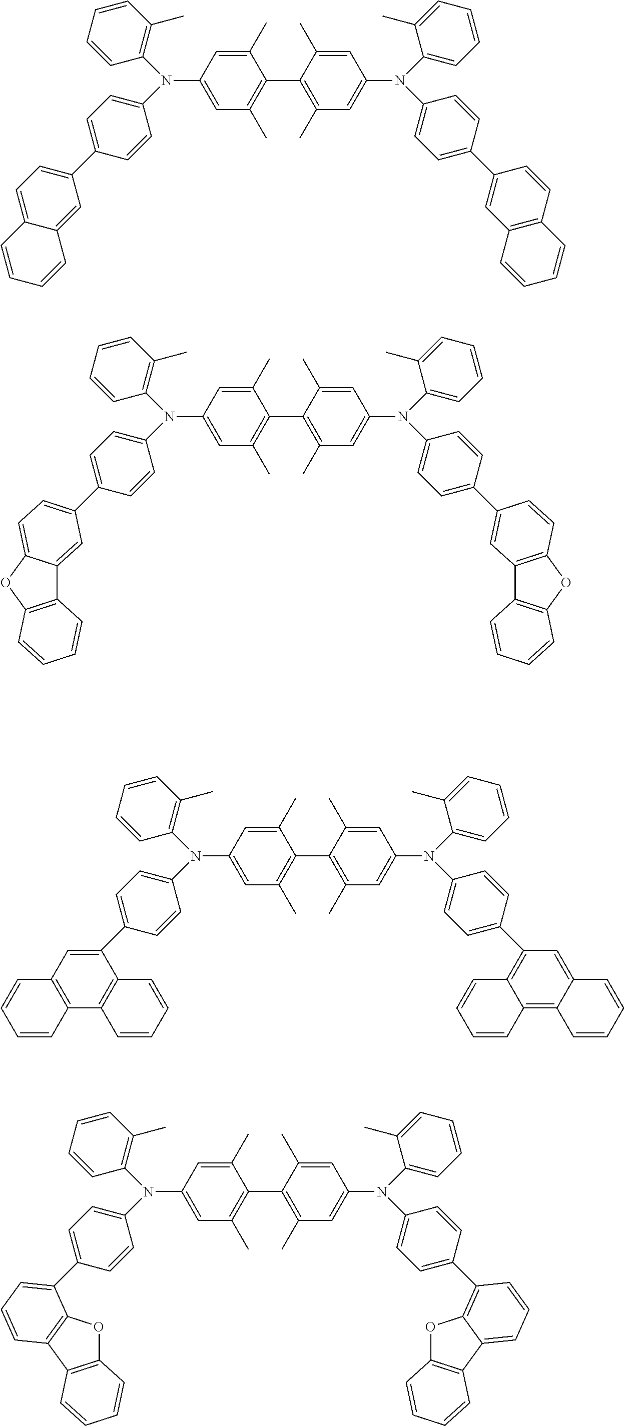 [SMILES: Cc1ccccc1N(c1ccc(-c2ccc3ccccc3c2)cc1)c1cc(C)c(-c2c(C)cc(N(c3ccc(-c4ccc5ccccc5c4)cc3)c3ccccc3C)cc2C)c(C)c1 ; Cc1ccccc1N(c1ccc(-c2ccc3oc4ccccc4c3c2)cc1)c1cc(C)c(-c2c(C)cc(N(c3ccc(-c4ccc5oc6ccccc6c5c4)cc3)c3ccccc3C)cc2C)c(C)c1 ; Cc1ccccc1N(c1ccc(-c2cc3ccccc3c3ccccc23)cc1)c1cc(C)c(-c2c(C)cc(N(c3ccc(-c4cc5ccccc5c5ccccc45)cc3)c3ccccc3C)cc2C)c(C)c1 ; Cc1ccccc1N(c1ccc(-c2cccc3c2oc2ccccc23)cc1)c1cc(C)c(-c2c(C)cc(N(c3ccc(-c4cccc5c4oc4ccccc45)cc3)c3ccccc3C)cc2C)c(C)c1]

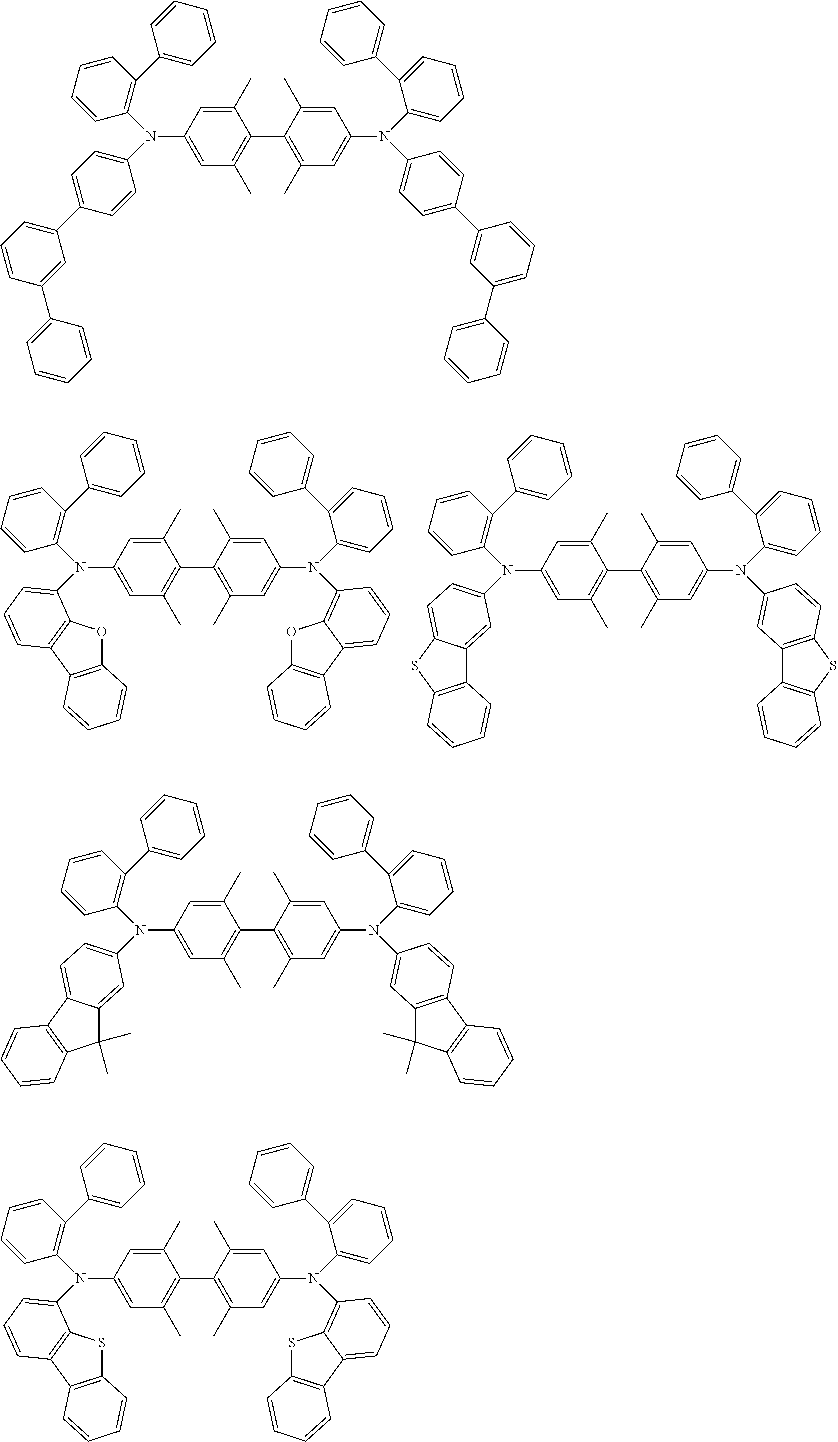

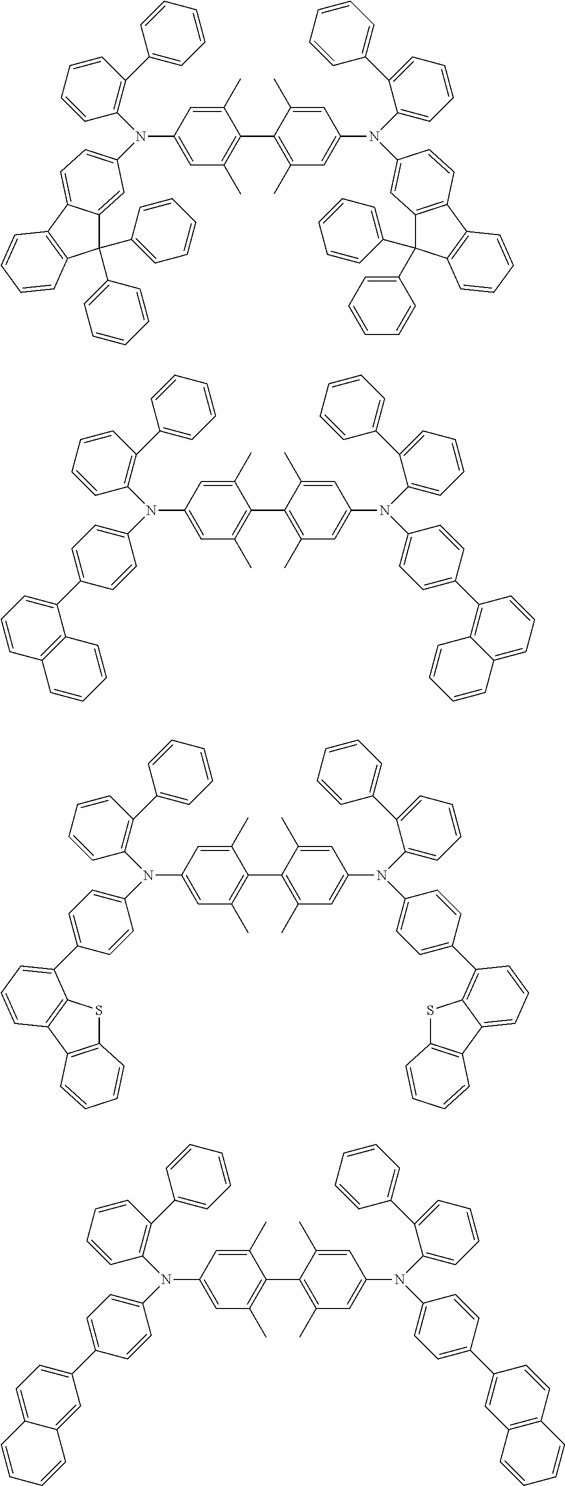

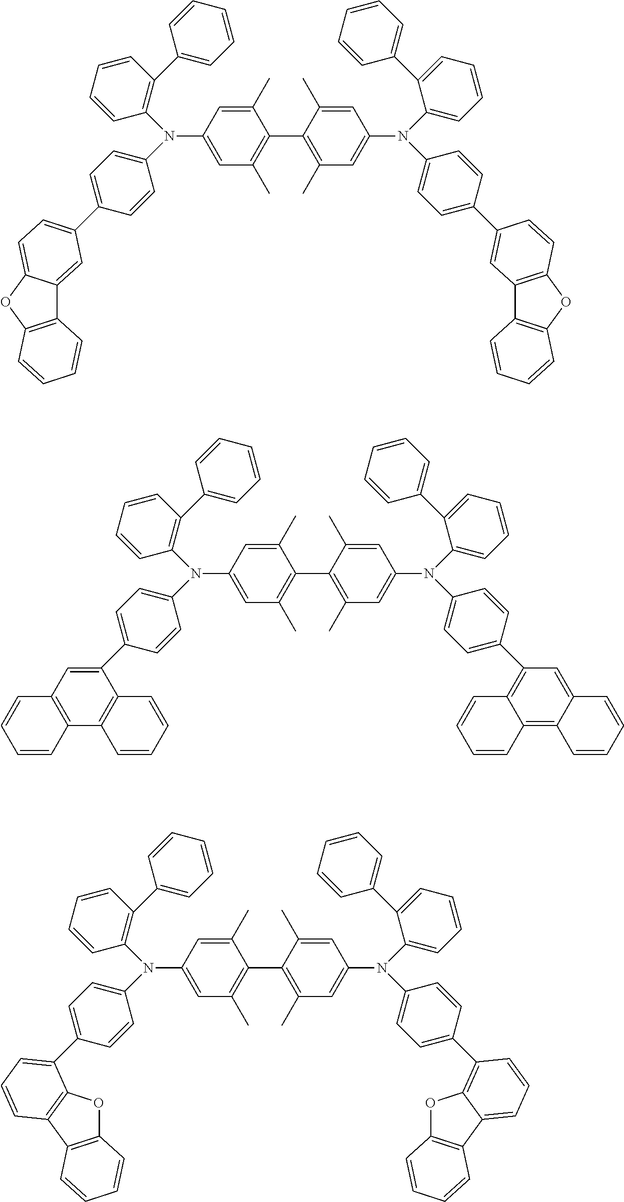 [SMILES: Cc1cc(N(c2ccc(-c3ccc4oc5ccccc5c4c3)cc2)c2ccccc2-c2ccccc2)cc(C)c1-c1c(C)cc(N(c2ccc(-c3ccc4oc5ccccc5c4c3)cc2)c2ccccc2-c2ccccc2)cc1C ; Cc1cc(N(c2ccc(-c3cc4ccccc4c4ccccc34)cc2)c2ccccc2-c2ccccc2)cc(C)c1-c1c(C)cc(N(c2ccc(-c3cc4ccccc4c4ccccc34)cc2)c2ccccc2-c2ccccc2)cc1C ; Cc1cc(N(c2ccc(-c3cccc4c3oc3ccccc34)cc2)c2ccccc2-c2ccccc2)cc(C)c1-c1c(C)cc(N(c2ccc(-c3cccc4c3oc3ccccc34)cc2)c2ccccc2-c2ccccc2)cc1C]

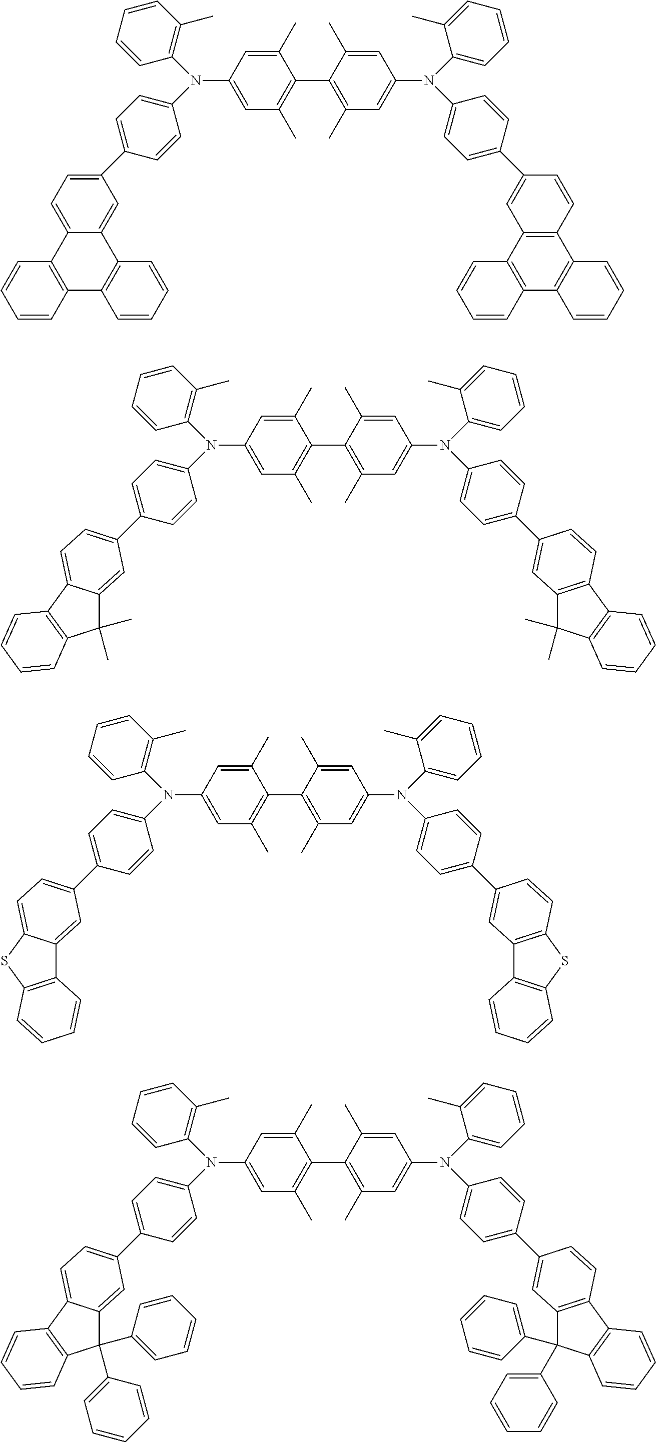

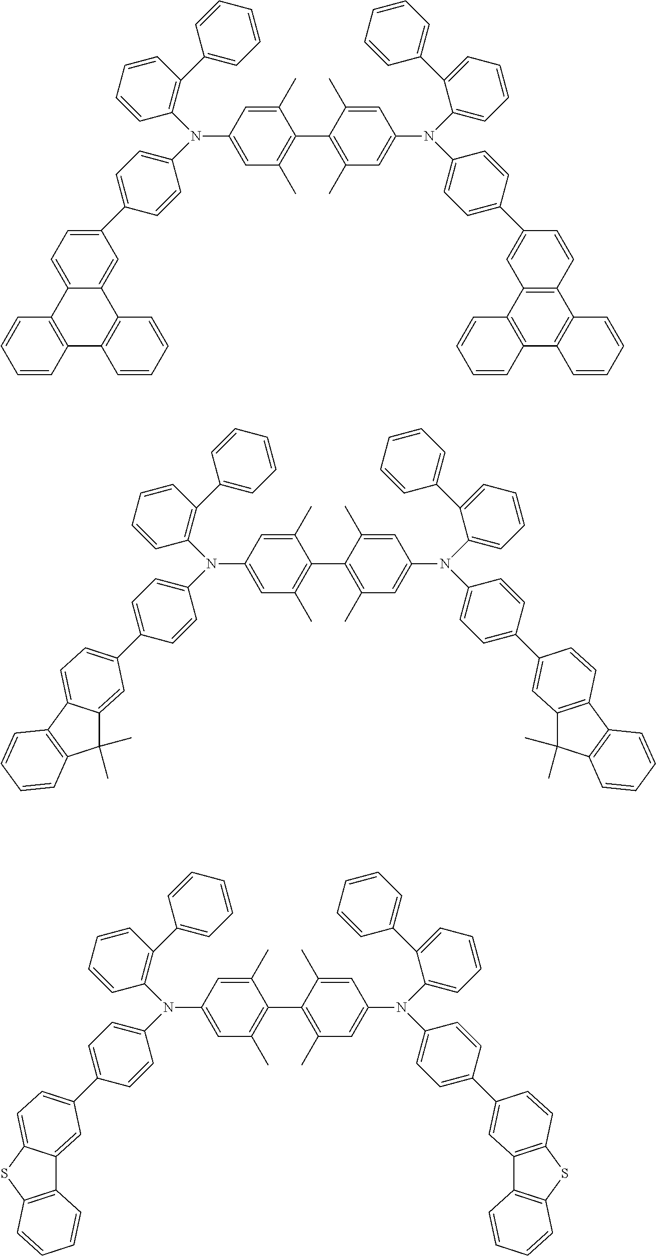 [SMILES: Cc1cc(N(c2ccc(-c3ccc4c5ccccc5c5ccccc5c4c3)cc2)c2ccccc2-c2ccccc2)cc(C)c1-c1c(C)cc(N(c2ccc(-c3ccc4c5ccccc5c5ccccc5c4c3)cc2)c2ccccc2-c2ccccc2)cc1C ; Cc1cc(N(c2ccc(-c3ccc4c(c3)C(C)(C)c3ccccc3-4)cc2)c2ccccc2-c2ccccc2)cc(C)c1-c1c(C)cc(N(c2ccc(-c3ccc4c(c3)C(C)(C)c3ccccc3-4)cc2)c2ccccc2-c2ccccc2)cc1C ; Cc1cc(N(c2ccc(-c3ccc4sc5ccccc5c4c3)cc2)c2ccccc2-c2ccccc2)cc(C)c1-c1c(C)cc(N(c2ccc(-c3ccc4sc5ccccc5c4c3)cc2)c2ccccc2-c2ccccc2)cc1C]

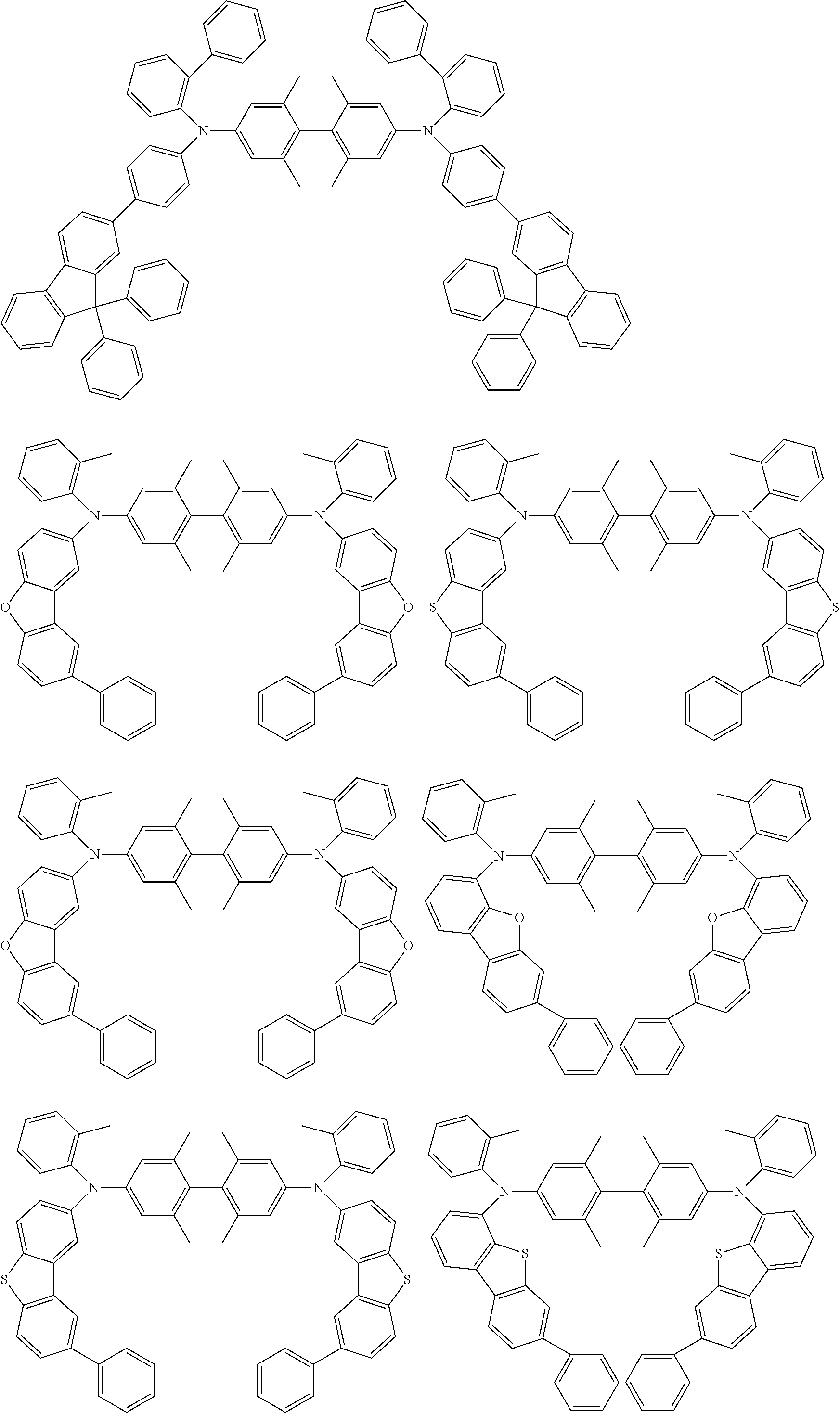

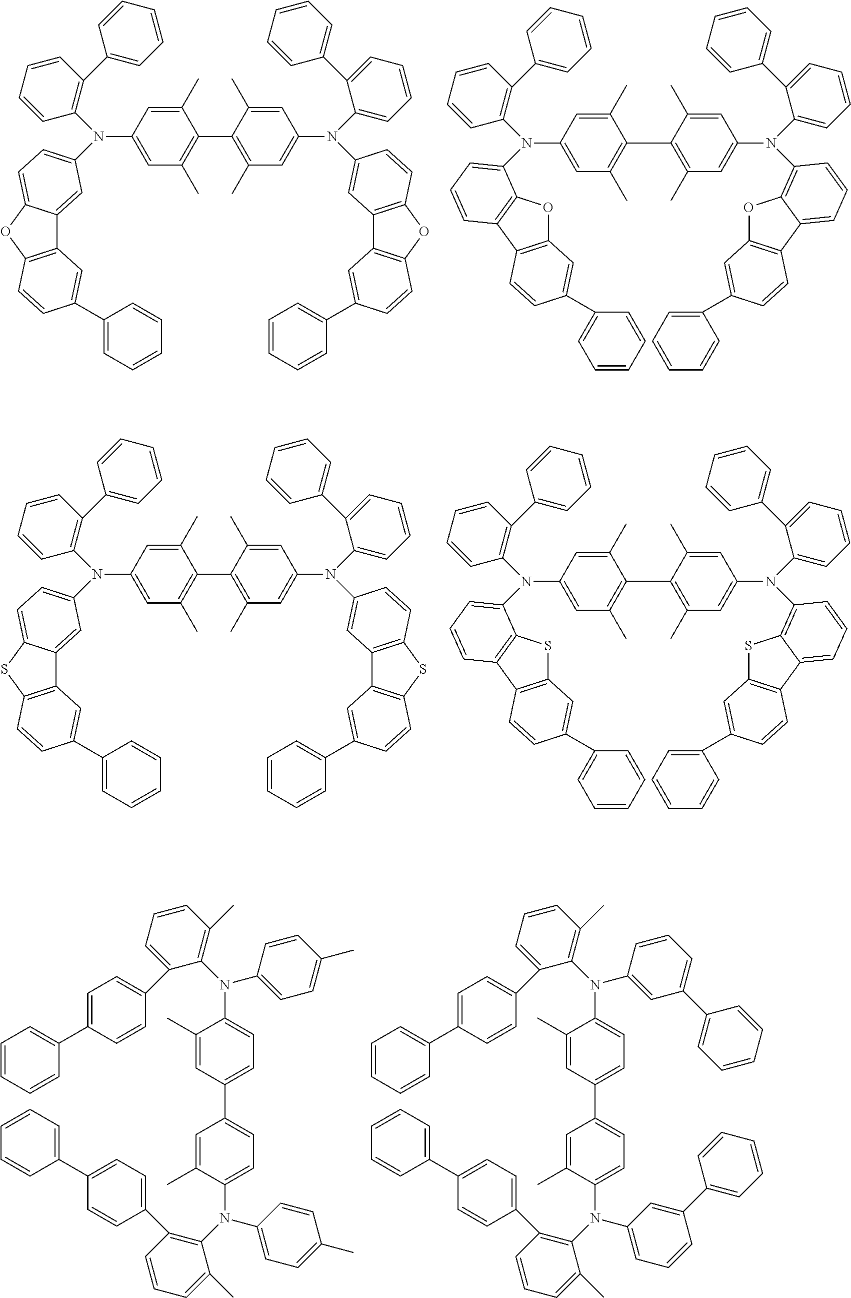

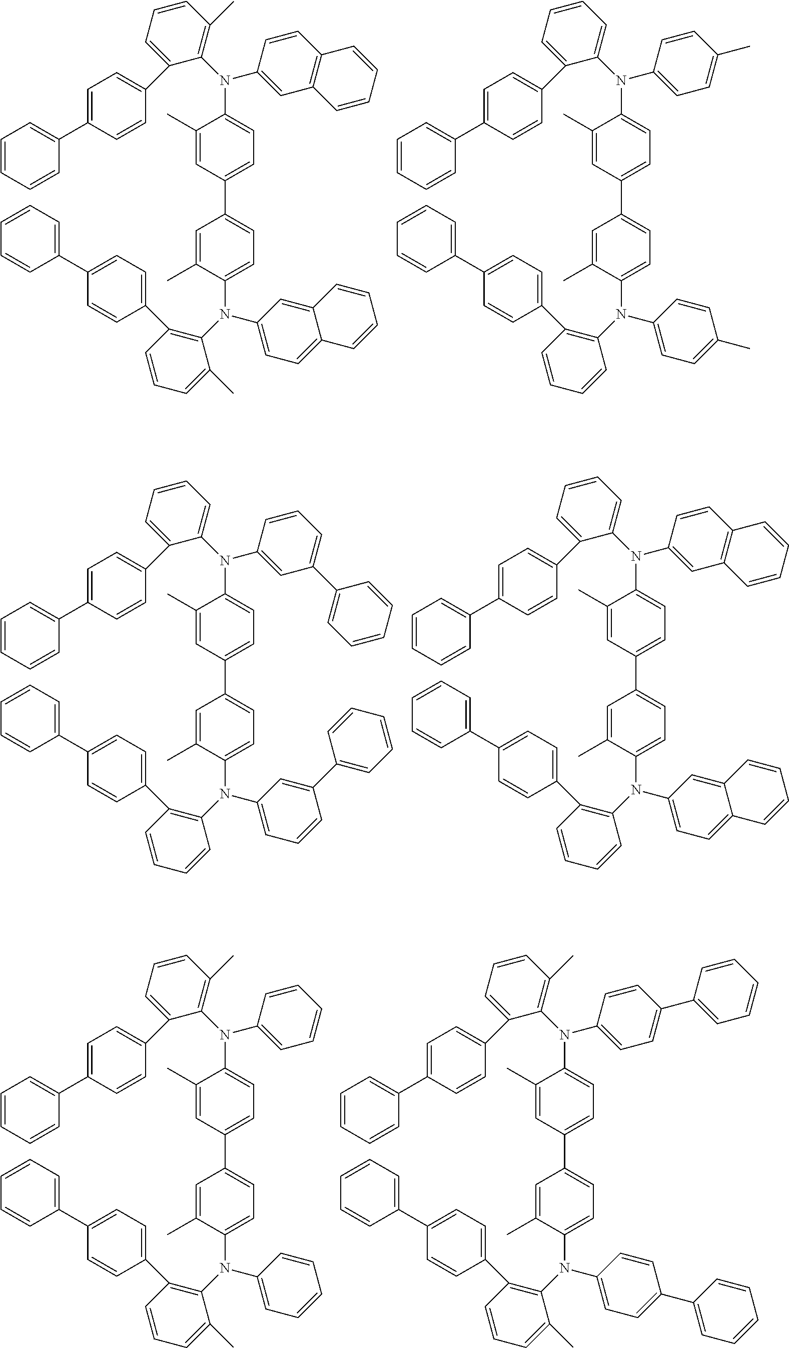

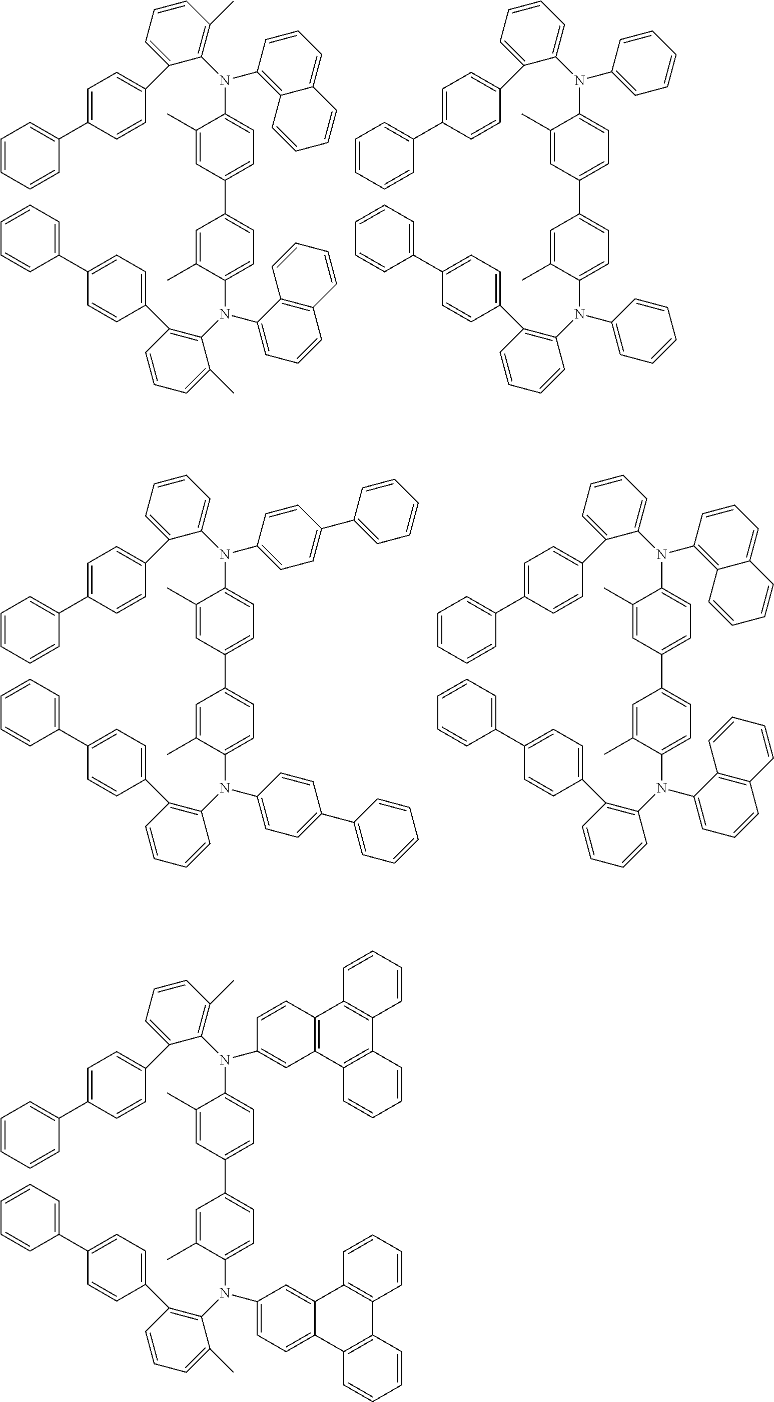

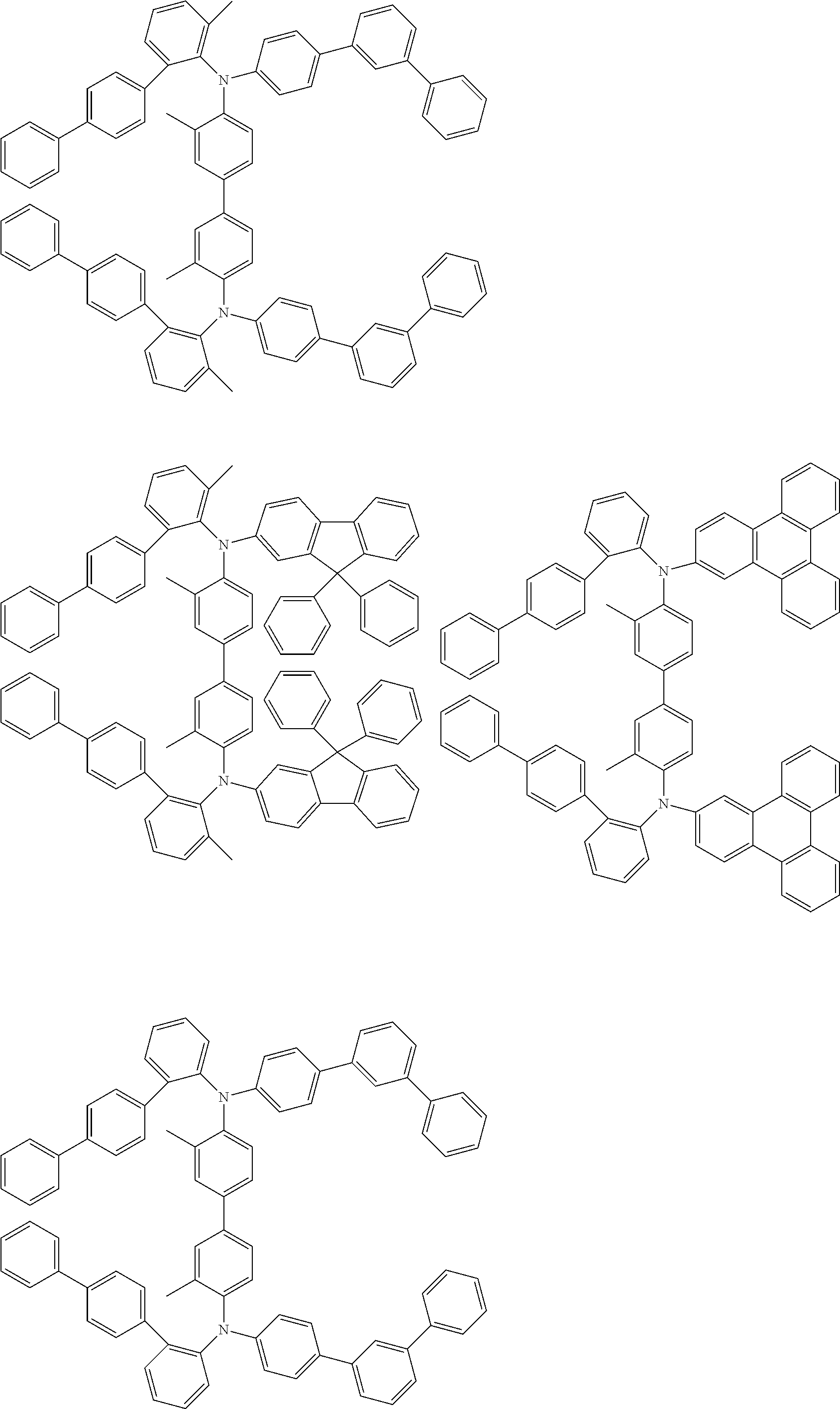

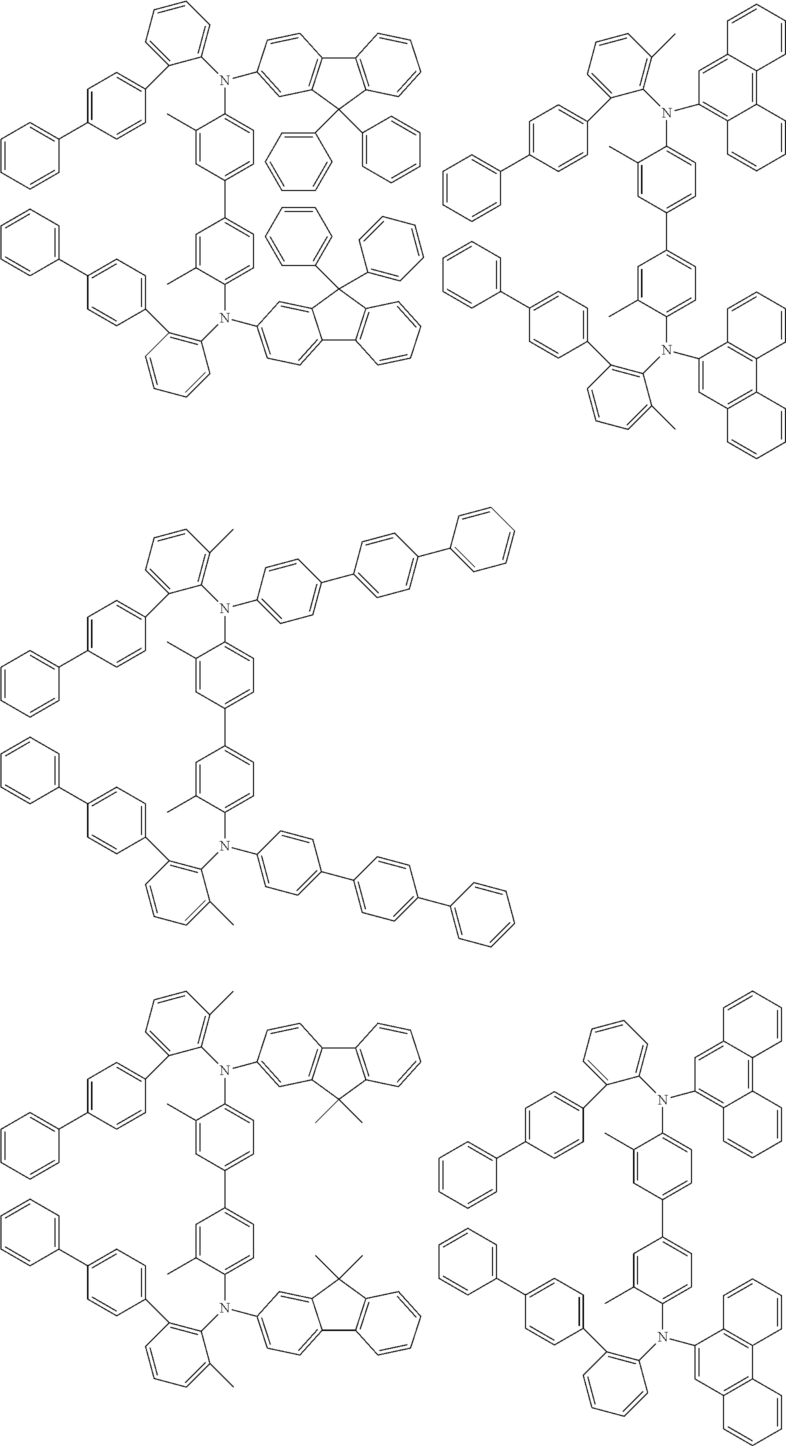

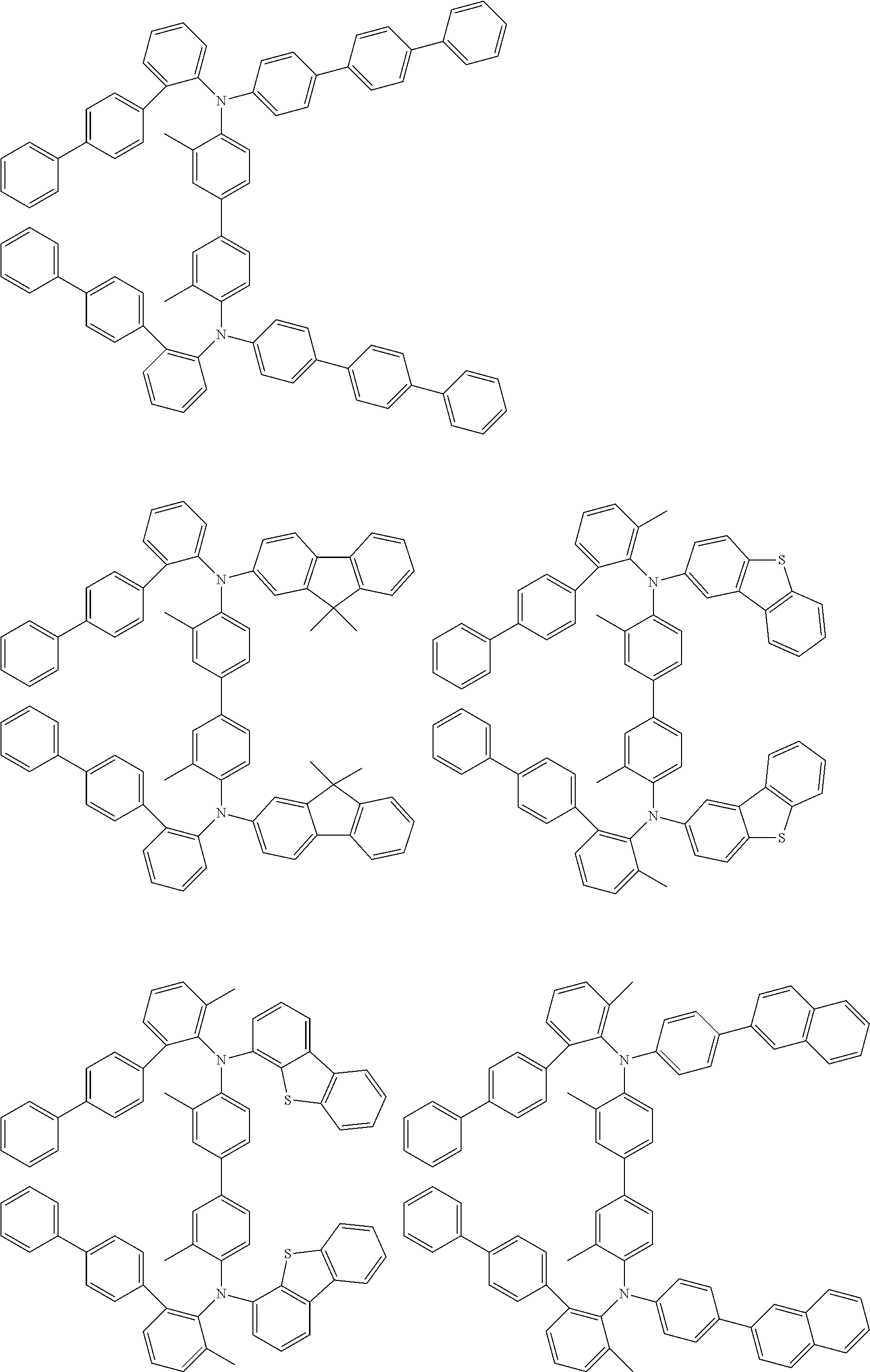

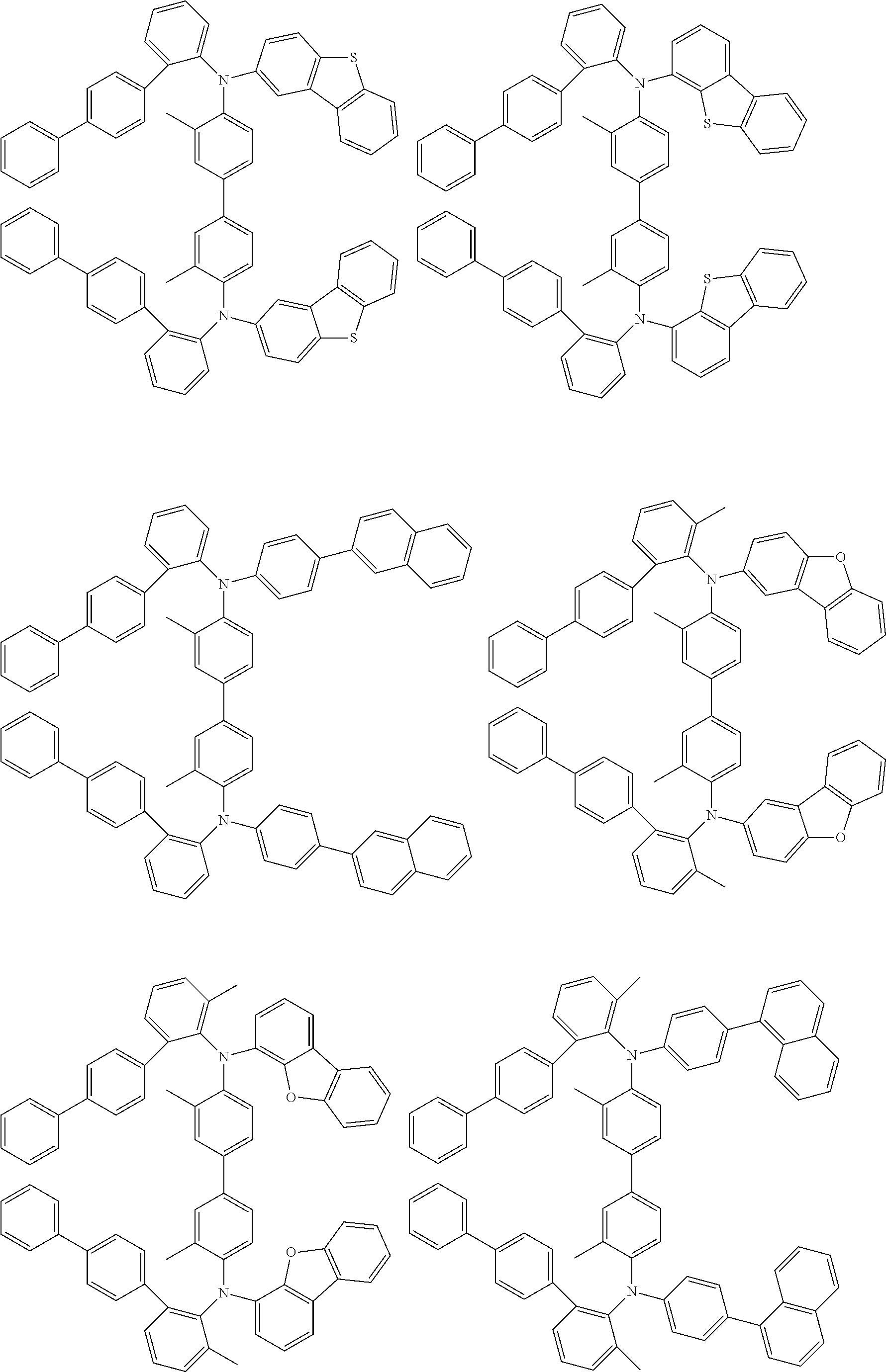

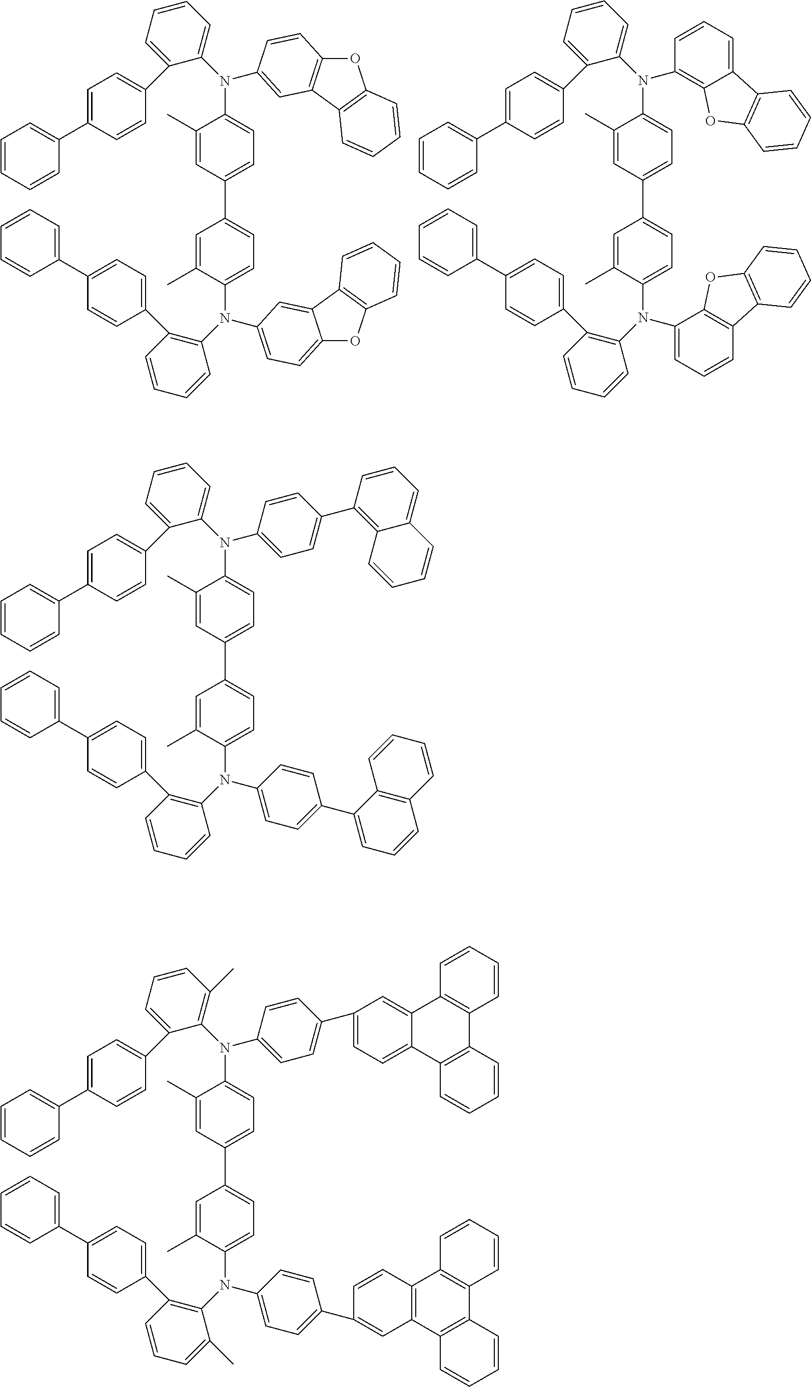

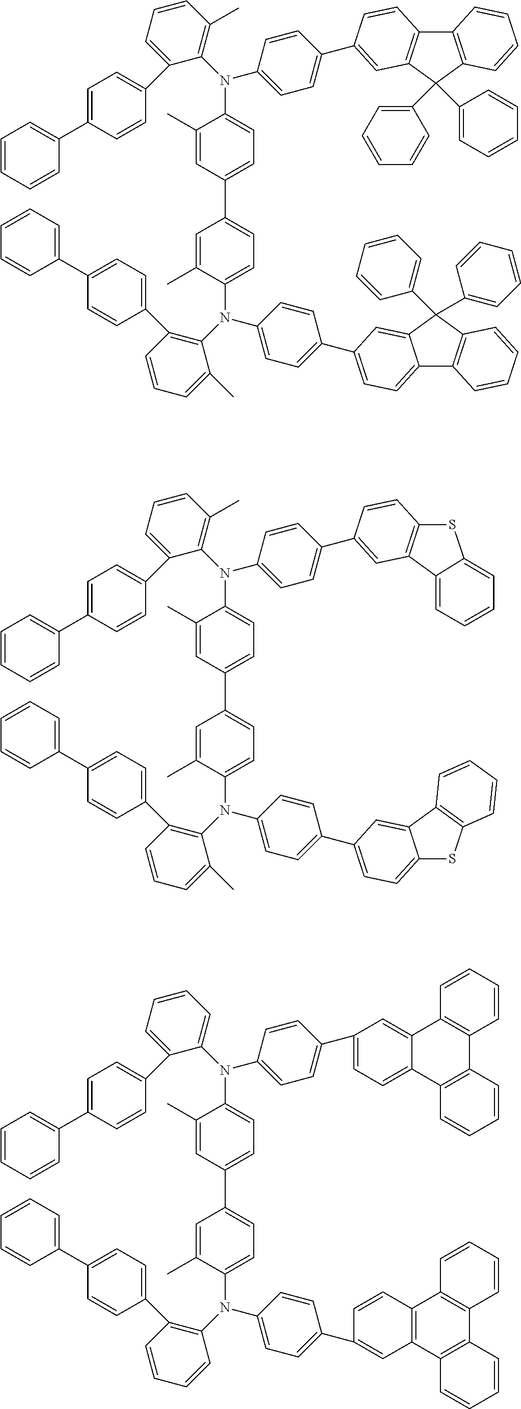

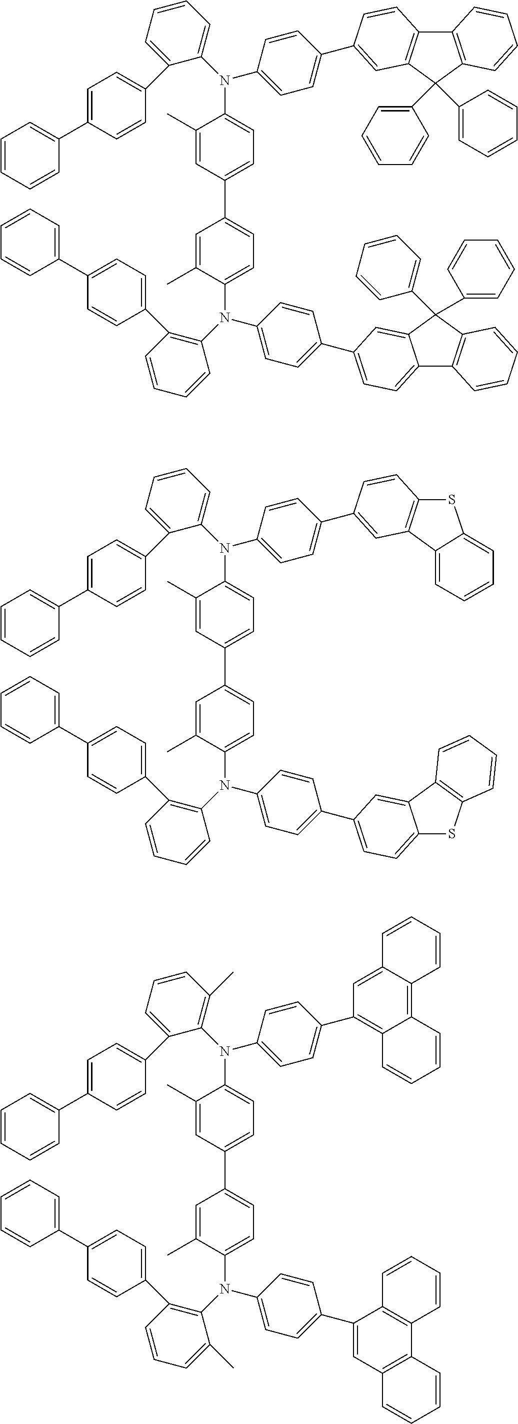

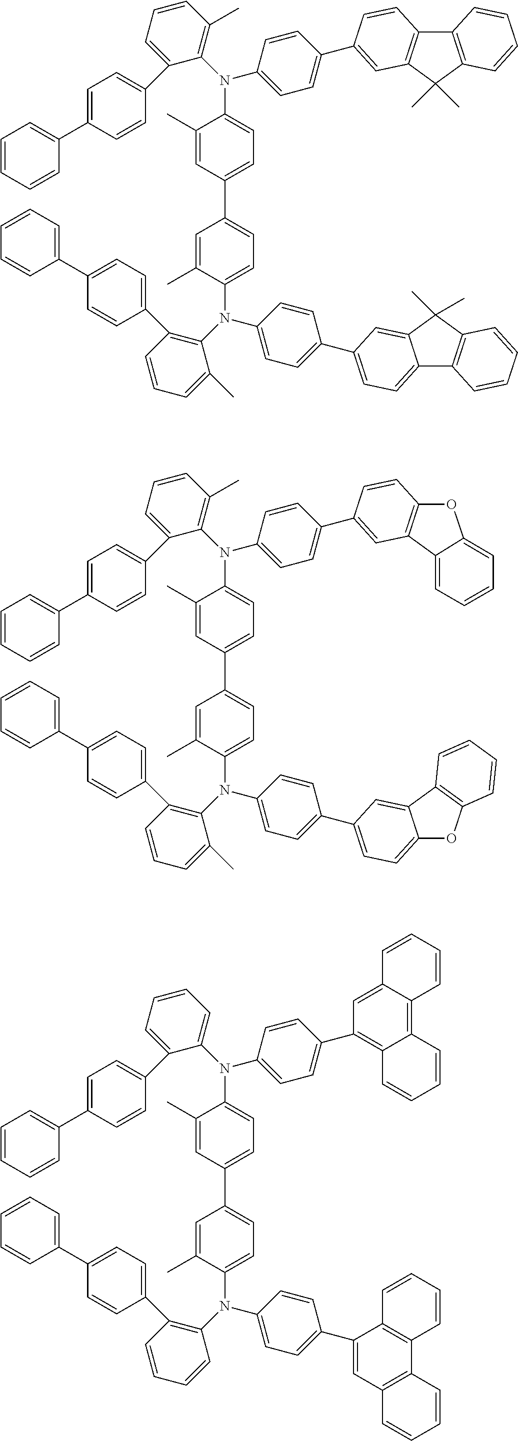 [SMILES: Cc1cc(-c2ccc(N(c3ccc(-c4ccc5c(c4)C(C)(C)c4ccccc4-5)cc3)c3c(C)cccc3-c3ccc(-c4ccccc4)cc3)c(C)c2)ccc1N(c1ccc(-c2ccc3c(c2)C(C)(C)c2ccccc2-3)cc1)c1c(C)cccc1-c1ccc(-c2ccccc2)cc1 ; Cc1cc(-c2ccc(N(c3ccc(-c4ccc5oc6ccccc6c5c4)cc3)c3c(C)cccc3-c3ccc(-c4ccccc4)cc3)c(C)c2)ccc1N(c1ccc(-c2ccc3oc4ccccc4c3c2)cc1)c1c(C)cccc1-c1ccc(-c2ccccc2)cc1 ; Cc1cc(-c2ccc(N(c3ccc(-c4cc5ccccc5c5ccccc45)cc3)c3ccccc3-c3ccc(-c4ccccc4)cc3)c(C)c2)ccc1N(c1ccc(-c2cc3ccccc3c3ccccc23)cc1)c1ccccc1-c1ccc(-c2ccccc2)cc1]

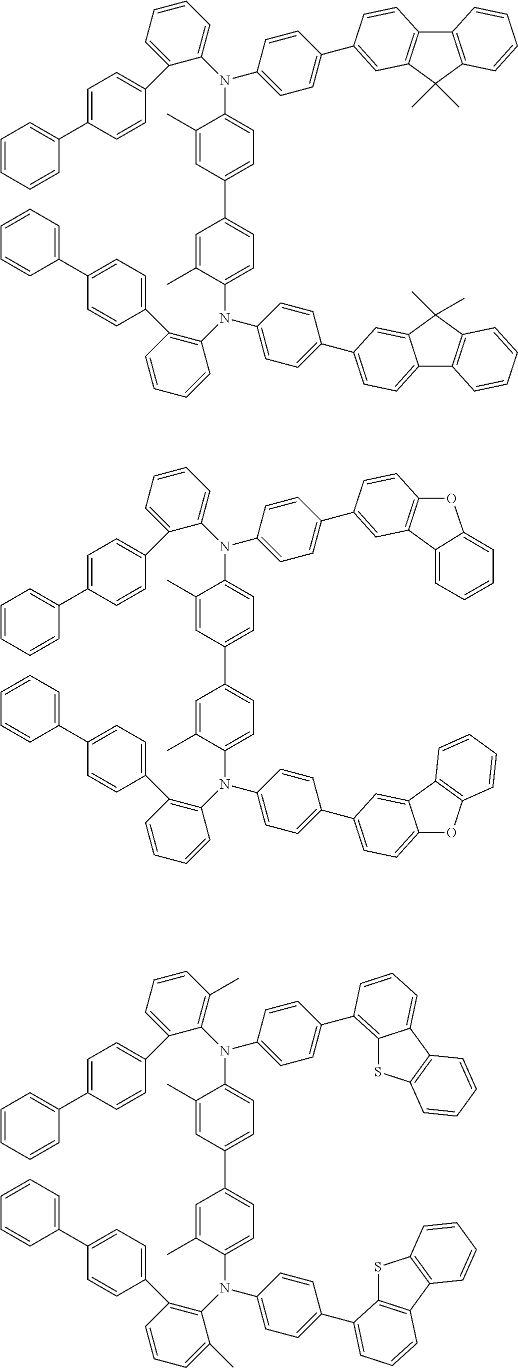 [SMILES: Cc1cc(-c2ccc(N(c3ccc(-c4ccc5c(c4)C(C)(C)c4ccccc4-5)cc3)c3ccccc3-c3ccc(-c4ccccc4)cc3)c(C)c2)ccc1N(c1ccc(-c2ccc3c(c2)C(C)(C)c2ccccc2-3)cc1)c1ccccc1-c1ccc(-c2ccccc2)cc1 ; Cc1cc(-c2ccc(N(c3ccc(-c4ccc5oc6ccccc6c5c4)cc3)c3ccccc3-c3ccc(-c4ccccc4)cc3)c(C)c2)ccc1N(c1ccc(-c2ccc3oc4ccccc4c3c2)cc1)c1ccccc1-c1ccc(-c2ccccc2)cc1 ; Cc1cc(-c2ccc(N(c3ccc(-c4cccc5c4sc4ccccc45)cc3)c3c(C)cccc3-c3ccc(-c4ccccc4)cc3)c(C)c2)ccc1N(c1ccc(-c2cccc3c2sc2ccccc23)cc1)c1c(C)cccc1-c1ccc(-c2ccccc2)cc1]

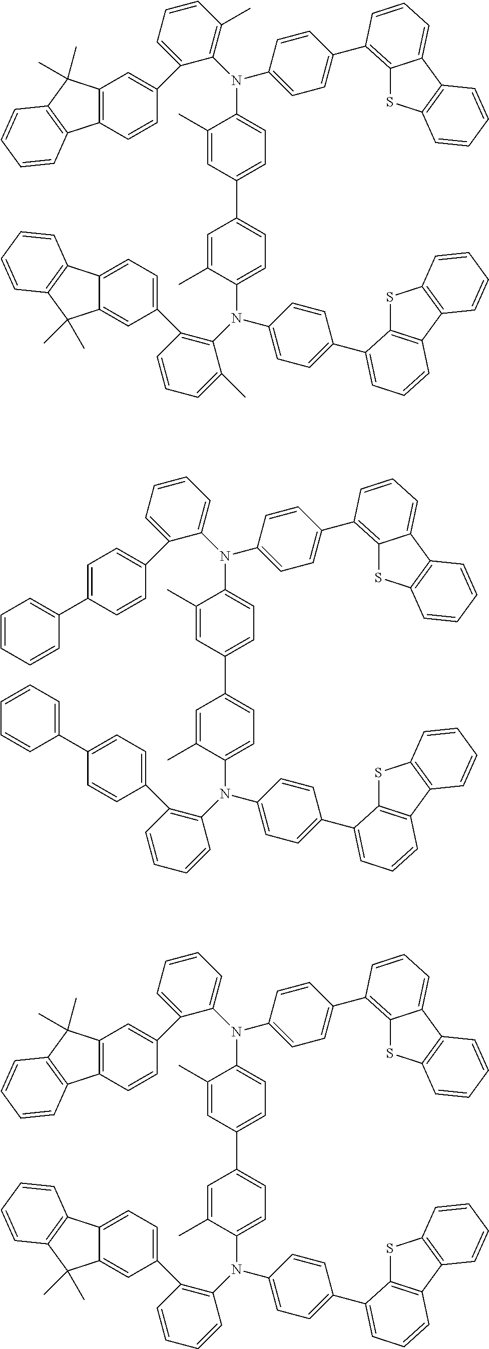 [SMILES: Cc1cc(-c2ccc(N(c3ccc(-c4cccc5c4sc4ccccc45)cc3)c3c(C)cccc3-c3ccc4c(c3)C(C)(C)c3ccccc3-4)c(C)c2)ccc1N(c1ccc(-c2cccc3c2sc2ccccc23)cc1)c1c(C)cccc1-c1ccc2c(c1)C(C)(C)c1ccccc1-2 ; Cc1cc(-c2ccc(N(c3ccc(-c4cccc5c4sc4ccccc45)cc3)c3ccccc3-c3ccc(-c4ccccc4)cc3)c(C)c2)ccc1N(c1ccc(-c2cccc3c2sc2ccccc23)cc1)c1ccccc1-c1ccc(-c2ccccc2)cc1 ; Cc1cc(-c2ccc(N(c3ccc(-c4cccc5c4sc4ccccc45)cc3)c3ccccc3-c3ccc4c(c3)C(C)(C)c3ccccc3-4)c(C)c2)ccc1N(c1ccc(-c2cccc3c2sc2ccccc23)cc1)c1ccccc1-c1ccc2c(c1)C(C)(C)c1ccccc1-2]

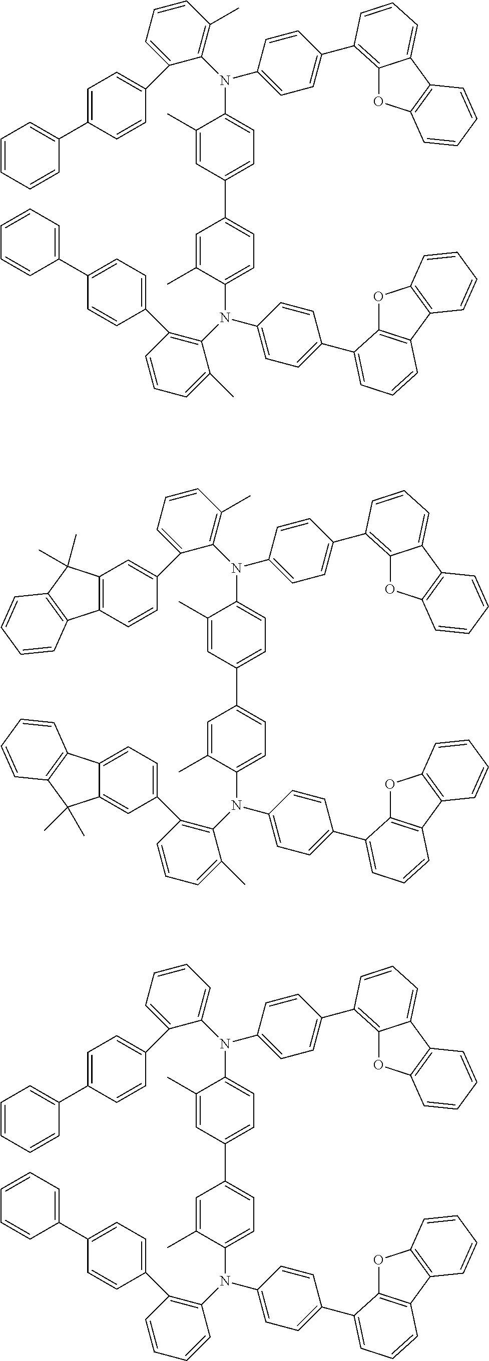 [SMILES: Cc1cc(-c2ccc(N(c3ccc(-c4cccc5c4oc4ccccc45)cc3)c3c(C)cccc3-c3ccc(-c4ccccc4)cc3)c(C)c2)ccc1N(c1ccc(-c2cccc3c2oc2ccccc23)cc1)c1c(C)cccc1-c1ccc(-c2ccccc2)cc1 ; Cc1cc(-c2ccc(N(c3ccc(-c4cccc5c4oc4ccccc45)cc3)c3c(C)cccc3-c3ccc4c(c3)C(C)(C)c3ccccc3-4)c(C)c2)ccc1N(c1ccc(-c2cccc3c2oc2ccccc23)cc1)c1c(C)cccc1-c1ccc2c(c1)C(C)(C)c1ccccc1-2 ; Cc1cc(-c2ccc(N(c3ccc(-c4cccc5c4oc4ccccc45)cc3)c3ccccc3-c3ccc(-c4ccccc4)cc3)c(C)c2)ccc1N(c1ccc(-c2cccc3c2oc2ccccc23)cc1)c1ccccc1-c1ccc(-c2ccccc2)cc1]

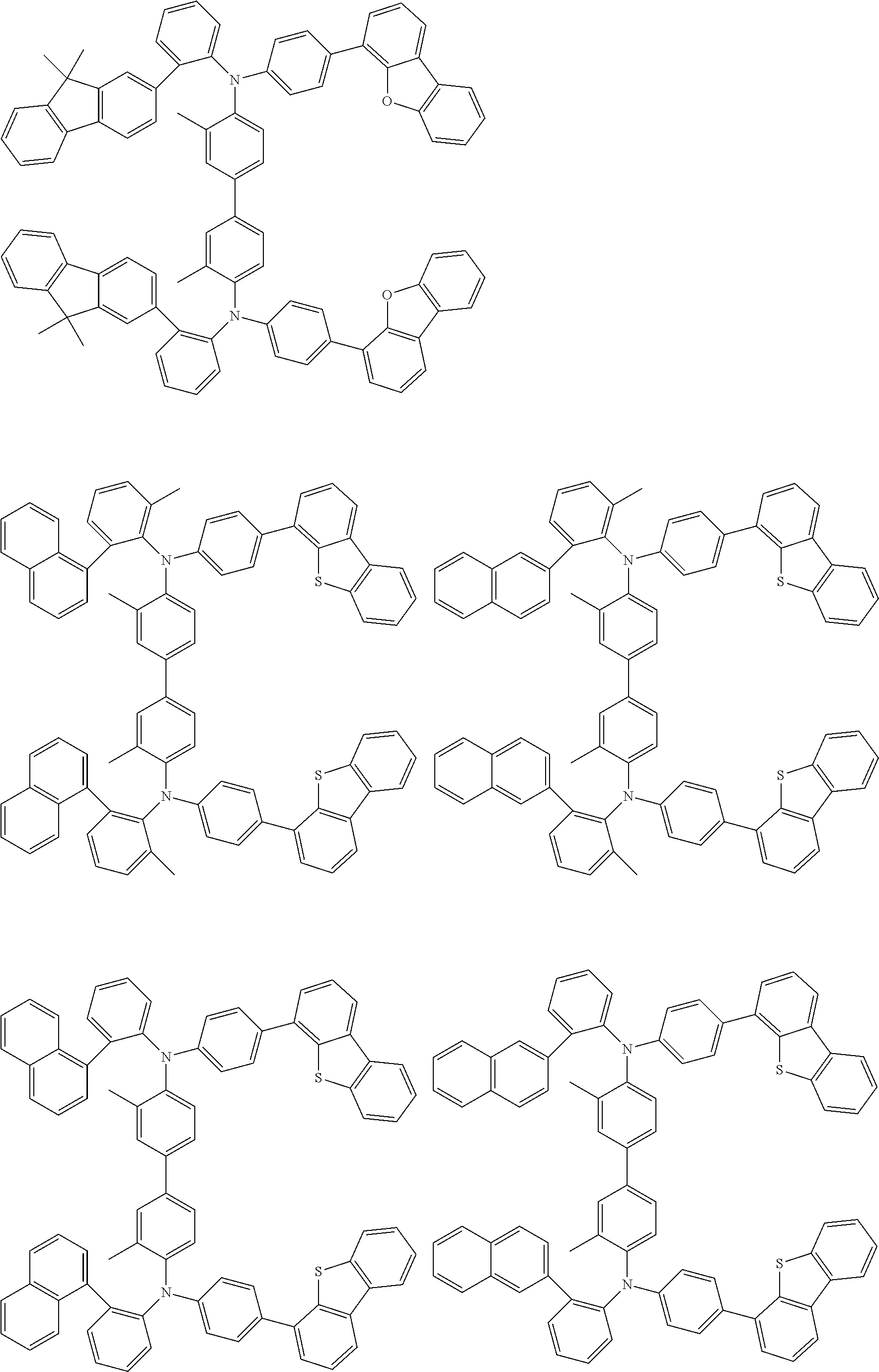

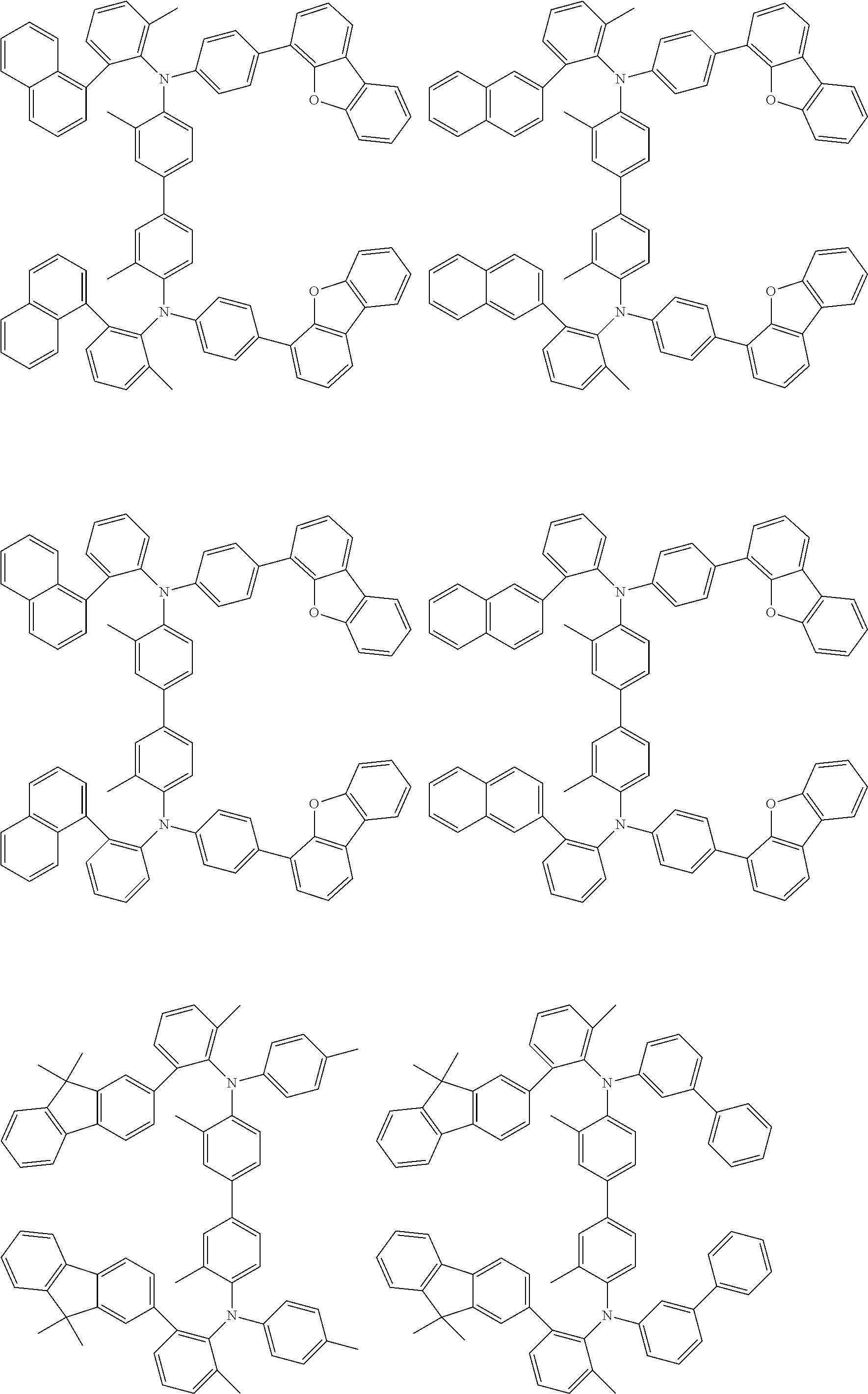

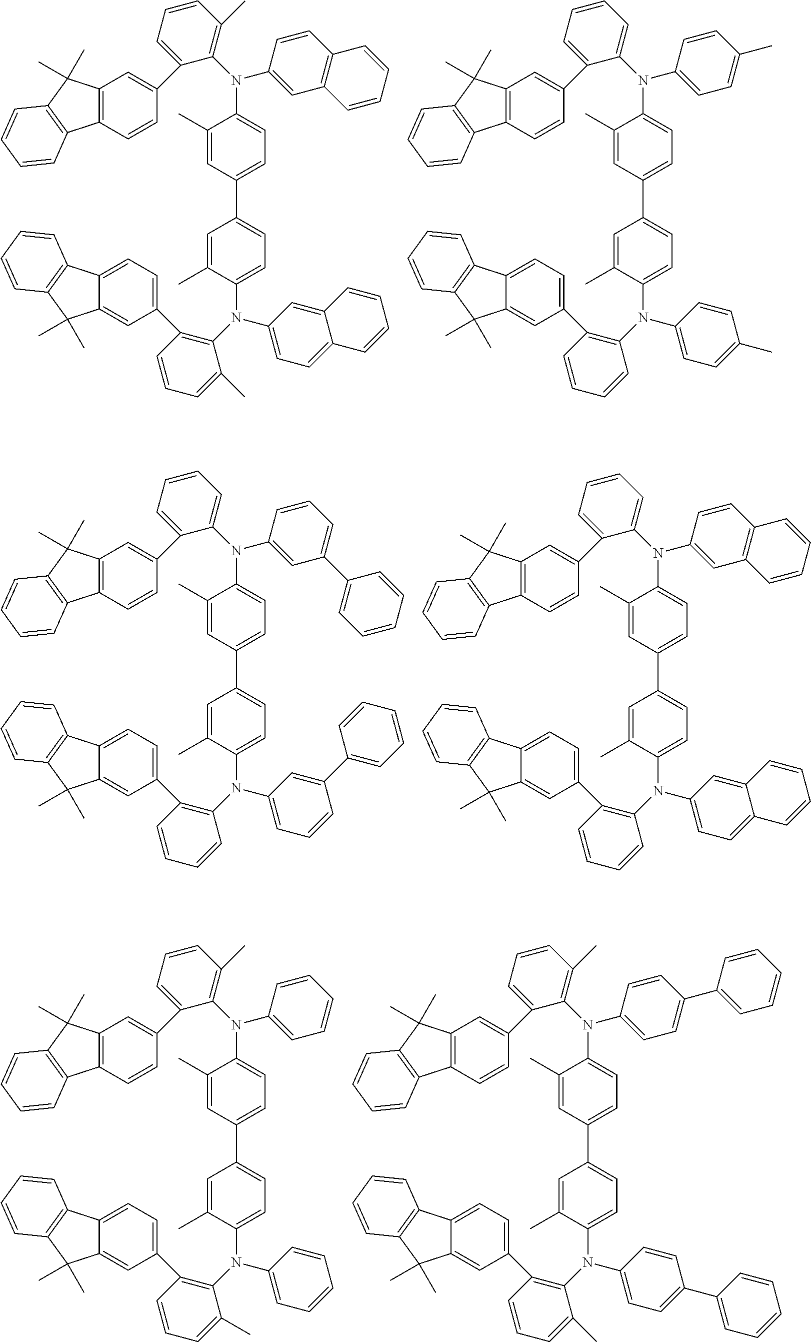

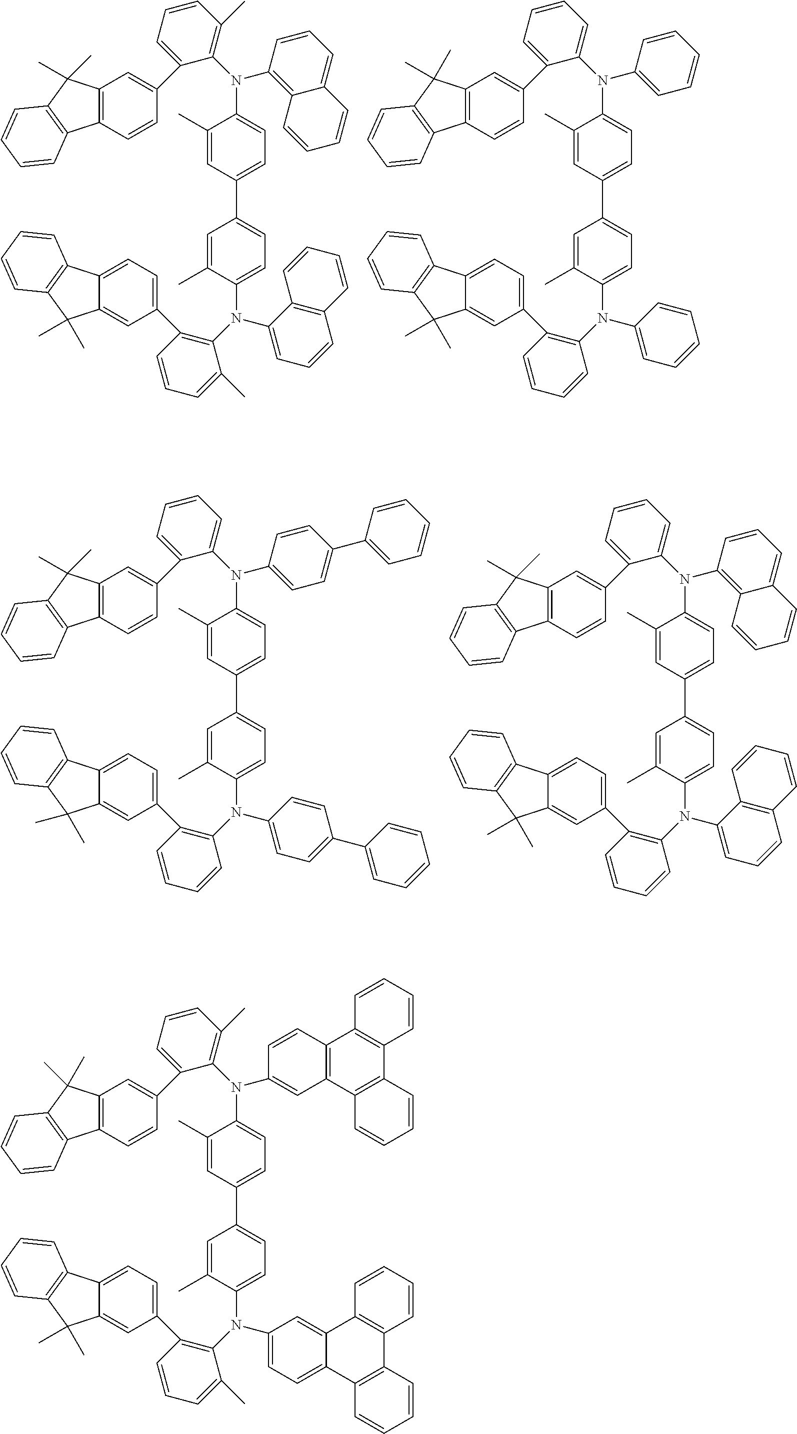

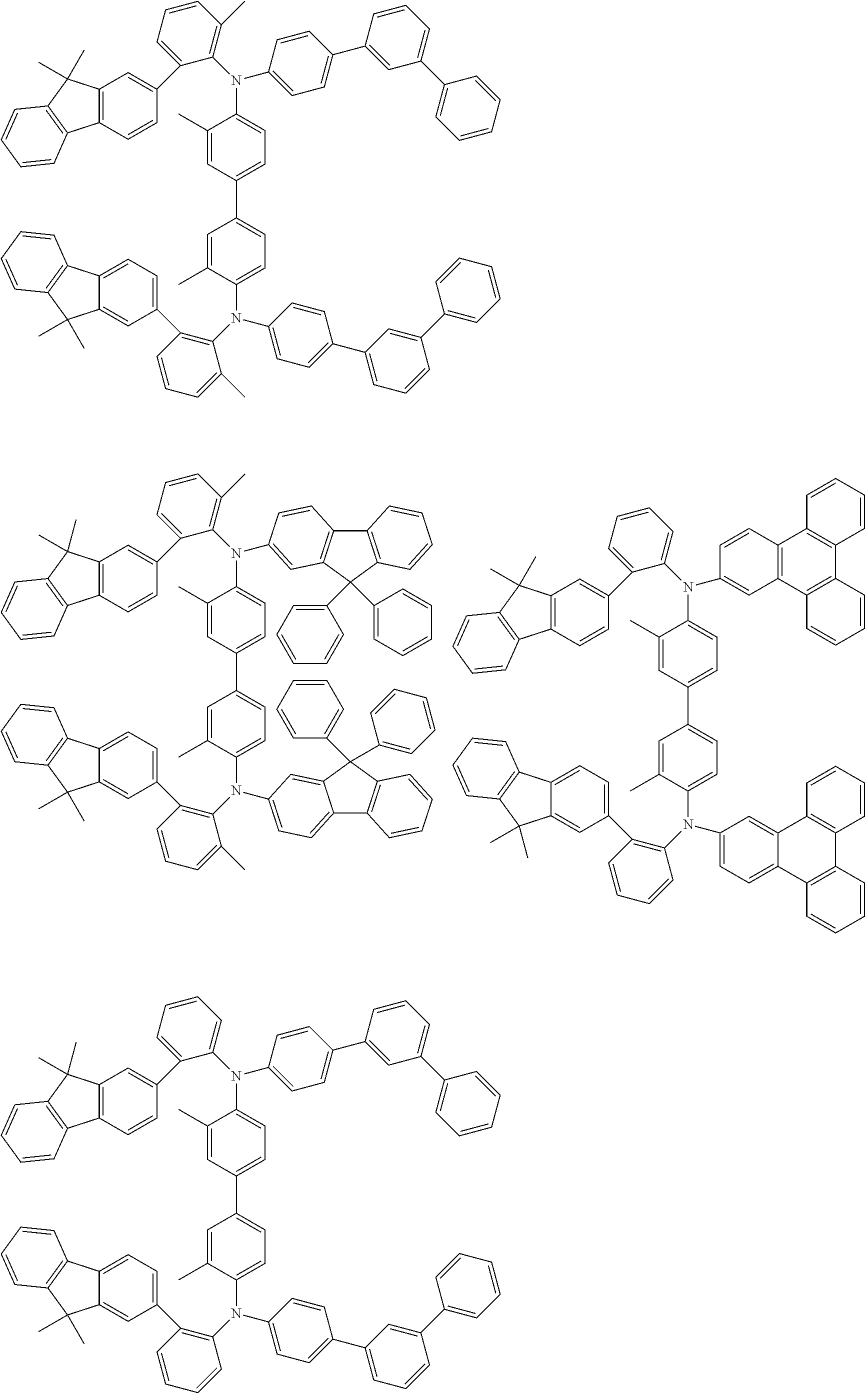

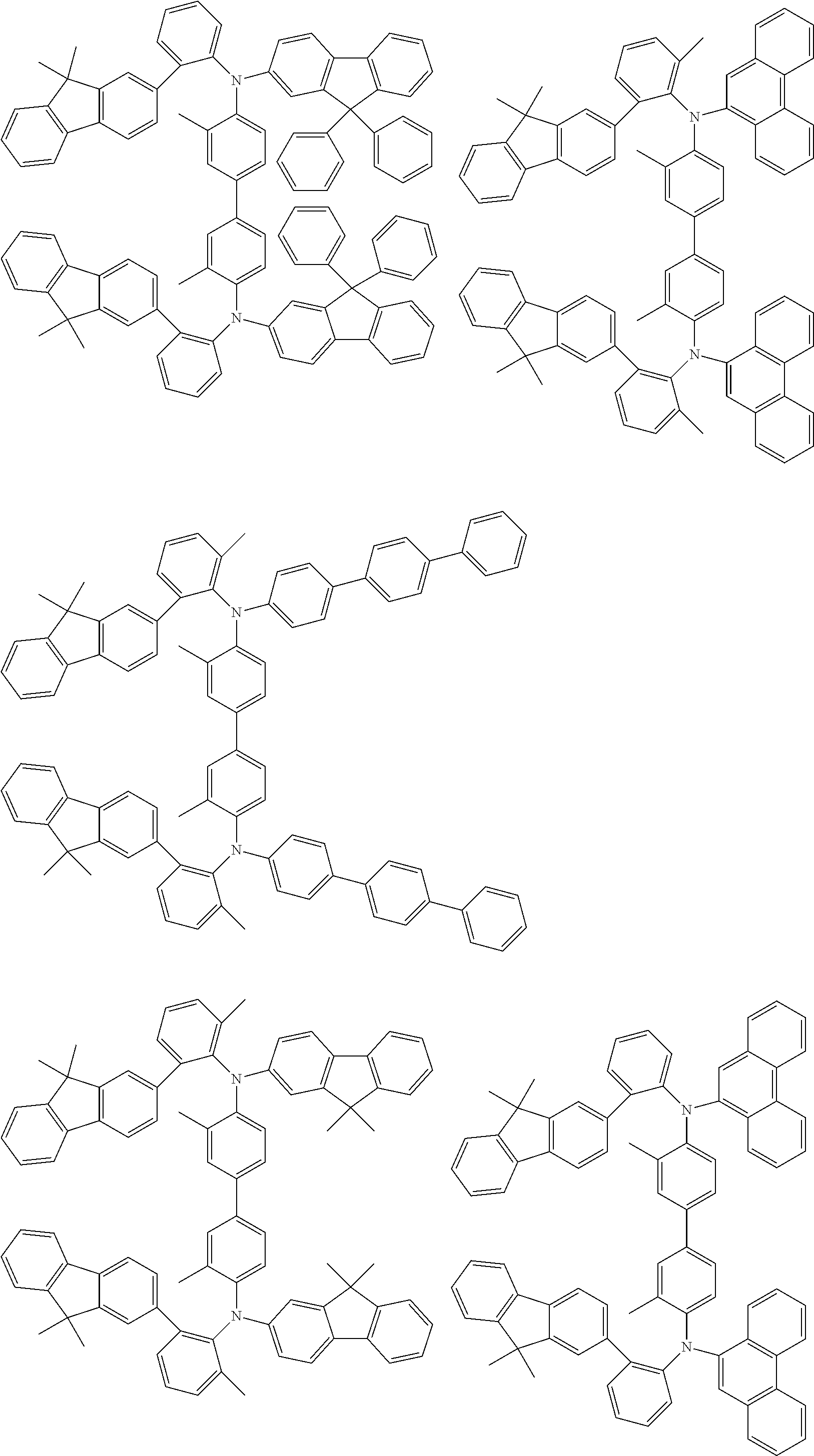

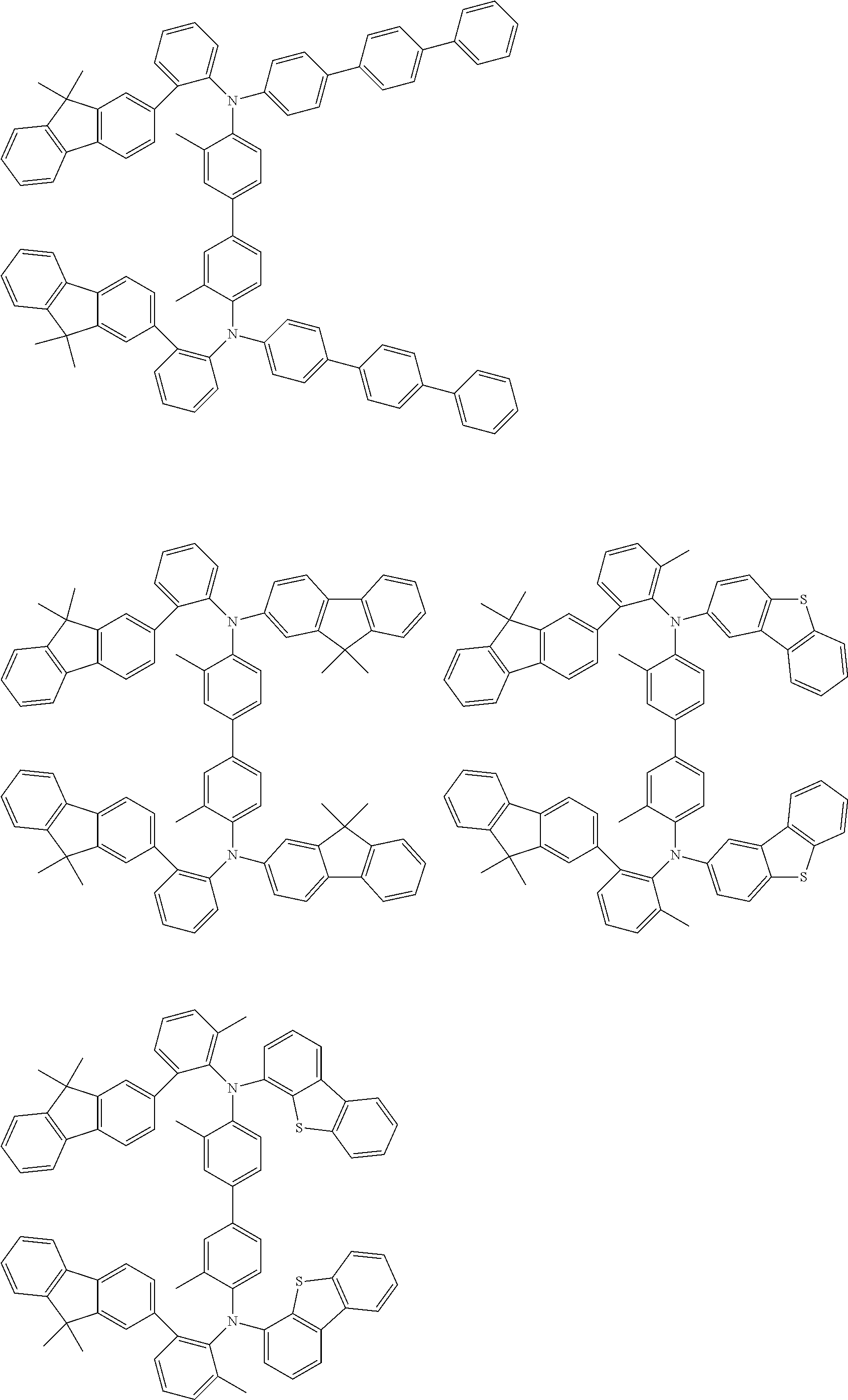

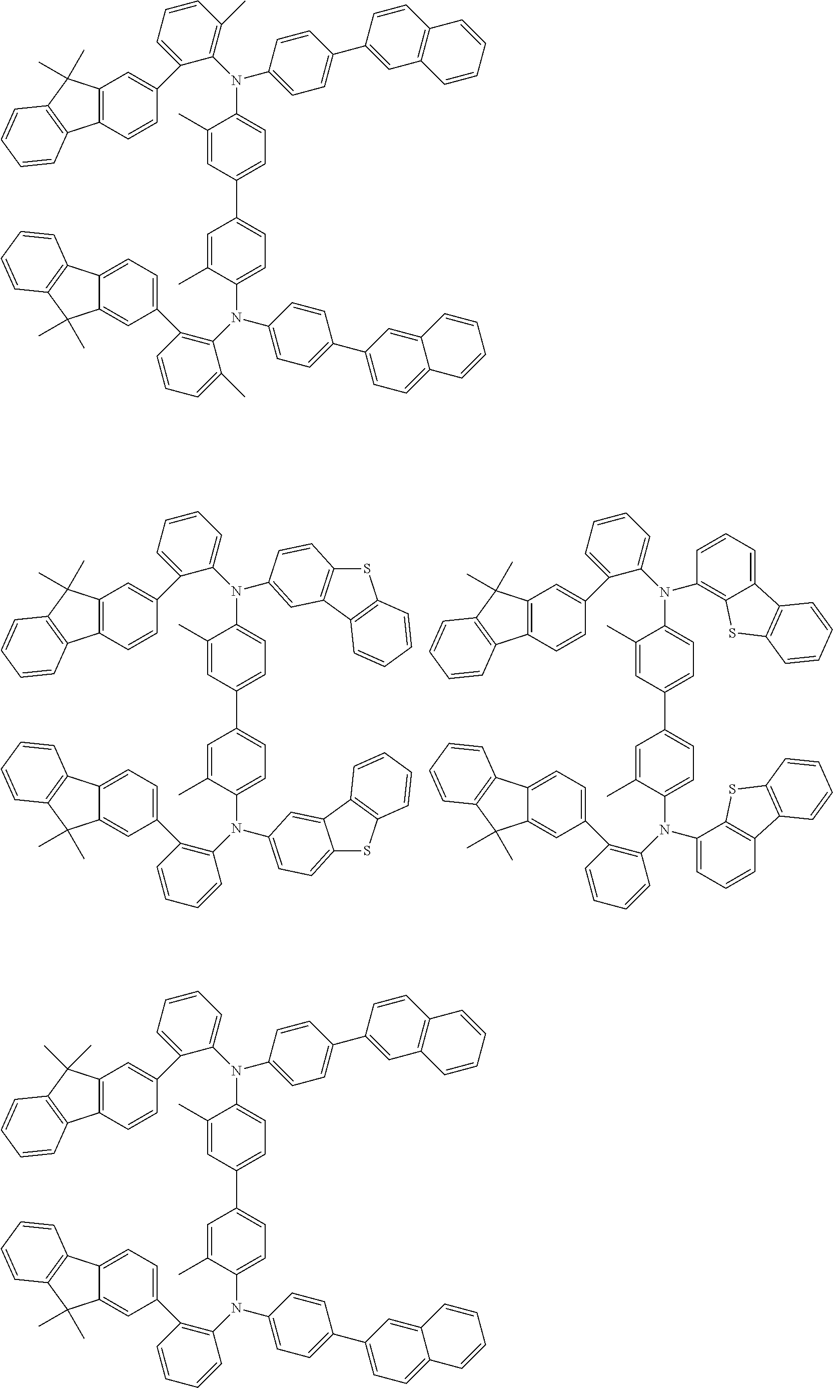

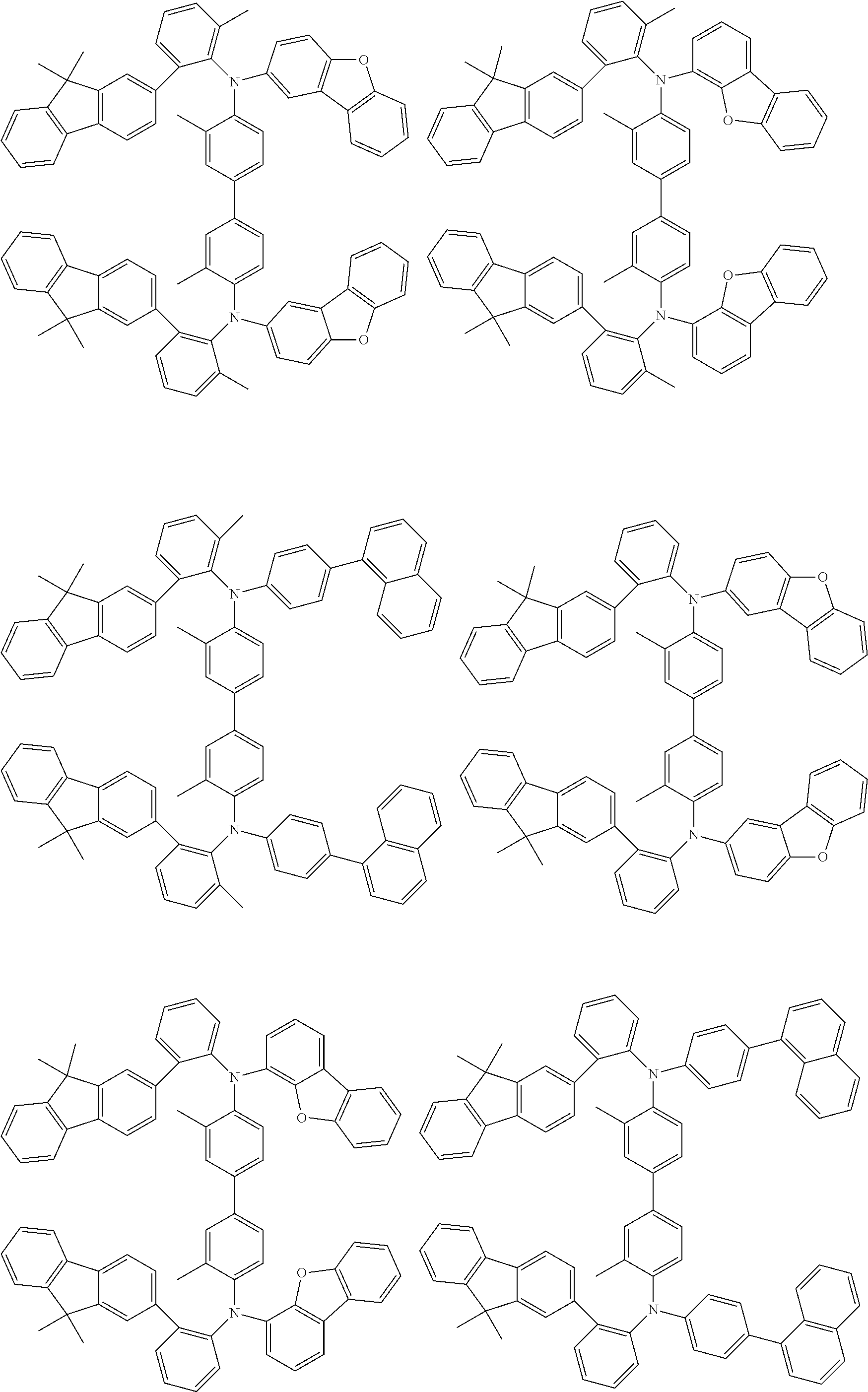

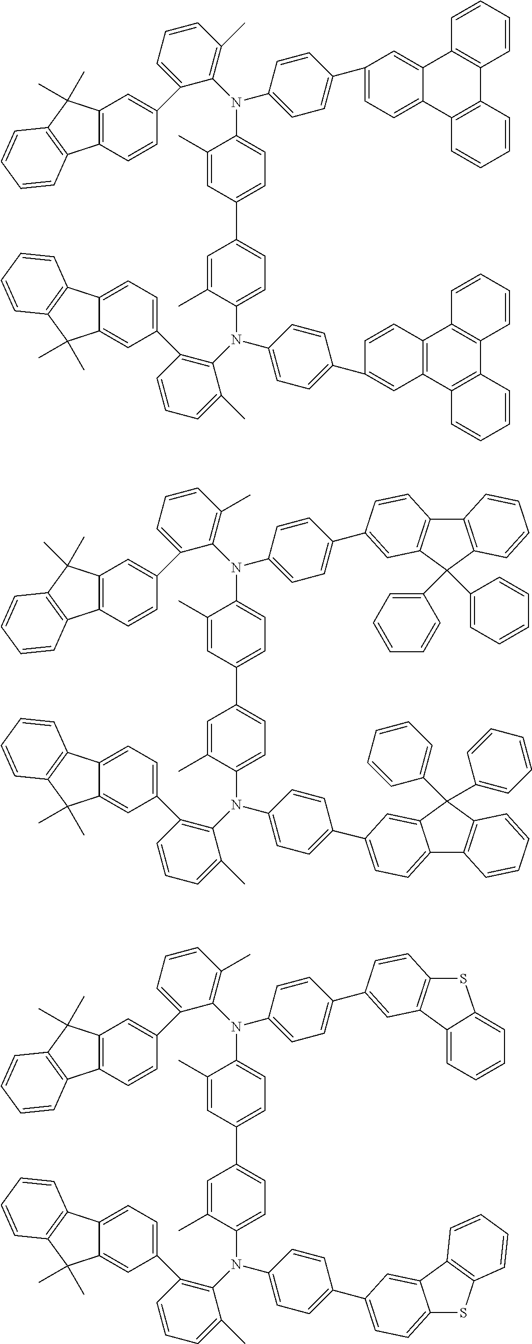

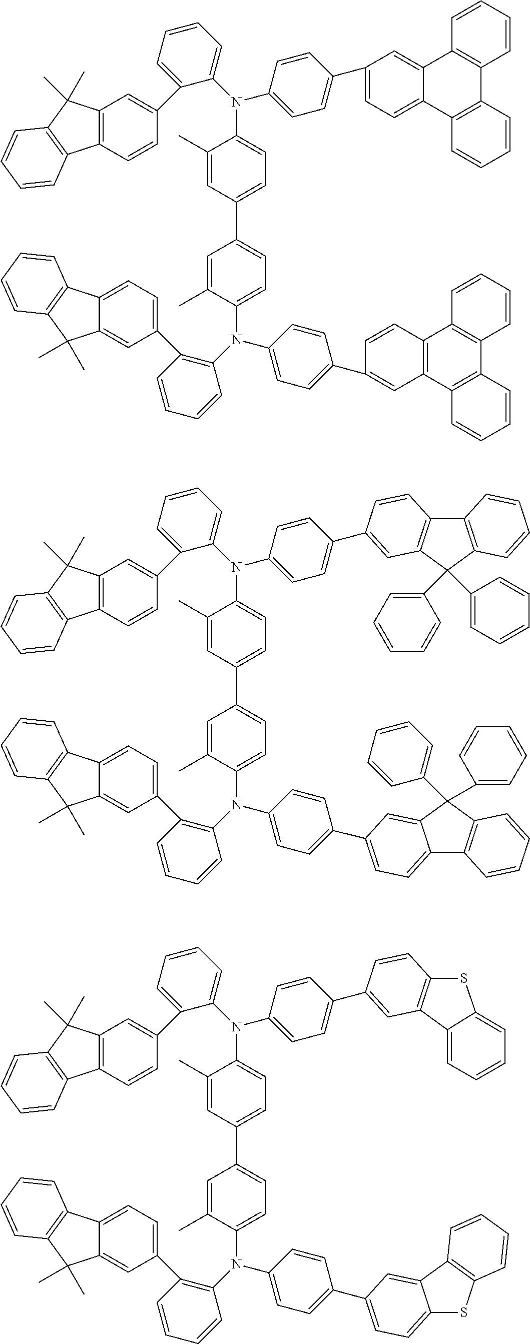

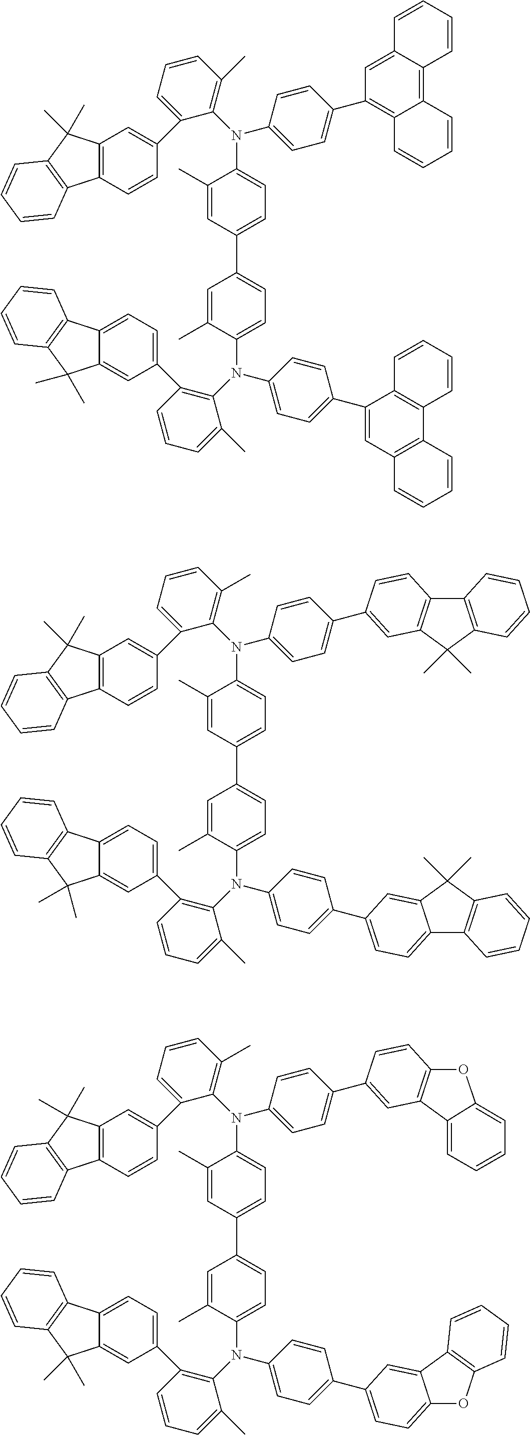

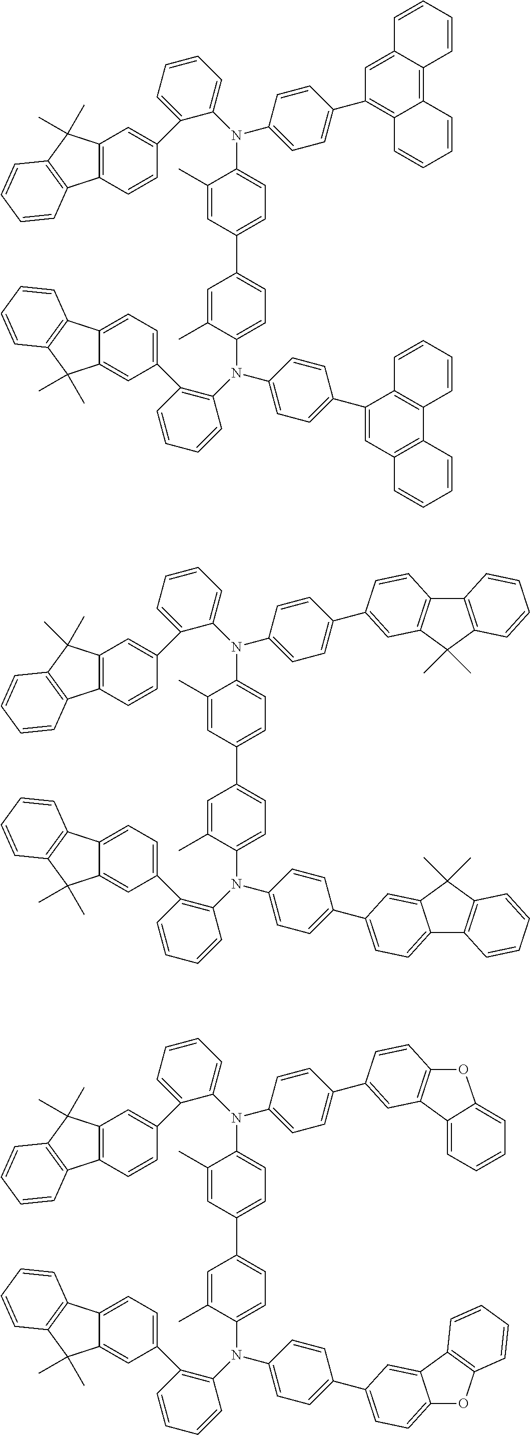

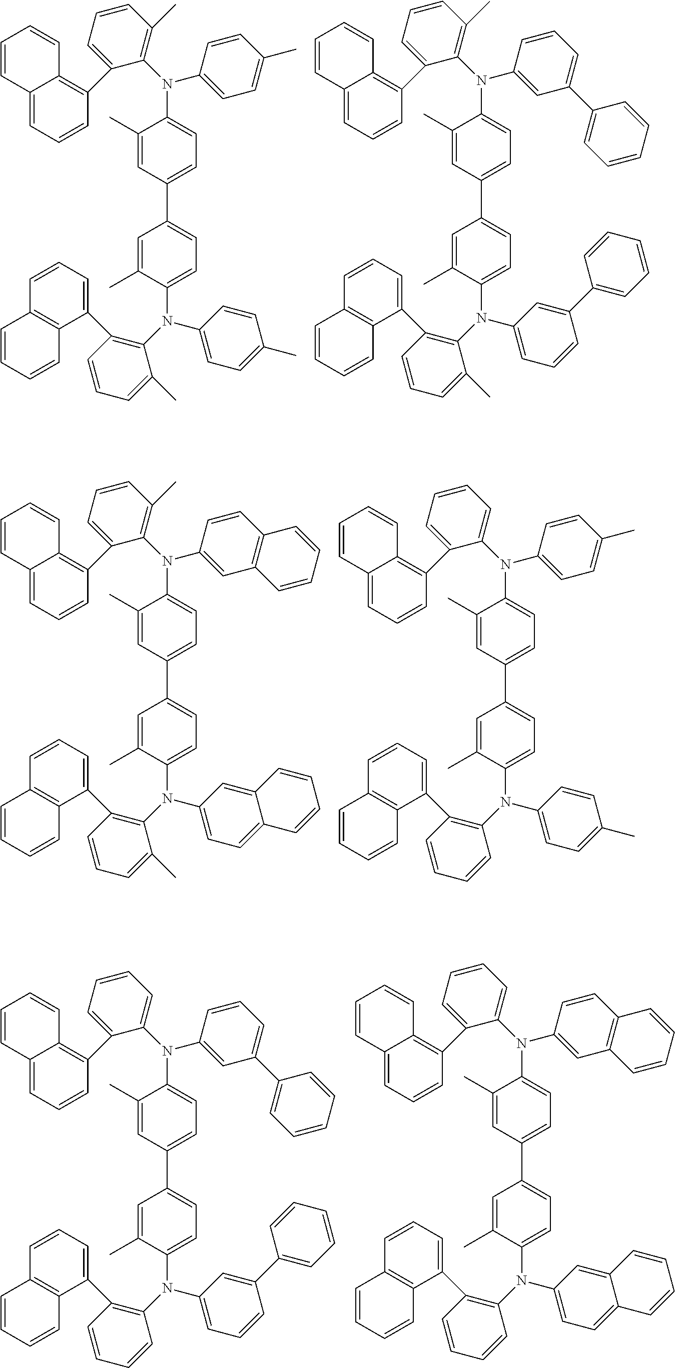

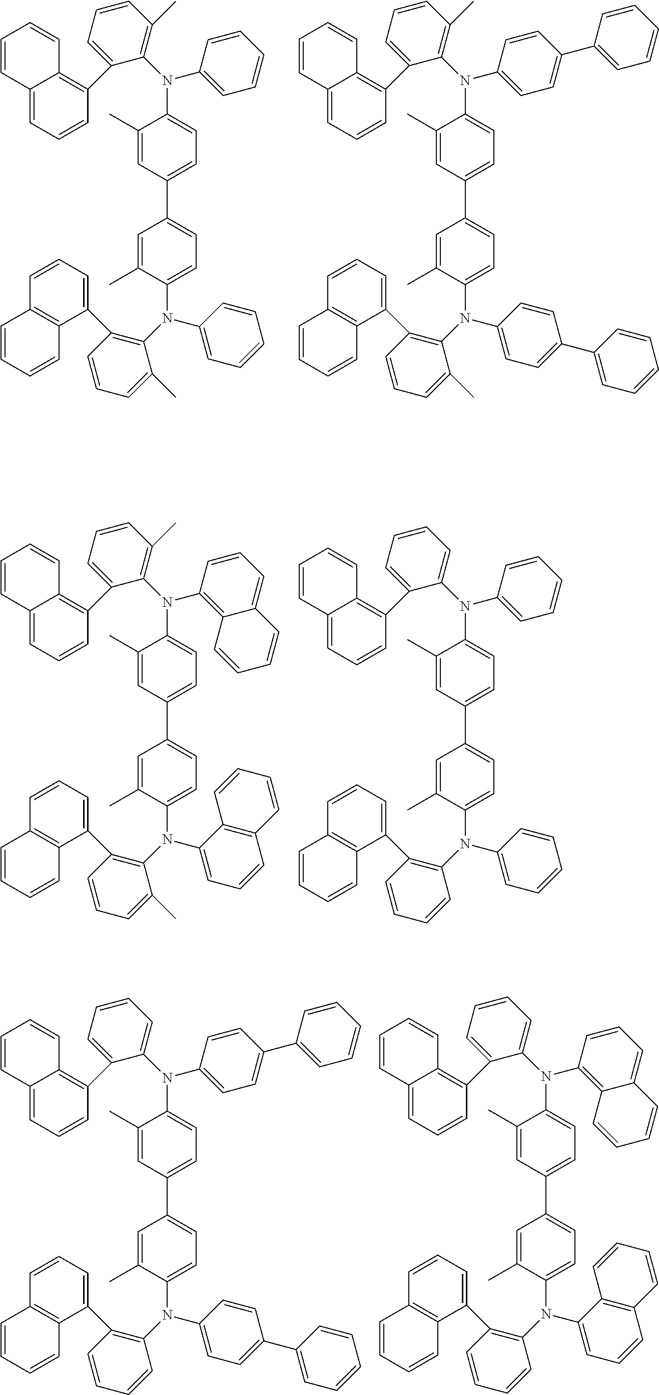

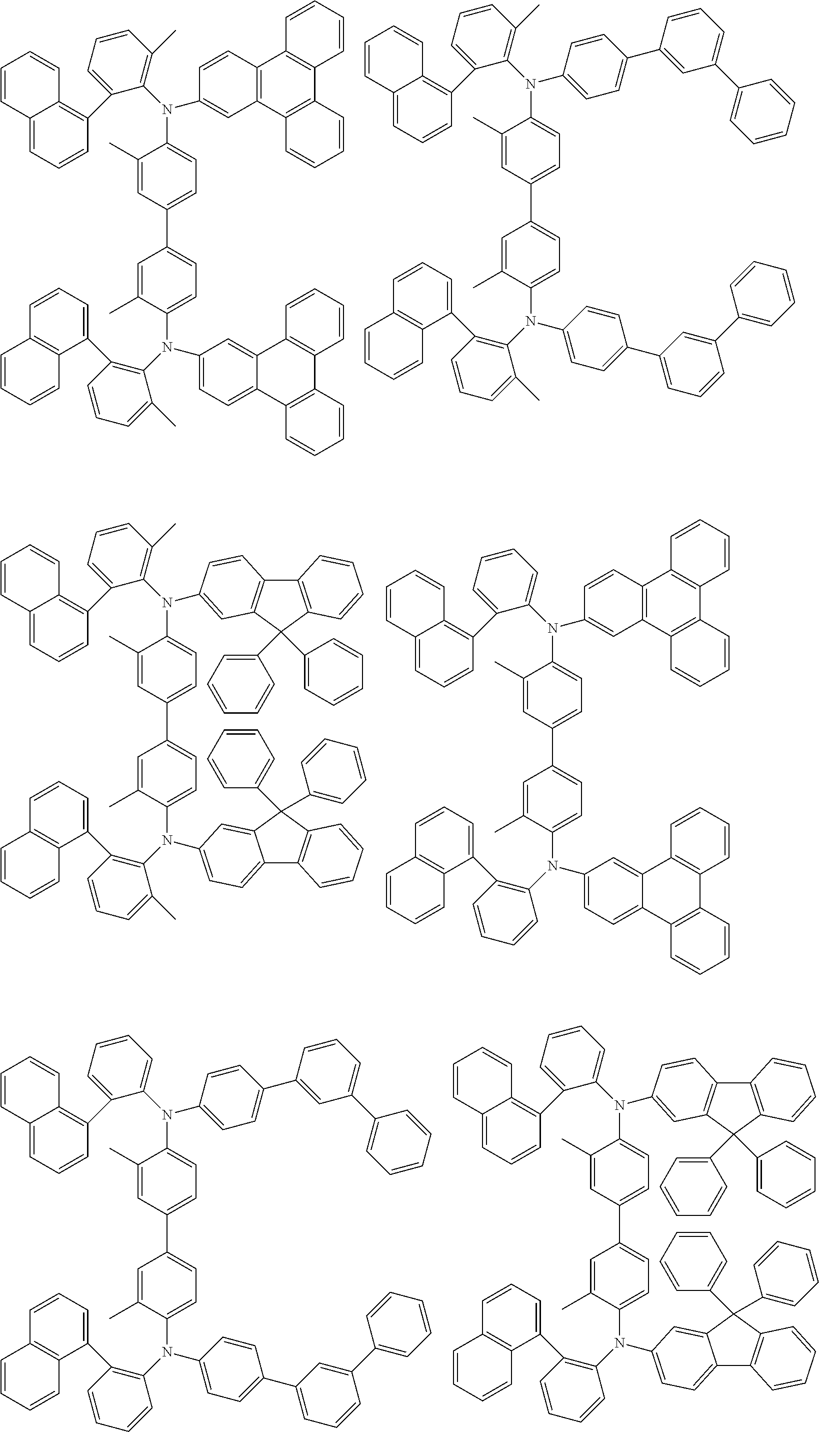

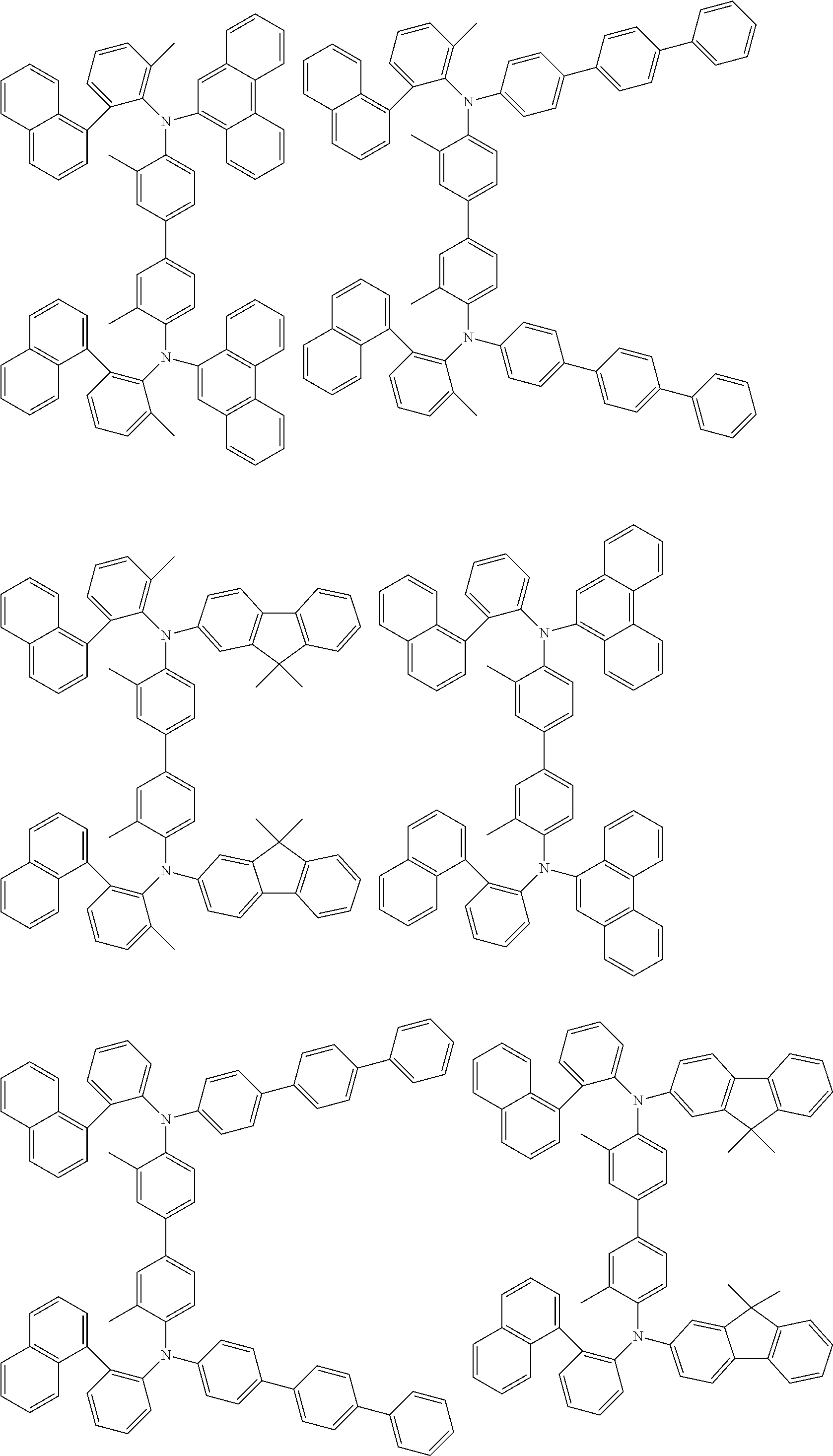

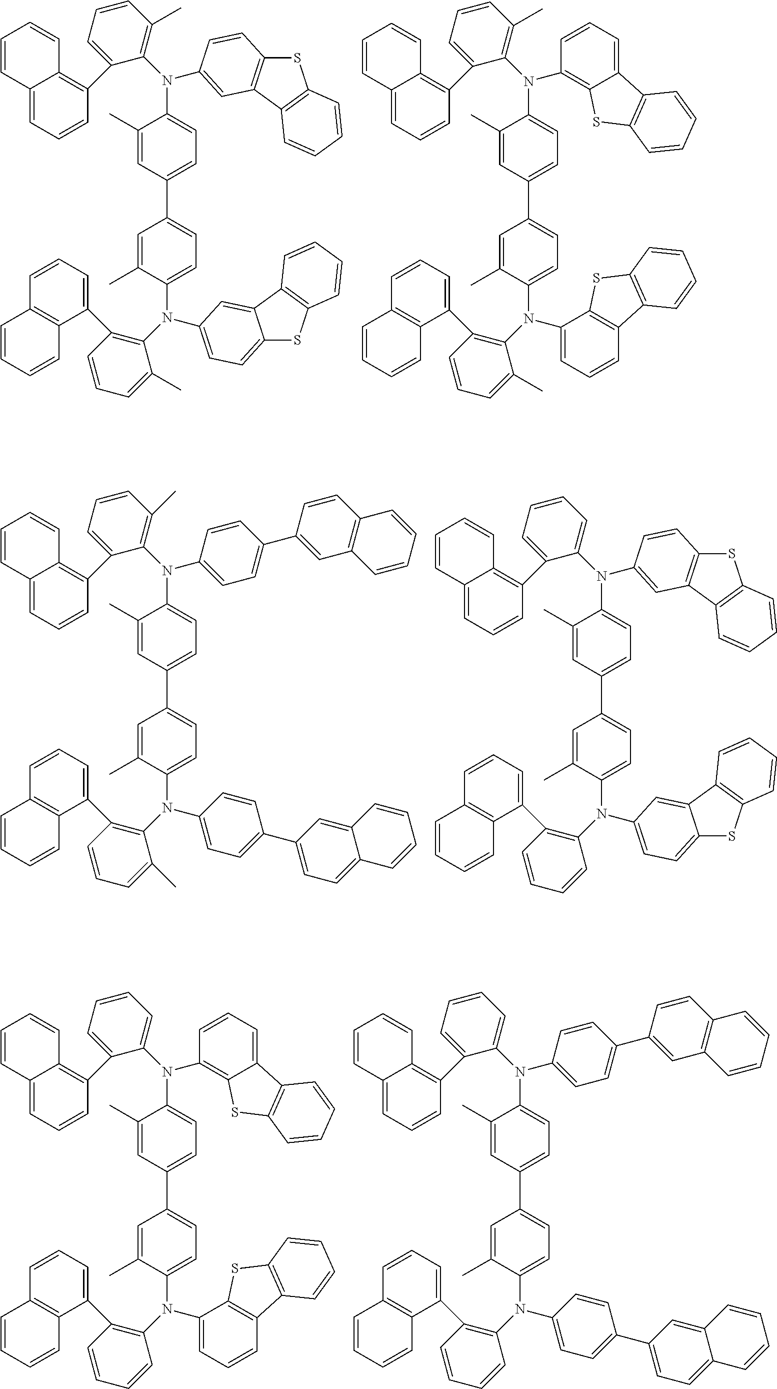

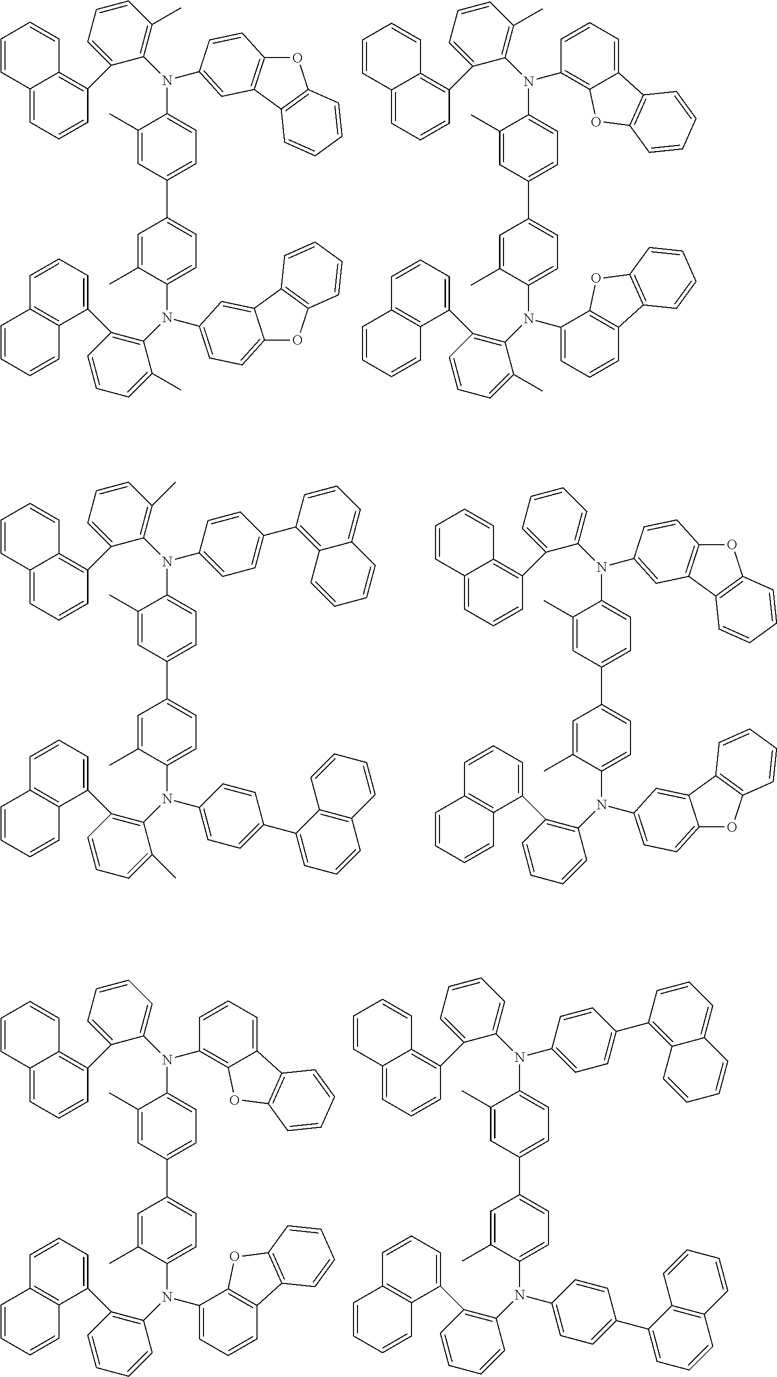

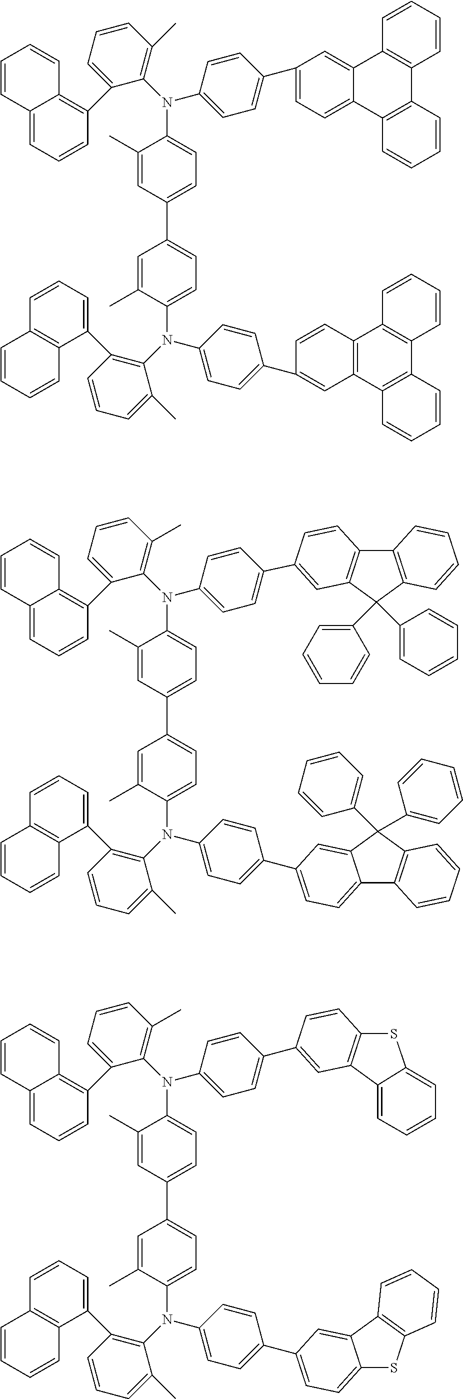 [SMILES: Cc1cc(-c2ccc(N(c3ccc(-c4ccc5c6ccccc6c6ccccc6c5c4)cc3)c3c(C)cccc3-c3cccc4ccccc34)c(C)c2)ccc1N(c1ccc(-c2ccc3c4ccccc4c4ccccc4c3c2)cc1)c1c(C)cccc1-c1cccc2ccccc12 ; Cc1cc(-c2ccc(N(c3ccc(-c4ccc5c(c4)C(c4ccccc4)(c4ccccc4)c4ccccc4-5)cc3)c3c(C)cccc3-c3cccc4ccccc34)c(C)c2)ccc1N(c1ccc(-c2ccc3c(c2)C(c2ccccc2)(c2ccccc2)c2ccccc2-3)cc1)c1c(C)cccc1-c1cccc2ccccc12 ; Cc1cc(-c2ccc(N(c3ccc(-c4ccc5sc6ccccc6c5c4)cc3)c3c(C)cccc3-c3cccc4ccccc34)c(C)c2)ccc1N(c1ccc(-c2ccc3sc4ccccc4c3c2)cc1)c1c(C)cccc1-c1cccc2ccccc12]

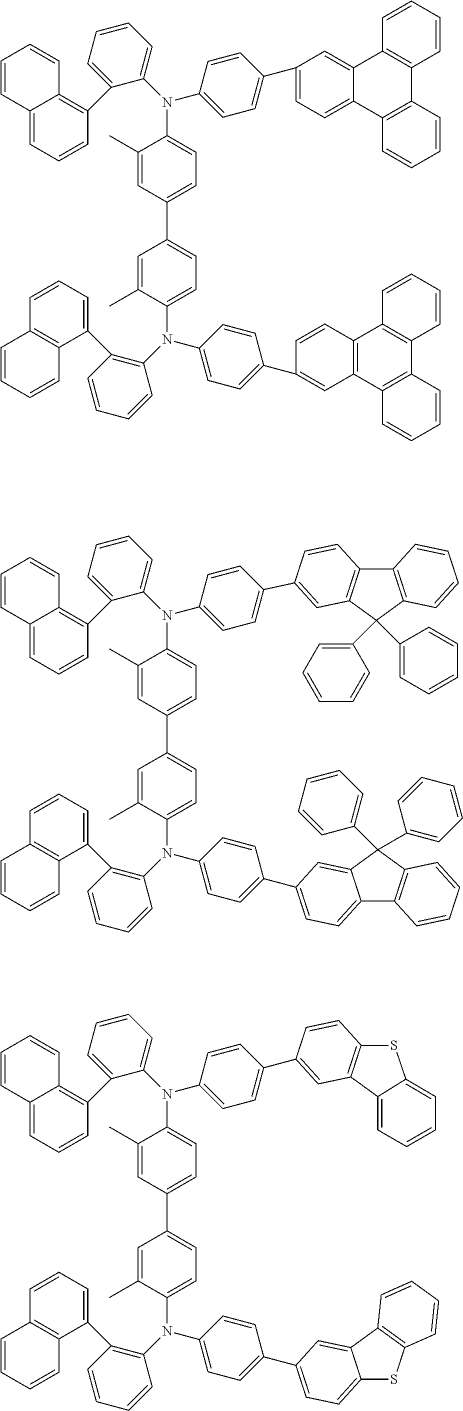 [SMILES: Cc1cc(-c2ccc(N(c3ccc(-c4ccc5c6ccccc6c6ccccc6c5c4)cc3)c3ccccc3-c3cccc4ccccc34)c(C)c2)ccc1N(c1ccc(-c2ccc3c4ccccc4c4ccccc4c3c2)cc1)c1ccccc1-c1cccc2ccccc12 ; Cc1cc(-c2ccc(N(c3ccc(-c4ccc5c(c4)C(c4ccccc4)(c4ccccc4)c4ccccc4-5)cc3)c3ccccc3-c3cccc4ccccc34)c(C)c2)ccc1N(c1ccc(-c2ccc3c(c2)C(c2ccccc2)(c2ccccc2)c2ccccc2-3)cc1)c1ccccc1-c1cccc2ccccc12 ; Cc1cc(-c2ccc(N(c3ccc(-c4ccc5sc6ccccc6c5c4)cc3)c3ccccc3-c3cccc4ccccc34)c(C)c2)ccc1N(c1ccc(-c2ccc3sc4ccccc4c3c2)cc1)c1ccccc1-c1cccc2ccccc12]

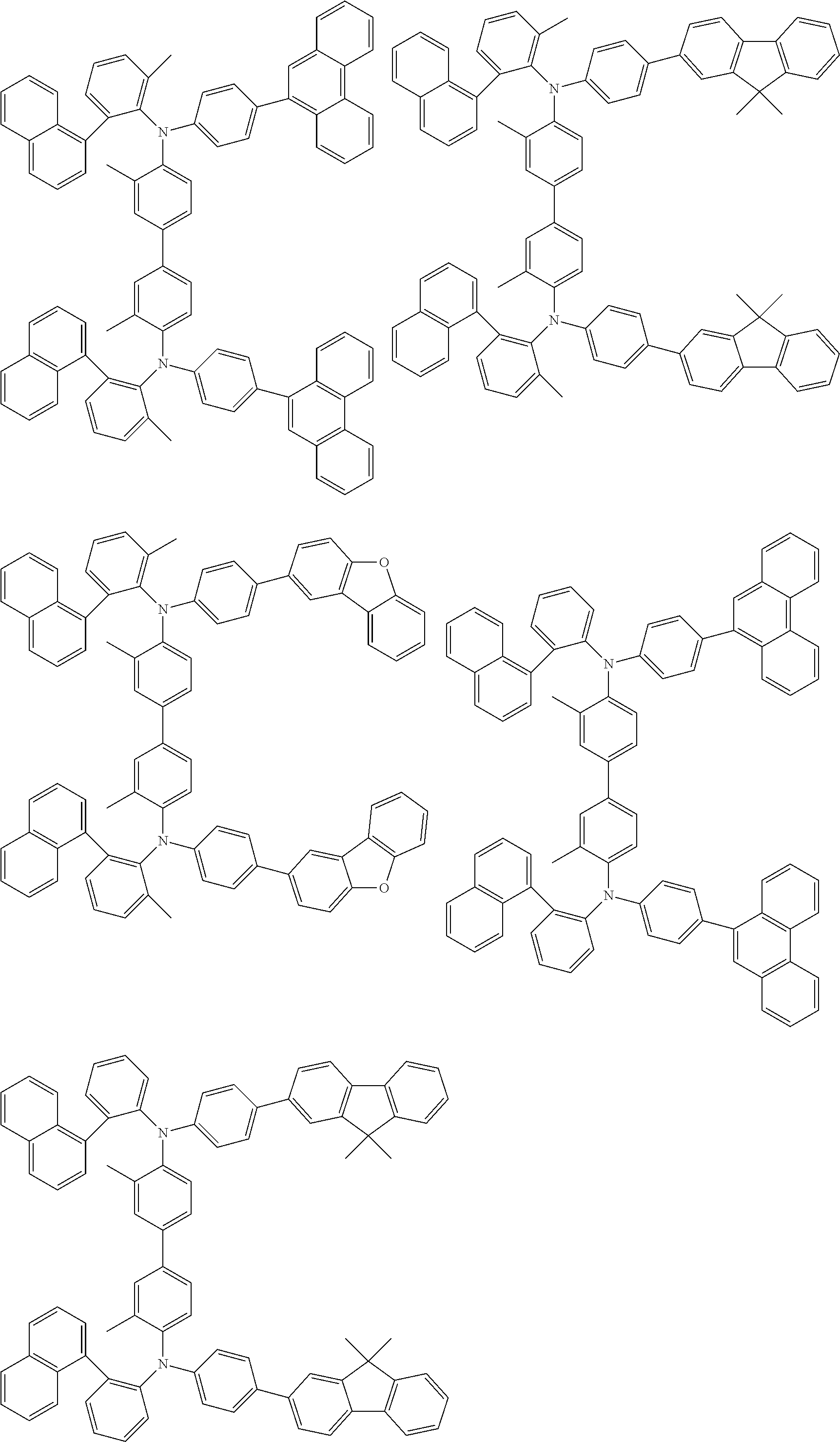

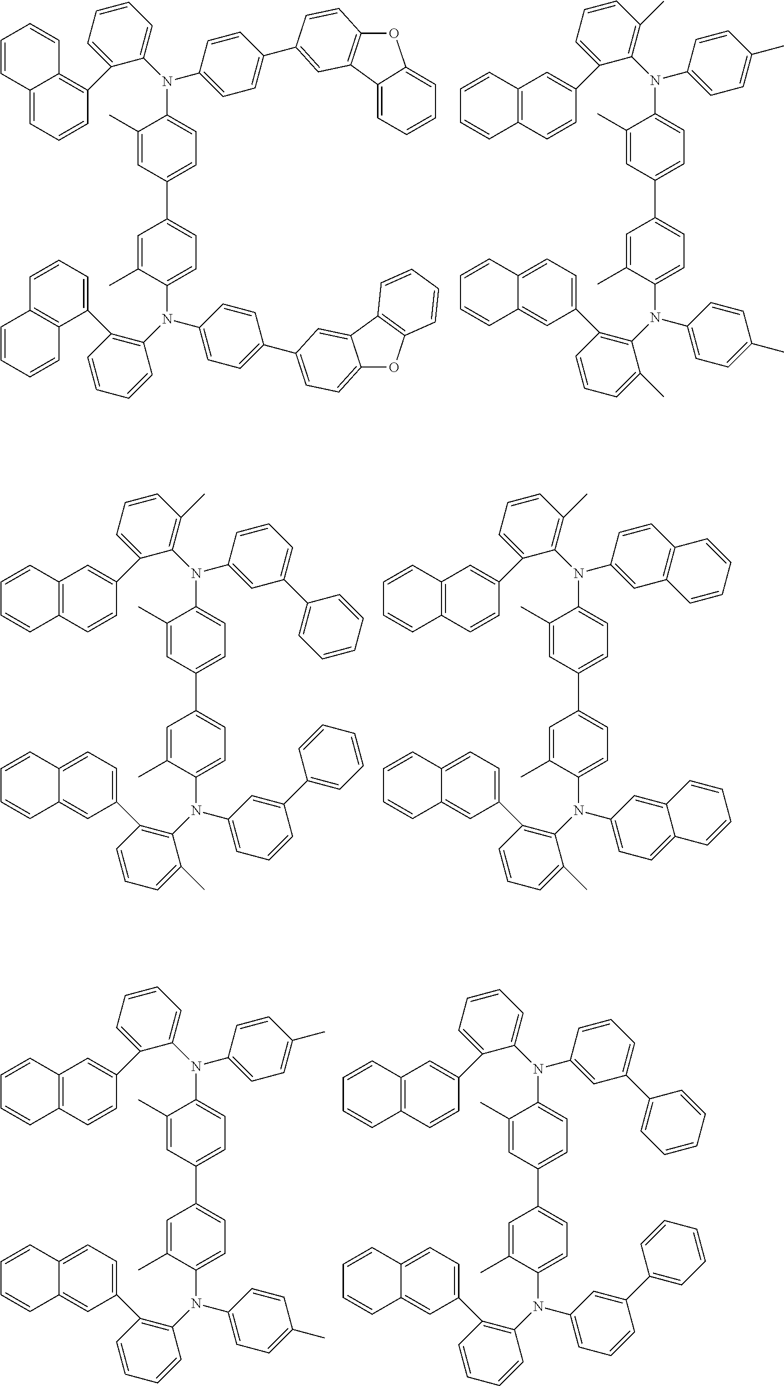

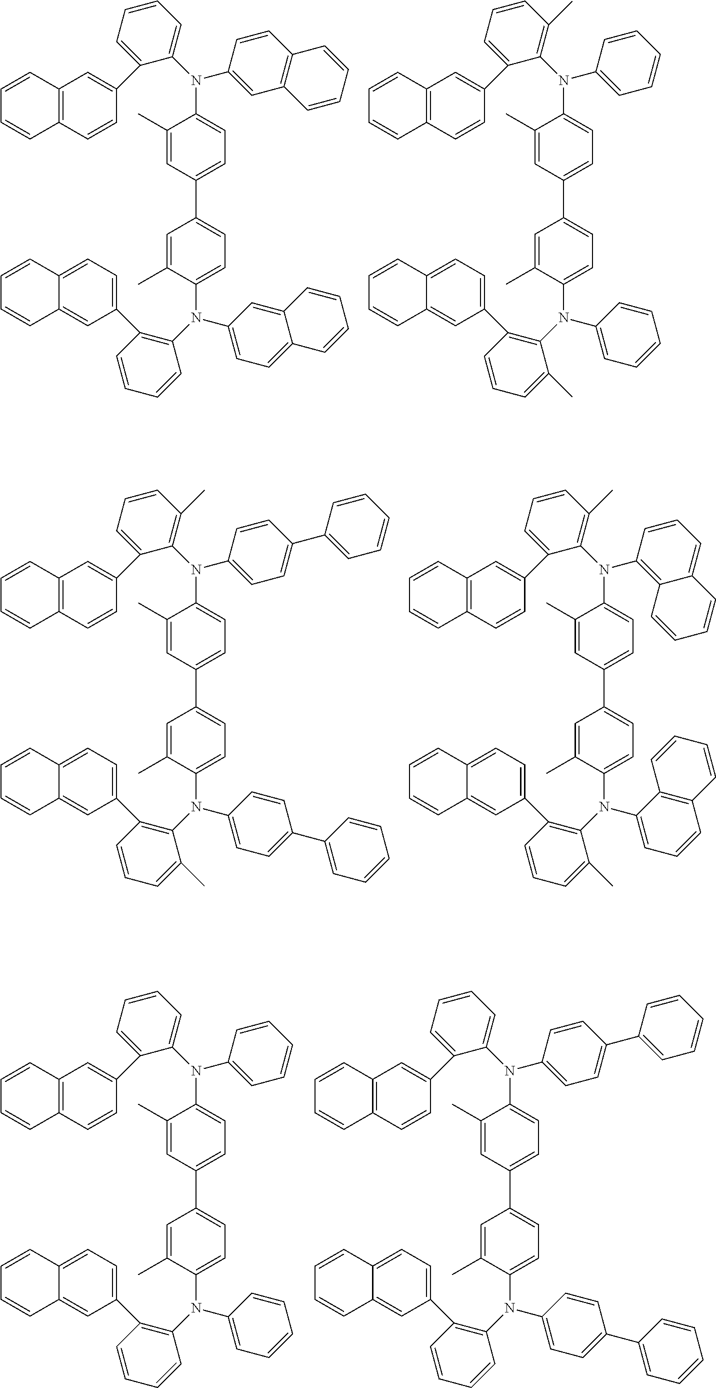

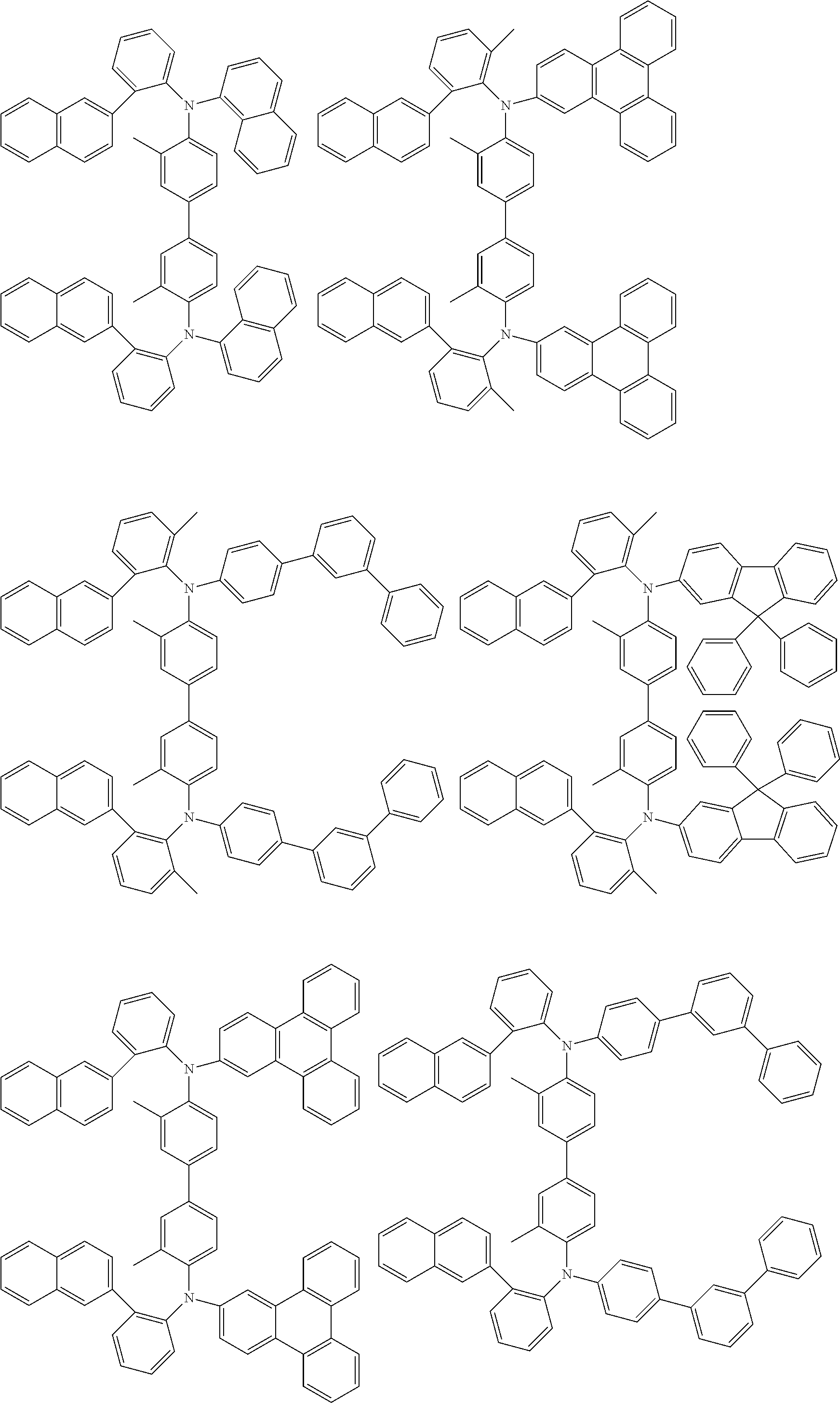

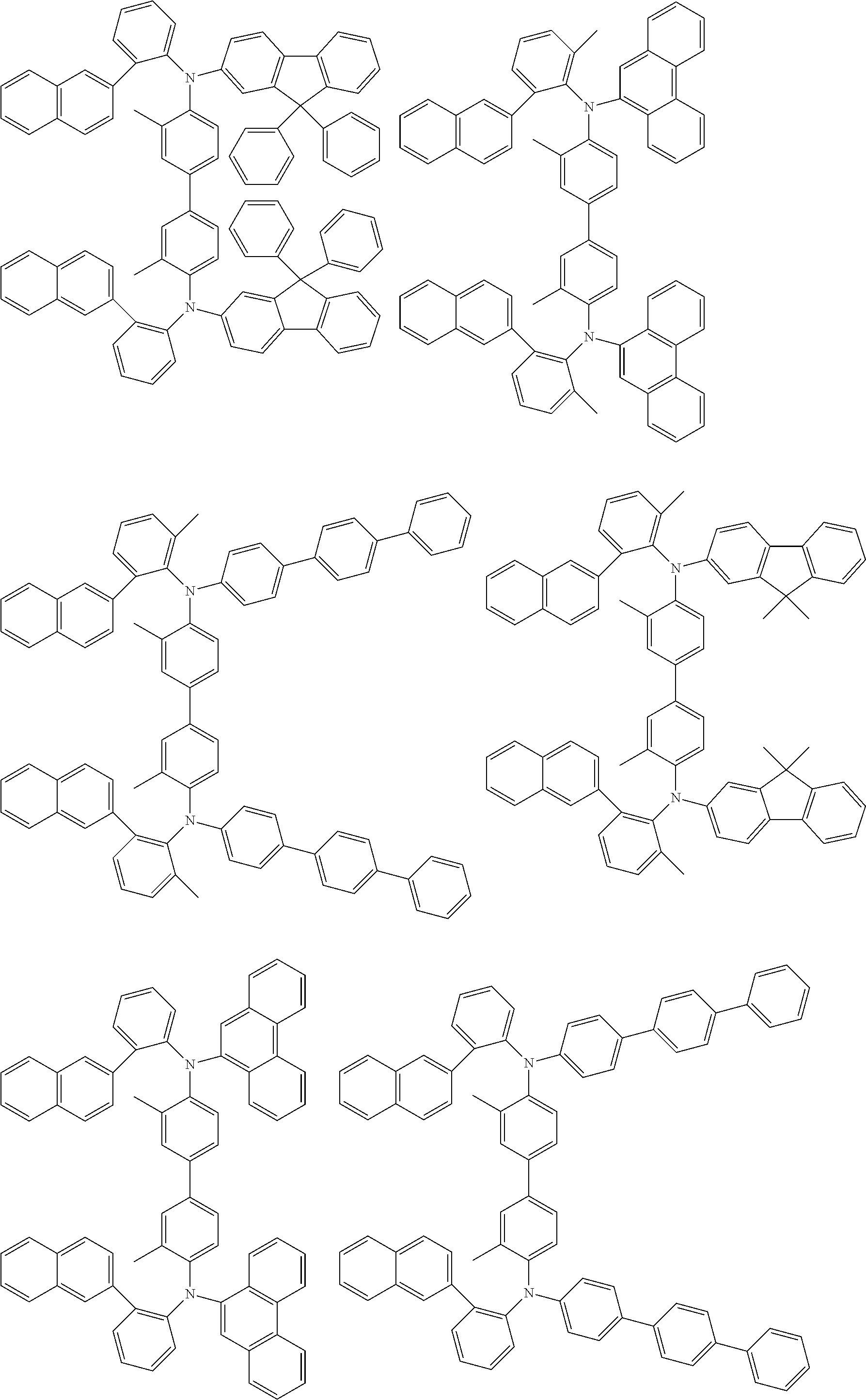

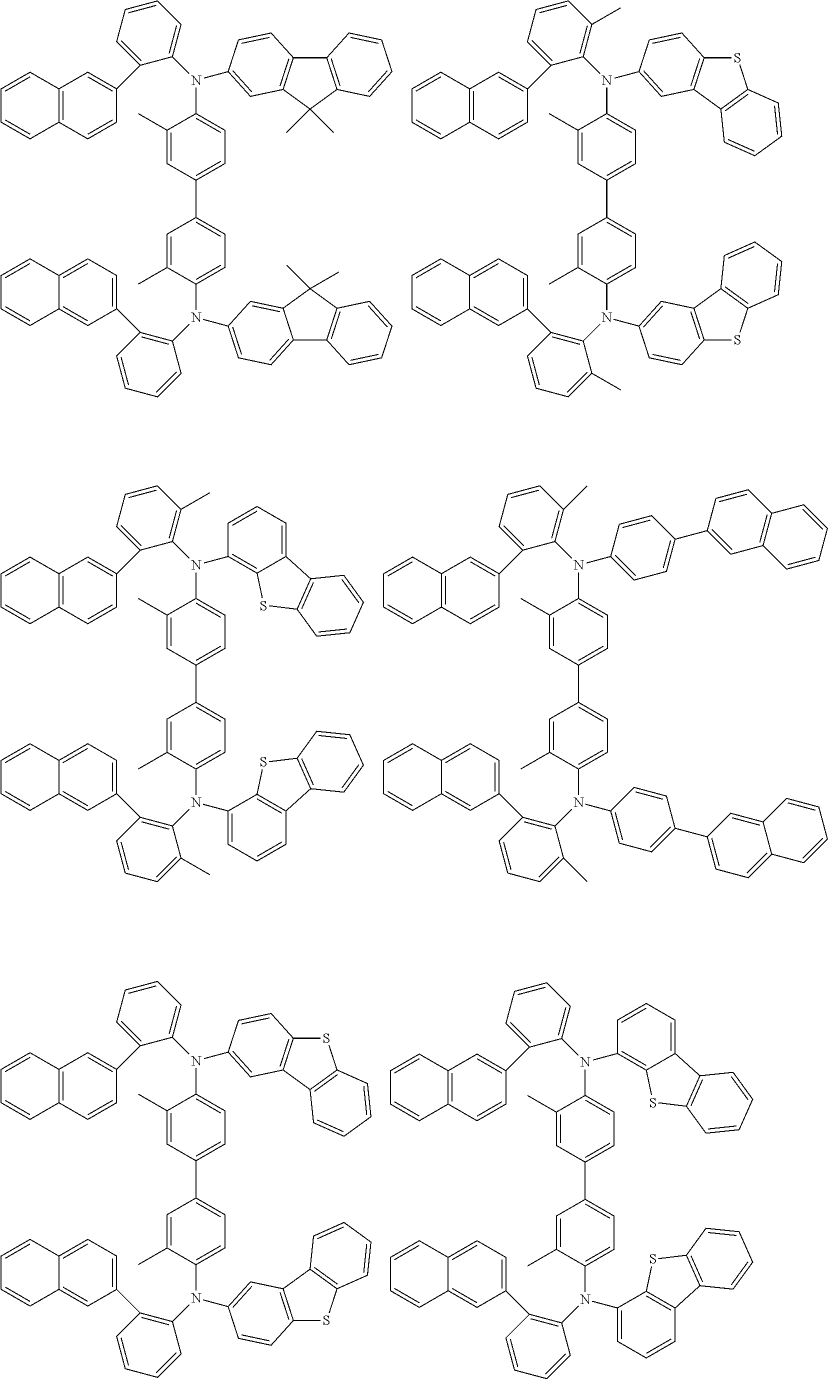

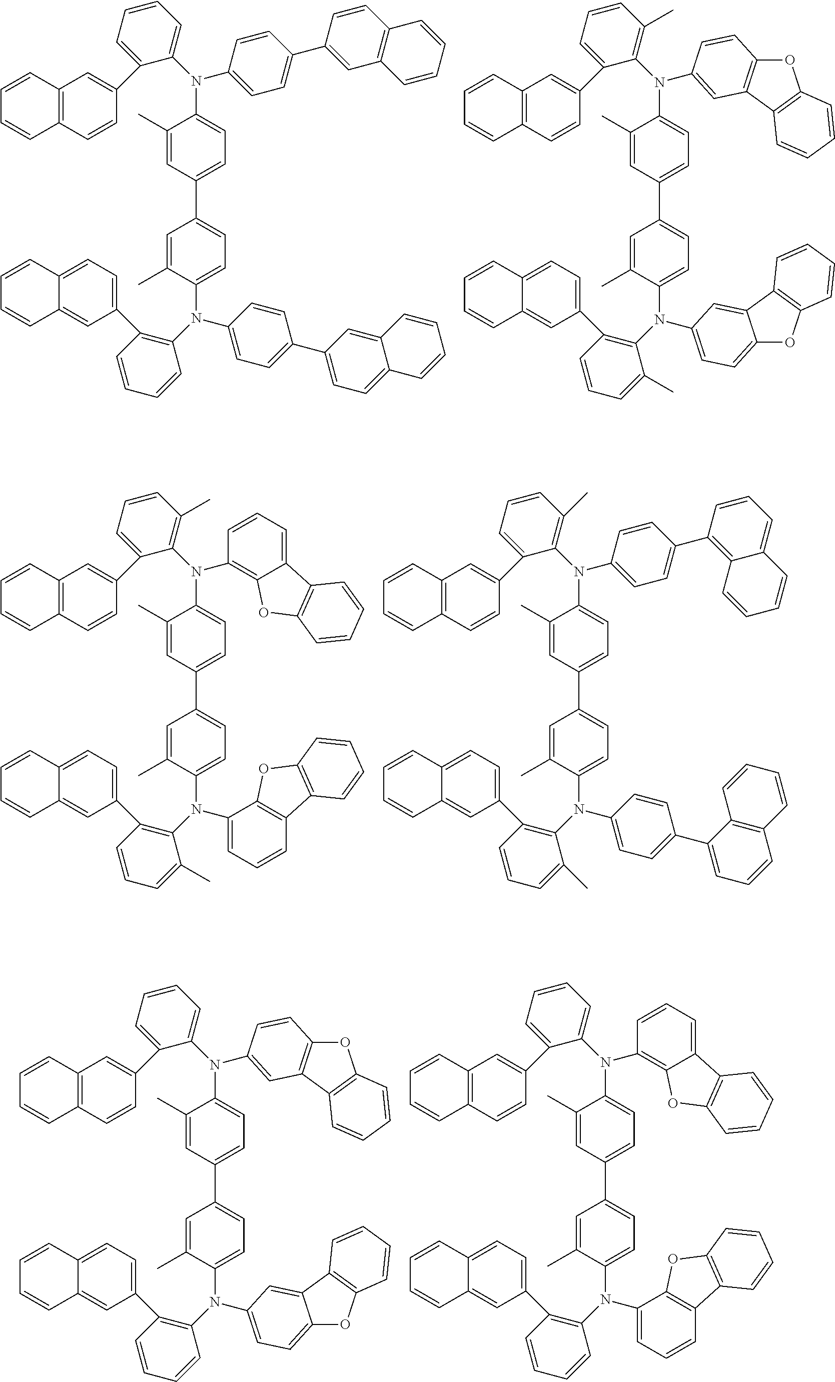

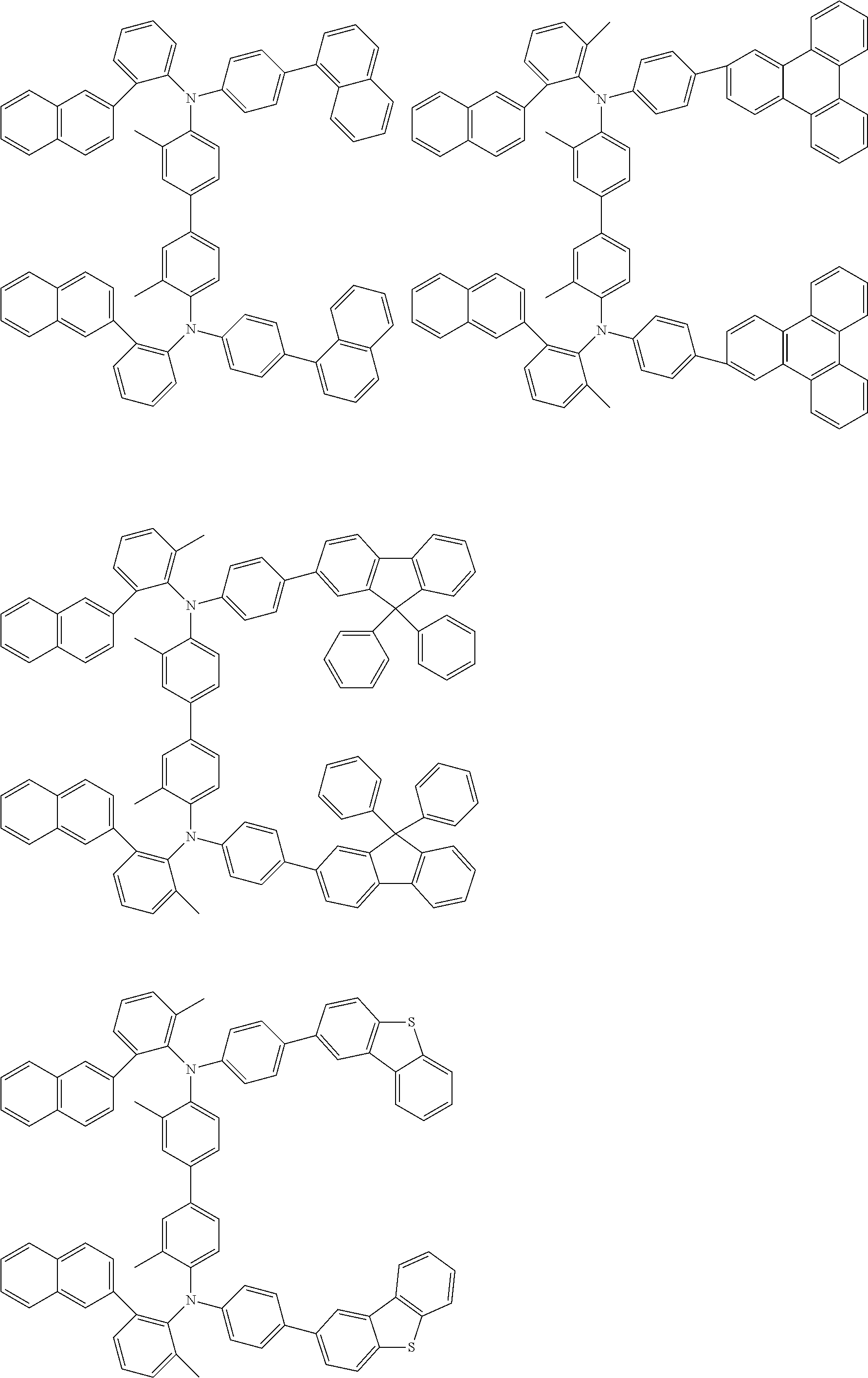

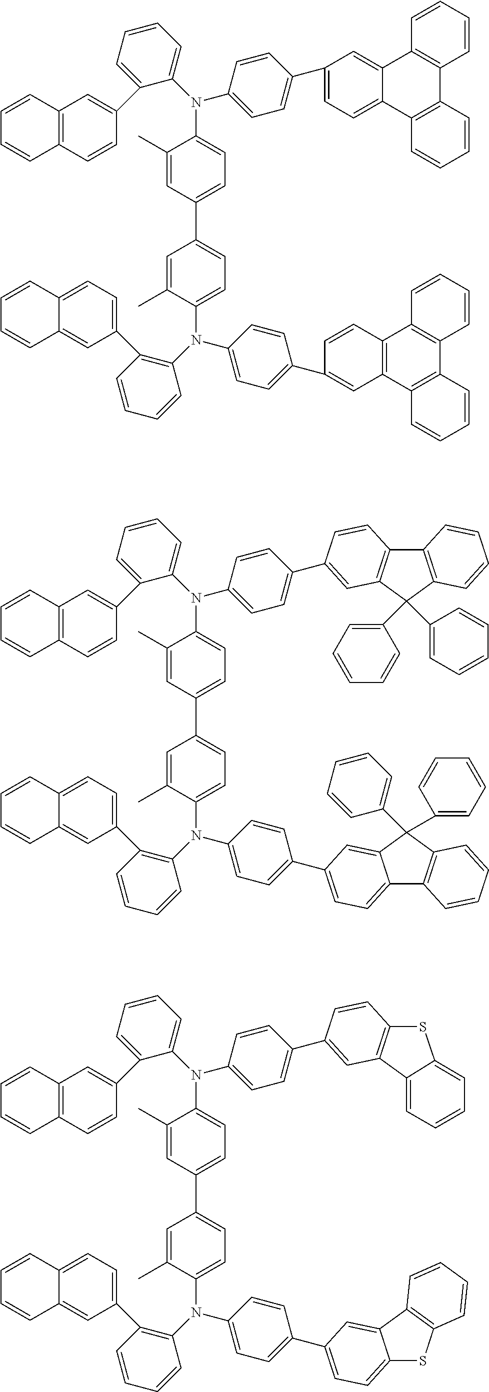 [SMILES: Cc1cc(-c2ccc(N(c3ccc(-c4ccc5c6ccccc6c6ccccc6c5c4)cc3)c3ccccc3-c3ccc4ccccc4c3)c(C)c2)ccc1N(c1ccc(-c2ccc3c4ccccc4c4ccccc4c3c2)cc1)c1ccccc1-c1ccc2ccccc2c1 ; Cc1cc(-c2ccc(N(c3ccc(-c4ccc5c(c4)C(c4ccccc4)(c4ccccc4)c4ccccc4-5)cc3)c3ccccc3-c3ccc4ccccc4c3)c(C)c2)ccc1N(c1ccc(-c2ccc3c(c2)C(c2ccccc2)(c2ccccc2)c2ccccc2-3)cc1)c1ccccc1-c1ccc2ccccc2c1 ; Cc1cc(-c2ccc(N(c3ccc(-c4ccc5sc6ccccc6c5c4)cc3)c3ccccc3-c3ccc4ccccc4c3)c(C)c2)ccc1N(c1ccc(-c2ccc3sc4ccccc4c3c2)cc1)c1ccccc1-c1ccc2ccccc2c1]

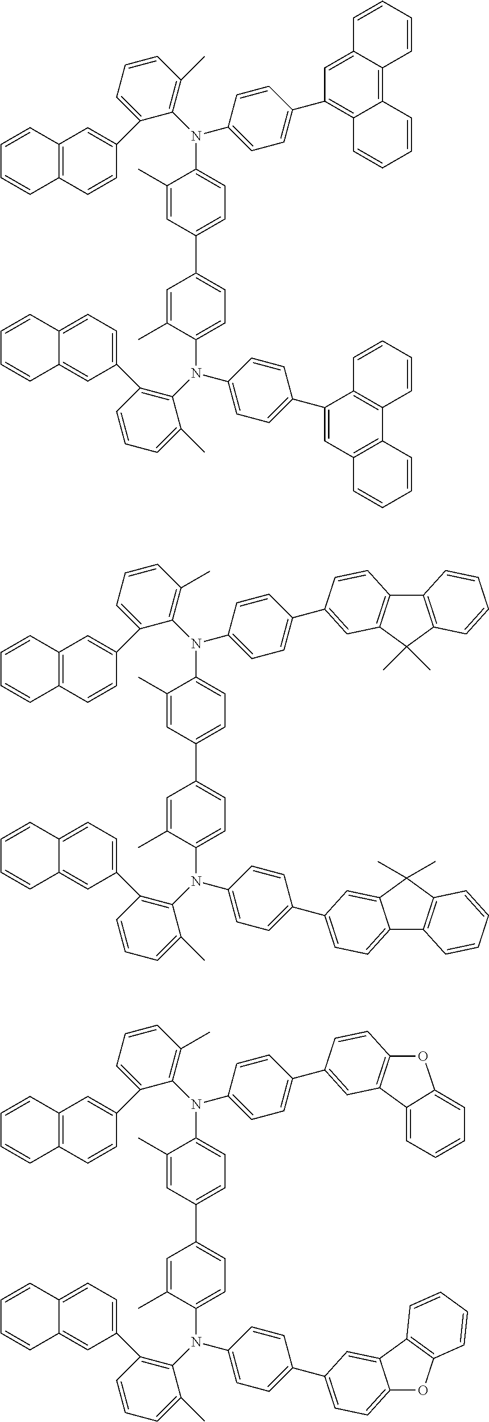 [SMILES: Cc1cc(-c2ccc(N(c3ccc(-c4cc5ccccc5c5ccccc45)cc3)c3c(C)cccc3-c3ccc4ccccc4c3)c(C)c2)ccc1N(c1ccc(-c2cc3ccccc3c3ccccc23)cc1)c1c(C)cccc1-c1ccc2ccccc2c1 ; Cc1cc(-c2ccc(N(c3ccc(-c4ccc5c(c4)C(C)(C)c4ccccc4-5)cc3)c3c(C)cccc3-c3ccc4ccccc4c3)c(C)c2)ccc1N(c1ccc(-c2ccc3c(c2)C(C)(C)c2ccccc2-3)cc1)c1c(C)cccc1-c1ccc2ccccc2c1 ; Cc1cc(-c2ccc(N(c3ccc(-c4ccc5oc6ccccc6c5c4)cc3)c3c(C)cccc3-c3ccc4ccccc4c3)c(C)c2)ccc1N(c1ccc(-c2ccc3oc4ccccc4c3c2)cc1)c1c(C)cccc1-c1ccc2ccccc2c1]

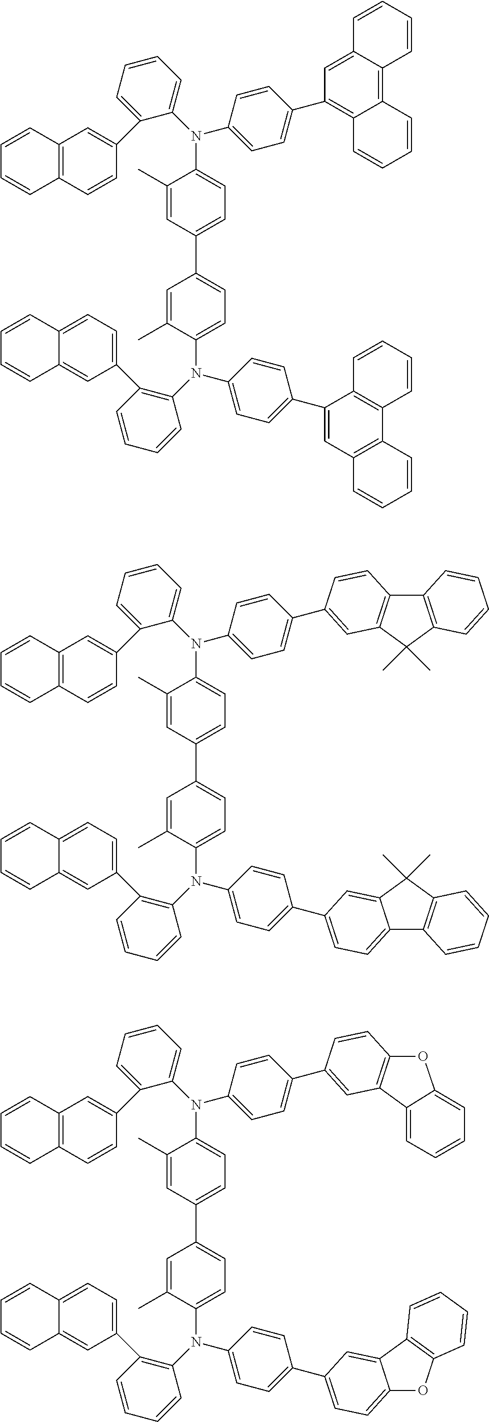 [SMILES: Cc1cc(-c2ccc(N(c3ccc(-c4cc5ccccc5c5ccccc45)cc3)c3ccccc3-c3ccc4ccccc4c3)c(C)c2)ccc1N(c1ccc(-c2cc3ccccc3c3ccccc23)cc1)c1ccccc1-c1ccc2ccccc2c1 ; Cc1cc(-c2ccc(N(c3ccc(-c4ccc5c(c4)C(C)(C)c4ccccc4-5)cc3)c3ccccc3-c3ccc4ccccc4c3)c(C)c2)ccc1N(c1ccc(-c2ccc3c(c2)C(C)(C)c2ccccc2-3)cc1)c1ccccc1-c1ccc2ccccc2c1 ; Cc1cc(-c2ccc(N(c3ccc(-c4ccc5oc6ccccc6c5c4)cc3)c3ccccc3-c3ccc4ccccc4c3)c(C)c2)ccc1N(c1ccc(-c2ccc3oc4ccccc4c3c2)cc1)c1ccccc1-c1ccc2ccccc2c1]

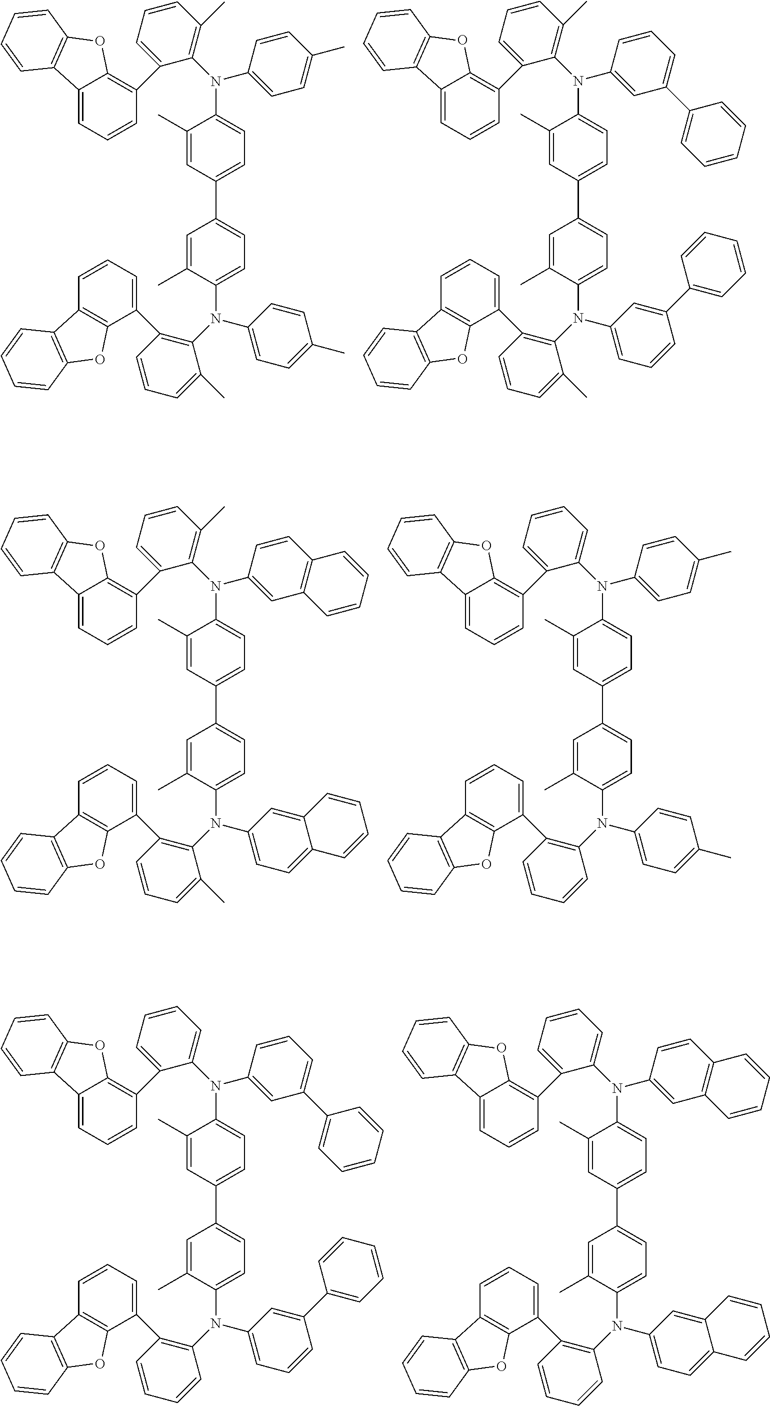

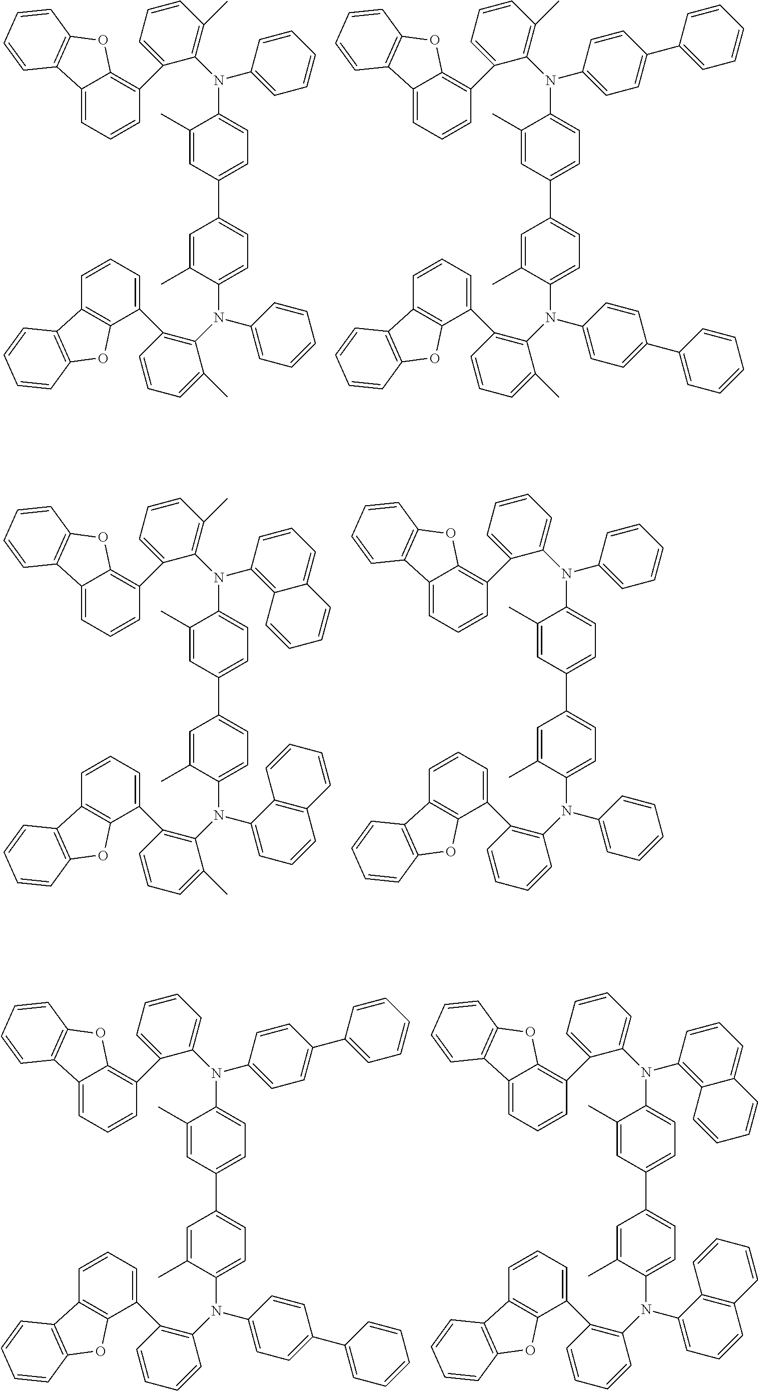

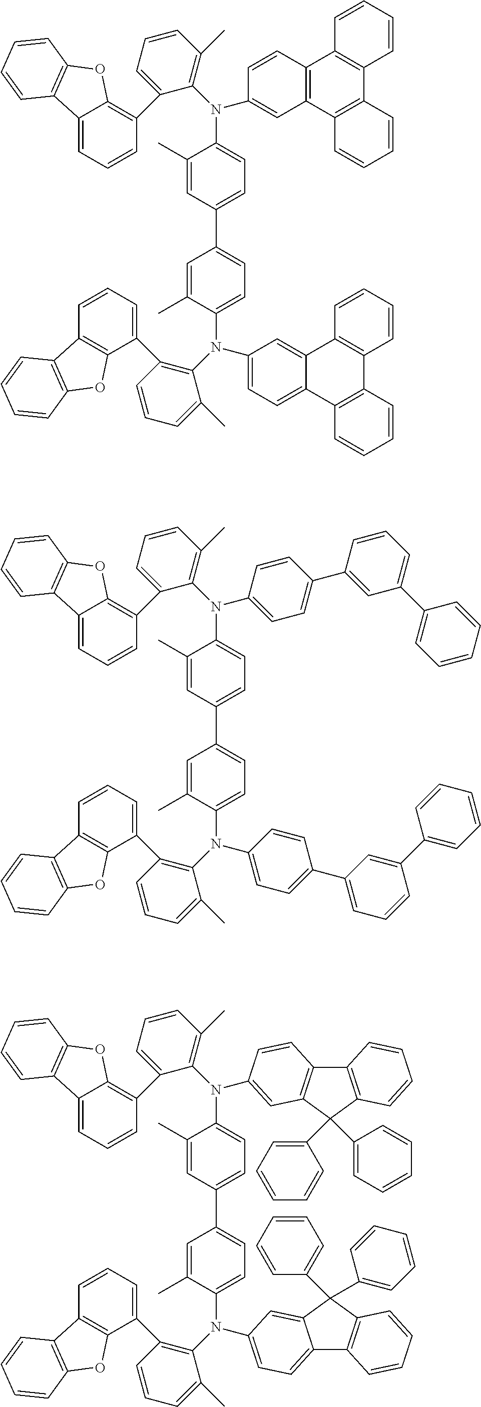 [SMILES: Cc1cc(-c2ccc(N(c3ccc4c5ccccc5c5ccccc5c4c3)c3c(C)cccc3-c3cccc4c3oc3ccccc34)c(C)c2)ccc1N(c1ccc2c3ccccc3c3ccccc3c2c1)c1c(C)cccc1-c1cccc2c1oc1ccccc12 ; Cc1cc(-c2ccc(N(c3ccc(-c4cccc(-c5ccccc5)c4)cc3)c3c(C)cccc3-c3cccc4c3oc3ccccc34)c(C)c2)ccc1N(c1ccc(-c2cccc(-c3ccccc3)c2)cc1)c1c(C)cccc1-c1cccc2c1oc1ccccc12 ; Cc1cc(-c2ccc(N(c3ccc4c(c3)C(c3ccccc3)(c3ccccc3)c3ccccc3-4)c3c(C)cccc3-c3cccc4c3oc3ccccc34)c(C)c2)ccc1N(c1ccc2c(c1)C(c1ccccc1)(c1ccccc1)c1ccccc1-2)c1c(C)cccc1-c1cccc2c1oc1ccccc12]

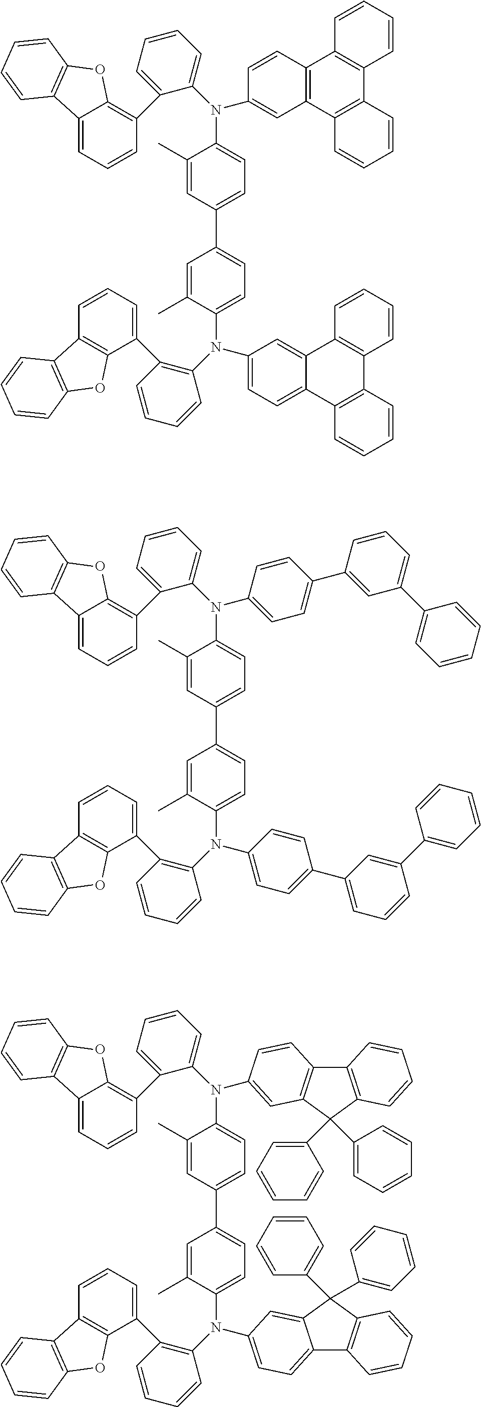 [SMILES: Cc1cc(-c2ccc(N(c3ccc4c5ccccc5c5ccccc5c4c3)c3ccccc3-c3cccc4c3oc3ccccc34)c(C)c2)ccc1N(c1ccc2c3ccccc3c3ccccc3c2c1)c1ccccc1-c1cccc2c1oc1ccccc12 ; Cc1cc(-c2ccc(N(c3ccc(-c4cccc(-c5ccccc5)c4)cc3)c3ccccc3-c3cccc4c3oc3ccccc34)c(C)c2)ccc1N(c1ccc(-c2cccc(-c3ccccc3)c2)cc1)c1ccccc1-c1cccc2c1oc1ccccc12 ; Cc1cc(-c2ccc(N(c3ccc4c(c3)C(c3ccccc3)(c3ccccc3)c3ccccc3-4)c3ccccc3-c3cccc4c3oc3ccccc34)c(C)c2)ccc1N(c1ccc2c(c1)C(c1ccccc1)(c1ccccc1)c1ccccc1-2)c1ccccc1-c1cccc2c1oc1ccccc12]

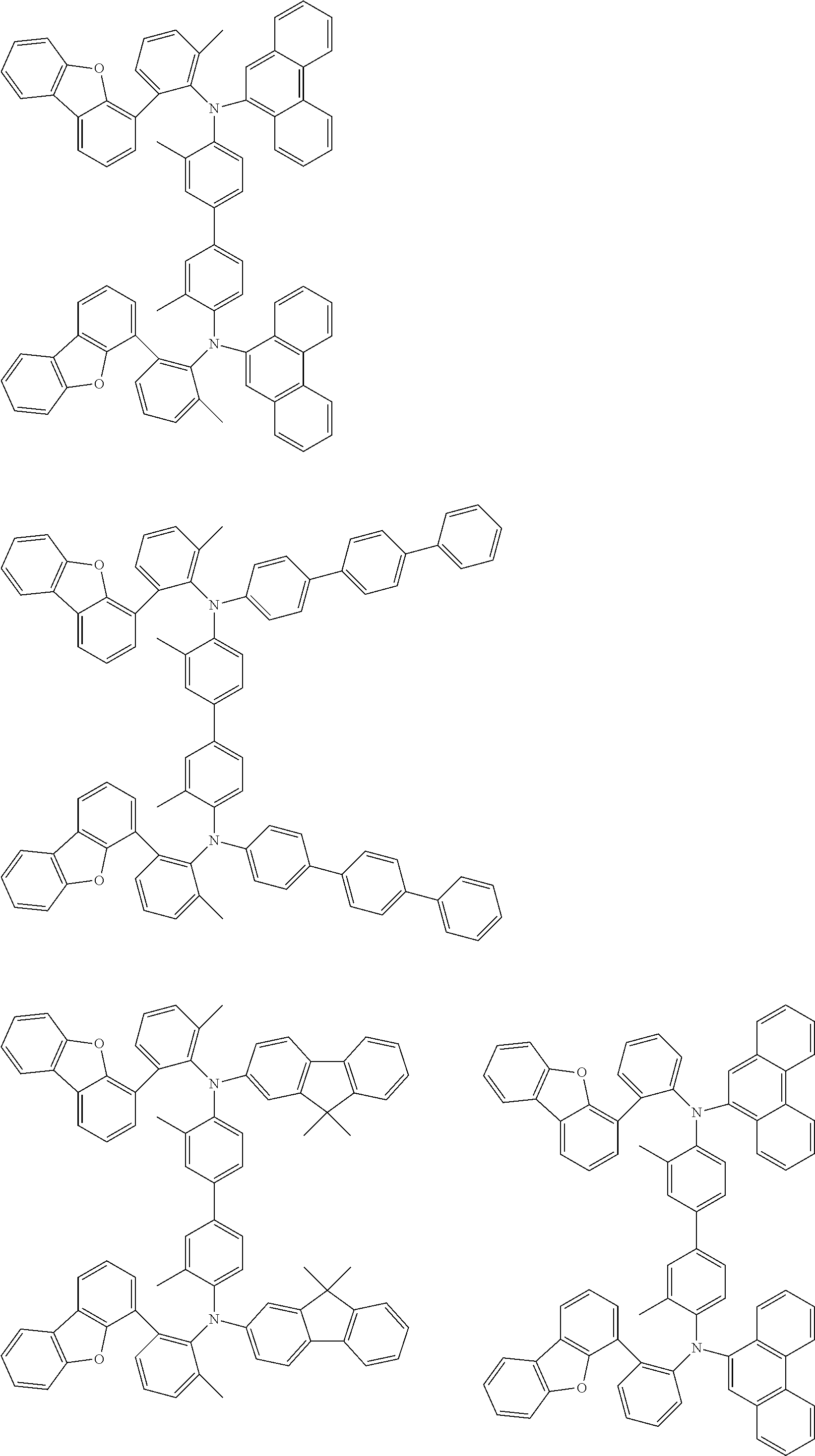

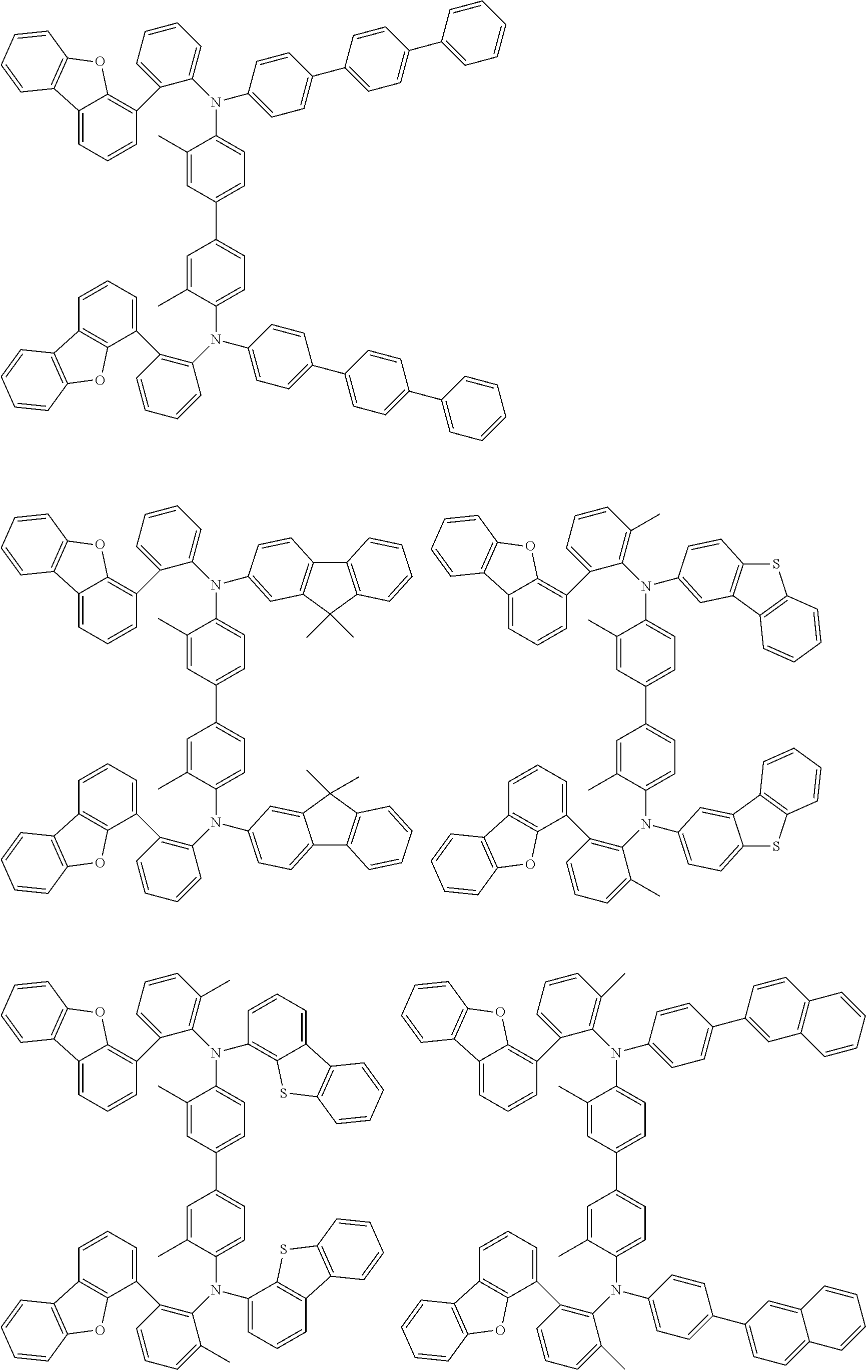

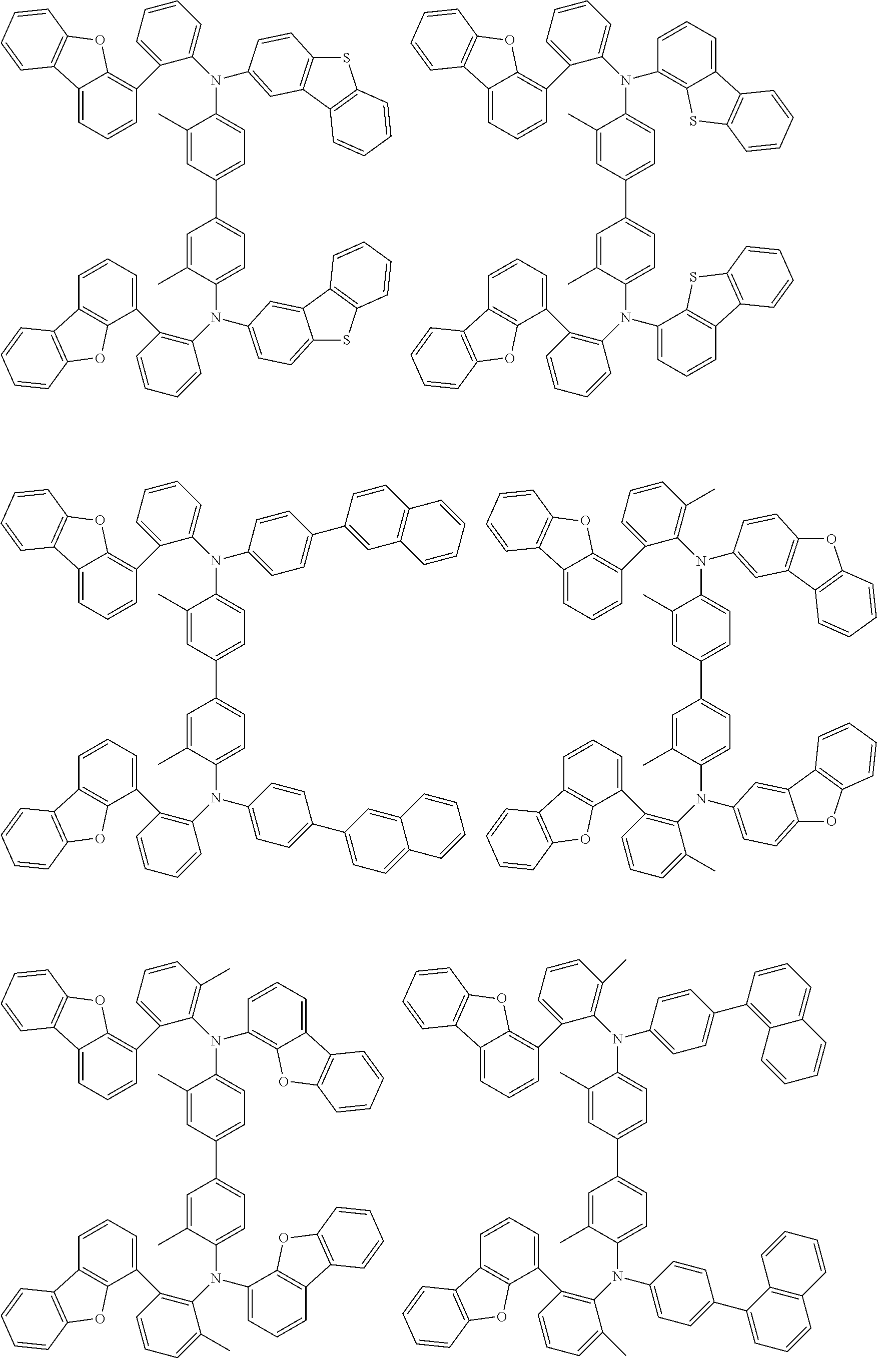

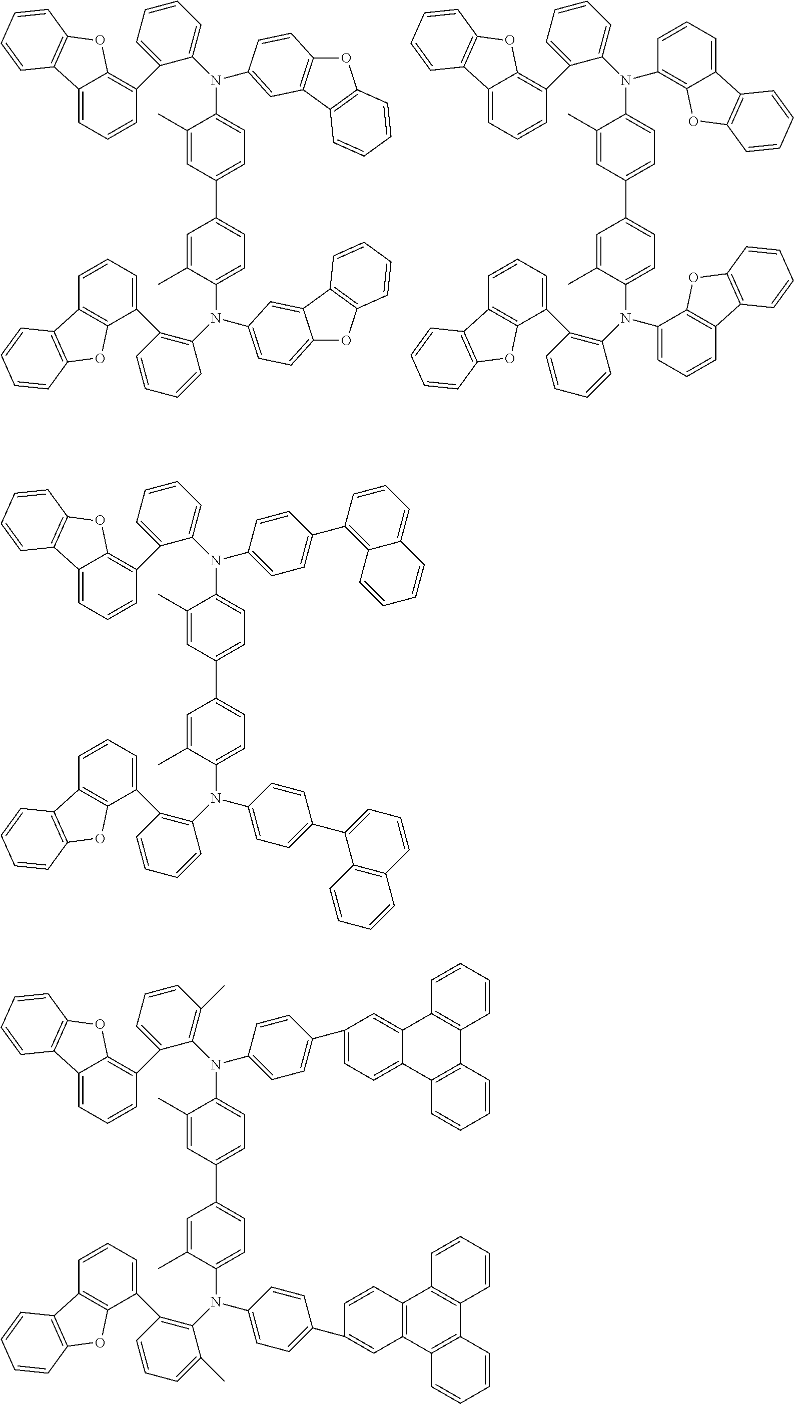

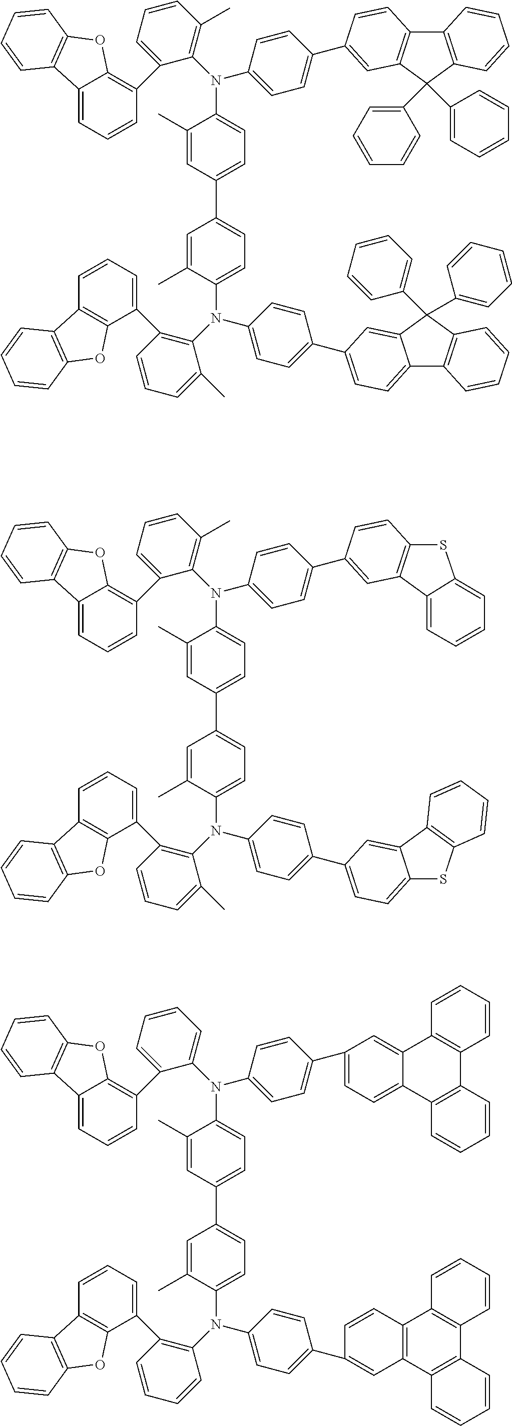

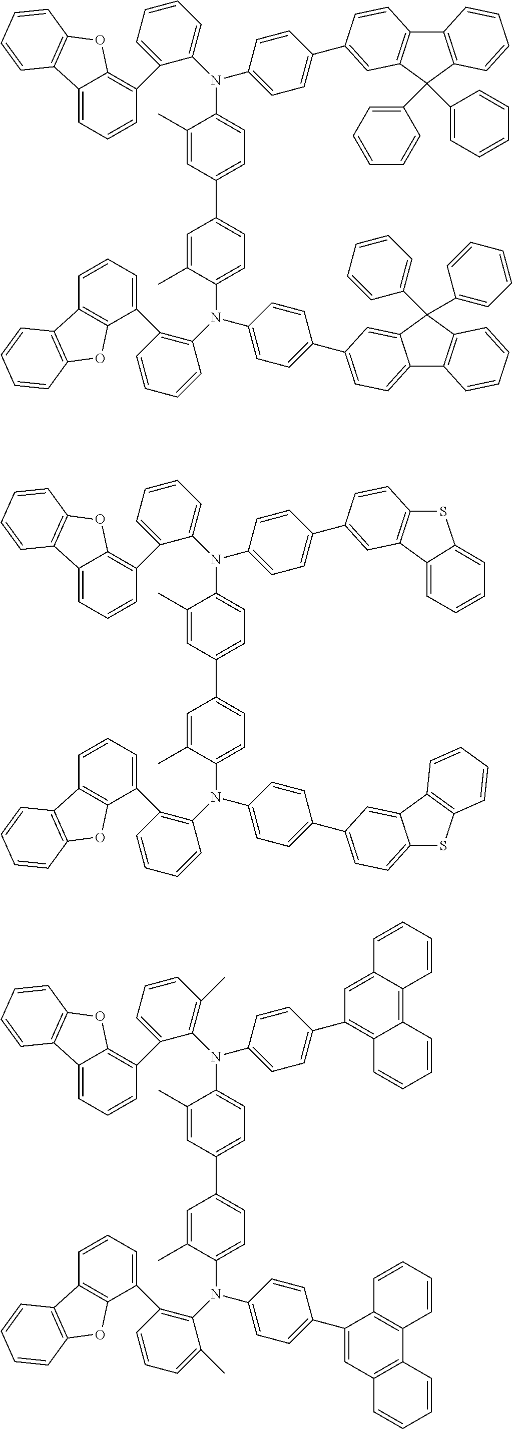

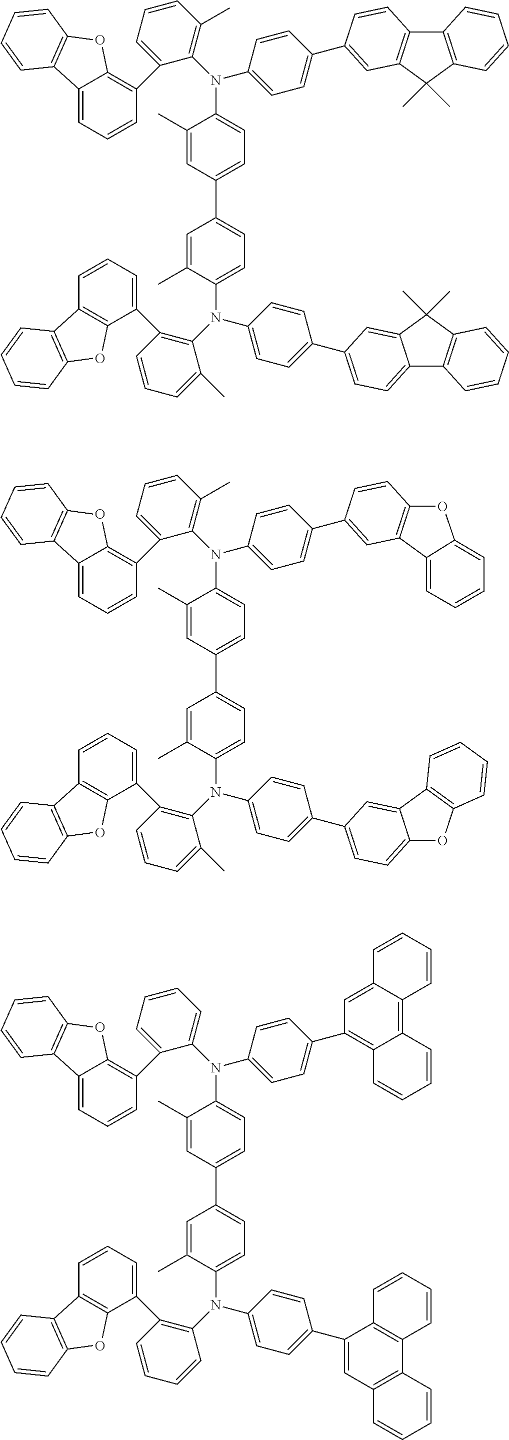 [SMILES: Cc1cc(-c2ccc(N(c3ccc(-c4ccc5c(c4)C(C)(C)c4ccccc4-5)cc3)c3c(C)cccc3-c3cccc4c3oc3ccccc34)c(C)c2)ccc1N(c1ccc(-c2ccc3c(c2)C(C)(C)c2ccccc2-3)cc1)c1c(C)cccc1-c1cccc2c1oc1ccccc12 ; Cc1cc(-c2ccc(N(c3ccc(-c4ccc5oc6ccccc6c5c4)cc3)c3c(C)cccc3-c3cccc4c3oc3ccccc34)c(C)c2)ccc1N(c1ccc(-c2ccc3oc4ccccc4c3c2)cc1)c1c(C)cccc1-c1cccc2c1oc1ccccc12 ; Cc1cc(-c2ccc(N(c3ccc(-c4cc5ccccc5c5ccccc45)cc3)c3ccccc3-c3cccc4c3oc3ccccc34)c(C)c2)ccc1N(c1ccc(-c2cc3ccccc3c3ccccc23)cc1)c1ccccc1-c1cccc2c1oc1ccccc12]

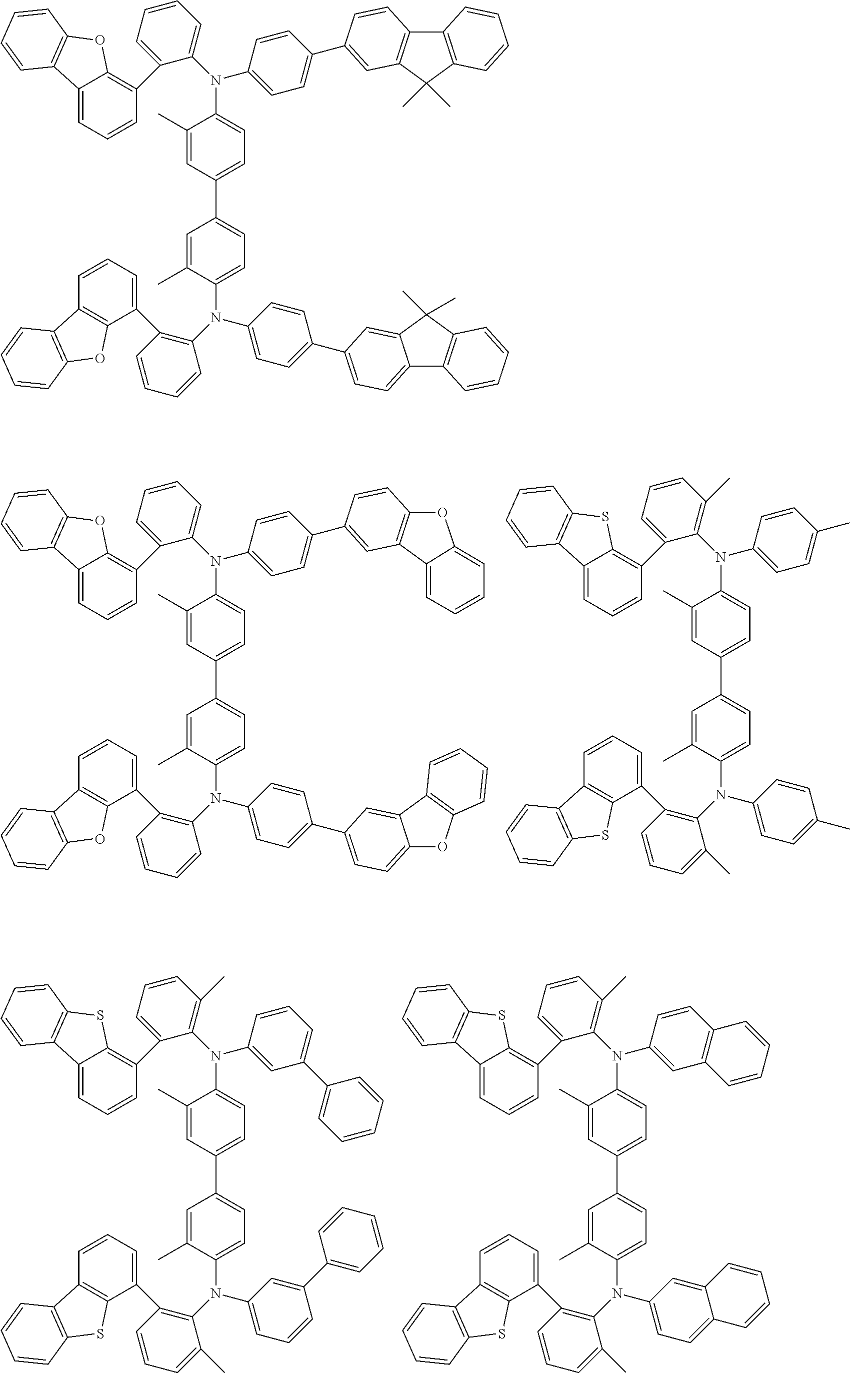

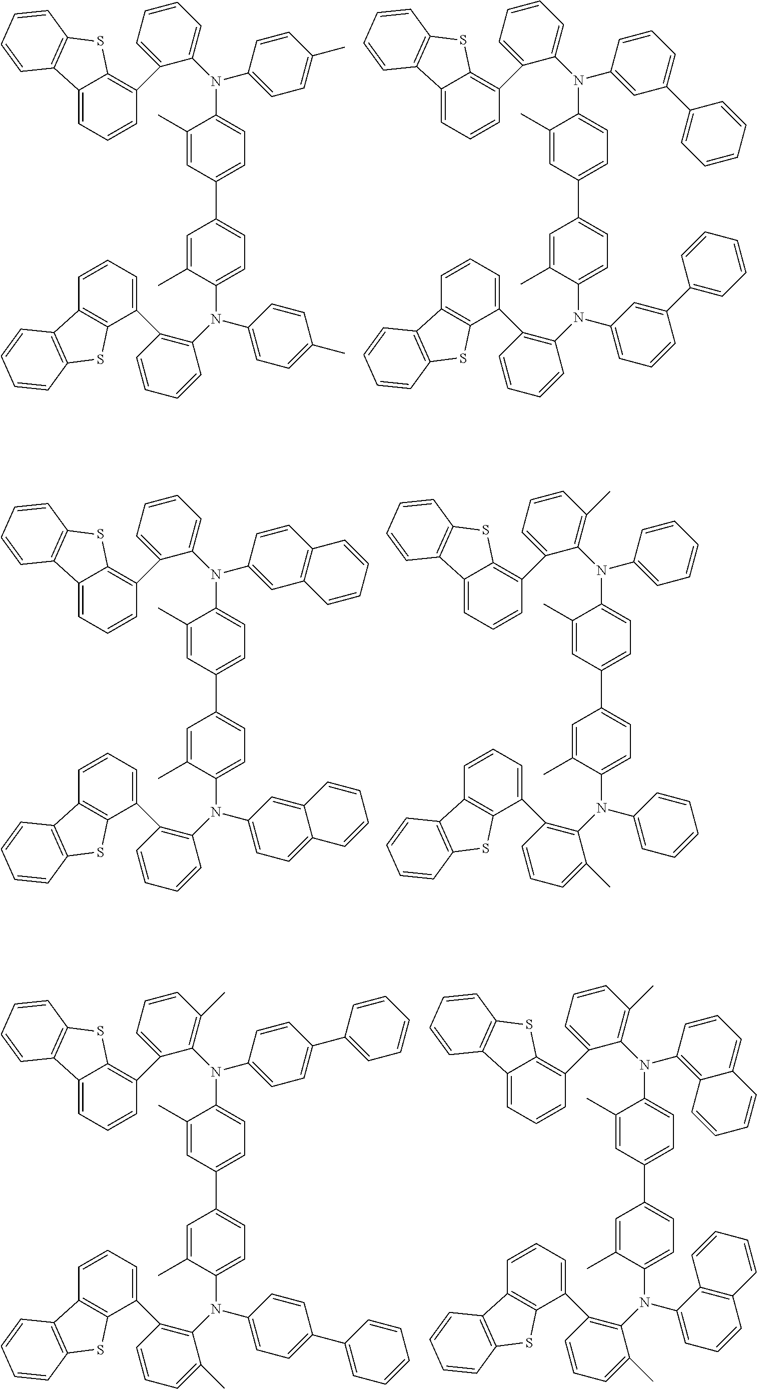

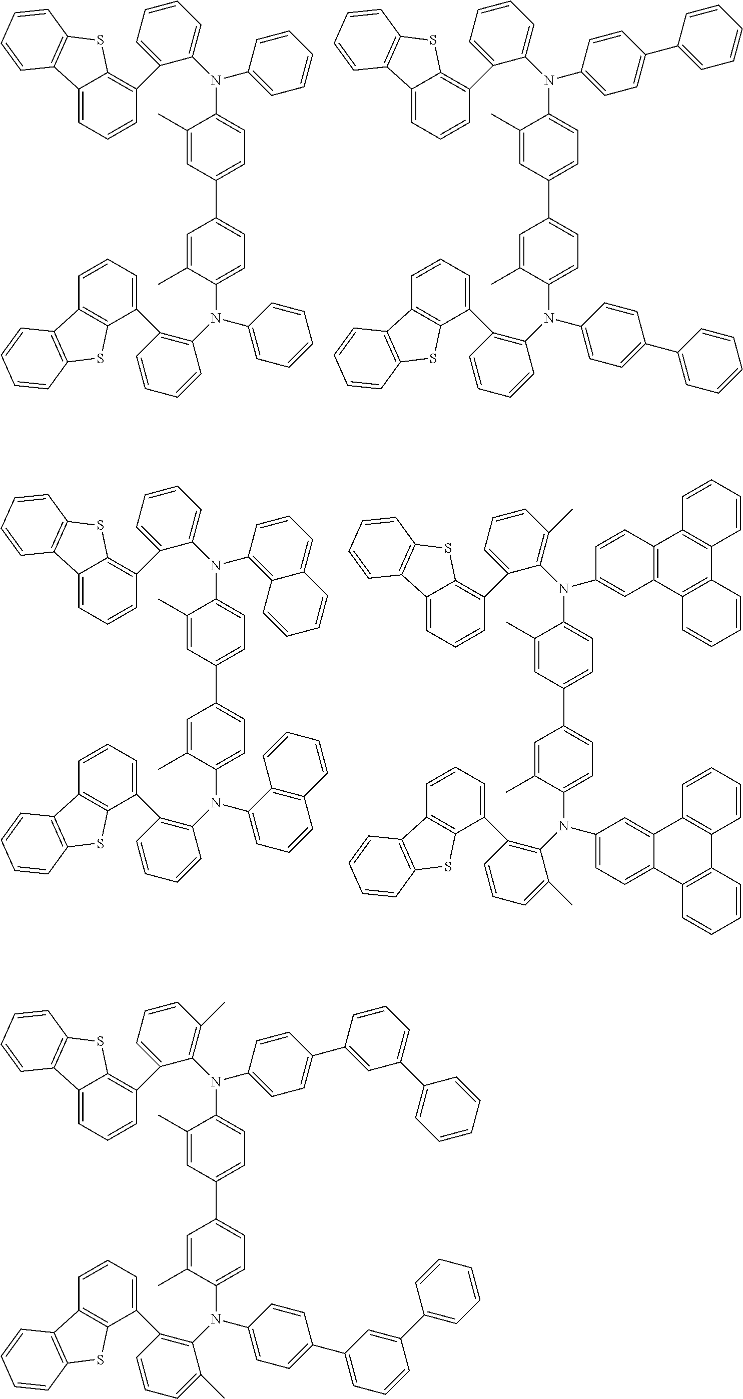

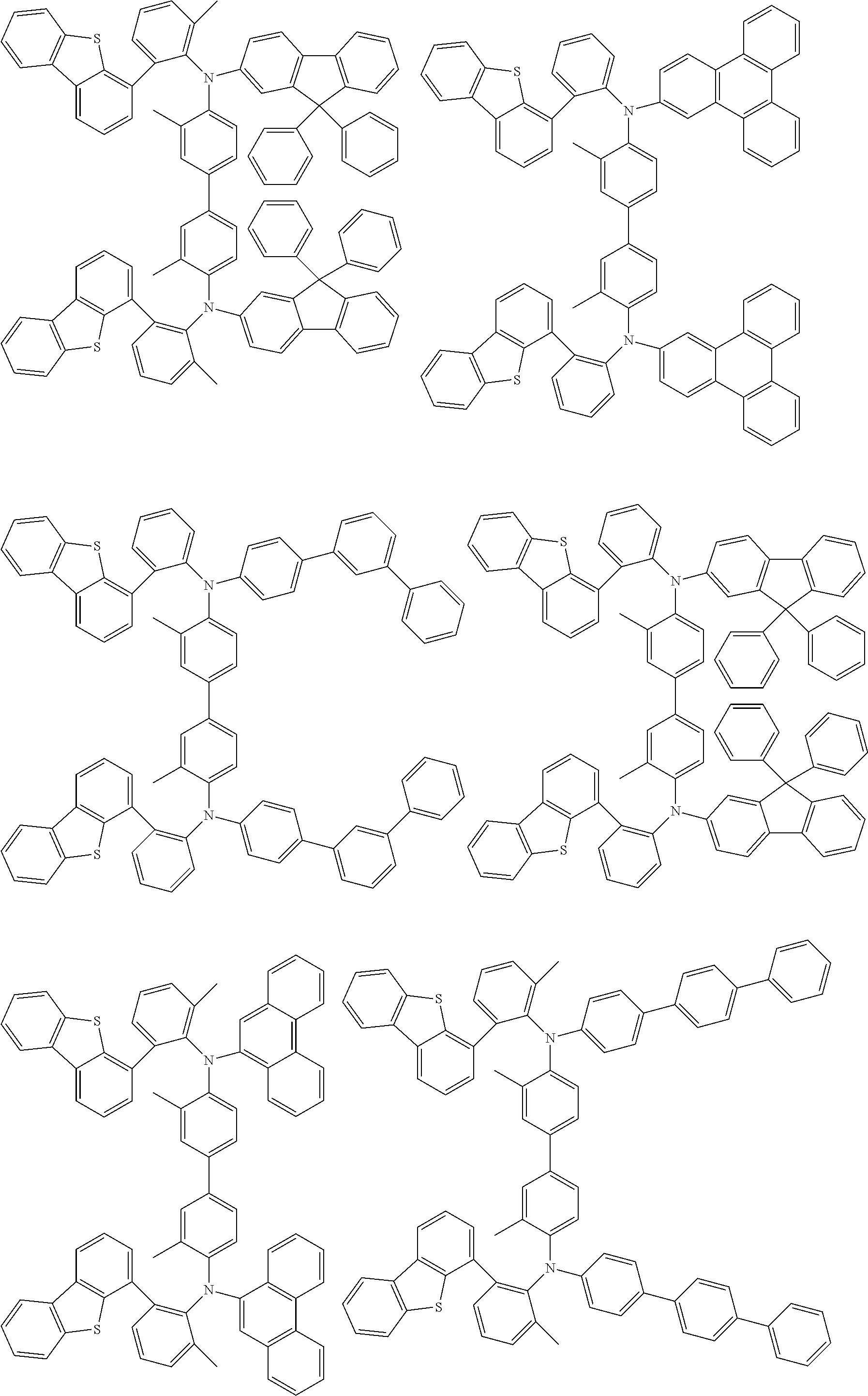

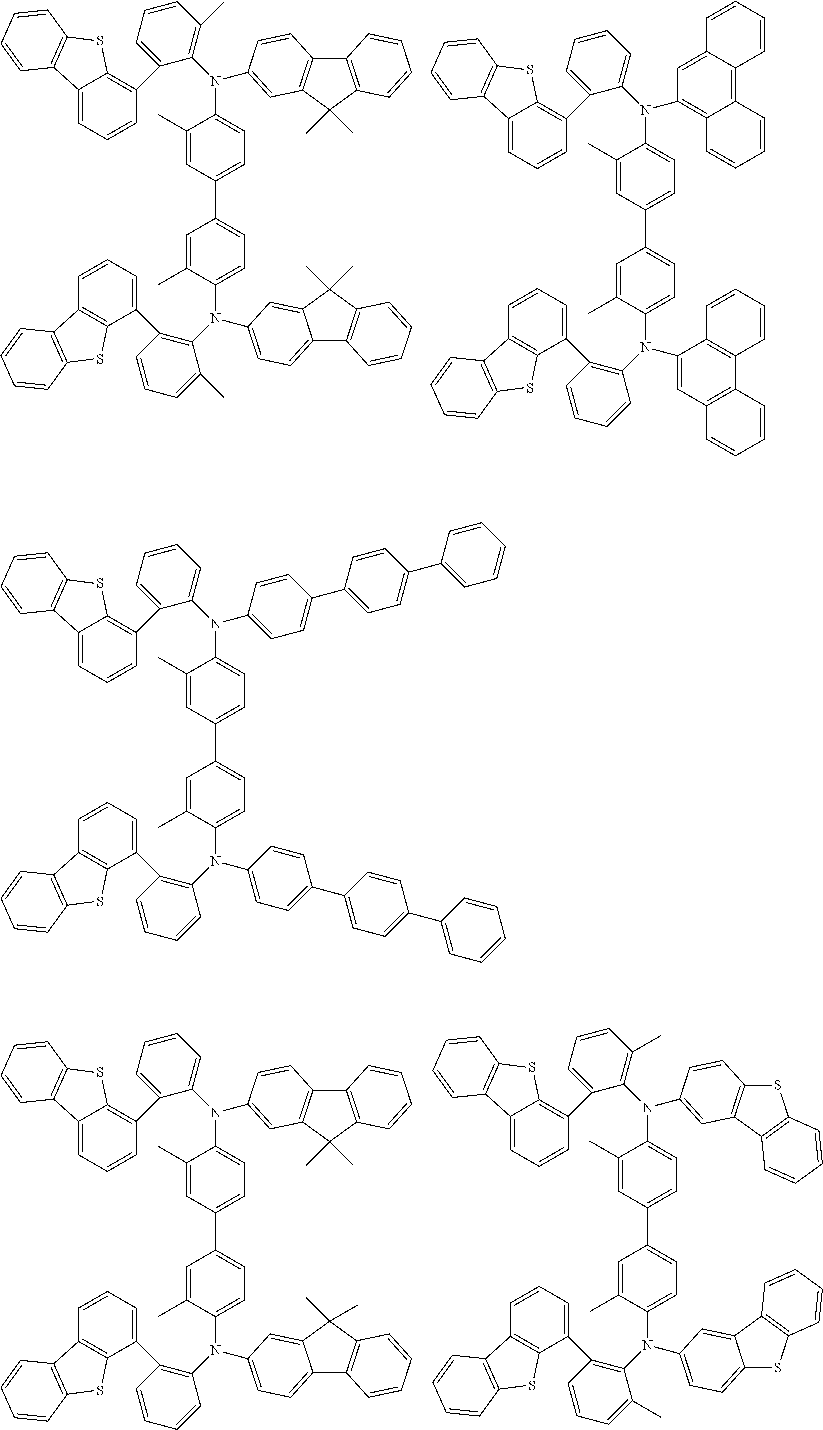

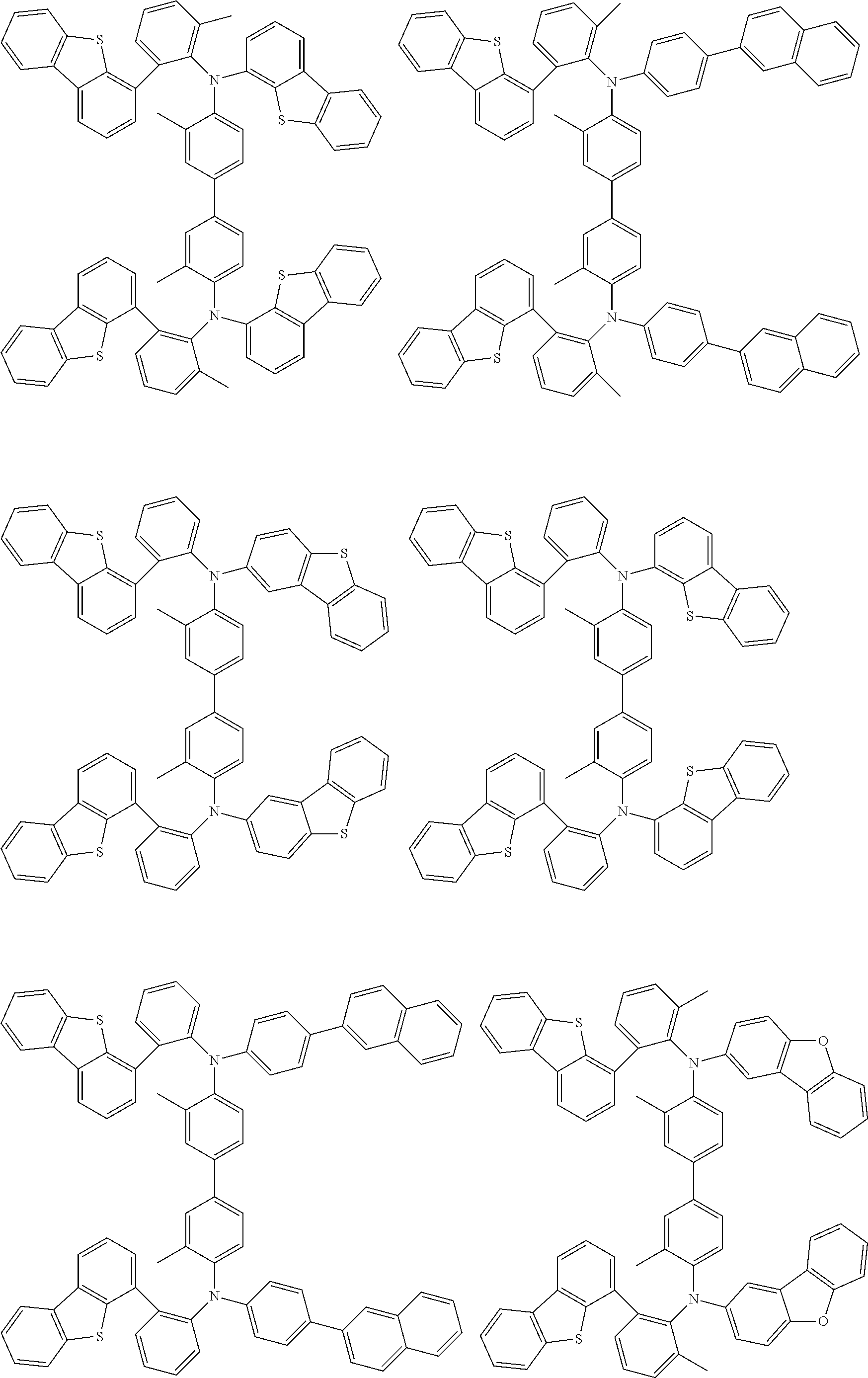

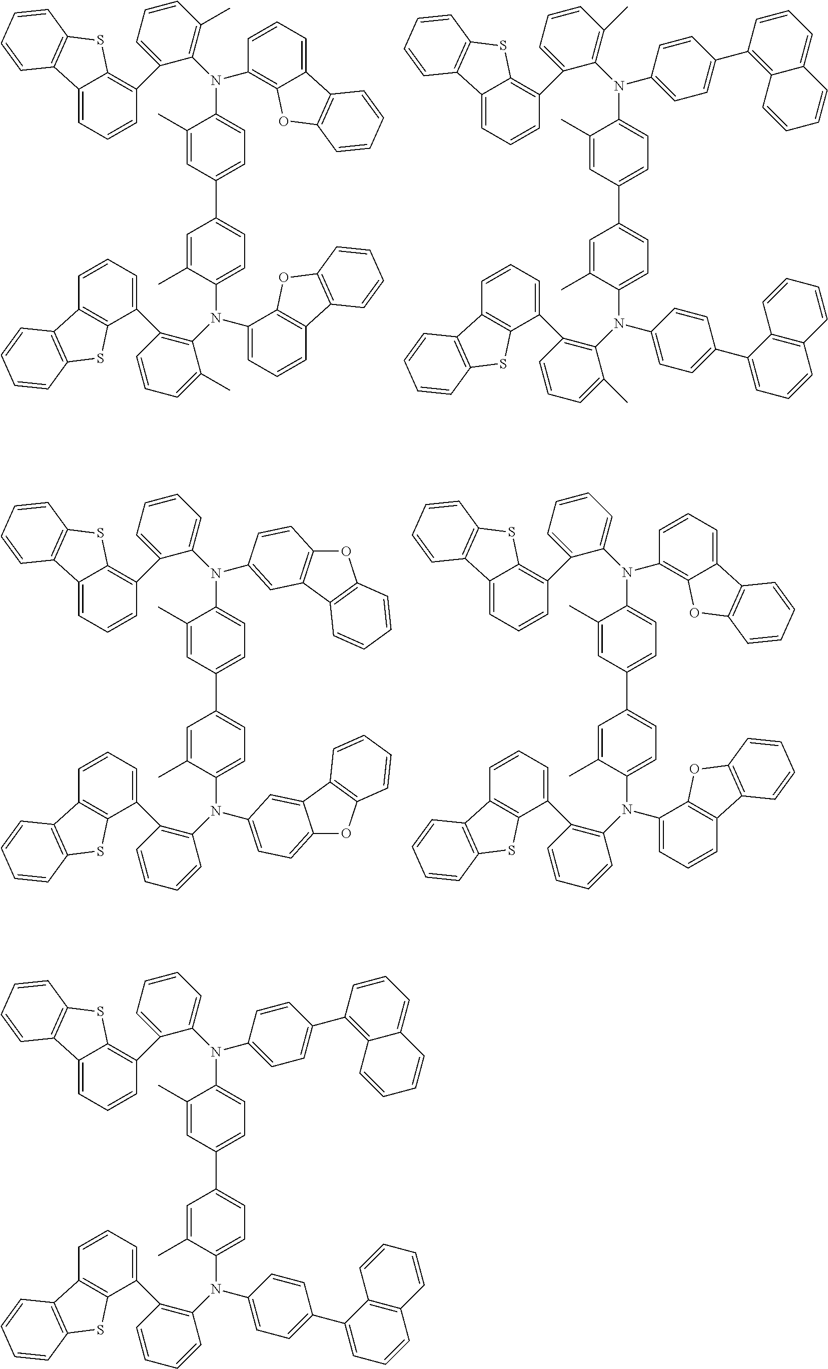

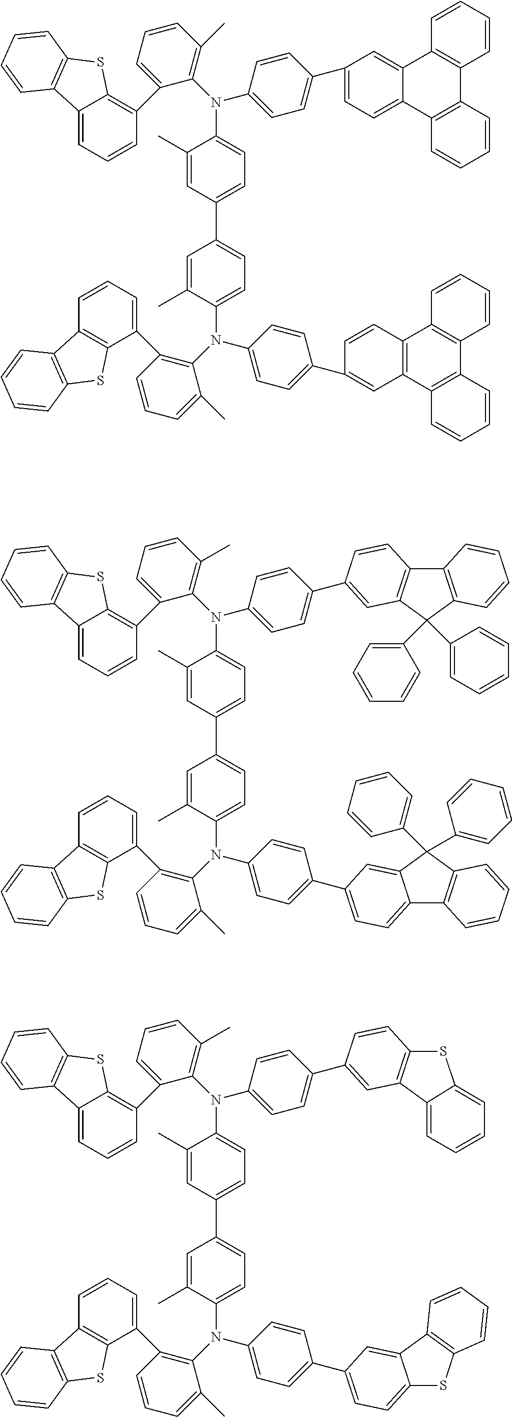

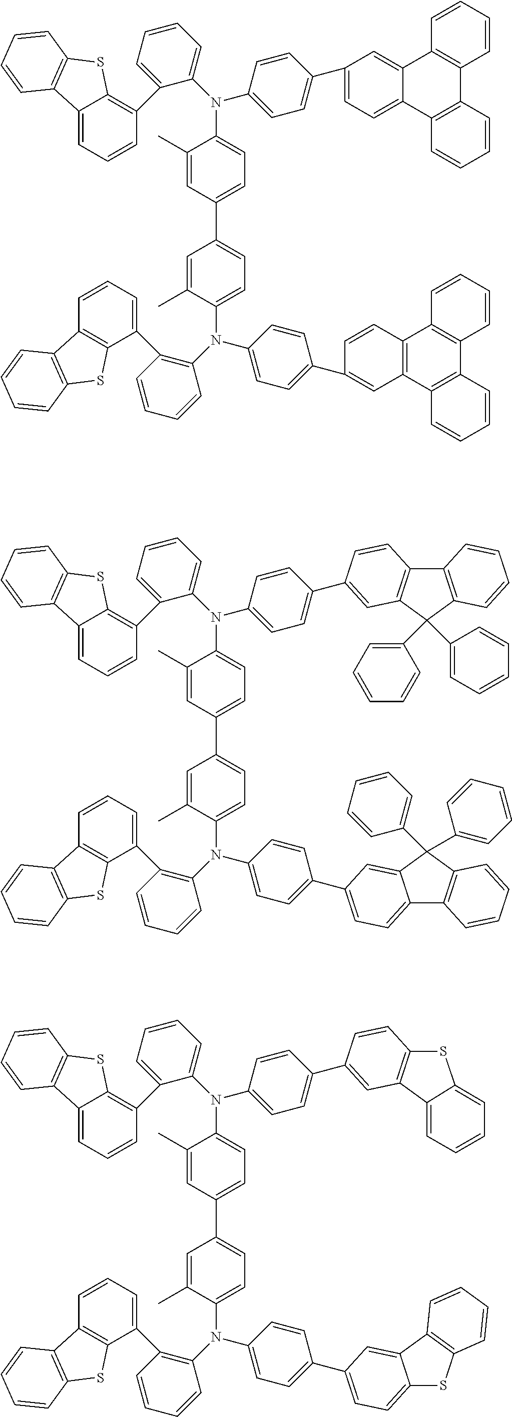

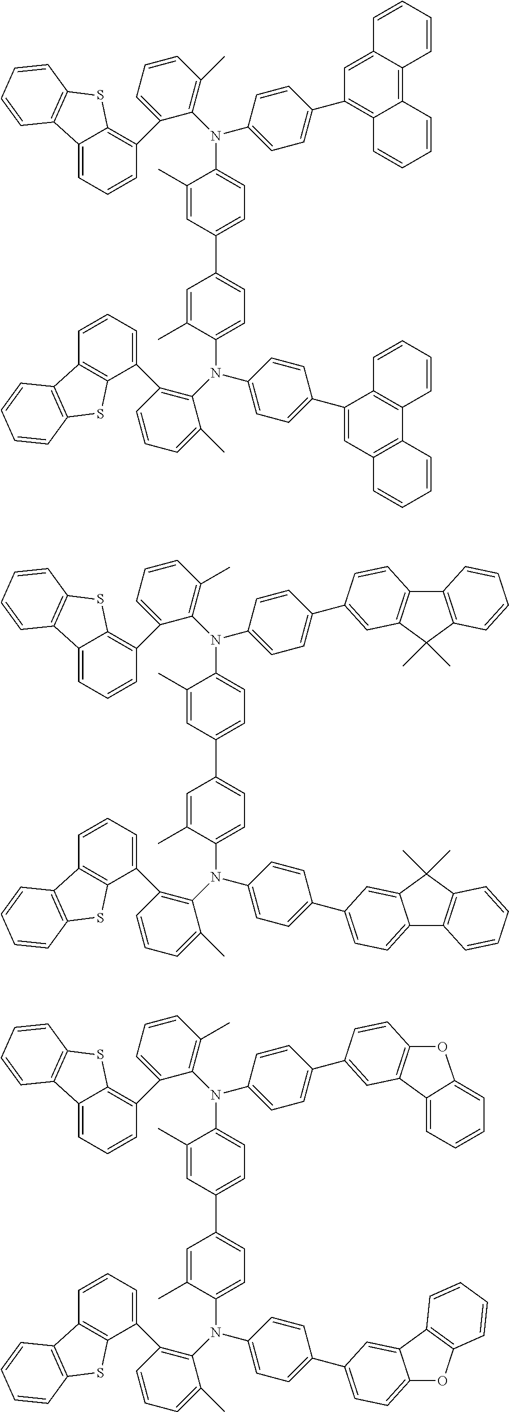 [SMILES: Cc1cc(-c2ccc(N(c3ccc(-c4cc5ccccc5c5ccccc45)cc3)c3c(C)cccc3-c3cccc4c3sc3ccccc34)c(C)c2)ccc1N(c1ccc(-c2cc3ccccc3c3ccccc23)cc1)c1c(C)cccc1-c1cccc2c1sc1ccccc12 ; Cc1cc(-c2ccc(N(c3ccc(-c4ccc5c(c4)C(C)(C)c4ccccc4-5)cc3)c3c(C)cccc3-c3cccc4c3sc3ccccc34)c(C)c2)ccc1N(c1ccc(-c2ccc3c(c2)C(C)(C)c2ccccc2-3)cc1)c1c(C)cccc1-c1cccc2c1sc1ccccc12 ; Cc1cc(-c2ccc(N(c3ccc(-c4ccc5oc6ccccc6c5c4)cc3)c3c(C)cccc3-c3cccc4c3sc3ccccc34)c(C)c2)ccc1N(c1ccc(-c2ccc3oc4ccccc4c3c2)cc1)c1c(C)cccc1-c1cccc2c1sc1ccccc12]

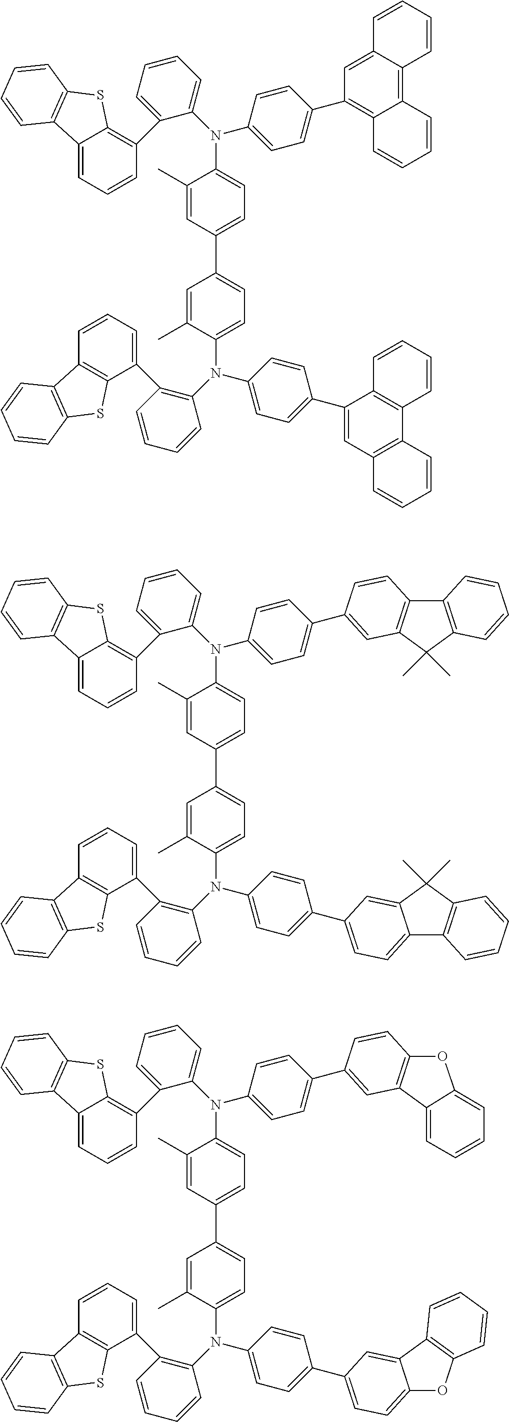 [SMILES: Cc1cc(-c2ccc(N(c3ccc(-c4cc5ccccc5c5ccccc45)cc3)c3ccccc3-c3cccc4c3sc3ccccc34)c(C)c2)ccc1N(c1ccc(-c2cc3ccccc3c3ccccc23)cc1)c1ccccc1-c1cccc2c1sc1ccccc12 ; Cc1cc(-c2ccc(N(c3ccc(-c4ccc5c(c4)C(C)(C)c4ccccc4-5)cc3)c3ccccc3-c3cccc4c3sc3ccccc34)c(C)c2)ccc1N(c1ccc(-c2ccc3c(c2)C(C)(C)c2ccccc2-3)cc1)c1ccccc1-c1cccc2c1sc1ccccc12 ; Cc1cc(-c2ccc(N(c3ccc(-c4ccc5oc6ccccc6c5c4)cc3)c3ccccc3-c3cccc4c3sc3ccccc34)c(C)c2)ccc1N(c1ccc(-c2ccc3oc4ccccc4c3c2)cc1)c1ccccc1-c1cccc2c1sc1ccccc12]

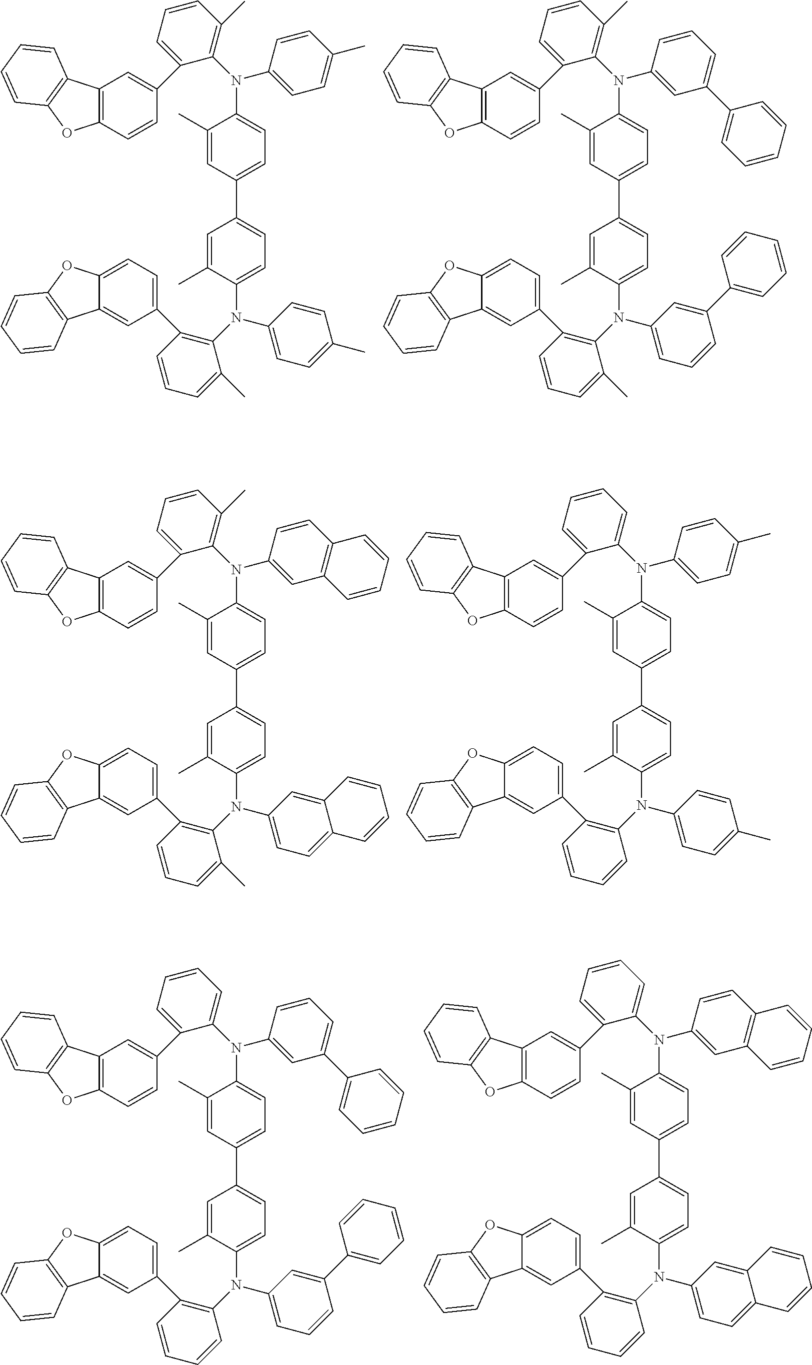

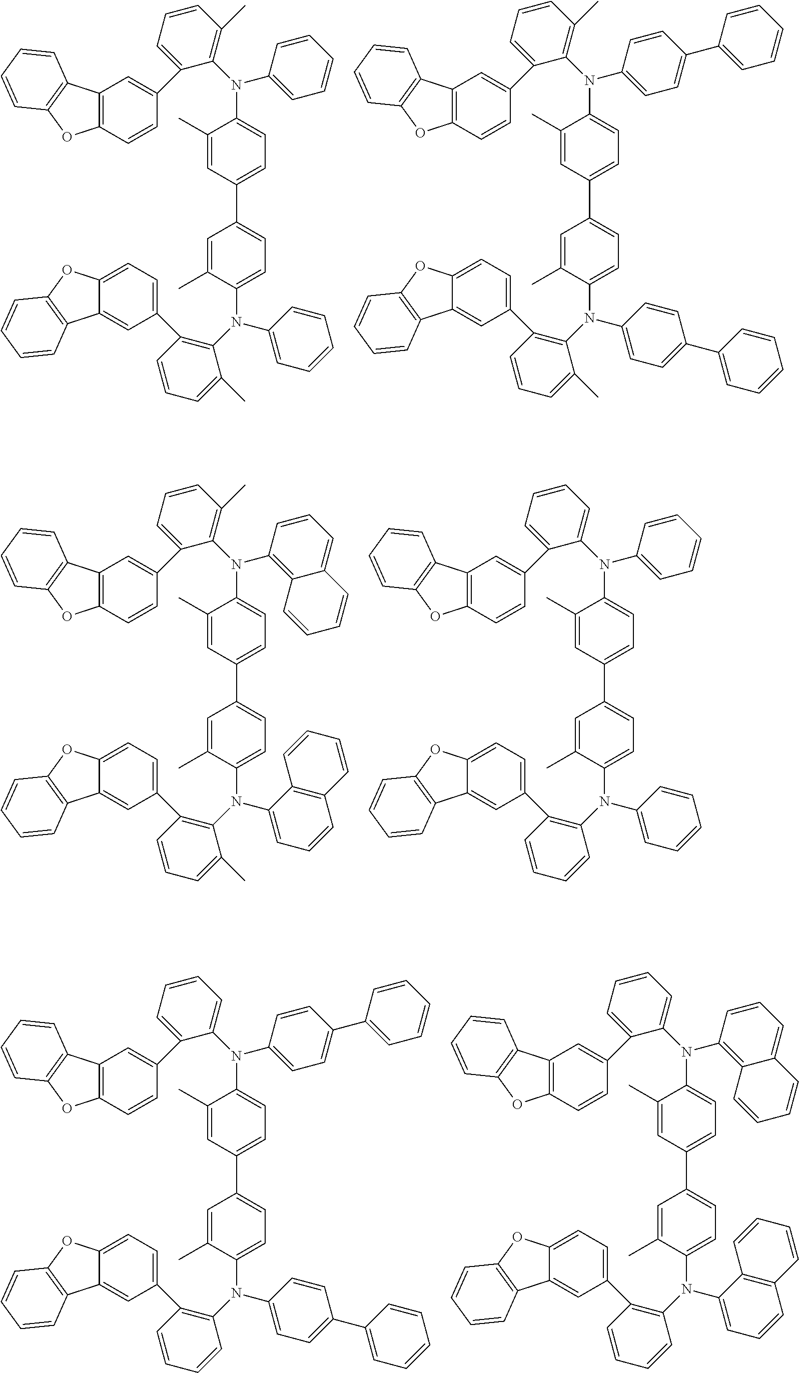

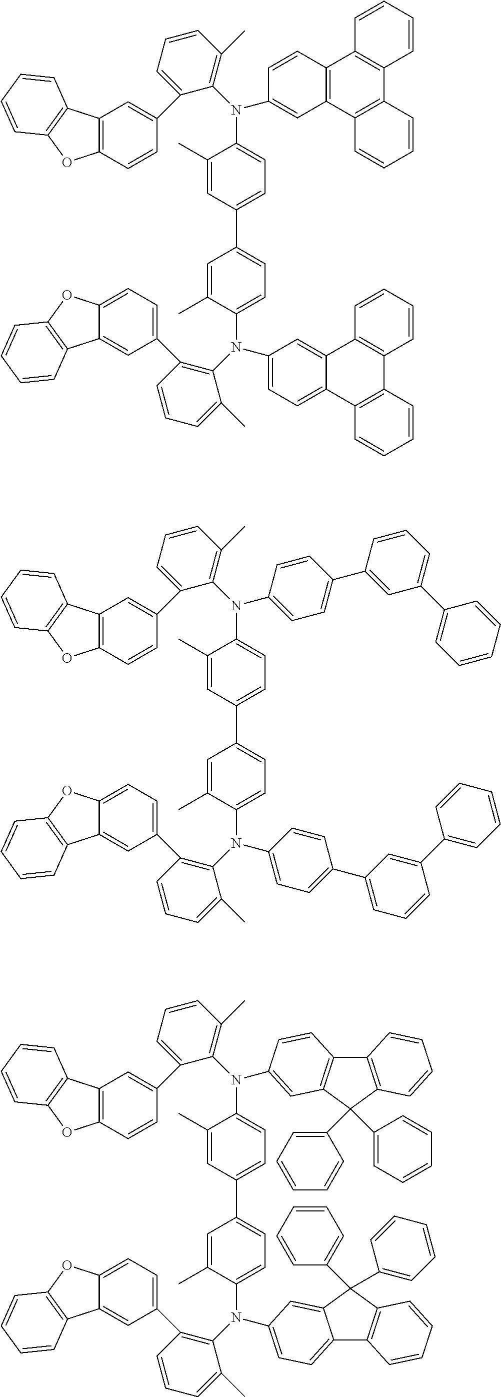 [SMILES: Cc1cc(-c2ccc(N(c3ccc4c5ccccc5c5ccccc5c4c3)c3c(C)cccc3-c3ccc4oc5ccccc5c4c3)c(C)c2)ccc1N(c1ccc2c3ccccc3c3ccccc3c2c1)c1c(C)cccc1-c1ccc2oc3ccccc3c2c1 ; Cc1cc(-c2ccc(N(c3ccc(-c4cccc(-c5ccccc5)c4)cc3)c3c(C)cccc3-c3ccc4oc5ccccc5c4c3)c(C)c2)ccc1N(c1ccc(-c2cccc(-c3ccccc3)c2)cc1)c1c(C)cccc1-c1ccc2oc3ccccc3c2c1 ; Cc1cc(-c2ccc(N(c3ccc4c(c3)C(c3ccccc3)(c3ccccc3)c3ccccc3-4)c3c(C)cccc3-c3ccc4oc5ccccc5c4c3)c(C)c2)ccc1N(c1ccc2c(c1)C(c1ccccc1)(c1ccccc1)c1ccccc1-2)c1c(C)cccc1-c1ccc2oc3ccccc3c2c1]

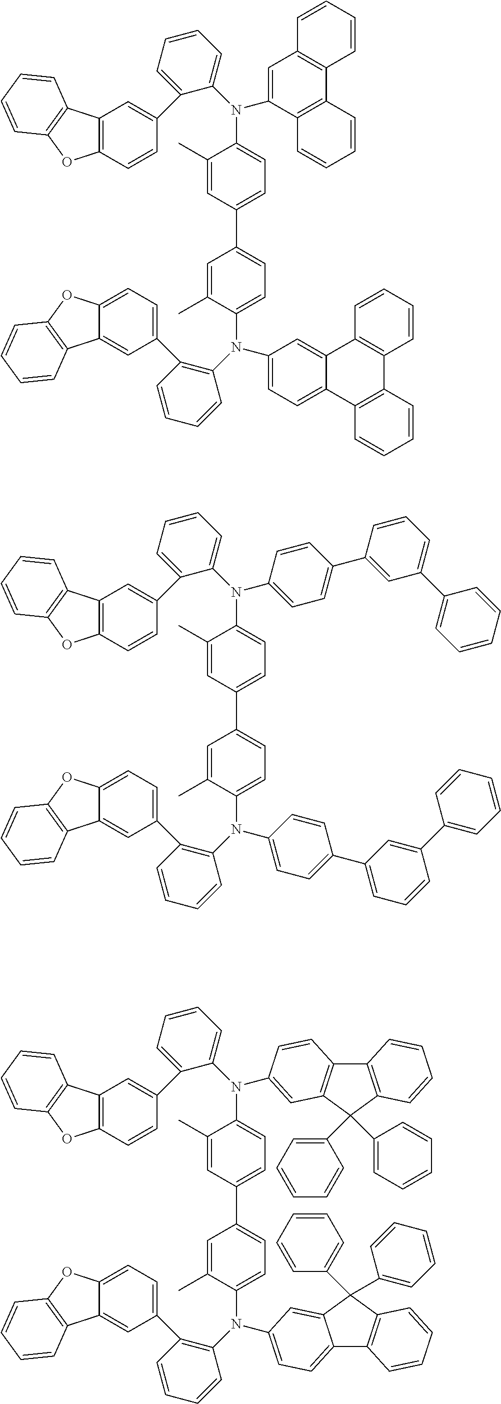 [SMILES: Cc1cc(-c2ccc(N(c3ccccc3-c3ccc4oc5ccccc5c4c3)c3cc4ccccc4c4ccccc34)c(C)c2)ccc1N(c1ccc2c3ccccc3c3ccccc3c2c1)c1ccccc1-c1ccc2oc3ccccc3c2c1 ; Cc1cc(-c2ccc(N(c3ccc(-c4cccc(-c5ccccc5)c4)cc3)c3ccccc3-c3ccc4oc5ccccc5c4c3)c(C)c2)ccc1N(c1ccc(-c2cccc(-c3ccccc3)c2)cc1)c1ccccc1-c1ccc2oc3ccccc3c2c1 ; Cc1cc(-c2ccc(N(c3ccc4c(c3)C(c3ccccc3)(c3ccccc3)c3ccccc3-4)c3ccccc3-c3ccc4oc5ccccc5c4c3)c(C)c2)ccc1N(c1ccc2c(c1)C(c1ccccc1)(c1ccccc1)c1ccccc1-2)c1ccccc1-c1ccc2oc3ccccc3c2c1]

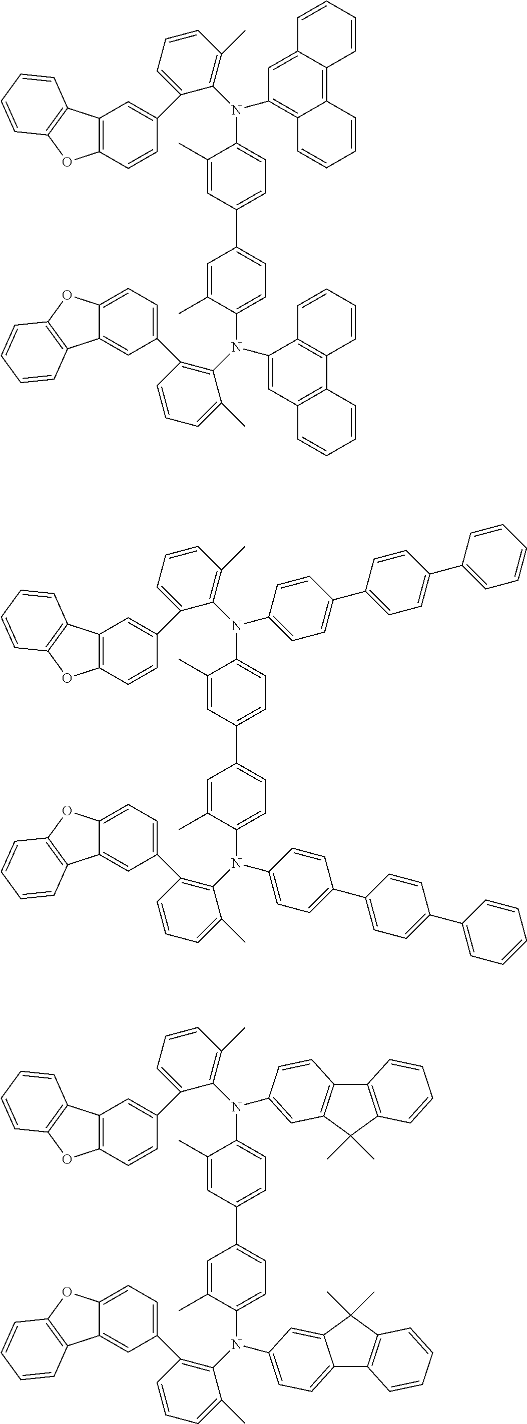 [SMILES: Cc1cc(-c2ccc(N(c3c(C)cccc3-c3ccc4oc5ccccc5c4c3)c3cc4ccccc4c4ccccc34)c(C)c2)ccc1N(c1c(C)cccc1-c1ccc2oc3ccccc3c2c1)c1cc2ccccc2c2ccccc12 ; Cc1cc(-c2ccc(N(c3ccc(-c4ccc(-c5ccccc5)cc4)cc3)c3c(C)cccc3-c3ccc4oc5ccccc5c4c3)c(C)c2)ccc1N(c1ccc(-c2ccc(-c3ccccc3)cc2)cc1)c1c(C)cccc1-c1ccc2oc3ccccc3c2c1 ; Cc1cc(-c2ccc(N(c3ccc4c(c3)C(C)(C)c3ccccc3-4)c3c(C)cccc3-c3ccc4oc5ccccc5c4c3)c(C)c2)ccc1N(c1ccc2c(c1)C(C)(C)c1ccccc1-2)c1c(C)cccc1-c1ccc2oc3ccccc3c2c1]

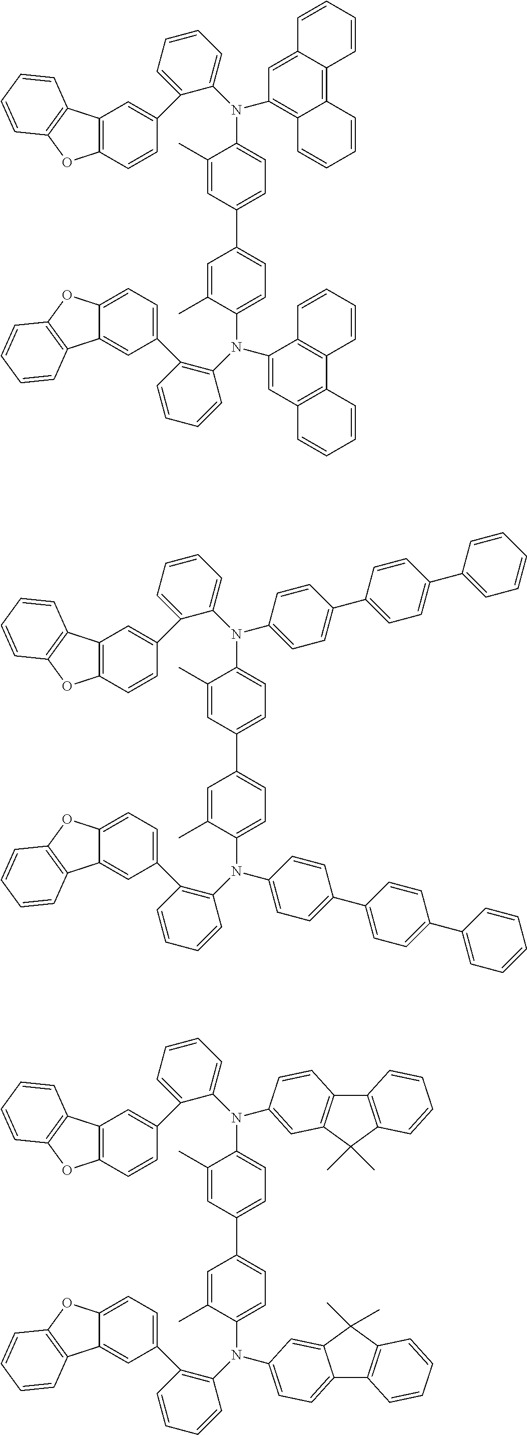 [SMILES: Cc1cc(-c2ccc(N(c3ccccc3-c3ccc4oc5ccccc5c4c3)c3cc4ccccc4c4ccccc34)c(C)c2)ccc1N(c1ccccc1-c1ccc2oc3ccccc3c2c1)c1cc2ccccc2c2ccccc12 ; Cc1cc(-c2ccc(N(c3ccc(-c4ccc(-c5ccccc5)cc4)cc3)c3ccccc3-c3ccc4oc5ccccc5c4c3)c(C)c2)ccc1N(c1ccc(-c2ccc(-c3ccccc3)cc2)cc1)c1ccccc1-c1ccc2oc3ccccc3c2c1 ; Cc1cc(-c2ccc(N(c3ccc4c(c3)C(C)(C)c3ccccc3-4)c3ccccc3-c3ccc4oc5ccccc5c4c3)c(C)c2)ccc1N(c1ccc2c(c1)C(C)(C)c1ccccc1-2)c1ccccc1-c1ccc2oc3ccccc3c2c1]

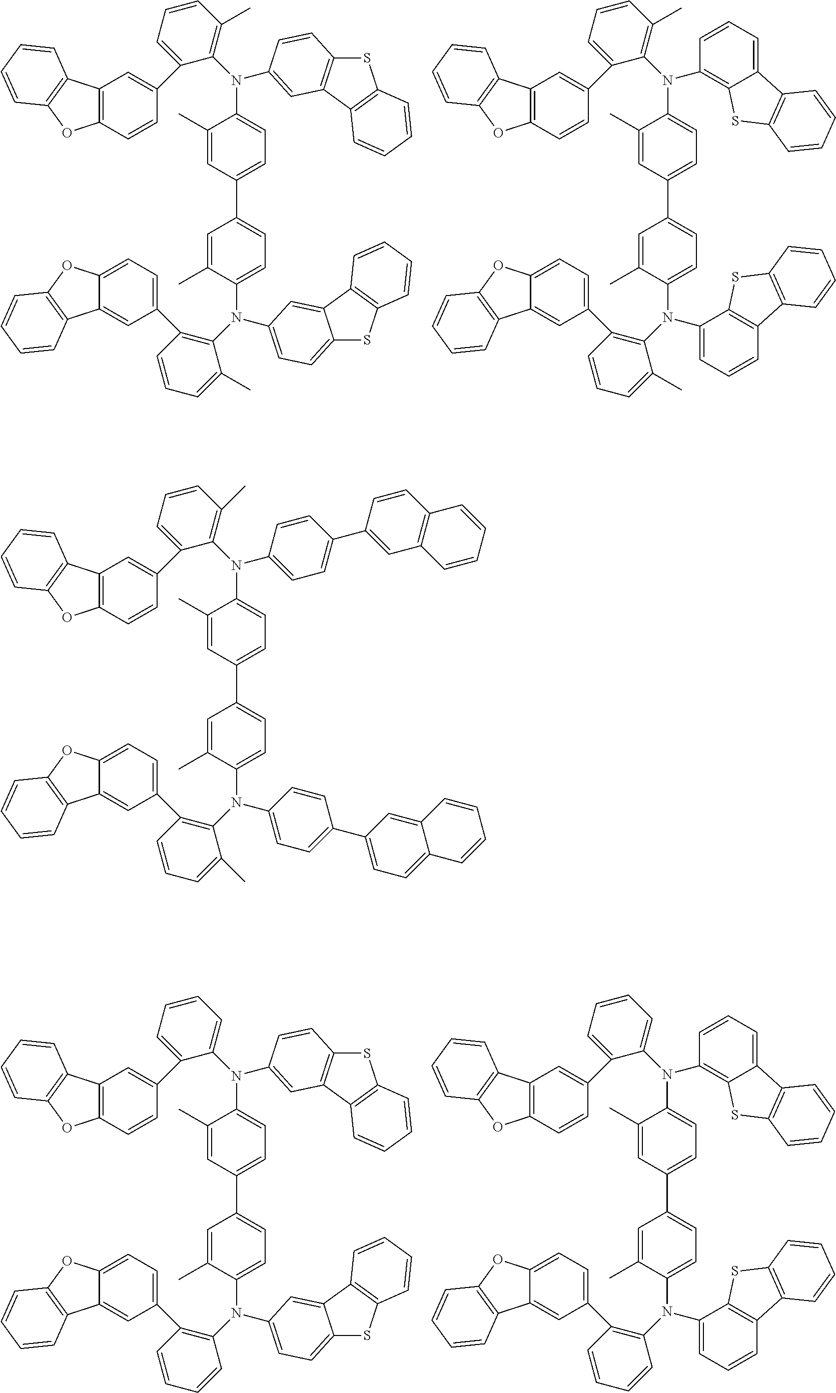

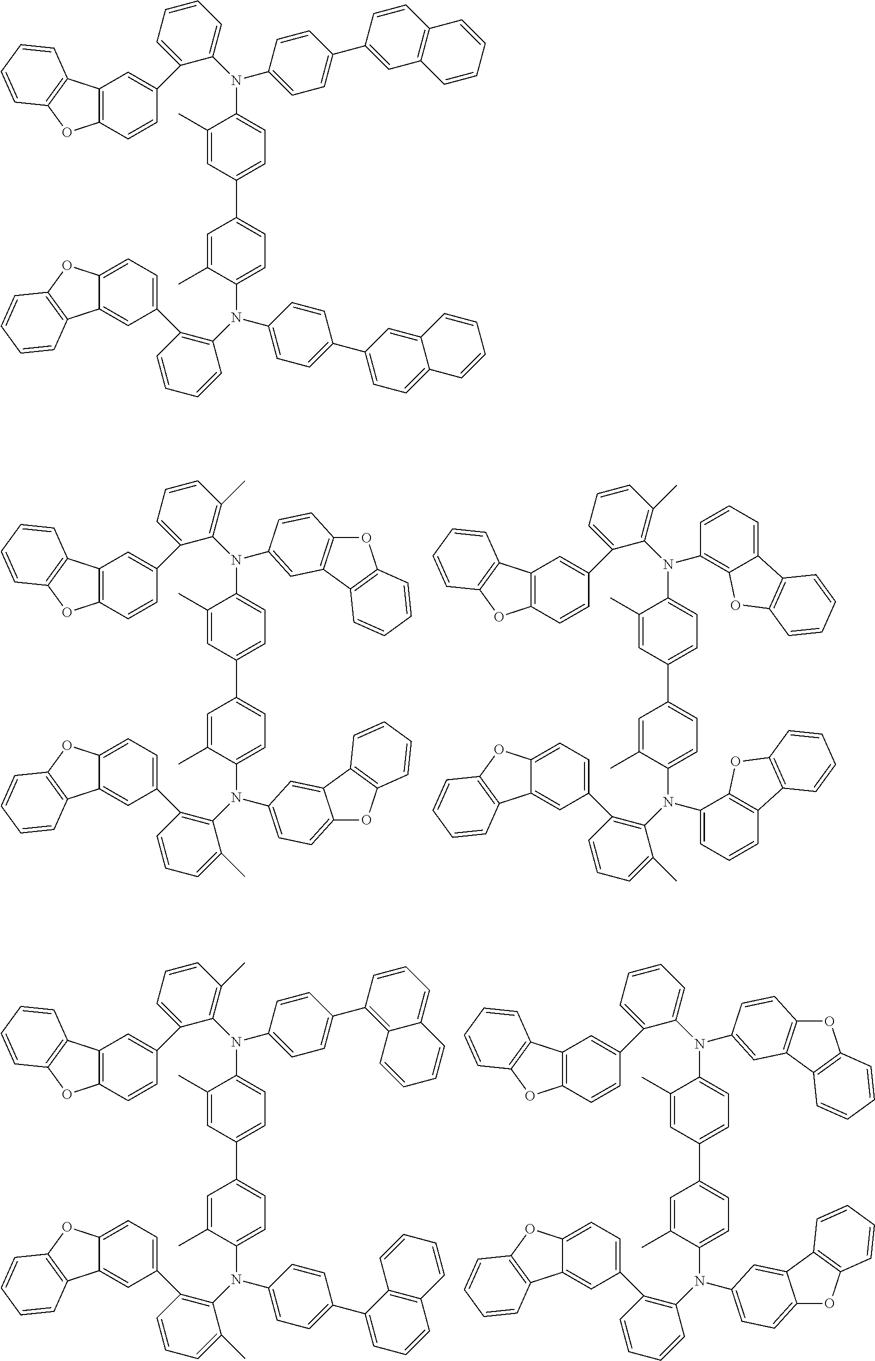

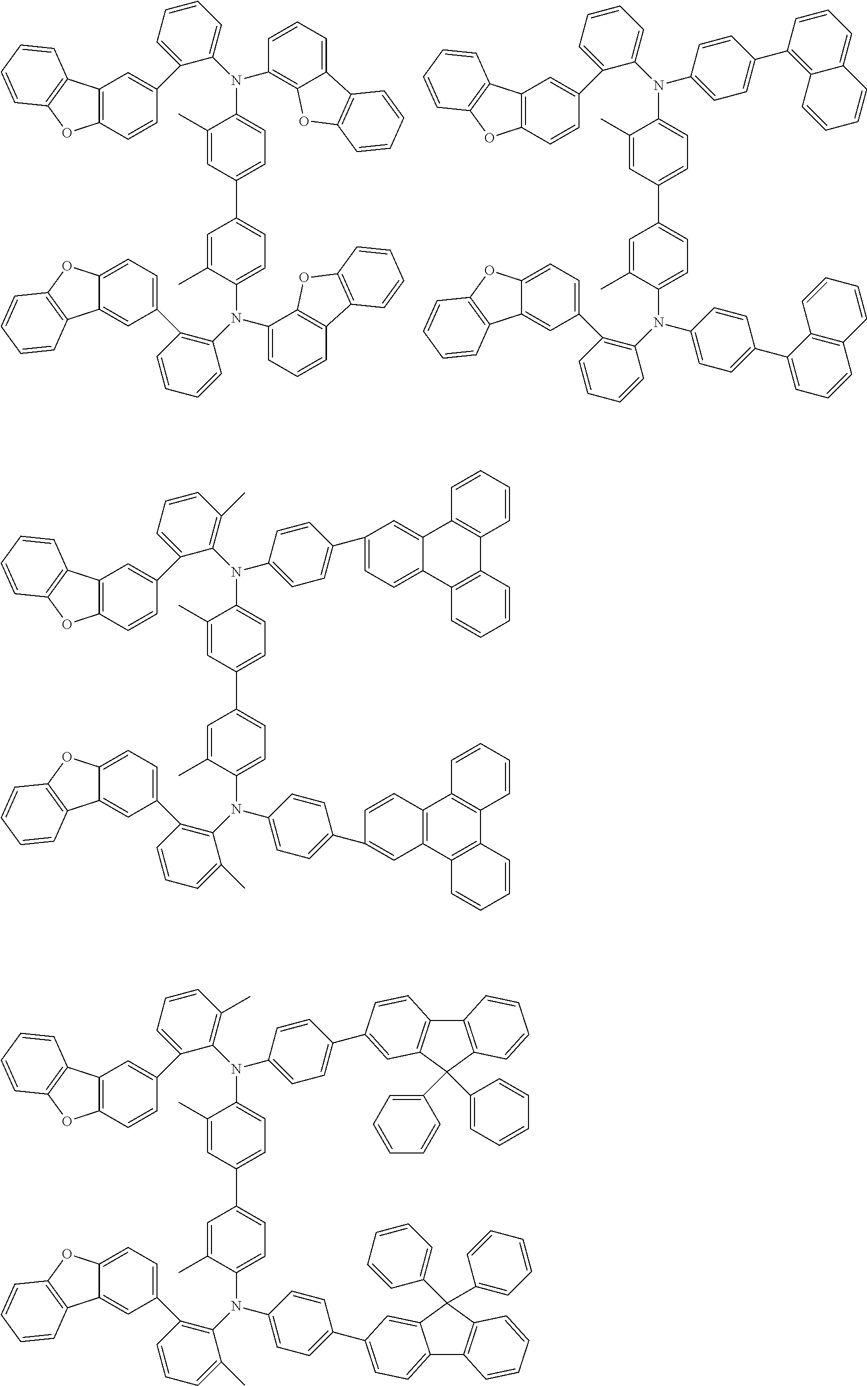

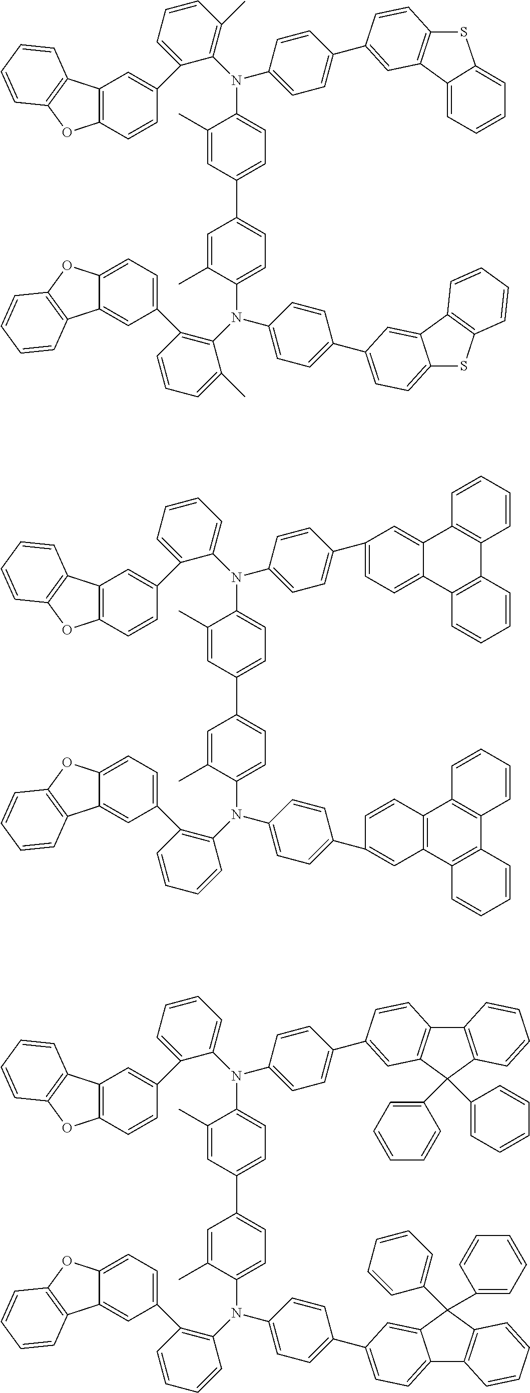

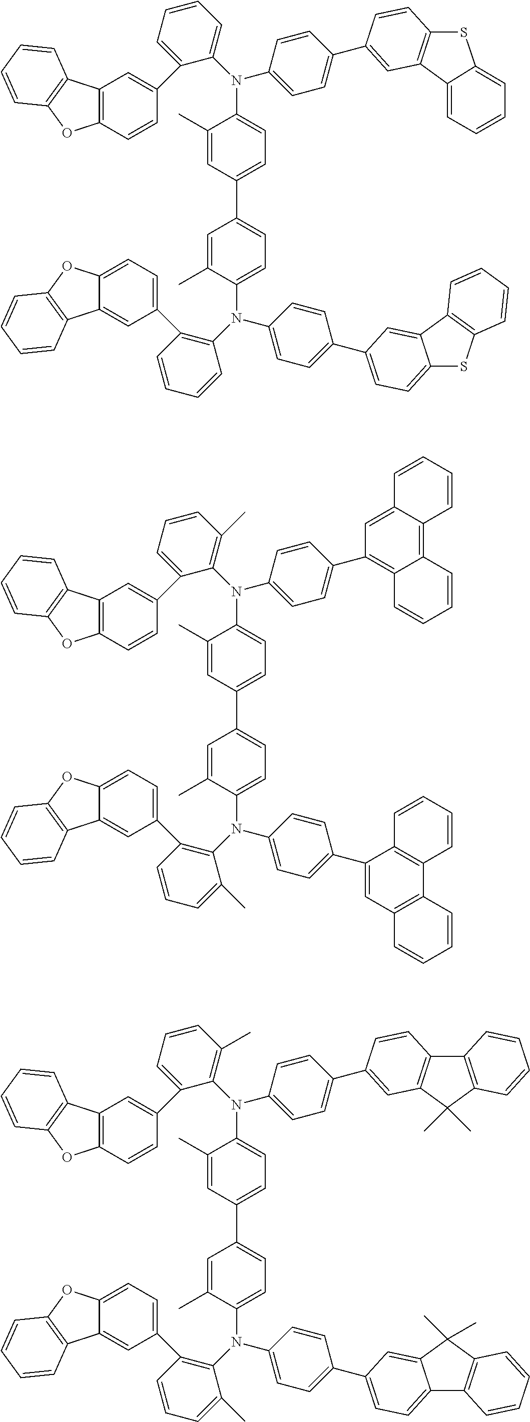 [SMILES: Cc1cc(-c2ccc(N(c3ccc(-c4ccc5sc6ccccc6c5c4)cc3)c3ccccc3-c3ccc4oc5ccccc5c4c3)c(C)c2)ccc1N(c1ccc(-c2ccc3sc4ccccc4c3c2)cc1)c1ccccc1-c1ccc2oc3ccccc3c2c1 ; Cc1cc(-c2ccc(N(c3ccc(-c4cc5ccccc5c5ccccc45)cc3)c3c(C)cccc3-c3ccc4oc5ccccc5c4c3)c(C)c2)ccc1N(c1ccc(-c2cc3ccccc3c3ccccc23)cc1)c1c(C)cccc1-c1ccc2oc3ccccc3c2c1 ; Cc1cc(-c2ccc(N(c3ccc(-c4ccc5c(c4)C(C)(C)c4ccccc4-5)cc3)c3c(C)cccc3-c3ccc4oc5ccccc5c4c3)c(C)c2)ccc1N(c1ccc(-c2ccc3c(c2)C(C)(C)c2ccccc2-3)cc1)c1c(C)cccc1-c1ccc2oc3ccccc3c2c1]

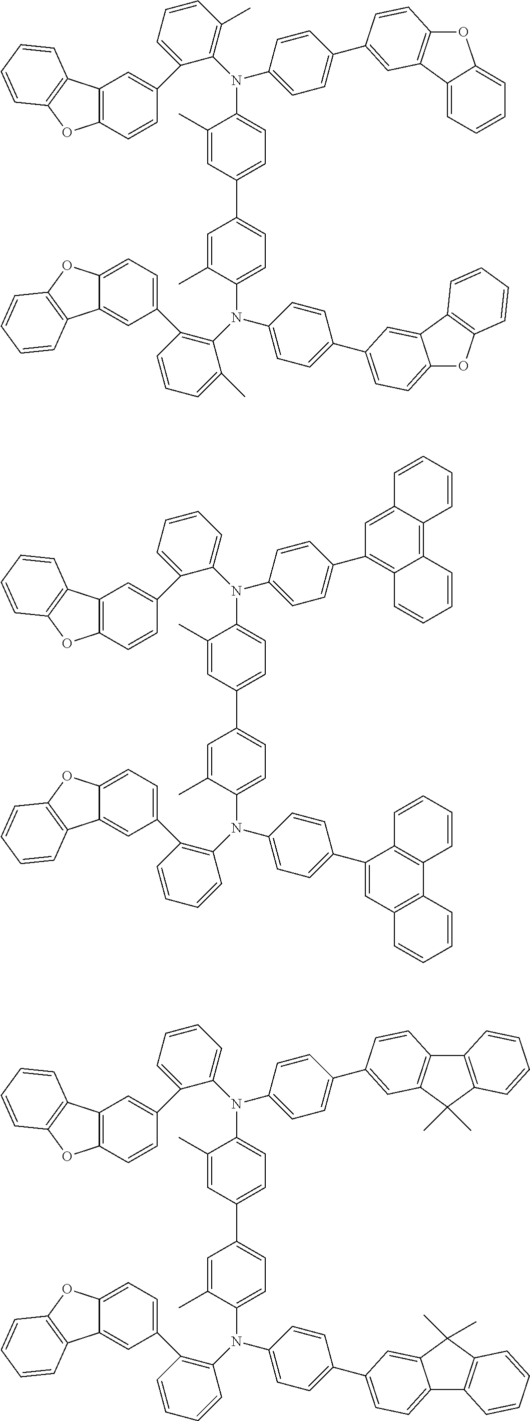 [SMILES: Cc1cc(-c2ccc(N(c3ccc(-c4ccc5oc6ccccc6c5c4)cc3)c3c(C)cccc3-c3ccc4oc5ccccc5c4c3)c(C)c2)ccc1N(c1ccc(-c2ccc3oc4ccccc4c3c2)cc1)c1c(C)cccc1-c1ccc2oc3ccccc3c2c1 ; Cc1cc(-c2ccc(N(c3ccc(-c4cc5ccccc5c5ccccc45)cc3)c3ccccc3-c3ccc4oc5ccccc5c4c3)c(C)c2)ccc1N(c1ccc(-c2cc3ccccc3c3ccccc23)cc1)c1ccccc1-c1ccc2oc3ccccc3c2c1 ; Cc1cc(-c2ccc(N(c3ccc(-c4ccc5c(c4)C(C)(C)c4ccccc4-5)cc3)c3ccccc3-c3ccc4oc5ccccc5c4c3)c(C)c2)ccc1N(c1ccc(-c2ccc3c(c2)C(C)(C)c2ccccc2-3)cc1)c1ccccc1-c1ccc2oc3ccccc3c2c1]

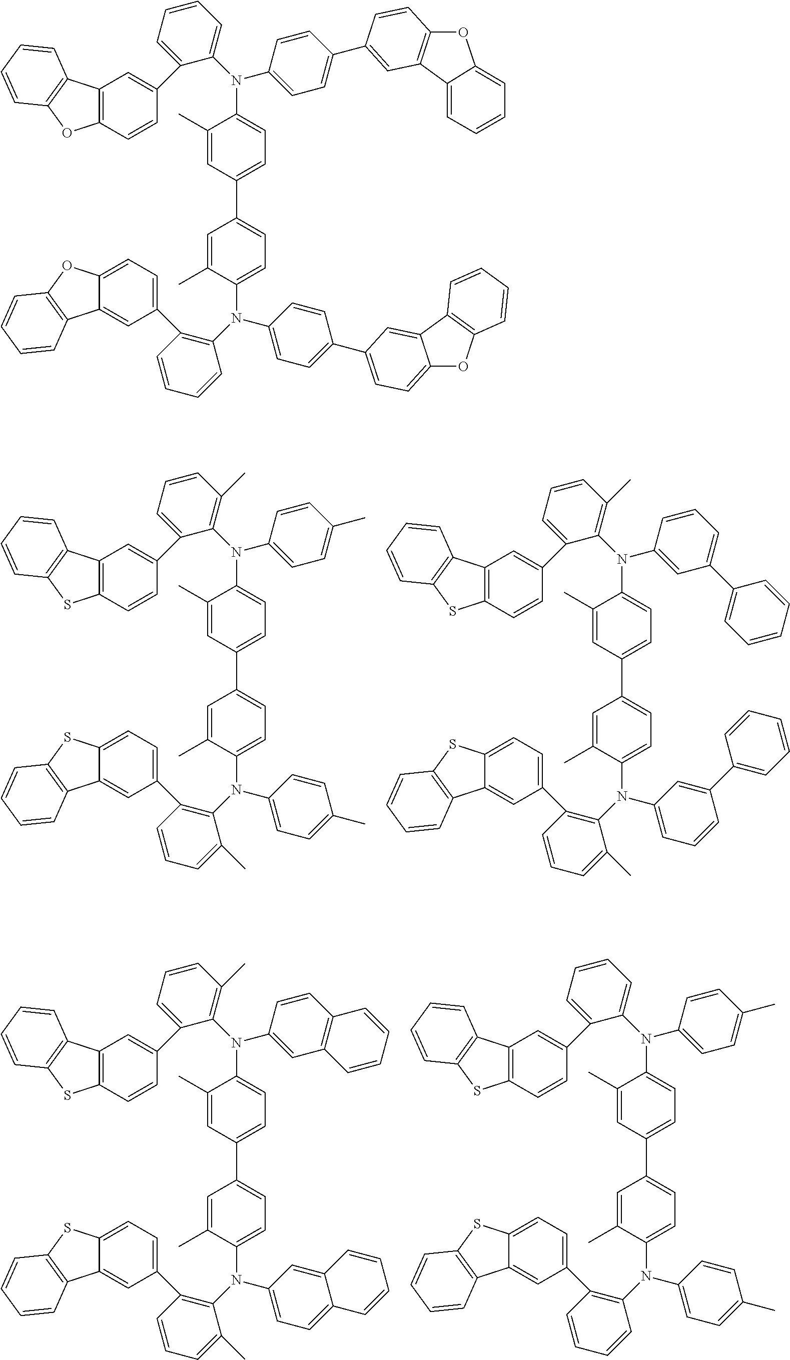

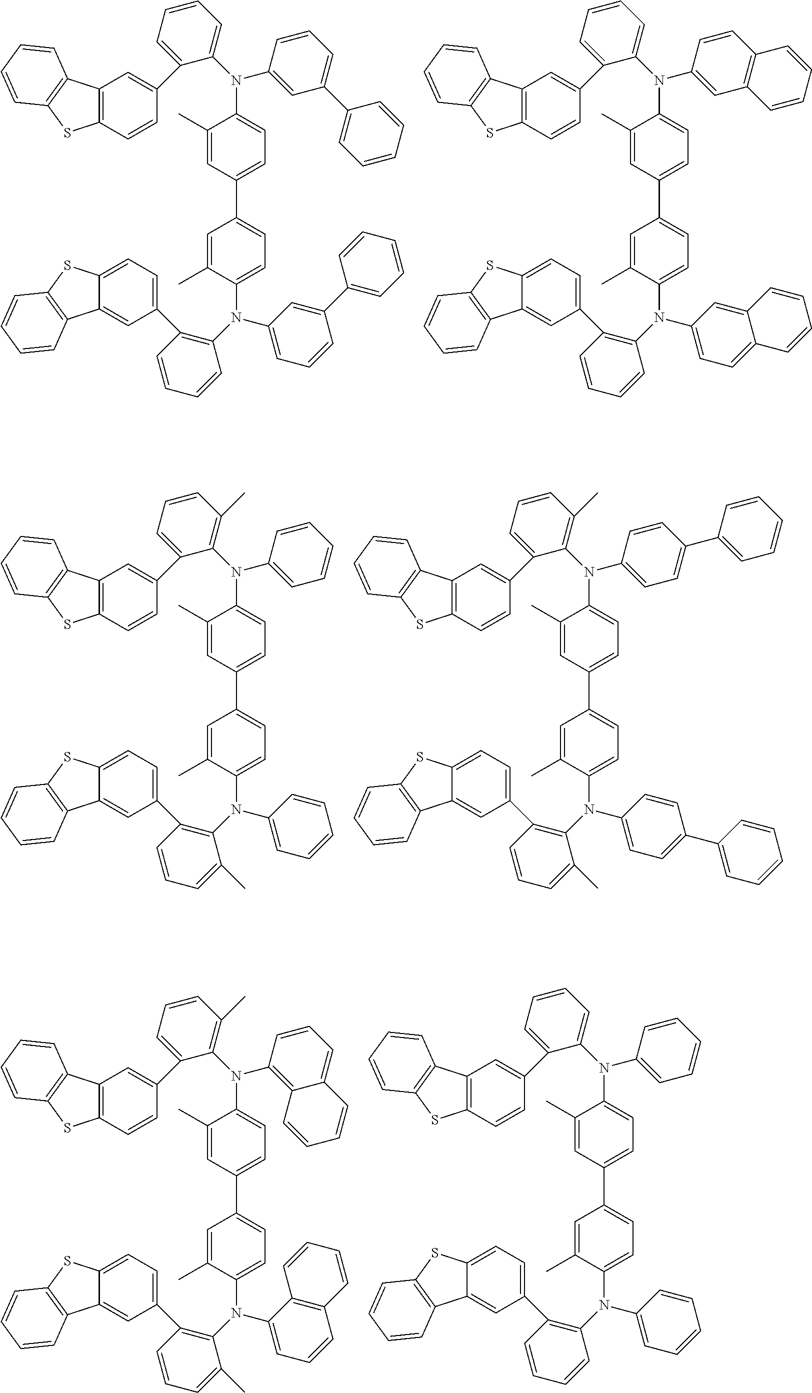

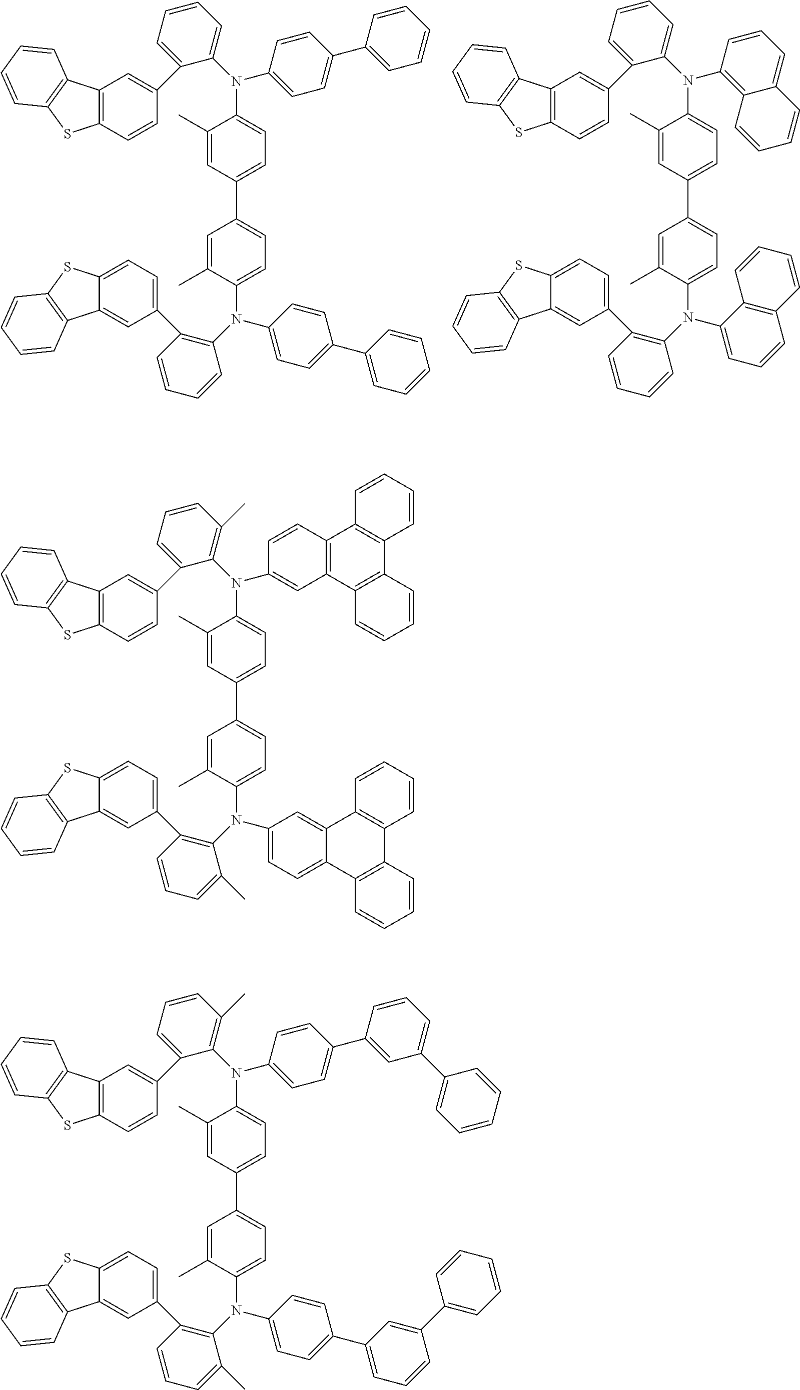

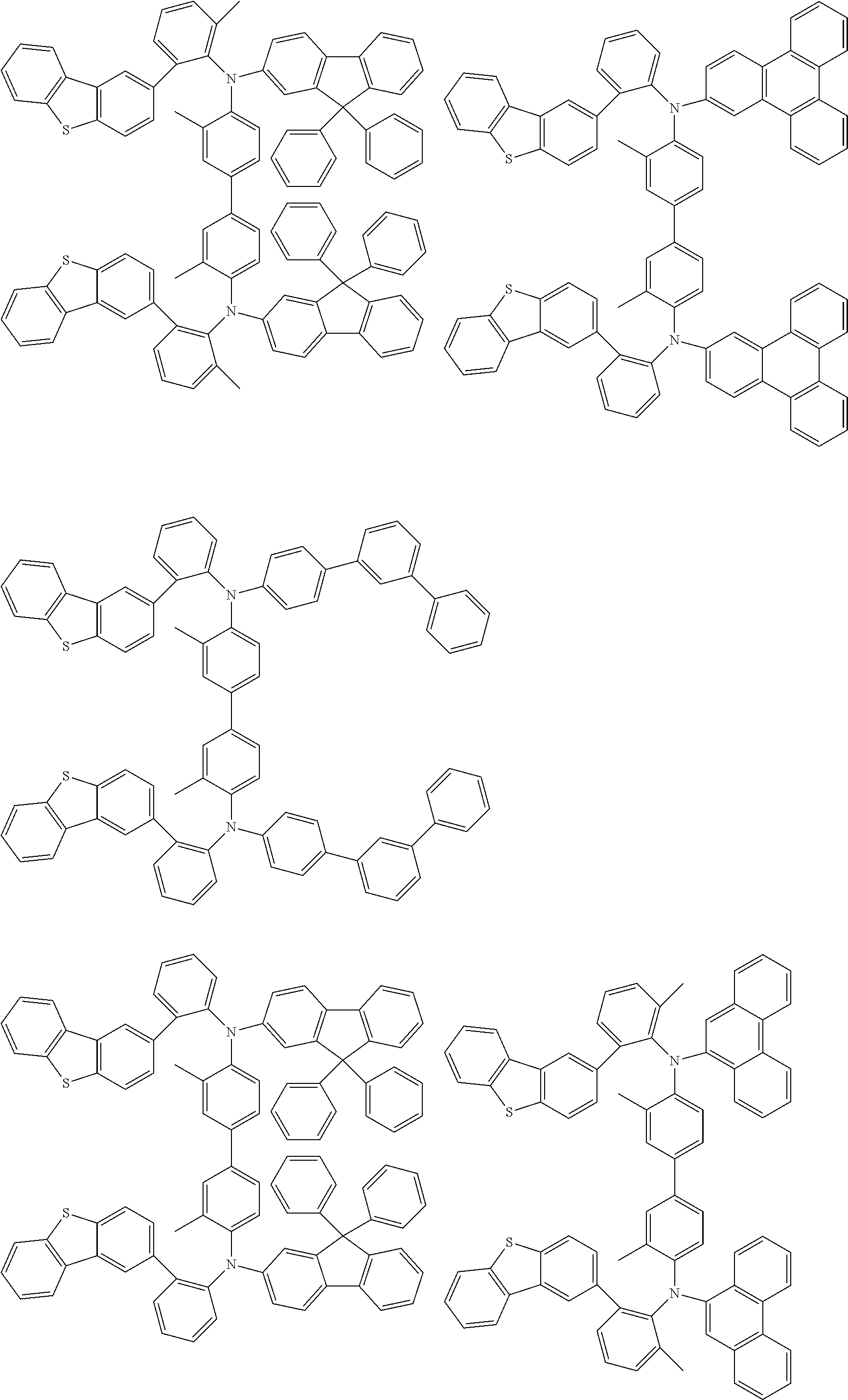

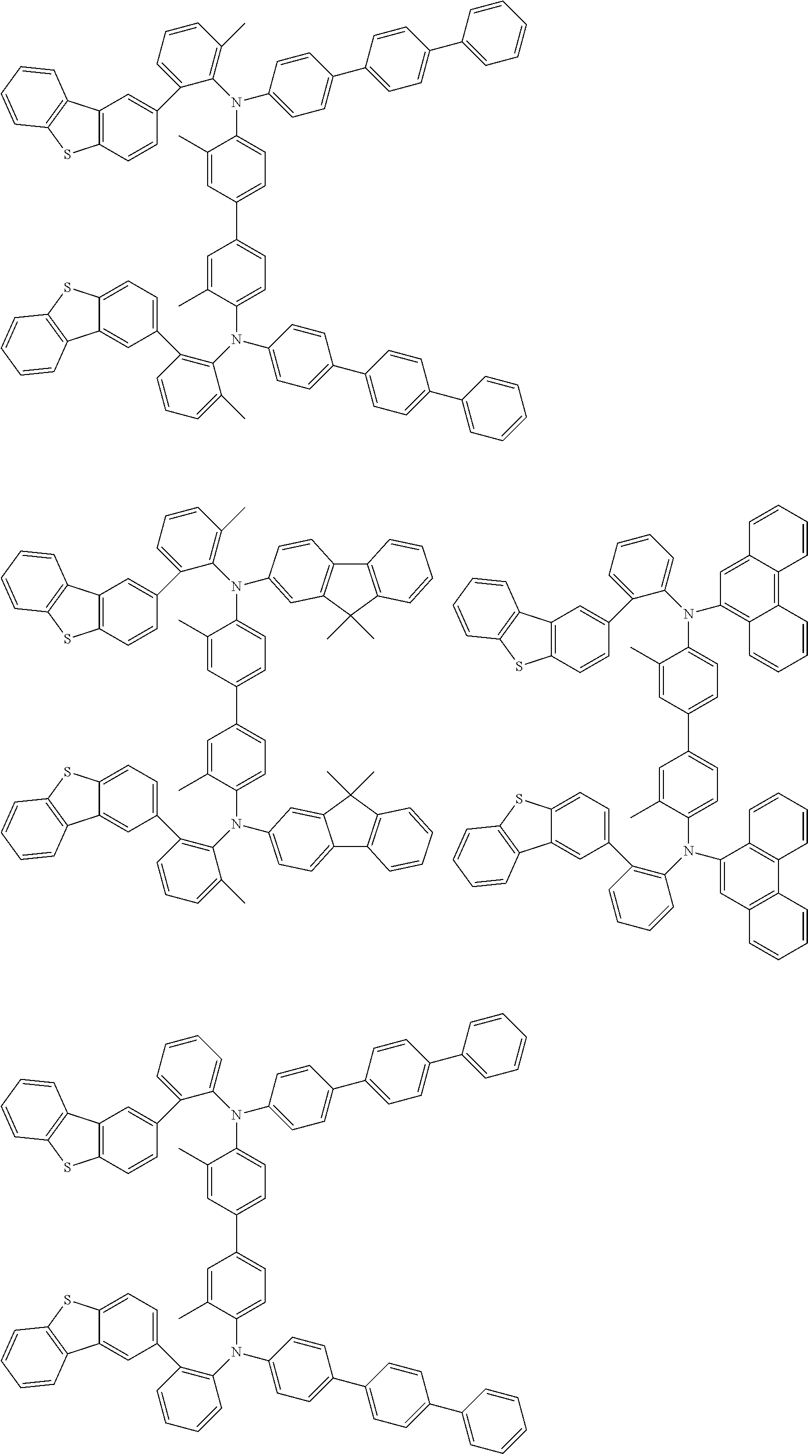

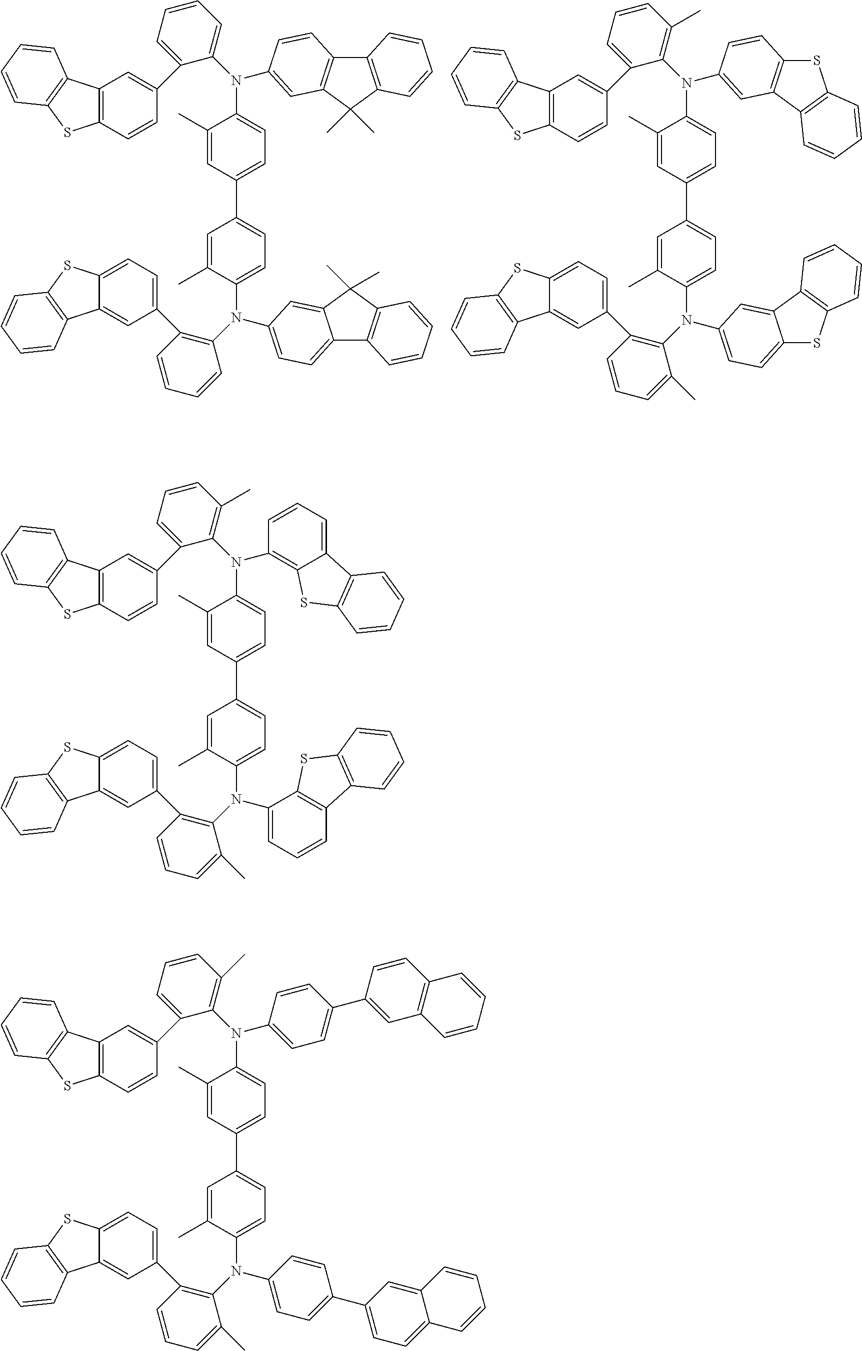

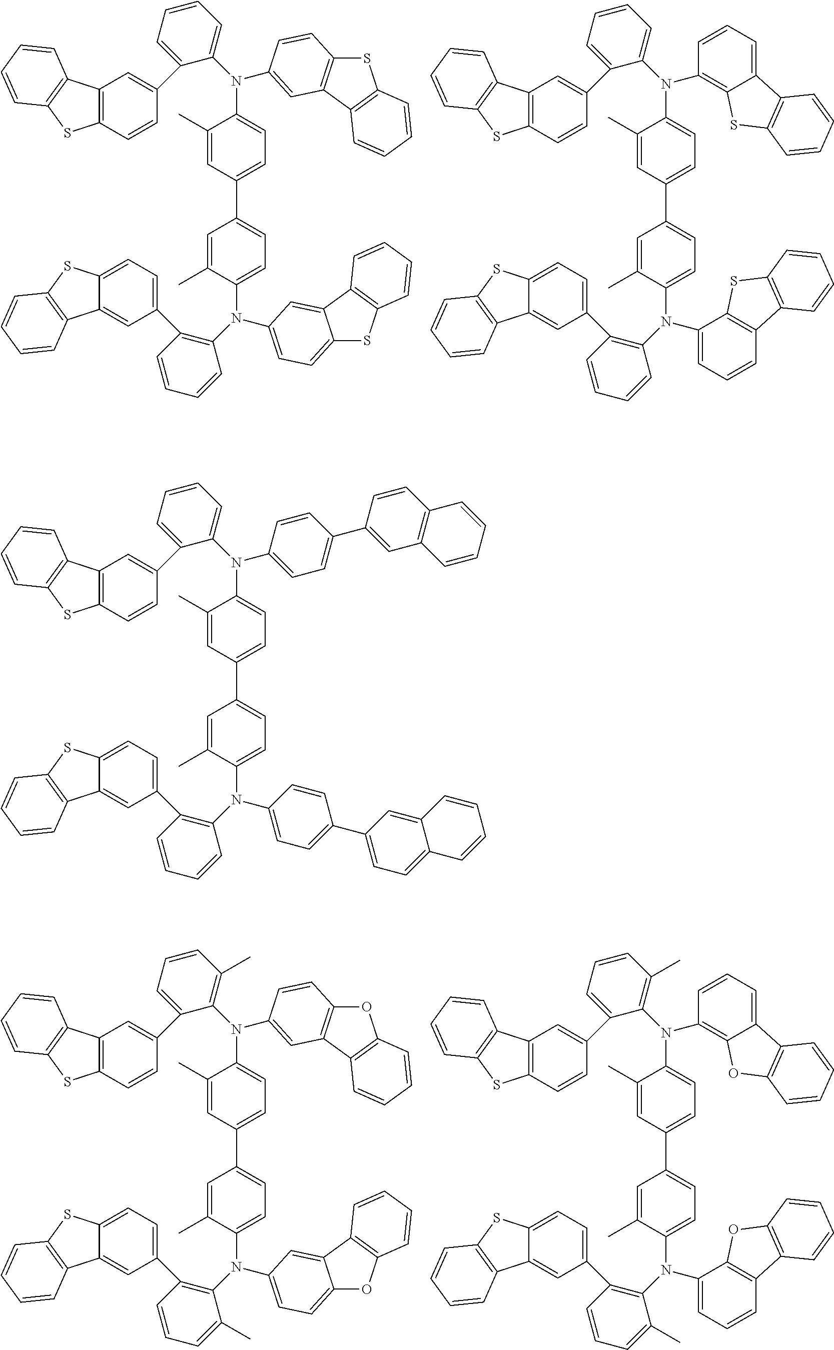

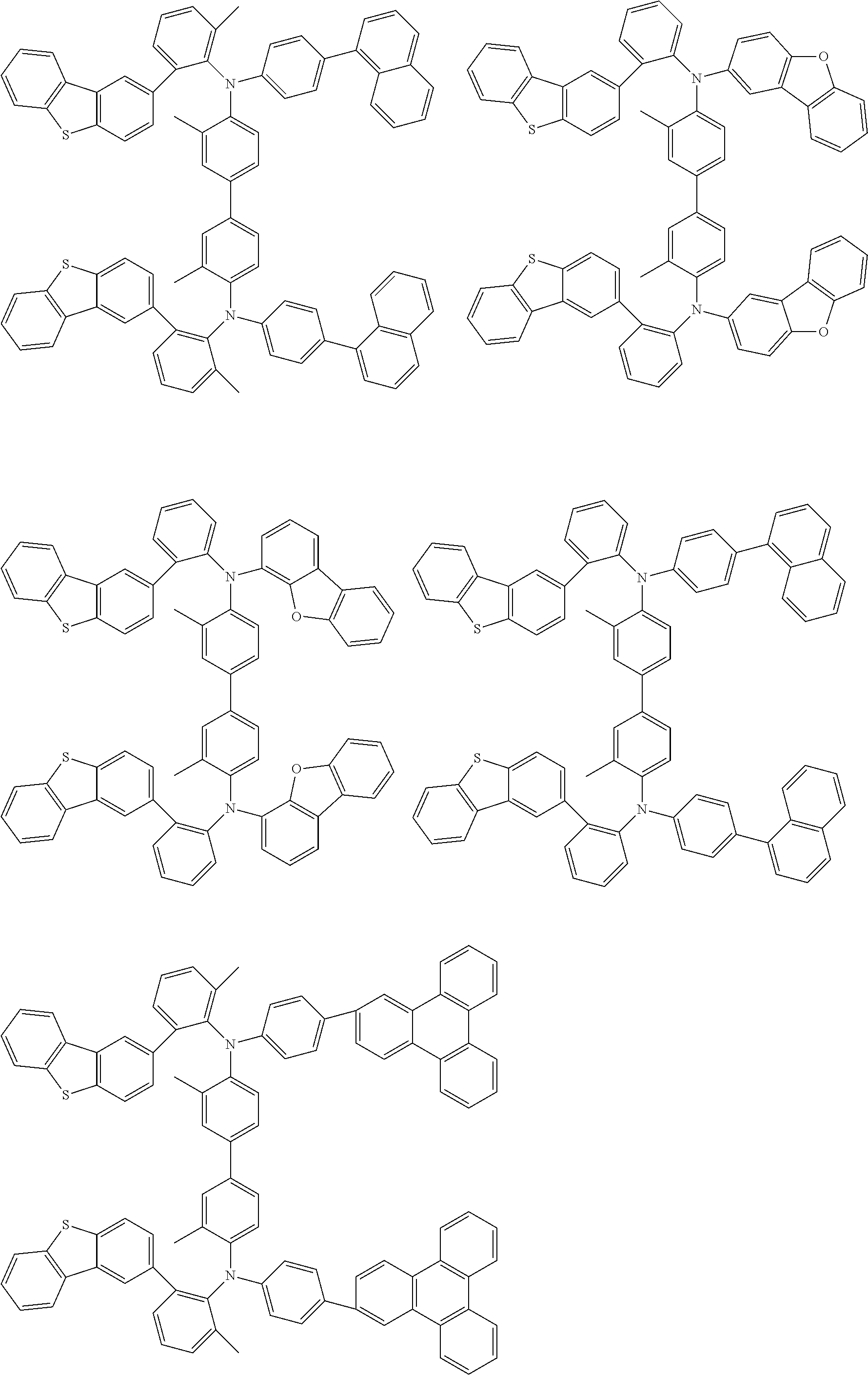

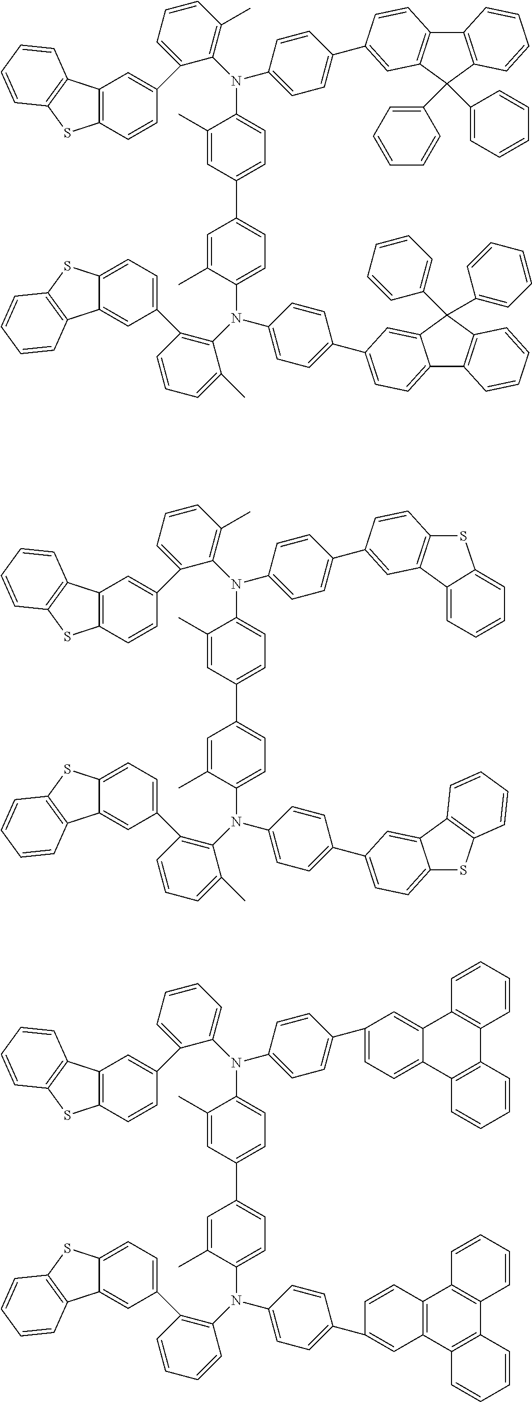

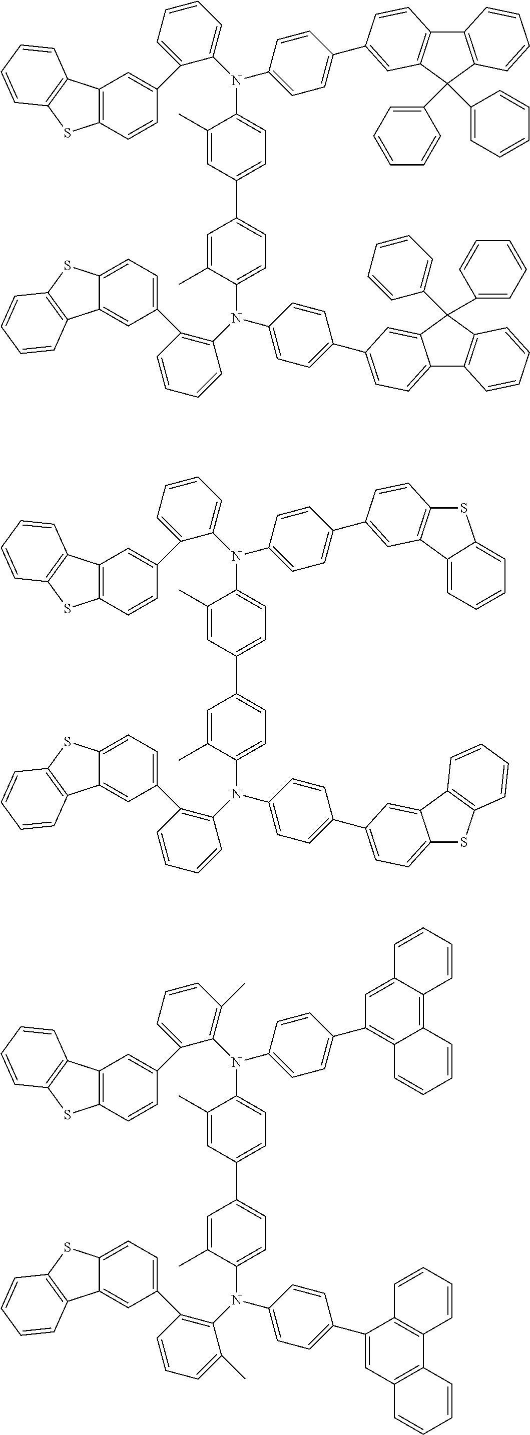

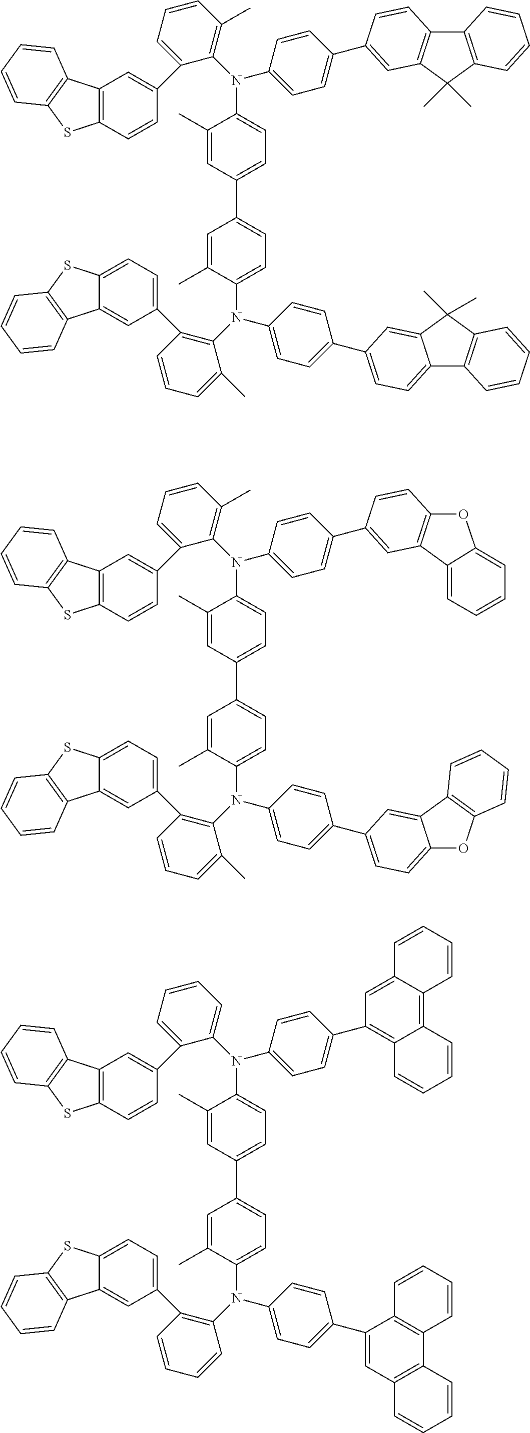 [SMILES: Cc1cc(-c2ccc(N(c3ccc(-c4ccc5c(c4)C(C)(C)c4ccccc4-5)cc3)c3c(C)cccc3-c3ccc4sc5ccccc5c4c3)c(C)c2)ccc1N(c1ccc(-c2ccc3c(c2)C(C)(C)c2ccccc2-3)cc1)c1c(C)cccc1-c1ccc2sc3ccccc3c2c1 ; Cc1cc(-c2ccc(N(c3ccc(-c4ccc5oc6ccccc6c5c4)cc3)c3c(C)cccc3-c3ccc4sc5ccccc5c4c3)c(C)c2)ccc1N(c1ccc(-c2ccc3oc4ccccc4c3c2)cc1)c1c(C)cccc1-c1ccc2sc3ccccc3c2c1 ; Cc1cc(-c2ccc(N(c3ccc(-c4cc5ccccc5c5ccccc45)cc3)c3ccccc3-c3ccc4sc5ccccc5c4c3)c(C)c2)ccc1N(c1ccc(-c2cc3ccccc3c3ccccc23)cc1)c1ccccc1-c1ccc2sc3ccccc3c2c1]

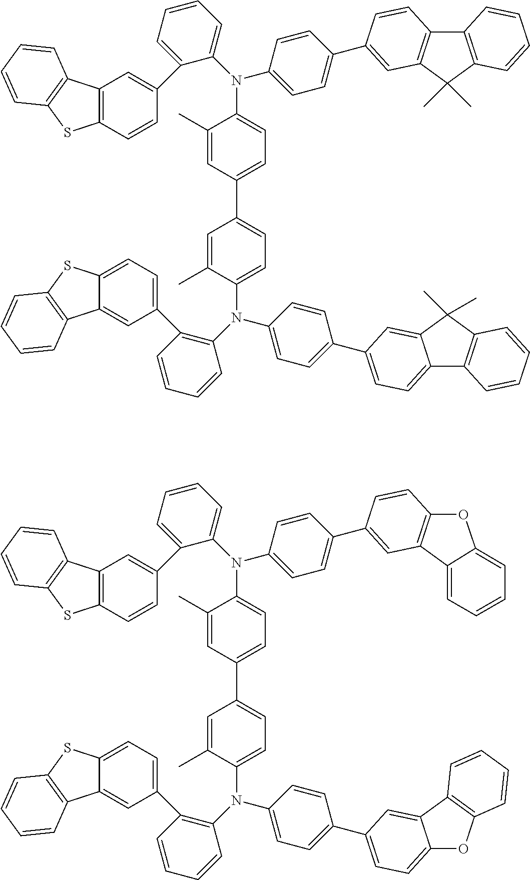 [SMILES: Cc1cc(-c2ccc(N(c3ccc(-c4ccc5c(c4)C(C)(C)c4ccccc4-5)cc3)c3ccccc3-c3ccc4sc5ccccc5c4c3)c(C)c2)ccc1N(c1ccc(-c2ccc3c(c2)C(C)(C)c2ccccc2-3)cc1)c1ccccc1-c1ccc2sc3ccccc3c2c1 ; Cc1cc(-c2ccc(N(c3ccc(-c4ccc5oc6ccccc6c5c4)cc3)c3ccccc3-c3ccc4sc5ccccc5c4c3)c(C)c2)ccc1N(c1ccc(-c2ccc3oc4ccccc4c3c2)cc1)c1ccccc1-c1ccc2sc3ccccc3c2c1]

The aromatic amine derivative of the invention is used preferably as a material for organic EL devices and more preferably as a hole transporting material for organic EL devices.

The aromatic amine derivative is an aromatic diamine derivative including a 4,4′-biphenylene linker. Generally, in an aromatic diamine including a 4,4′-biphenylene linker, the lone pair on the nitrogen atom overlaps with the π-electrons of the benzene ring of the linker and therefore two nitrogen atoms electronically interact with each other. In the aromatic amine derivative represented by formula (1) wherein R⁵ and R⁶ each represent a group other than a hydrogen atom, R⁵ and R⁶ cause steric hindrance with the groups on the nitrogen atoms. Similarly, in the aromatic amine derivative represented by formula (1) wherein R⁷ and R⁸ each represent a group other than a hydrogen atom, R⁷ and R⁸ cause steric hindrance with the groups on the nitrogen atoms. Therefore, the carbon-nitrogen bond between the nitrogen atom and the benzene ring thereon is distorted and the overlap between the lone pairs on the nitrogen atom with the π-electrons of the benzene ring of the linker is reduced, thereby reducing the electronic interaction between two nitrogen atoms.

Therefore, the aromatic amine derivative of the invention exhibits a nature as a monoamine derivative strongly, although it is structurally a diamine derivative. With such a nature as a monoamine derivative, the aromatic amine derivative of the invention combines a high energy gap and a high hole mobility and therefore is suitable as a hole transporting material for improving the lifetime and the efficiency of organic EL device and reducing its driving voltage.

In addition, R¹ and R² at ortho positions with respect to the respective nitrogen atom increase the steric hindrance between the groups on each nitrogen atom. Therefore, the electronic interaction between the groups is suppressed to increase the energy gap. Further, the short physical distance between the nitrogen atoms in the diamine derivative is advantageous for hole hopping and increases the hole mobility.

Thus, by the effects of the 4,4′-biphenylene linker and R¹ and R² at ortho positions with respect to the respective nitrogen atoms, the amine derivative of the invention combines a high energy gap and a high hole mobility and therefore is suitable as a hole transporting material for improving the lifetime and the efficiency of organic EL device and reducing its driving voltage.

Next, the organic EL device of the invention will be described. The organic EL device of the invention comprises an organic thin film layer between a cathode and an anode. The organic thin film layer comprises one or more layers and comprises a light emitting layer, and at least one layer of the organic thin film layer comprises the aromatic amine derivative described above alone or as a component of a mixture.

In a preferred organic EL device of the invention, the organic thin film layer comprises a hole transporting layer and the hole transporting layer comprises the aromatic amine derivative of the invention alone or as a component of a mixture. The hole transporting layer preferably comprises the aromatic amine derivative of the invention as a main component.

In another preferred organic EL device of the invention, the organic thin film layer comprises a hole transporting layer and one of an electron transporting layer and an electron injecting layer, wherein the hole transporting layer comprises the aromatic amine derivative of the invention alone or as a component of a mixture and the electron transporting layer or the electron injecting layer comprises a nitrogen-containing heterocyclic compound. The hole transporting layer preferably comprises the aromatic amine derivative of the invention as a main component.

The light emitting layer of the organic EL device of the invention preferably comprises an arylamine compound and/or a styrylamine compound.

Examples of the arylamine compound for use in the light emitting layer include a compound represented by formula (I):

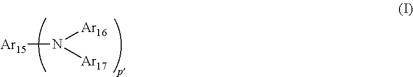

wherein Ar₁₅ is selected from the group consisting of a phenyl group, a biphenyl group, a terphenyl group, a stilbene group, and a distyrylaryl group; Ar₁₆ and Ar₁₇ are each independently represent a hydrogen atom or an aromatic group having 6 to 20 carbon atoms and each may have a substituent; and p′ represents an integer of 1 to 4.

Preferably, Ar₁₆ and/or Ar₁₇ have a styryl substituent. Preferred examples of the aromatic group having 6 to 20 carbon atoms include a phenyl group, a naphthyl group, an anthranyl group, a phenanthryl group, and a terphenyl group.

Examples of the styrylamine compound for use in the light emitting layer include a compound represented by formula (II):

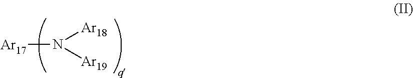

wherein Ar₁₇ to Ar₁₉ each independently represent a substituted or unsubstituted aryl group having 5 to 40 ring atoms, and q′ represents an integer of 1 to 4.

The aryl group having 5 to 40 ring atoms is preferably a phenyl group, a naphthyl group, an anthranyl group, a phenanthryl group, a pyrenyl group, a coronyl group, a biphenyl group, a terphenyl group, a pyrrolyl group, a furanyl group, a thiophenyl group, a benzothiophenyl group, an oxadiazolyl group, a diphenylanthranyl group, an indolyl group, a carbazolyl group, a pyridyl group, a benzoquinolyl group, a fluoranthenyl group, an acenaphthofluoranthenyl group, or a stilbene group.

The aryl group having 5 to 40 ring atoms may have a substituent. Preferred examples of the substituent include an alkyl group having 1 to 6 carbon atoms, such as an ethyl group, a methyl group, an isopropyl group, a n-propyl group, a s-butyl group, a t-butyl group, a pentyl group, a hexyl group, a cyclopentyl group, and a cyclohexyl group; an alkoxy group having 1 to 6 carbon atoms, such as an ethoxy group, a methoxy group, an isopropoxy group, a n-propoxy group, a s-butoxy group, a t-butoxy group, a pentoxy group, a hexyloxy group, a cyclopentoxy group, and a cyclohexyloxy group; an aryl group having 5 to 40 ring atoms; an amino group having an aryl substituent having 5 to 40 ring atoms; an ester group having an aryl group having 5 to 40 ring atoms; an ester group having an alkyl group having 1 to 6 carbon atoms; a cyano group; a nitro group; and a halogen atom, such as chlorine, bromine, and iodine.

The device structure of the organic EL device of the invention will be described below.

### Structure of Organic EL Device

Typical examples of the device structure of the organic EL device of the invention are shown below. Of the following device structures, the structure (8) is preferably used, although not limited to the following structures.

(1) anode/light emitting layer/cathode;
(2) anode/hole injecting layer/light emitting layer/cathode;
(3) anode/light emitting layer/electron injecting layer/cathode;
(4) anode/hole injecting layer/light emitting layer/electron injecting layer/cathode;
(5) anode/organic semiconductor layer/light emitting layer/cathode;
(6) anode/organic semiconductor layer/electron blocking layer/light emitting layer/cathode
(7) anode/organic semiconductor layer/light emitting layer/adhesion improving layer/cathode;
(8) anode/hole injecting layer/hole transporting layer/light emitting layer/electron injecting layer/cathode;
(9) anode/insulating layer/light emitting layer/insulating layer/cathode;
(10) anode/inorganic semiconductor layer/insulating layer/light emitting layer/insulating layer/cathode;
(11) anode/organic semiconductor layer/insulating layer/light emitting layer/insulating layer/cathode;
(12) anode/insulating layer/hole injecting layer/hole transporting layer/light emitting layer/insulating layer/cathode; and
(13) anode/insulating layer/hole injecting layer/hole transporting layer/light emitting layer/electron injecting layer/cathode.

### Light-Transmissive Substrate

The organic EL device of the invention is formed on a light-transmissive substrate by depositing the layers which constitute the above layered structure. The light-transmissive substrate serves as a support for the organic EL device and preferably a flat substrate having a transmittance of 50% or more to 400 to 700 nm visible light.

Examples of the substrate include a glass plate and a polymer plate. The glass plate may include a plate made of soda-lime glass, barium-strontium-containing glass, lead glass, aluminosilicate glass, borosilicate glass, barium borosilicate glass, or quartz. The polymer plate may include a plate made of polycarbonate, acryl, polyethylene terephthalate, polyether sulfide, or polysulfone.

### Anode

The anode of the organic EL device injects holes to a hole transporting layer or a light emitting layer, and an anode having a work function of 4.0 eV or more is suitable. Examples of material for anode include carbon, aluminum, vanadium, iron, cobalt, tungsten, silver, gold, platinum, palladium, alloys of these metals, a metal oxide, such as an indium tin oxide alloy (ITO), a tin oxide (NESA), and an indium-zinc oxide alloy (IZO), and an organic conductive polymer, such as a polythiophene and a polypyrrole. The anode may be formed by making the above electrode material into a thin film by a method, such as a vapor deposition method and a sputtering method.

When getting the light emitted from a light emitting layer through an anode, the transmittance of the anode to visible light is preferably 10% or more. The sheet resistance of the anode is preferably several hundreds Ω/□ or less. The film thickness of the anode depends upon the kind of material and generally 10 nm to 1 μm, preferably 10 to 200 nm.

### Cathode

The cathode of the organic EL device injects electrons to an electron injecting/transporting layer or a light emitting layer, and a cathode having a work function of 4.0 eV or less is suitable. Examples of the cathode material include magnesium, calcium, tin, lead, titanium, yttrium, lithium, ruthenium, manganese, aluminum, lithium fluoride, and alloys of these metals, although not limited thereto. Representative examples of the alloy include magnesium/silver, magnesium/indium, and lithium/aluminum, although not limited thereto. The ratio of the alloying metals is suitably selected according to temperature of deposition source, atmosphere, and degree of vacuum. The cathode may be formed by making the electrode material described above into a thin film by a method, such as a vapor deposition method and a sputtering method.

When the light emitted from a light emitting layer is taken through the cathode, the transmittance of the cathode to the emitted light is preferably 10% or more. The sheet resistivity of the cathode is preferably several hundreds Ω/□ or less and the thickness of the cathode is generally 10 nm to 1 μm and preferably 50 to 200 nm.

The anode and the cathode may be formed into a two or more layered structure, if necessary.

### Light Emitting Layer

The light emitting layer of the organic EL device combines the following functions (1) to (3):

(1) injecting function: function of injecting holes from the anode or the hole injecting layer and injecting electrons from the cathode or the electron injecting layer when an electric field is applied,
(2) transporting function: function of transporting injected charges (electrons and holes) by the force of the electric field, and
(3) light emitting function: function of providing the field for recombination of electrons and holes to allow the emission of light.

The light emitting layer may be different in the hole injection ability and the electron injection ability, and also may be different in the hole transporting ability and the electron transporting ability each being expressed by mobility, but it is preferred to transport either of hole or electron dominantly.

In addition to the aromatic amine derivative of the invention, the light emitting layer may comprise, if necessary, a known light emitting material, a known doping material, a known hole injecting material or a known electron injecting material. By forming the light emitting layer into a laminated structure of organic thin film layers, the decrease in the luminance and lifetime due to quenching can be prevented. By using a doping material in the light emitting layer, the luminance and emission efficiency can be improved and a red emission and a blue emission can be obtained.

The light emitting materials or the doping material usable in the light emitting layer together with the aromatic amine derivative of the invention includes, for example, anthracene, naphthalene, phenanthrene, pyrene, tetracene, coronene, chrysene, fluorescein, perylene, phthaloperylene, naphthaloperylene, perinone, phthaloperinone, naphthaloperinone, diphenylbutadiene, tetraphenylbutadiene, coumarin, oxadiazole, aldazine, bisbenzoxazoline, bisstyryl, pyrazine, cyclopentadiene, quinoline metal complex, aminoquinoline metal complex, benzoquinoline metal complex, imine, diphenylethylene, vinylanthracene, diaminecarbazol, pyran, thiopyran, polymethyne, merocyanine, imidazol chelate oxinoid compound, quinacridone, rubrene and fluorescent dye, although not limited thereto.

The host material usable with the aromatic amine derivative of the invention is preferably a compound represented by any of formulae (i) to (ix):

an asymmetric anthracene represented by formula (i):

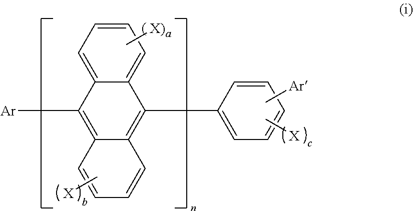

wherein Ar represents a substituted or unsubstituted fused aromatic group having 10 to 50 ring carbon atoms;

Ar′ represents a substituted or unsubstituted aromatic group having 6 to 50 ring carbon atoms;

X represents a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 1 to 50 carbon atoms, a carboxyl group, a halogen atom, a cyano group, a nitro group, or a hydroxy group;

a, b, and c each represent an integer of 0 to 4; and

n represents an integer of 1 to 3, and when n represents 2 or more, the structures in [ ] may be the same or different;

an a symmetric monoanthracene derivative represented by formula (ii):

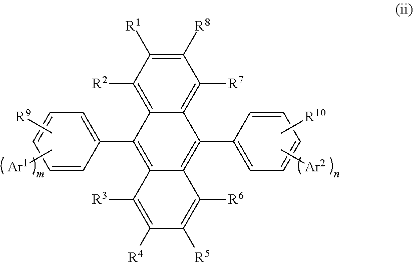

wherein Ar¹ and Ar² each independently represent a substituted or unsubstituted aromatic group having 6 to 50 ring carbon atoms;

m and n each represent an integer of 1 to 4, provided that Ar¹ and Ar² are not the same when m=n=1 and positions at which Ar¹ and Ar² are bonded to the benzene ring are bilaterally symmetric, and m and n represent different integers when m or n represents an integer of 2 to 4; and

R¹ to R¹⁰ each independently represent a hydrogen atom, a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxy group;

an asymmetric pyrene derivative represented by formula (iii):

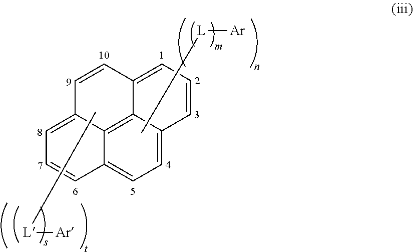

wherein Ar and Ar′ each represent a substituted or unsubstituted aromatic group having 6 to 50 ring carbon atoms;

L and L′ each represent a substituted or unsubstituted phenylene group, a substituted or unsubstituted naphthalenylene group, a substituted or unsubstituted fluorenylene group, or a substituted or unsubstituted dibenzosilolylene group; m represents an integer of 0 to 2;

n represents an integer of 1 to 4; s represents an integer of 0 to 2; t represents an integer of 0 to 4; and

L or Ar bonds to any one of 1- to 5-positions of pyrene, and L′ or Ar′ bonds to any one of 6- to 10-positions of pyrene;

provided that when n+t represents an even number, Ar, Ar′, L, and L′ satisfy the following requirements (1) or (2):

(1) Ar≠Ar′ and/or L≠L′ (wherein ≠ means that the groups are structurally different from each other); and
(2) when Ar═Ar′ and L=L′,

(2-1) m≠s and/or n≠t, or

(2-2) when m=s and n=t,
(2-2-1) both L and L′ or the pyrene ring are respectively bonded to different positions of Ar and Ar′, or
(2-2-2) when both L and L′ or the pyrene ring are respectively bonded to the same position of Ar and Ar′, a compound in which both L and L′ or both Ar and Ar′ are bonded to 1- and 6-positions or 2- and 7-positions of the pyrene ring is excluded;
   an asymmetric anthracene derivative represented by formula (iv):

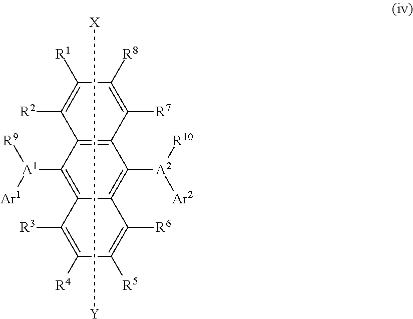

wherein A¹ and A² each independently represent a substituted or unsubstituted fused aromatic ring group having 10 to 20 ring carbon atoms;

Ar¹ and Ar² each independently represent a hydrogen atom or a substituted or unsubstituted aromatic group having 6 to 50 ring carbon atoms;

R¹ to R¹⁰ each independently represent a hydrogen atom, a substituted or unsubstituted aromatic group having 6 to 50 ring carbon atoms, a substituted or unsubstituted aromatic heterocyclic group having 5 to 50 ring atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 1 to 50 carbon atoms, a substituted or unsubstituted silyl group, carboxyl group, a halogen atom, a cyano group, a nitro group, or a hydroxyl group; and

the number of each of Ar¹, Ar², R⁹, and R¹⁰ may be two or more, and adjacent groups may form a saturated or unsaturated cyclic structure,

provided that the groups boned to 9- and 10-positions of the central anthracene are asymmetric with respect to the X-Y axis;

an anthracene derivative represented by formula (v):

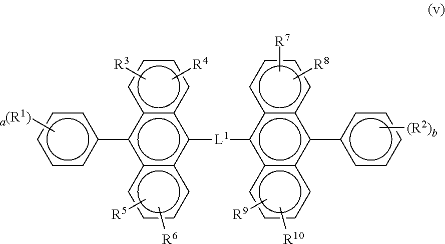

wherein R¹ to R¹⁰ each independently represent a hydrogen atom, an alkyl group, a cycloalkyl group, an aryl group which may be substituted, an alkoxyl group, an aryloxy group, an alkylamino group, an alkenyl group, an arylamino group, or a heterocyclic group which may be substituted;

a and b each represent an integer of 1 to 5, and when a or b represents 2 or more, R¹ groups or R² groups are the same or different and R¹ groups or R² groups may be bonded to each other to form a ring;

R³ and R⁴, R⁵ and R⁶, R⁷ and R⁸, or R⁹ and R¹⁰ may be bonded to each other to form a ring; and

L¹ represents a single bond, —O—, —S—, —N(R)— (wherein R represents an alkyl group or an aryl group which may be substituted), an alkylene group, or an arylene group;

an anthracene derivative represented by formula (vi):

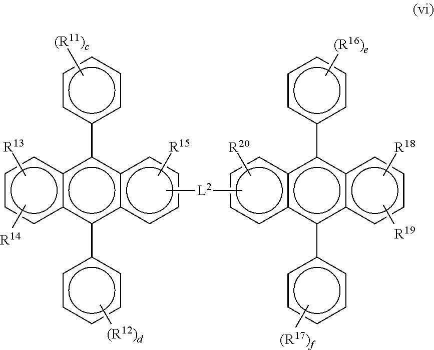

wherein R¹¹ to R²⁰ each independently represent a hydrogen atom, an alkyl group, a cycloalkyl group, an aryl group, an alkoxyl group, an aryloxy group, an alkylamino group, an arylamino group, or a heterocyclic group which may be substituted;

c, d, e, and f each represent an integer of 1 to 5, and when any one of c, d, e, and f represents 2 or more, R¹¹ groups, R¹² groups, R¹⁶ groups, or R¹⁷ groups are the same or different and R¹¹ groups, R¹² groups, R¹⁶ groups, or R¹⁷ groups may be bonded to each other to form a ring;

R¹³ and R¹⁴ or R¹⁸ and R¹⁹ may be bonded to each other to form a ring; and

L² represents a single bond, —O—, —S—, —N(R)— (wherein R represents an alkyl group or an aryl group which may be substituted), an alkylene group, or an arylene group;

a spirofluorene derivative represented by formula (vii):

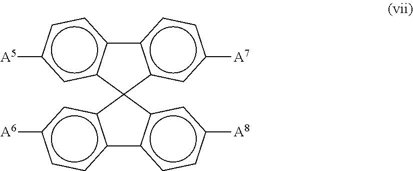

wherein A⁵ to A⁸ each independently represent a substituted or unsubstituted biphenylyl group or a substituted or unsubstituted naphthyl group;

a fused ring-containing compound represented by formula (viii):

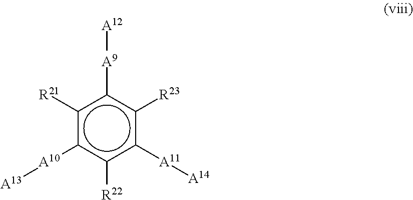

wherein A⁹ to A¹⁴ are the same as defined above;

R²¹ to R²³ each independently represent a hydrogen atom, an alkyl group having 1 to 6 carbon atoms, a cycloalkyl group having 3 to 6 carbon atoms, an alkoxyl group having 1 to 6 carbon atoms, an aryloxy group having 5 to 18 carbon atoms, an aralkyloxy group having 7 to 18 carbon atoms, an arylamino group having 5 to 16 carbon atoms, a nitro group, a cyano group, an ester group having 1 to 6 carbon atoms, or a halogen atom; and

at least one of A⁹ to A¹⁴ represents a group having three or more fused aromatic rings; and

a fluorene compound represented by formula (ix):

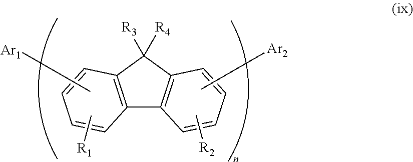

wherein R₁ and R₂ each represent a hydrogen atom, a substituted or unsubstituted alkyl group, a substituted or unsubstituted aralkyl group, a substituted or unsubstituted aryl group, a substituted or unsubstituted heterocyclic group, a substituted amino group, cyano group, or a halogen atom;

R₁ groups or R₂ groups bonded to different fluorene groups are the same or different, and R₁ and R₂ bonded to the same fluorene group are the same or different;

R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group;

R₃ groups or R₄ groups bonded to different fluorene groups are the same or different, and R₃ and R₄ bonded to the same fluorene group are the same or different;

Ar₁ and Ar₂ are the same or different and each represent a substituted or unsubstituted fused polycyclic aromatic group having three or more benzene rings in total or a substituted or unsubstituted fused polycyclic heterocyclic group bonded to a fluorene group via carbon atom wherein a total of the benzene ring and the heterocyclic ring is 3 or more; and

n represents an integer of 1 to 10.

Of the above host materials, an anthracene derivative is preferable, a monoanthracene derivative is more preferable, and an asymmetric anthracene is particularly preferable.

In addition, a phosphorescent compound may be used as a dopant. The phosphorescent compound is preferably used together with a host material comprising a compound including a carbazole ring. The dopant is a compound capable of emitting light from triplet exciton. The dopant is not particularly limited as long as it emits light from triplet exciton, and preferably a metal complex containing at least one metal selected from the group consisting of Ir, Ru, Pd, Pt, Os and Re, more preferably a porphyrin metal complex or an orthometalated complex.

A host compound suitable for phosphorescence, which comprises a compound including a carbazole ring, is a compound capable of causing the emission of phosphorescent compound by transferring energy from its excited state to the phosphorescent compound. The host compound is not limited as long as it is capable of transferring the exciton energy to the phosphorescent compound and can be appropriately selected according to the purpose. The host compound may include a hetero cyclic ring in addition to the carbazole ring.

Examples of the host compound include carbazole derivatives, triazole derivatives, oxazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, styrylanthracene derivatives, fluorenone derivatives, hydrazone derivatives, stilbene derivatives, silazane derivatives, aromatic tertiary amine compounds, styrylamine compounds, aromatic dimethylidene compounds, porphyrin compounds, anthraquinodimethane derivatives, anthrone derivatives, diphenylquinone derivatives, thiopyrane dioxide derivatives, carbodiimide derivatives, fluorenylidene methane derivatives, distyrylpyrazine derivatives, heterocyclic tetracarboxylic anhydrides such as naphthaleneperylene, phthalocyanine derivatives, metal complex polysilane compounds such as metal complexes of 8-quinolinol derivatives and metal complexes having metal phthalocyanine, benzooxazole or benzothiazole as a ligand, poly(N-vinylcarbazole) derivatives, aniline copolymers, electroconductive high molecular weight oligomers such as thiophene oligomers or polythiophene, and polymeric compounds such as polythiophene derivatives, polyphenylene derivatives, polyphenylene vinylene derivatives, and polyfluorene derivatives. The host compound may be used alone or in combination of two or more.

Examples of the host compound are shown below.

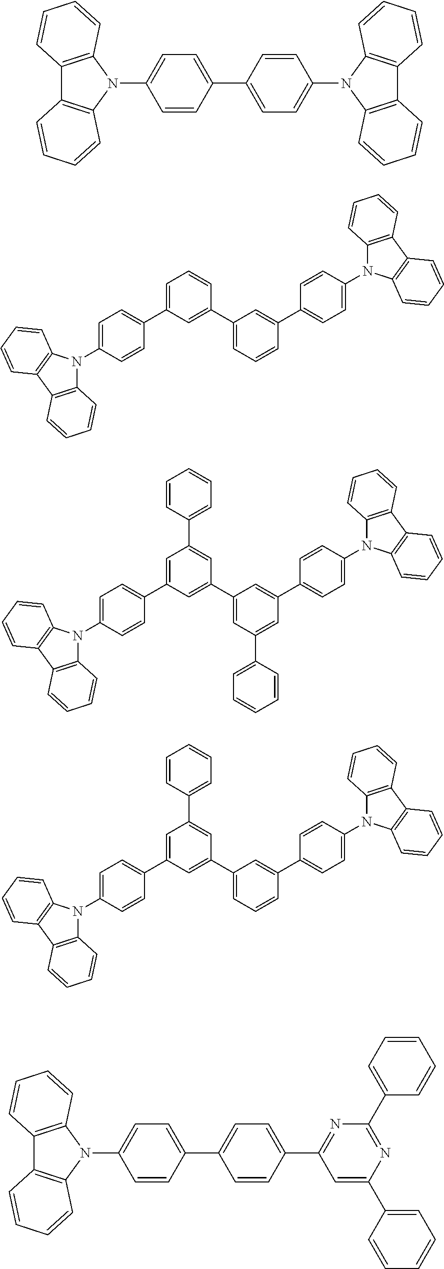

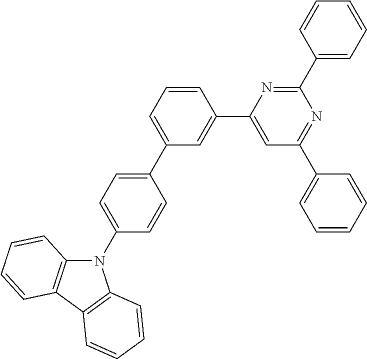 [SMILES: c1ccc(-c2cc(-c3cccc(-c4ccc(-n5c6ccccc6c6ccccc65)cc4)c3)nc(-c3ccccc3)n2)cc1]

The phosphorescent dopant is a compound capable of emitting light from the triplet exciton. The phosphorescent dopant is not restricted as long as it emits light from the triplet exciton, and preferably a metal complex containing at least one metal selected from the group consisting of Ir, Ru, Pd, Pt, Os and Re, more preferably a porphyrin metal complex or an orthometalated metal complex. As the porphyrin metal complex, a porphyrin platinum complex is preferable. The phosphorescent compound may be used alone or in combination of two more.

Various ligands form the orthometalated metal complex, and preferred examples thereof include 2-phenylpyridine derivatives, 7,8-benzoquinoline derivatives, 2-(2-thienyl)pyridine derivatives, 2-(1-naphthyl)pyridine derivatives, and 2-phenylquinoline derivatives. These derivatives may have a substituent, if necessary. In particular, a dopant introduced with a fluoride or a trifluoromethyl group is preferable as the blue-emitting dopant. In addition, a ligand other than the above ligands, such as acetylacetonate and picric acid, may be introduced as a co-ligand.

The content of the phosphorescent dopant in the light emitting layer may be appropriately selected without particular limitation, for example, it may be 0.1 to 70% by mass, preferably 1 to 30% by mass. If being less than 0.1% by mass, the light emission is extremely weak and the effect of using the dopant is not sufficiently obtained. If exceeding 70% by mass, the concentration quenching becomes remarkable and consequently the device performance is deteriorated.

The light emitting layer may contain a hole transporting material, an electron transporting material or a polymer binder, if necessary.

The thickness of the light emitting layer is preferably 5 to 50 nm, more preferably 7 to 50 nm, and most preferably 10 to 50 nm. If less than 5 nm, the light emitting layer is difficult to form and the control of color is difficult. If exceeding 50 nm, the driving voltage may increase.

### Hole Injecting/Transporting Layer (Hole Transporting Region)

The hole injecting/transporting layer is a layer which facilitates the injection of holes into a light emitting layer and transports holes to a light emitting region. The hole injecting/transporting layer has a large hole mobility and an ionization energy generally as small as 5.5 eV or lower. The hole injecting/transporting layer is preferably made from a material capable of transporting holes to the light emitting layer at a low electric field strength. The hole mobility is preferably at least 10⁻⁴ cm²/V·sec under an electric field of 10⁴ to 10⁶ V/cm.

When the aromatic amine derivative of the invention is used in the hole transporting region, the hole injecting/transporting layer may be formed by the aromatic amine derivative alone or in combination with another material. The material for forming the hole injecting/transporting layer in combination with the aromatic amine derivative of the invention is not particularly limited as long as the material has the property described above and may be selected from materials which are generally used as a hole transporting material for photoconductive materials and known compounds which have been used for a hole injecting/transporting layer of organic EL devices.

Examples of the above compounds include triazole derivatives (U.S. Pat. No. 3,112,197); oxadiazole derivatives (U.S. Pat. No. 3,189,447); imidazole derivatives (JP-B-37-16096); polyarylalkane derivatives (U.S. Pat. No. 3,615,402, U.S. Pat. No. 3,820,989, U.S. Pat. No. 3,542,544, JP-B-45-555, JP-B-51-10983, JP-A-51-93224, JP-A-55-17105, JP-A-56-4148, JP-A-55-108667, JP-A-55-156953, and JP-A-56-36656); pyrazoline derivatives and pyrazolone derivatives (U.S. Pat. No. 3,180,729, U.S. Pat. No. 4,278,746, JP-A-55-88064, JP-A-55-88065, JP-A-49-105537, JP-A-55-51086, JP-A-56-80051, JP-A-56-88141, JP-A-57-45545, JP-A-54-112637, and JP-A-55-74546); phenylenediamine derivatives (U.S. Pat. No. 3,615,404, JP-B-51-10105, JP-B-46-3712, JP-B-47-25336, JP-A-54-53435, JP-A-54-110536, and JP-A-54-119925); arylamine derivatives (U.S. Pat. No. 3,567,450, U.S. Pat. No. 3,180,703, U.S. Pat. No. 3,240,597, U.S. Pat. No. 3,658,520, U.S. Pat. No. 4,232,103, U.S. Pat. No. 4,175,961, U.S. Pat. No. 4,012,376, JP-B-49-35702, JP-B-39-27577, JP-A-55-144250, JP-A-56-119132, JP-A-56-22437, and DE 1,110,518); amino-substituted chalcone derivatives (U.S. Pat. No. 3,526,501); oxazole derivatives (U.S. Pat. No. 3,257,203); styrylanthracene derivatives (JP-A-56-46234); fluorenone derivatives (JP-A-54-110837); hydrazone derivatives (U.S. Pat. No. 3,717,462, JP-A-54-59143, JP-A-55-52063, JP-A-55-52064, JP-A-55-46760, JP-A-55-85495, JP-A-57-11350, JP-A-57-148749, and JP-A-2-311591); stilbene derivatives (JP-A-61-210363, JP-A-61-228451, JP-A-61-14642, JP-A-61-72255, JP-A-62-47646, JP-A-62-36674, JP-A-62-10652, JP-A-62-30255, JP-A-60-93455, JP-A-60-94462, JP-A-60-174749, and JP-A-60-175052); silazane derivatives (U.S. Pat. No. 4,950,950); polysilane-based copolymers (JP-A-2-204996); aniline-based copolymers (JP-A-2-282263), and electroconductive high molecular weight oligomers (particularly thiophene oligomers) (JP-A-1-211399).

The hole injecting/transporting material is preferably a porphyrin compound (JP-A-63-295695), and an aromatic tertiary amine compound and a styrylamine compound (U.S. Pat. No. 4,127,412, JP-A-53-27033, JP-A-54-58445, JP-A-54-149634, JP-A-54-64299, JP-A-55-79450, JP-A-55-144250, JP-A-56-119132, JP-A-61-295558, JP-A-61-98353, and JP-A-63-295695), and particularly preferably an aromatic tertiary amine compound.

Further, a compound having two fused aromatic rings in its molecule, for example, 4,4′-bis(N-(1-naphthyl)-N-phenylamino)biphenyl (NPD) as disclosed in U.S. Pat. No. 5,061,569, and a compound in which three triphenylamine units are bonded together in a star-burst shape, such as 4,4′,4″-tris(N-(3-methylphenyl)-N-phenylamino)triphenylamine (MTDATA) as disclosed in JP-A-4-308688 may be used as the hole injecting/transporting material.

In addition to the aromatic dimethylidene compound described as the material for the light emitting layer, an inorganic compound of p-type Si or p-type SiC is also usable as the material for the hole injecting/transporting layer.

The hole injecting/transporting layer may be formed by making the aromatic amine derivative of the invention into a thin film by a known method, such as a vacuum vapor deposition method, a spin coating method, a casting method, and LB method. The thickness of the hole injecting/transporting layer is generally 5 nm to 5 μm, although not particularly limited thereto. The hole injecting/transporting layer may be a single layer made of one or more kinds of the above materials or may be a laminate with a different hole injecting/transporting layer made of a different compound, as long as the hole transporting region contains the aromatic amine derivative of the present invention.

To enhance the injection of holes or electrons into the light emitting layer, an organic semiconductor layer may be provided. The electrical conductivity thereof is preferably 10⁻¹⁰ S/cm or more. Examples of the material for the organic semiconductor layer include an electrically conductive oligomer, such as a thiophene-containing oligomer and an arylamine-containing oligomer disclosed in JP 8-193191A and an electrically conductive dendrimer, such as an arylamine-containing dendrimer.

The organic EL device of the invention preferably includes an acceptor layer comprising an acceptor material between the anode and the hole transporting layer. The hole transporting layer comprising the aromatic amine derivative represented by formula (1) may be adjacent to the acceptor layer. The hole transporting layer adjacent to the acceptor layer is adjacent to the cathode side of the acceptor layer.

The compound represented by formula (A), (B) or (C) having a highly planar skeleton is preferably used as the acceptor material, because the acceptor layer is well boned to the hole transporting layer comprising the aromatic amine derivative represented by formula (1) and a further improvement of device performance is expected.

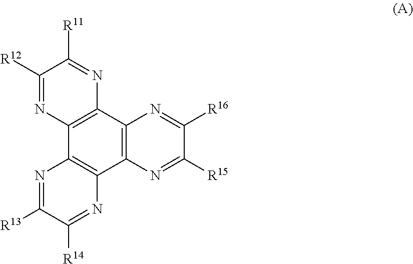

wherein each of R¹¹ to R¹⁶ independently represents a cyano group, —CONH₂, a carboxyl group, or —COOR¹⁷, wherein R¹⁷ represents an alkyl group having 1 to 20 carbon atoms; or R¹¹ and R¹², R¹³ and R¹⁴, and R¹⁵ and R¹⁶ may be bonded to each other to form a group represented by —CO—O—CO—.

Examples of the alkyl group for R¹⁷ include a methyl group, an ethyl group, a n-propyl group, an isopropyl group, a n-butyl group, an isobutyl group, a tert-butyl group, a cyclopentyl group, and a cyclohexyl group.

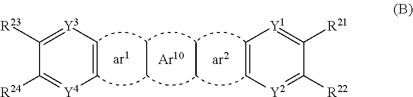

wherein:

R²¹ to R²⁴ may be the same or different and each represents a hydrogen atom, a substituted or unsubstituted alkyl group having 1 to 20 carbon atoms, a substituted or unsubstituted aryl group having 6 to 50 carbon atoms, a substituted or unsubstituted heterocyclic group having 3 to 50 ring atoms, a halogen atom, a substituted or unsubstituted fluoroalkyl group having 1 to 20 carbon atoms, a substituted or unsubstituted alkoxy group having 1 to 20 carbon atoms, a substituted or unsubstituted aryloxy group having 6 to 50 carbon atoms, or a cyano group, provided that adjacent groups of R²¹ to R²⁴ may be bonded to each other to form a ring;

Y¹ to Y⁴ may be the same or different and each represents —N═, —CH═, or —C(R²⁵)═, wherein R²⁵ represents a substituted or unsubstituted alkyl group having 1 to 20 carbon atoms, a substituted or unsubstituted aryl group having 6 to 50 carbon atoms, a substituted or unsubstituted heterocyclic group having 3 to 50 ring atoms, a halogen atom, a substituted or unsubstituted alkoxy group having 1 to 20 carbon atoms, a substituted or unsubstituted aryloxy group having 6 to 50 ring carbon atoms, or a cyano group;

Ar¹⁰ represents a fused ring having 6 to 24 ring carbon atoms or a hetero cyclic ring having 6 to 24 ring atoms; and

each of ar¹ and ar² independently represents a ring represented by formula (i) or (ii):

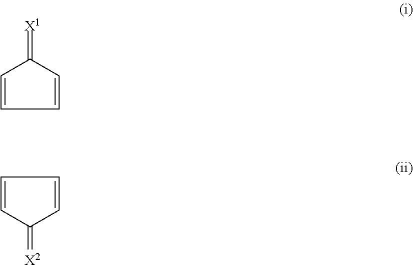

wherein X¹ and X² may be the same or different and each represents a divalent group represented by any one of formulae (a) to (g):

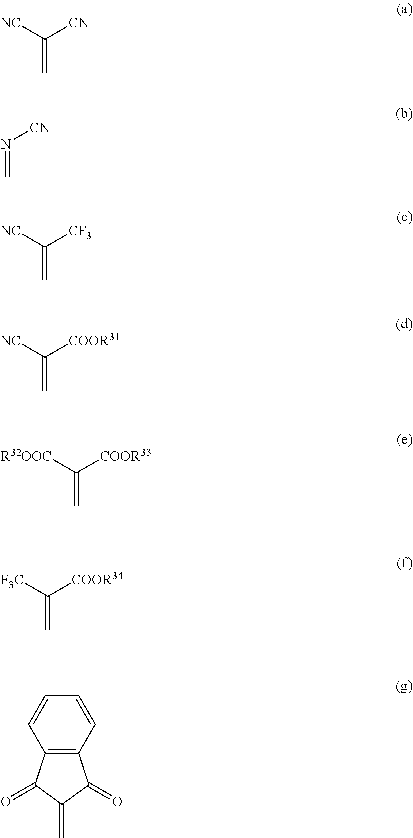

wherein R³¹ to R³⁴ may be the same or different and each represents a hydrogen atom, a substituted or unsubstituted alkyl group having 1 to 20 carbon atoms, a substituted or unsubstituted aryl group having 6 to 50 carbon atoms, or a substituted or unsubstituted heterocyclic group having 3 to 50 ring atoms, provided that R³² and R³³ may be bonded to each other to form a ring.

Examples of the groups for R²¹ to R²⁴ and R³¹ to R³⁴ are described below. Examples of the alkyl group include a methyl group, an ethyl group, a n-propyl group, an isopropyl group, a n-butyl group, an isobutyl group, a tert-butyl group, a cyclopentyl group, and a cyclohexyl group. Examples of the aryl group include a phenyl group, a biphenyl group, and a naphthyl group. Examples of the heterocyclic group include residues of pyridine, pyrazine, furan, imidazole, benzimidazole, and thiophene. Examples of the halogen atom include a fluorine atom, a chlorine atom, a bromine atom, and an iodine atom. Examples of the alkoxy group include a methoxy group and an ethoxy group. Examples of the aryloxy group may include a phenyloxy group and a pentaphenyloxy group.

The above groups may have a substituent. Examples of the substituted aryl group include a haloaryl group, such as a monofluorophenyl group and a trifluoromethylphenyl group, and an aryl group having an alkyl substituent having 1 to 10, preferably 1 to 5 carbon atoms, such as a tolyl group and a 4-t-butylphenyl group. Examples of the substituted alkyl group include a haloalkyl group, such as trifluoromethyl group, a pentafluoroethyl group, a perfluorocyclohexyl group, and a perfluoroadamantyl group. Examples of the substituted aryloxy group include a haloaryloxy group, such as a 4-trifluorophenyloxy group and a pentafluorophenyloxy group, and an aryloxy group having an alkyl substituent having 1 to 10, preferably 1 to 5 carbon atoms, such as a 4-t-butylphenoxy group.

Adjacent groups of R²¹ to R²⁴ may be bonded to each other to form a ring, such as a benzene ring, a naphthalene ring, a pyrazine ring, a pyridine ring, and a furan ring.

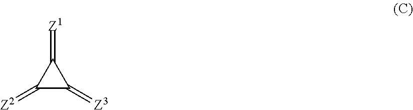

wherein each of Z¹ to Z³ independently represents a divalent group represented by formula (h):

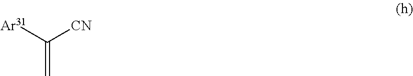

wherein Ar³¹ represents a substituted or unsubstituted aryl group having 6 to 50 carbon atoms or a substituted or unsubstituted heteroaryl group having 3 to 50 ring atoms.

The aryl group may include a phenyl group and a naphthyl group.

The heteroaryl group may include a pyridine, a pyrazine, a pyrimidine, a quinoline, and an isoquinoline.

The substituent for these groups may include an electron-withdrawing group, such as a cyano group, a fluorine atom, a trifluoromethyl group, a chlorine atom, and a bromine atom.

### Electron Injecting/Transporting Layer

The electron injecting/transporting layer is a layer which facilitates the injection of electrons into the light emitting layer, transports the electrons to the light emitting region, and has a large electron mobility. An adhesion improving layer is an electron injecting layer made of a material having a good adhesion particularly to a cathode.

It has been known that the emitted light from a light emitting layer is reflected by an electrode (cathode in this case), so the emitted light directly coming through an anode and the emitted light coming through the anode after reflected by the cathode interfere with each other. To effectively utilize this interference effect, the thickness of the electron transporting layer is appropriately selected from several nanometers to several micrometers. When the thickness is large, the electron mobility is preferably at least 10⁻⁵ cm²/Vs or more at an electric field of 10⁴ to 10⁶ V/cm in order to avoid an increase in voltage.

A metal complex of 8-hydroxyquinoline or its derivative or an oxadiazole derivative is suitable as the material for an electron injecting layer. Examples of the metal complex of 8-hydroxyquinoline or its derivative include metal chelate oxynoid compounds each containing a chelate of oxine (generally 8-quinolinol or 8-hydroxyquinoline), such as tris(8-quinolinol)aluminum.

Examples of the oxadiazole derivative include electron transfer compounds represented by the following formula:

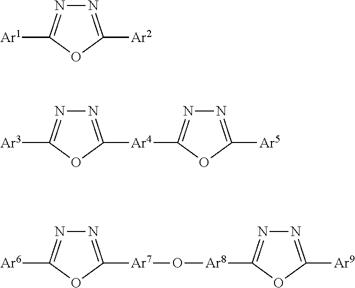

wherein Ar¹, Ar², Ar³, Ar⁵, Ar⁶ and Ar⁹ are the same or different and each represent a substituted or unsubstituted aryl group; and Ar⁴, Ar⁷ and Ar⁸ are the same or different and each represent a substituted or unsubstituted arylene group. Examples of the aryl group include phenyl group, biphenyl group, anthryl group, perylenyl group, and pyrenyl group. Examples of the arylene group include phenylene group, naphthylene group, biphenylene group, anthranylene group, perylenylene group, and pyrenylene group. Examples of the substituent include an alkyl group having 1 to 10 carbon atoms, an alkoxyl group having 1 to 10 carbon atoms, and a cyano group. The electron transfer compound is preferably a thin film-forming compound.

Examples of the electron transfer compound are shown below.

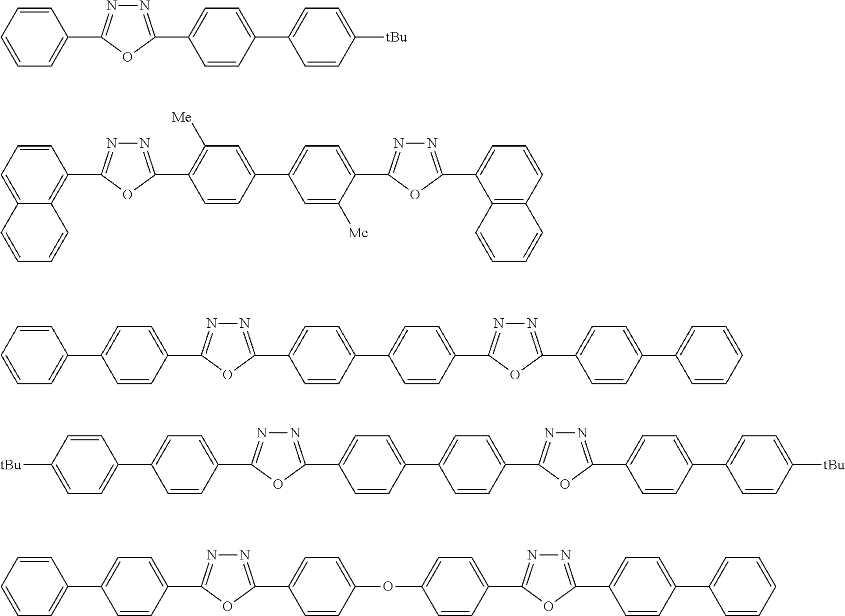 [SMILES: CC(C)(C)c1ccc(-c2ccc(-c3nnc(-c4ccccc4)o3)cc2)cc1 ; Cc1cc(-c2ccc(-c3nnc(-c4cccc5ccccc45)o3)c(C)c2)ccc1-c1nnc(-c2cccc3ccccc23)o1 ; c1ccc(-c2ccc(-c3nnc(-c4ccc(-c5ccc(-c6nnc(-c7ccc(-c8ccccc8)cc7)o6)cc5)cc4)o3)cc2)cc1 ; CC(C)(C)c1ccc(-c2ccc(-c3nnc(-c4ccc(-c5ccc(-c6nnc(-c7ccc(-c8ccc(C(C)(C)C)cc8)cc7)o6)cc5)cc4)o3)cc2)cc1 ; c1ccc(-c2ccc(-c3nnc(-c4ccc(Oc5ccc(-c6nnc(-c7ccc(-c8ccccc8)cc7)o6)cc5)cc4)o3)cc2)cc1]

The compounds represented by formulae (A) to (F) may be also usable as the material for the electron injecting layer and the electron transporting layer.

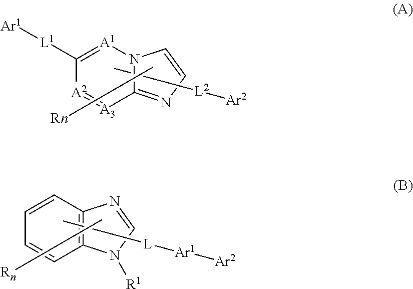

wherein A¹ to A³ each independently represent a nitrogen atom or a carbon atom;

Ar¹ represents a substituted or unsubstituted aryl group having 6 to 60 ring carbon atoms or a substituted or unsubstituted heteroaryl group having 3 to 60 ring atoms;

Ar² represents a hydrogen atom, a substituted or unsubstituted aryl group having 6 to 60 ring carbon atoms, a substituted or unsubstituted heteroaryl group having 3 to 60 ring atoms, a substituted or unsubstituted alkyl group having 1 to 20 carbon atoms, a substituted or unsubstituted alkoxy group having 1 to 20 carbon atoms, or a divalent group of the preceding groups;

provided that at least one selected from Ar¹ and Ar² is a substituted or unsubstituted fused ring group having 10 to 60 ring carbon atoms or a substituted or unsubstituted monohetero fused ring group having 3 to 60 ring atoms;

L¹, L², and L³ each independently represent a single bond, a substituted or unsubstituted arylene group having 6 to 60 ring carbon atoms, a substituted or unsubstituted heteroarylene group having 3 to 60 ring atoms, or a substituted or unsubstituted fluorenylene group;

R and R¹ each independently represent a hydrogen atom, a substituted or unsubstituted aryl group having 6 to 60 ring carbon atoms, a substituted or unsubstituted heteroaryl group having 3 to 60 ring atoms, a substituted or unsubstituted alkyl group having 1 to 20 carbon atoms, or a substituted or unsubstituted alkoxy group having 1 to 20 carbon atoms; and

n represents an integer of 0 to 5, when n is 2 or more, R groups may be the same or different and adjacent R groups may bond to each other to form an aliphatic hydrocarbon ring or an aromatic hydrocarbon ring.

HAr-L-Ar¹—Ar² (C)

wherein HAr represents a substituted or unsubstituted nitrogen-containing heterocyclic group having 3 to 40 carbon atoms; L represents a single bond, a substituted or unsubstituted arylene group having 6 to 60 carbon atoms, a substituted or unsubstituted heteroarylene group having 3 to 60 carbon atoms, or a substituted or unsubstituted fluorenylene group; Ar¹ represents a substituted or unsubstituted divalent aromatic hydrocarbon group having 6 to 60 carbon atoms; and Ar² represents a substituted or unsubstituted aryl group having 6 to 60 carbon atoms or a substituted or unsubstituted heteroaryl group having 3 to 60 carbon atoms.

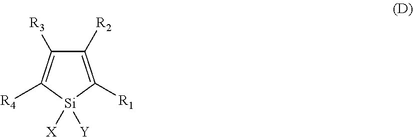

wherein X and Y each independently represent a saturated or unsaturated hydrocarbon group having 1 to 6 carbon atoms, an alkoxy group, an alkenyloxy group, an alkynyloxy group, a hydroxyl group, a substituted or unsubstituted aryl group, or a substituted or unsubstituted hetero cyclic ring, or X and Y represent a saturated or unsaturated ring by bonding to each other; R₁ to R₄ each independently represent a hydrogen atom, a halogen atom, a substituted or unsubstituted alkyl group having 1 to 6 carbon atoms, an alkoxy group, an aryloxy group, a perfluoroalkyl group, a perfluoroalkoxy group, an amino group, an alkylcarbonyl group, an arylcarbonyl group, an alkoxycarbonyl group, an aryloxycarbonyl group, an azo group, an alkylcarbonyloxy group, an arylcarbonyloxy group, an alkoxycarbonyloxy group, an aryloxycarbonyloxy group, a sulfinyl group, a sulfonyl group, a sulfanyl group, a silyl group, a carbamoyl group, an aryl group, a heterocyclic group, an alkenyl group, an alkynyl group, a nitro group, a formyl group, a nitroso group, a formyloxy group, an isocyano group, a cyanate group, an isocyanate group, a thiocyanate group, an isothiocyanate group, or a cyano group, provided that adjacent groups may form a substituted or unsubstituted fused ring.

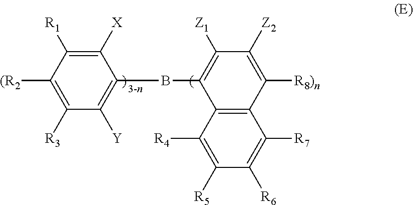

wherein R₁ to R₈ and Z₂ each independently represent a hydrogen atom, a saturated or unsaturated hydrocarbon group, an aromatic group, a heterocyclic group, a substituted amino group, a substituted boryl group, an alkoxy group, or an aryloxy group; X, Y and Z, each independently represent a saturated or unsaturated hydrocarbon group, an aromatic group, a heterocyclic group, a substituted amino group, an alkoxy group, or an aryloxy group; Z¹ and Z² may be bonded to each other to form a fused ring; and n represents an integer of 1 to 3, when n is 2 or more, groups Z₁ may be different; provided that a compound wherein n is 1, X, Y and R₂ each represent a methyl group, and R₈ is a hydrogen atom or a substituted boryl group and a compound wherein n is 3 and Z₁ is a methyl group are excluded.

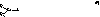

wherein Q¹ and Q² each independently represent a ligand represented by formula (G), L represents a halogen atom, a substituted or unsubstituted alkyl group, a substituted or unsubstituted cycloalkyl group, a substituted or unsubstituted aryl group, a substituted or unsubstituted heterocyclic group, —OR¹ wherein R¹ represents a hydrogen atom, a substituted or unsubstituted alkyl group, a substituted or unsubstituted cycloalkyl group, a substituted or unsubstituted aryl group, or a substituted or unsubstituted heterocyclic group, or a ligand represented by —O—Ga-Q³(Q⁴) wherein Q³ and Q⁴ are as defined in Q¹ and Q².

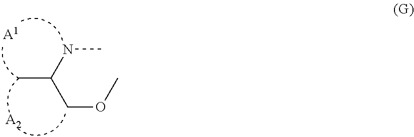

wherein rings A¹ and A² each represent a substituted or unsubstituted fused six-membered aryl ring.

This metal complex strongly exhibits a character of n-type semiconductor and has a large electron injection ability. Since the energy of forming complex is small, the metal and the ligand in resulting metal complex bond strongly to each other, to increase the fluorescence quantum efficiency of light emitting material.

Examples of the substituents of rings A¹ and A² each forming the ligand represented by formula (G) include a halogen atom, such as chlorine, bromine, iodine, and fluorine; a substituted or unsubstituted alkyl group, such as a methyl group, an ethyl group, a propyl group, a butyl group, a s-butyl group, a t-butyl group, a pentyl group, a hexyl group, a heptyl group, an octyl group, a stearyl group, and a trichloromethyl group; a substituted or unsubstituted aryl group, such as a phenyl group, a naphthyl group, a 3-methylphenyl group, a 3-methoxyphenyl group, a 3-fluorophenyl group, a 3-trichloromethylphenyl group, a 3-trifluoromethylphenyl group, and a 3-nitrophenyl group; a substituted or unsubstituted alkoxyl group, such as a methoxy group, a n-butoxy group, a t-butoxy group, a trichloromethoxy group, a trifluoroethoxy group, a pentafluoropropoxy group, a 2,2,3,3-tetrafluoropropoxy group, a 1,1,1,3,3,3-hexafluoro-2-propoxy group, and a 6-(perfluoroethyl)hexyloxy group; a substituted or unsubstituted aryloxy group, such as a phenoxy group, a p-nitrophenoxy group, a p-t-butylphenoxy group, a 3-fluorophenoxy group, a pentafluorophenyl group, and a 3-trifluoromethylphenoxy group; a substituted or unsubstituted alkylthio group, such as a methylthio group, an ethylthio group, a t-butylthio group, a hexylthio group, an octylthio group, and a trifluoromethylthio group; a substituted or unsubstituted arylthio group, such as a phenylthio group, a p-nitrophenylthio group, a p-t-butylphenylthio group, a 3-fluorophenylthio group, a pentafluorophenylthio group, and a 3-trifluoromethylphenylthio group; a cyano group; a nitro group; an amino group; a mono- or di-substituted amino group, such as a methylamino group, a diethylamino group, an ethylamino group, a diethylamino group, a dipropylamino group, a dibutyl amino group, and a diphenylamino group; an acylamino group, such as a bis(acetoxymethyl)amino group, a bis(acetoxyethyl)amino group, a bis(acetoxypropyl)amino group, and a bis(acetoxybutyl)amino group; a hydroxyl group; a siloxy group; an acyl group; a carbamoyl group, such as a methylcarbamoyl group, a dimethylcarbamoyl group, an ethylcarbamoyl group, a diethylcarbamoyl group, a propylcarbamoyl group, a butylcarbamoyl group, and a phenylcarbamoyl group; a carboxyl group; a sulfonic acid group; an imido group; a cycloalkyl group, such as a cyclopentyl group and a cyclohexyl group; an aryl group, such as a phenyl group, a naphthyl group, a biphenyl group, an anthranyl group, a phenanthryl group, a fluorenyl group, and a pyrenyl group; and a heterocyclic group, such as a pyridinyl group, a pyrazinyl group, a pyrimidinyl group, a pyridazinyl group, a triazinyl group, an indolinyl group, a quinolinyl group, an acridinyl group, a pyrrolidinyl group, a dioxanyl group, a piperidinyl group, a morpholidinyl group, a piperazinyl group, a triazinyl group, a carbazolyl group, a furanyl group, a thiophenyl group, an oxazolyl group, an oxadiazolyl group, a benzoxazolyl group, a thiazolyl group, a thiadiazolyl group, a benzothiazolyl group, a triazolyl group, an imidazolyl group, a benzimidazolyl group, and a furanyl group. The above substituents may bond to each other to form a six-membered aryl ring or hetero cyclic ring.

In a preferred embodiment of the organic EL device, a reductive dopant is included in an electron transporting region or an interfacial region between a cathode and an organic layer. The reductive dopant is defined as a substance capable of reducing an electron transporting compound. Therefore, a compound having a certain level of reducing ability may be used as the reductive dopant. Examples thereof include at least one compound selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an organic complex of alkali metal, an organic complex of alkaline earth metal, and an organic complex of rare earth metal.

Examples of the preferred reductive dopant include at least one alkali metal selected from the group consisting of Na (work function: 2.36 eV), K (work function: 2.28 eV), Rb (work function: 2.16 eV), and Cs (work function: 1.95 eV) or at least one alkaline earth metal selected from the group consisting of Ca (work function: 2.9 eV), Sr (work function: 2.0 to 2.5 eV) and Ba (work function: 2.52 eV). A reductive dopant having a work function of 2.9 eV or less is particularly preferred. Of the above, at least one alkali metal selected from the group consisting of K, Rb and Cs is more preferred, with Rb and Cs being still more preferred and Cs being most preferred.

Since these alkali metals have a particularly high reducing ability, the luminance and lifetime of organic EL device are improved by the addition thereof into an electron injection region in a relatively small amount. A combination of two or more alkali metals is also preferably used as the reductive dopant having a work function of 2.9 eV or smaller. A combination containing Cs, for example, Cs and Na, Cs and K, Cs and Rb, and Cs, Na and K, is particularly preferred. By combinedly containing Cs, the reductive dopant effectively exhibits a reducing ability and the luminance and lifetime of organic EL device are improved by the addition thereof into the electron injection region.

The organic EL device of the present invention may further comprise an electron injecting layer made of an insulating material or a semiconductor between the cathode and the organic layer. The electron injecting layer effectively prevents a leak of electric current, to improve the electron injection ability. The insulating material is preferably at least one metal compound selected from the group consisting of alkali metal chalcogenide, alkaline earth metal chalcogenide, alkali metal halide, and alkaline earth metal halide. When the electron injecting layer is made of the alkali metal chalcogenide, the electron injection ability is further improved.

Examples of preferred alkali metal chalcogenide include Li₂O, K₂O, Na₂S, Na₂Se, and NaO. Examples of preferred alkaline earth metal chalcogenide include CaO, BaO, SrO, BeO, BaS, and CaSe. Examples of preferred alkali metal halide include LiF, NaF, KF, LiCl, KCl, and NaCl. Examples of preferred alkaline earth metal halide include fluorides, such as CaF₂, BaF₂, SrF₂, MgF₂, and BeF₂, and halides other than fluorides.

Examples of the semiconductor for forming the electron transporting layer include oxides, nitrides and oxynitrides, alone or in combination of two or more thereof, each containing at least one element selected from Ba, Ca, Sr, Yb, Al, Ga, In, Li, Na, Cd, Mg, Si, Ta, Sb, and Zn. The electron transporting layer is preferably a microcrystalline or amorphous, insulating thin film of an inorganic compound. Since the electron transporting layer is made more uniform by forming it from such an insulating thin film, the pixel defects, such as dark spots, can be decreased. Examples of such an inorganic compound include the alkali metal chalcogenides, the alkaline earth metal chalcogenides, the alkali metal halides and the alkaline earth metal halides which are described above.

### Insulating Layer

Since an electric field is applied to the ultra-thin films of organic EL device, the pixel defects due to leak and short circuit tends to occur. To prevent the defects, an insulating thin film layer is preferably interposed between the pair of electrodes. Examples of the material for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. These materials may be used in combination two or more or may be made into laminated layers.

### Production of Organic EL Device

By using the materials and the production method mentioned above, the organic EL device may be produced, for example, by forming an anode, an optional hole injecting/transporting layer, a light emitting layer, and an optional electron injecting/transporting layer, and then forming a cathode. Alternatively, the organic EL device may be produced by forming each layer in a reverse order from a cathode to an anode.

Each layer of the organic EL device of the invention may be formed by any of known methods such as a vacuum vapor deposition method and a spin coating method, although not particularly limited thereto. The organic thin-film layer comprising the compound represented by formula (1) in the organic EL device of the invention is formed by a known method such as a vacuum vapor deposition method, a molecular beam epitaxy method (MBE method) and a coating method, for example, a dipping method, a spin coating method, a casting method, a bar coating method and a roll coating method each using a solution of the compound in a solvent.

The production of organic EL device will be described below with reference to the production of an organic EL device having a sequentially layered structure of anode/hole injecting layer/light emitting layer/electron injecting layer/cathode on a light-transmissive substrate.

First, on a suitable light-transmissive substrate, an anode is formed by making an anode material into a thin film having a thickness of 1 μm or less, preferably 10 to 200 nm by a method, such as vapor deposition and sputtering.

Then, a hole injecting layer is formed on the anode. The hole injecting layer may be formed by a vacuum vapor deposition method, a spin coating method, a casting method or LB method, with the vacuum vapor deposition method being preferred because a uniform film is easily obtained and pinholes are hardly formed. The conditions of the vacuum vapor deposition method for forming the hole injecting layer depend upon the compound to be used (hole injecting layer material), and the crystalline structure, recombination structure and other factors of the intended hole injecting layer, and the vacuum vapor deposition is conducted preferably under the conditions: a deposition source temperature of 50 to 450° C., a vacuum degree of 10⁻⁷ to 10⁻³ torr, a deposition speed of 0.01 to 50 nm/s, a substrate temperature of −50 to 300° C., and a film thickness of 5 nm to 5 μm.

Then, a light emitting layer is formed on the hole transporting layer by making an appropriate organic light emitting material into a thin film by a vacuum vapor deposition method, a spin coating method, or a casting method, with the vacuum vapor deposition method being preferred because a uniform film is easily obtained and pinholes are hardly formed. The conditions of the vacuum vapor deposition method for forming the light emitting layer depend upon the compound to be used, and generally selected from those mentioned with respect to the hole injecting layer.

Next, an electron injecting layer is formed on the light emitting layer. Like the formation of the hole injecting layer and the light emitting layer, the electron injecting layer is formed preferably by a vacuum vapor deposition method because a uniform thin film is needed. The conditions of the vacuum vapor deposition are selected from those mentioned with respect to the hole injecting layer and the light emitting layer.

In a vapor deposition method, the aromatic amine derivative of the invention may be co-deposited with another material, although depending on which layer of a light emitting region and a hole transporting region is to be formed. In a spin coating method, a mixture of the aromatic amine derivative and another material may be made into a thin film.

Finally, a cathode is deposited on the electron injecting layer, to obtain an organic EL device. The cathode is made of a metal and can be formed by a vapor deposition method or a sputtering method, with the vacuum vapor deposition method being preferred in view of preventing the underlying organic layers from being damaged during the film forming process.

In the production of organic EL device mentioned above, the layers from the anode to the cathode are successively formed preferably in a single evacuation operation.

The thickness of each organic thin film layer in the organic EL device is not particularly limited and preferably several nanometers to 1 μm because an excessively small thickness may cause defects, such as pin holes, and an excessively large thickness may require a high driving voltage.

When applying a direct voltage of 5 to 40 V to an organic EL device so that the anode has + polarity and the cathode has − polarity, an emission of light is observed. If a voltage is applied in the reverse polarity, no electric current flows and light is not emitted. When an alternating voltage is applied, the uniform light emission is observed only when the anode is + polarity and the cathode is − polarity. The wave shape of alternating voltage is not limited.

### EXAMPLES

The present invention will be described below in more detail with reference to the synthesis examples and the examples. However, it should be noted that the scope of the invention is not limited thereto.

### Intermediate Synthesis 1-1

### Synthesis of Intermediate 1-1

Under a nitrogen atmosphere, 100 ml of dehydrated toluene was added to a mixture of 7.10 g of 2-iodo-9,9-dimethylfluorene, 4.12 g of 2-aminobiphenyl, 2.97 g of sodium t-butoxide, 0.09 g of [1,1′-bis(diphenylphosphino)ferrocene]palladium(II) dichloride dichloromethane complex, and 0.12 g of 1,1′-bis(diphenylphosphino)ferrocene. The reaction was allowed to proceed at 90° C. for 3 h. After cooling, the toluene solution was washed with water, extracted, and then filtered through celite. The filtrate was concentrated under reduced pressure. The obtained residue was purified by silica gel column chromatography to obtain 4.15 g of a viscous solid, which was identified as the following intermediate 1-1 by FD-MS analysis (yield: 52%).

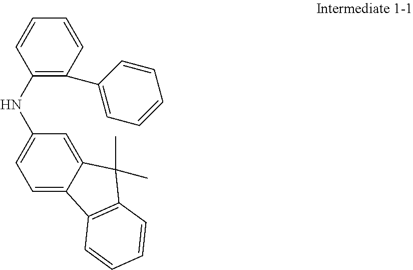 [SMILES: CC1(C)c2ccccc2-c2ccc(Nc3ccccc3-c3ccccc3)cc21]

### Intermediate Synthesis 1-2

### Synthesis of Intermediate 1-2

In the same manner as in Intermediate Synthesis 1-1 except for using 5.16 g of 4-bromobiphenyl in place of 2-iodo-9,9-dimethylfluorene, 4.26 g of a pale brown solid was obtained, which was identified as the following intermediate 1-2 by FD-MS analysis (yield: 60%).

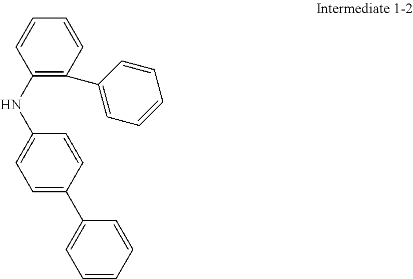 [SMILES: c1ccc(-c2ccc(Nc3ccccc3-c3ccccc3)cc2)cc1]

### Intermediate Synthesis 1-3

### Synthesis of Intermediate 1-3

Under a nitrogen atmosphere, 60 ml of dehydrated toluene was added to a mixture of 6.00 g of 3-methyl-4-(2-methylphenyl)aniline, 7.09 g of 2-bromobiphenyl, 4.10 g of sodium t-butoxide, 0.125 g of [1,1′-bis(diphenylphosphino)ferrocene]palladium(II) dichloride dichloromethane complex, and 0.169 g of 1,1′-bis(diphenylphosphino)ferrocene. The reaction was allowed to proceed at 90° C. for 6 h. After cooling, the toluene solution was washed with water, extracted, and then filtered through celite. The filtrate was concentrated under reduced pressure. The obtained residue was purified by silica gel column chromatography to obtain 10.3 g of a viscous solid, which was identified as the following intermediate 1-3 by FD-MS analysis (yield: 97%).

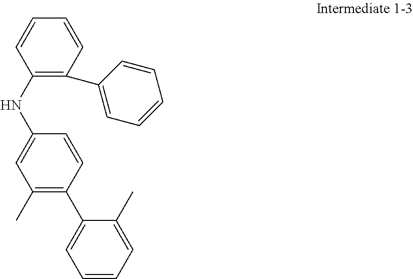 [SMILES: Cc1ccccc1-c1ccc(Nc2ccccc2-c2ccccc2)cc1C]

### Intermediate Synthesis 1-4

### Synthesis of Intermediate 1-4

In the same manner as in Intermediate Synthesis 1-3 except for using 5.57 g of 3-methyl-4-phenylaniline in place of 3-methyl-4-(2-methylphenyl)aniline, 8.07 g of a viscous solid was obtained, which was identified as the following intermediate 1-4 by FD-MS analysis (yield: 79%).

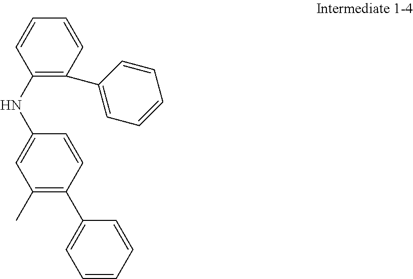 [SMILES: Cc1cc(Nc2ccccc2-c2ccccc2)ccc1-c1ccccc1]

### Intermediate Synthesis 1-5

### Synthesis of Intermediate 1-5

Under a nitrogen atmosphere, 50 ml of dehydrated toluene was added to a mixture of 5.38 g of 4-bromobiphenyl, 5.67 g of terphenyl-2-amine, 3.11 g of sodium t-butoxide, 0.0518 g of palladium acetate, and 0.256 g of 1,1′-bis(diphenylphosphino)ferrocene. The reaction was allowed to proceed at 90° C. for 3 h. After cooling, the toluene solution was washed with water, extracted, and then filtered through celite. The filtrate was concentrated under reduced pressure. The obtained residue was purified by silica gel column chromatography to obtain 7.61 g of a white solid, which was identified as the following intermediate 1-5 by FD-MS analysis (yield: 83%).

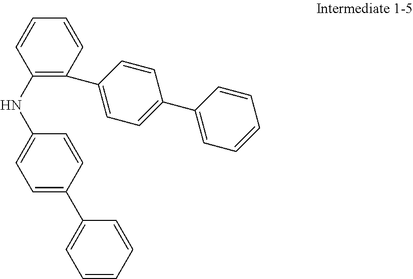 [SMILES: c1ccc(-c2ccc(Nc3ccccc3-c3ccc(-c4ccccc4)cc3)cc2)cc1]

### Intermediate Synthesis 1-6

### Synthesis of Intermediate 1-6

In the same manner as in Intermediate Synthesis 1-6 except for using 5.46 g of bromobenzene in place of 4-bromobiphenyl, 8.98 g of 2-(dibenzofuran-4-yl)aniline in place of terphenyl-2-amine, and using 4.67 g of sodium t-butoxide, 0.0777 g of palladium acetate, and 0.384 g of 1,1′-bis(diphenylphosphino)ferrocene, 10.4 g of a viscous solid was obtained, which was identified as the following intermediate 1-6 by FD-MS analysis (yield: 90%).

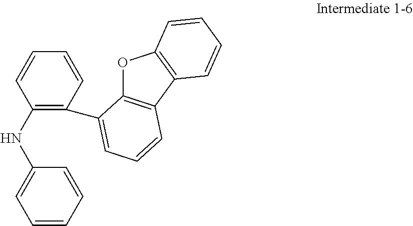 [SMILES: c1ccc(Nc2ccccc2-c2cccc3c2oc2ccccc23)cc1]

### Intermediate Synthesis 1-7

### Synthesis of Intermediate 1-7

Under a nitrogen atmosphere, 80 mL of dehydrated toluene was added to a mixture of 13.0 g of 4,4′-diiodo-3,3-dimethylbiphenyl (manufactured by Tokyo Chemical Industry Co., Ltd.), 10.44 g of terphenyl-2-amine, 8.06 g of sodium t-butoxide, 0.42 g of palladium acetate, and 1.09 g of tri-t-butylphosphonium tetrabromoborate. The reaction was allowed to proceed at 110° C. for 8 h. After cooling, the toluene solution was washed with water, extracted, and then filtered through celite. The filtrate was concentrated under reduced pressure to obtain a crude product. The obtained crude product was purified by silica gel column chromatography to obtain 12.2 g of a pale yellow solid, which was identified as the following intermediate 1-7 by FD-MS analysis (yield: 78.9%).

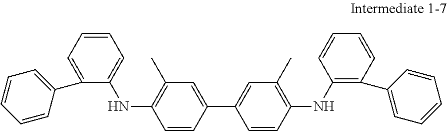 [SMILES: Cc1cc(-c2ccc(Nc3ccccc3-c3ccccc3)c(C)c2)ccc1Nc1ccccc1-c1ccccc1]

### Synthesis Example 1

Under a nitrogen atmosphere, 80 mL of dehydrated xylene was added to a mixture of 4.86 g of 4,4′-diiodo-3,3-dimethylbiphenyl (manufactured by Tokyo Chemical Industry Co., Ltd.), 8.10 g of the intermediate 1-1, 3.02 g of sodium t-butoxide, 0.0503 g of palladium acetate, and 0.130 g of tri-t-butylphosphonium tetrabromoborate. The reaction was allowed to proceed at 130° C. for 5 h. After cooling, the xylene solution was washed with water, extracted, and then filtered through celite. The filtrate was concentrated under reduced pressure to obtain a crude product. The obtained crude product was purified by silica gel column chromatography and then recrystallized from toluene to obtain 7.67 g a pale yellow solid, which was identified as the following aromatic amine derivative HT1 by FD-MS analysis (yield: 76%).

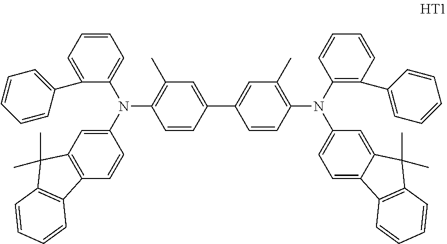 [SMILES: Cc1cc(-c2ccc(N(c3ccc4c(c3)C(C)(C)c3ccccc3-4)c3ccccc3-c3ccccc3)c(C)c2)ccc1N(c1ccc2c(c1)C(C)(C)c1ccccc1-2)c1ccccc1-c1ccccc1]

### Synthesis Example 2

In the same manner as in Synthesis Example 1 except for using 7.20 g of the intermediate 1-2 in place of the intermediate 1-1, 5.51 g of a pale yellow solid was obtained, which was identified as the following aromatic amine derivative HT2 by FD-MS analysis (yield: 60%).

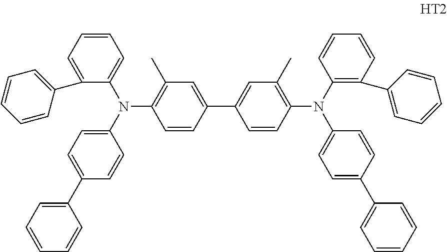 [SMILES: Cc1cc(-c2ccc(N(c3ccc(-c4ccccc4)cc3)c3ccccc3-c3ccccc3)c(C)c2)ccc1N(c1ccc(-c2ccccc2)cc1)c1ccccc1-c1ccccc1]

### Synthesis Example 3

In the same manner as in Synthesis Example 1 except for using 4,4′-diiodo-2,2′-dimethylbiphenyl (manufactured by Aldrich) in place of 4,4′-diiodo-3,3′-dimethylbiphenyl (manufactured by Tokyo Chemical Industry Co., Ltd.), 4.50 g of a pale yellow solid was obtained, which was identified as the following aromatic amine derivative HT3 by FD-MS analysis (yield: 49%).

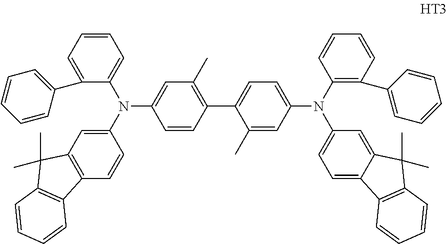 [SMILES: Cc1cc(N(c2ccc3c(c2)C(C)(C)c2ccccc2-3)c2ccccc2-c2ccccc2)ccc1-c1ccc(N(c2ccc3c(c2)C(C)(C)c2ccccc2-3)c2ccccc2-c2ccccc2)cc1C]

### Synthesis Example 4

Under a nitrogen atmosphere, 45 mL of dehydrated xylene was added to a mixture of 2.50 g of 4,4′-diiodo-3,3-dimethylbiphenyl (manufactured by Tokyo Chemical Industry Co., Ltd.), 4.03 g of the intermediate 1-3, 1.55 g of sodium t-butoxide, 0.0259 g of palladium acetate, and 0.0466 g of tri-t-butylphosphine. The reaction was allowed to proceed at 130° C. for 3 h. After cooling, the xylene solution was washed with water, phase-separated, and then filtered through celite. The filtrate was concentrated under reduced pressure to obtain a crude product. The obtained crude product was purified by silica gel column chromatography and then recrystallized from toluene/methanol to obtain 1.77 g of a pale yellow solid, which was identified as the following aromatic amine derivative HT4 by FD-MS analysis (yield: 35%).

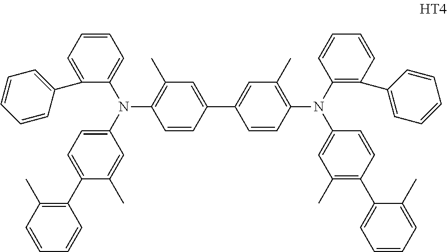 [SMILES: Cc1ccccc1-c1ccc(N(c2ccc(-c3ccc(N(c4ccc(-c5ccccc5C)c(C)c4)c4ccccc4-c4ccccc4)c(C)c3)cc2C)c2ccccc2-c2ccccc2)cc1C]

### Synthesis Example 5

In the same manner as in Synthesis Example 4 except for using 3.85 g of the intermediate 1-4 in place of the intermediate 1-3, 1.95 g of a pale yellow solid was obtained, which was identified as the following aromatic amine derivative HT5 by FD-MS analysis (yield: 40%).

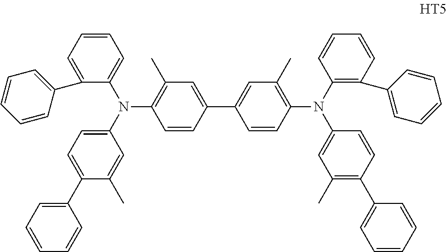 [SMILES: Cc1cc(N(c2ccc(-c3ccc(N(c4ccc(-c5ccccc5)c(C)c4)c4ccccc4-c4ccccc4)c(C)c3)cc2C)c2ccccc2-c2ccccc2)ccc1-c1ccccc1]

### Synthesis Example 6

In the same manner as in Synthesis Example 1 except for using 8.91 g of the intermediate 1-5 in place of the intermediate 1-1, 8.39 g of a pale yellow solid was obtained, which was identified as the following aromatic amine derivative HT6 by FD-MS analysis (yield: 77%).

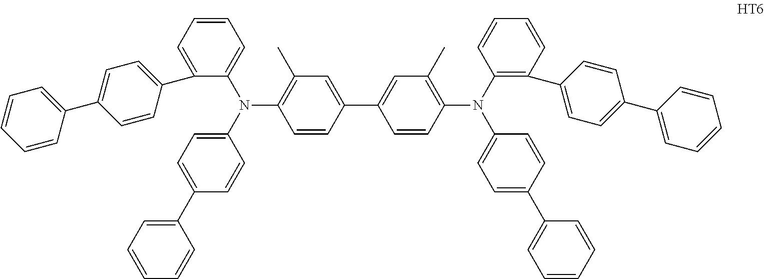 [SMILES: Cc1cc(-c2ccc(N(c3ccc(-c4ccccc4)cc3)c3ccccc3-c3ccc(-c4ccccc4)cc3)c(C)c2)ccc1N(c1ccc(-c2ccccc2)cc1)c1ccccc1-c1ccc(-c2ccccc2)cc1]

### Synthesis Example 7

In the same manner as in Synthesis Example 1 except for using 8.26 g of the intermediate 1-6 in place of the intermediate 1-1, 4.75 g of a pale yellow solid was obtained, which was identified as the following aromatic amine derivative HT7 by FD-MS analysis (yield: 50%).

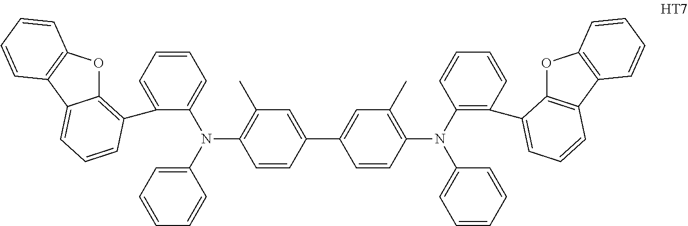 [SMILES: Cc1cc(-c2ccc(N(c3ccccc3)c3ccccc3-c3cccc4c3oc3ccccc34)c(C)c2)ccc1N(c1ccccc1)c1ccccc1-c1cccc2c1oc1ccccc12]

### Synthesis Example 8

Under a nitrogen atmosphere, 200 mL of dehydrated xylene was added to a mixture of 9.88 g of 2-bromodibenzofuran, 10.3 g of the intermediate 1-7, 4.6 g of sodium t-butoxide, 0.2 g of palladium acetate, and 0.45 g of tri-t-butylphosphonium tetrabromoborate. The reaction was allowed to proceed at 110° C. for 2 h. After cooling, the xylene solution was washed with water, extracted, and then filtered through celite. The filtrate was concentrated under reduced pressure to obtain a crude product. The obtained crude product was purified by silica gel column chromatography and then recrystallized from toluene to obtain 11.9 g of a pale yellow solid, which was identified as the following aromatic amine derivative HT8 by FD-MS analysis (yield: 70%).

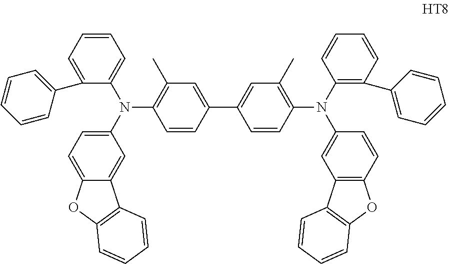 [SMILES: Cc1cc(-c2ccc(N(c3ccc4oc5ccccc5c4c3)c3ccccc3-c3ccccc3)c(C)c2)ccc1N(c1ccc2oc3ccccc3c2c1)c1ccccc1-c1ccccc1]

### Example 1-1

### Production of Organic EL Device

A glass substrate of 25 mm×75 mm×1.1 mm having an ITO transparent electrode line (product of Geomatec Company) was cleaned by ultrasonic cleaning in isopropyl alcohol for 5 min and then UV (ultraviolet)-ozone cleaning for 30 min.

The cleaned glass substrate having a transparent electrode line was mounted to a substrate holder of a vacuum vapor deposition apparatus. First, the following electron-accepting compound (A) was vapor-deposited so as to cover the transparent electrode to form a film A of 5 nm thick.

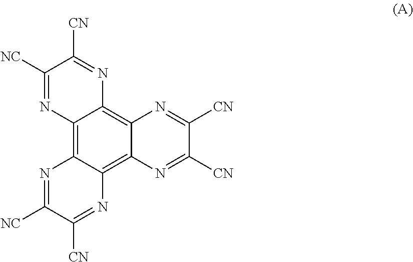 [SMILES: N#Cc1nc2c3nc(C#N)c(C#N)nc3c3nc(C#N)c(C#N)nc3c2nc1C#N]

On the film A, the following aromatic amine derivative (X1) (first hole transporting material) was vapor-deposited to form a first hole transporting layer of 160 nm thick. Successively after forming the first hole transporting layer, the following aromatic amine derivative (HT1) (second hole transporting material) was vapor-deposited to form a second hole transporting layer of 10 nm thick.

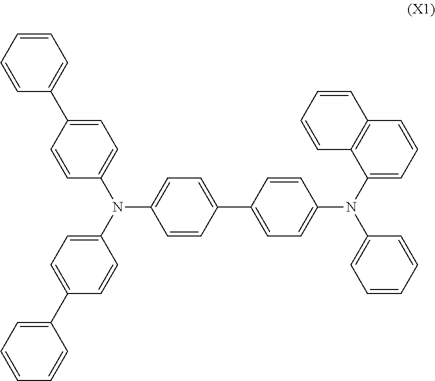 [SMILES: c1ccc(-c2ccc(N(c3ccc(-c4ccccc4)cc3)c3ccc(-c4ccc(N(c5ccccc5)c5cccc6ccccc56)cc4)cc3)cc2)cc1]

Then, the host compound (BH) and the dopant compound (BD) were vapor co-deposited into a thickness of 25 nm on the hole transporting layer to form a light emitting layer. The concentration of the dopant compound (BD) was 4% by mass.

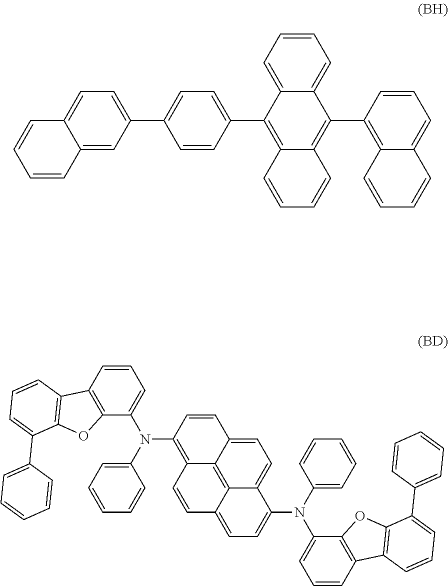

Thereafter, the following compound (ET1) was vapor-deposited on the light emitting layer into a thickness of 20 nm, the following compound (ET2) was vapor-deposited into a thickness of 5 nm, and then LiF was vapor-deposited into a thickness of 1 nm, thereby forming a electron transporting/injection layer. The LiF film (electron injecting electrode) was formed at a film-forming speed of 1 Å/min. Then, a metal Al was further deposited to form a cathode of 80 nm thick to produce an organic EL device.

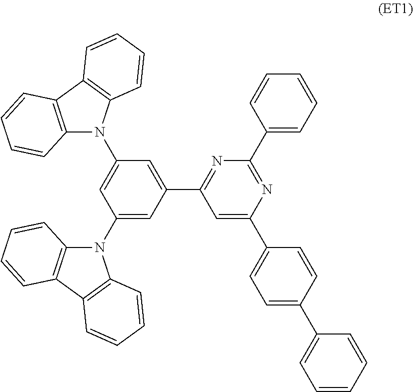 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

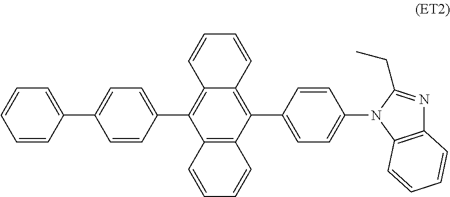 [SMILES: CCc1nc2ccccc2n1-c1ccc(-c2c3ccccc3c(-c3ccc(-c4ccccc4)cc3)c3ccccc23)cc1]

### Examples 1-2 to 1-7

In the same manner as in Example 1-1 except for using each aromatic amine derivative listed in Table 1 as the second hole transporting material, each organic EL device of Examples 1-2 to 1-7 was produced.

### Comparative Examples 1-1 and 1-2

In the same manner as in Example 1-1 except for using each aromatic amine derivative listed in Table 1 as the second hole transporting material, each organic EL device of Comparative Examples 1-1 and 1-2 was produced.

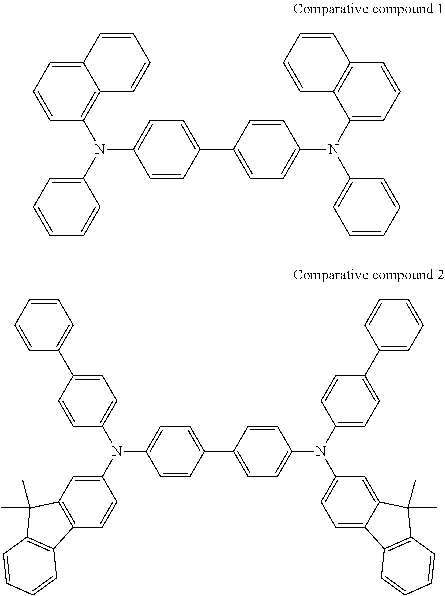

### Evaluation of Emission Performance of Organic EL Device

Each of the obtained organic EL devices was allowed to emit light by a direct current drive to measure the luminance (L) and the current density. Using the measured results, the current efficiency (L/J) and the driving voltage (V) each at a current density of 10 mA/cm² were determined. In addition, the device lifetime at a current density of 50 mA/cm² was determined. The “80% lifetime” is the time taken until the luminance was reduced to 80% of the initial luminance when driving at a constant current. The results are shown in Table 1.

TABLE 1

| | | Results of Measurement | | |
|---|---|---|---|---|
| | | Emission | | |
| | Second hole | efficiency | Driving voltage | 80% |
| | transporting | (cd/A) | (V) | Lifetime |
| | material | @10 mA/cm² | @10 mA/cm² | (h) |
| Examples | | | | |
| 1-1 | HT1 | 7.1 | 3.8 | 250 |
| 1-2 | HT2 | 6.5 | 4.0 | 290 |
| 1-3 | HT3 | 6.9 | 3.8 | 220 |
| 1-4 | HT4 | 7.2 | 4.1 | 270 |
| 1-5 | HT5 | 6.8 | 4.1 | 210 |
| 1-6 | HT6 | 6.8 | 3.8 | 210 |
| 1-7 | HT7 | 7.1 | 3.8 | 200 |
| Comparative | | | | |
| Examples | | | | |
| 1-1 | comparative | 5.5 | 4.0 | 250 |
| | compound 1 | | | |
| 1-2 | comparative | 5.2 | 3.8 | 170 |
| | compound 2 | | | |

As seen from Table 1, it can be found that an organic EL device which exhibits a high efficiency even when driving at a low voltage and exhibits a long lifetime is obtained by using the aromatic amine derivative of the invention.

### Example 2-1

### Production of Organic EL Device

A glass substrate of 25 mm×75 mm×1.1 mm having an ITO transparent electrode line (product of Geomatec Company) was cleaned by ultrasonic cleaning in isopropyl alcohol for 5 min and then UV (ultraviolet)-ozone cleaning for 30 min.

The cleaned glass substrate having a transparent electrode line was mounted to a substrate holder of a vacuum vapor deposition apparatus. First, the following electron-accepting compound (A) was vapor-deposited so as to cover the transparent electrode to form a film A of 5 nm thick.

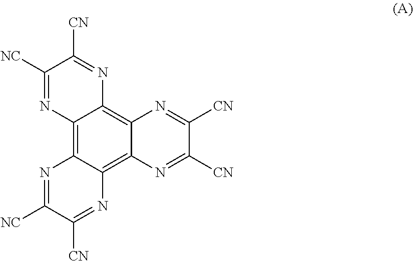 [SMILES: N#Cc1nc2c3nc(C#N)c(C#N)nc3c3nc(C#N)c(C#N)nc3c2nc1C#N]

On the film A, the above aromatic amine derivative (HT1) (first hole transporting material) was vapor-deposited to form a first hole transporting layer of 160 nm thick. Successively after forming the first hole transporting layer, the following aromatic amine derivative (Y1) (second hole transporting material) was vapor-deposited to form a second hole transporting layer of 10 nm thick.

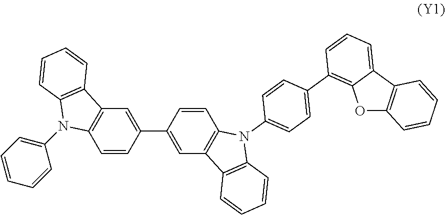 [SMILES: c1ccc(-n2c3ccccc3c3cc(-c4ccc5c(c4)c4ccccc4n5-c4ccc(-c5cccc6c5oc5ccccc56)cc4)ccc32)cc1]

Then, the host compound (BH) and the dopant compound (BD) were vapor co-deposited into a thickness of 25 nm on the hole transporting layer to form a light emitting layer. The concentration of the dopant compound (BD) was 4% by mass.

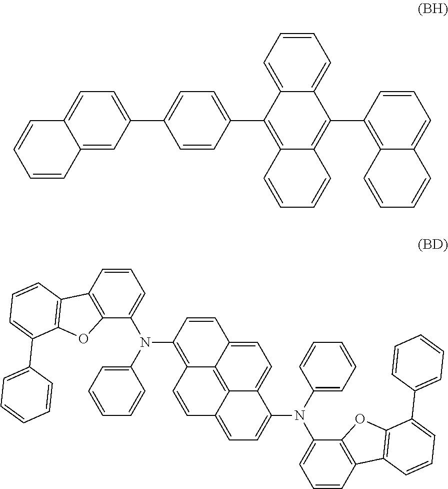

Thereafter, the following compound (ET1) was vapor-deposited on the light emitting layer into a thickness of 20 nm, the following compound (ET2) was vapor-deposited into a thickness of 5 nm, and then LiF was vapor-deposited into a thickness of 1 nm, thereby forming a electron transporting/injection layer. The LiF film (electron injecting electrode) was formed at a film-forming speed of 1 Å/min. Then, a metal Al was further deposited to form a cathode of 80 nm thick to produce an organic EL device.

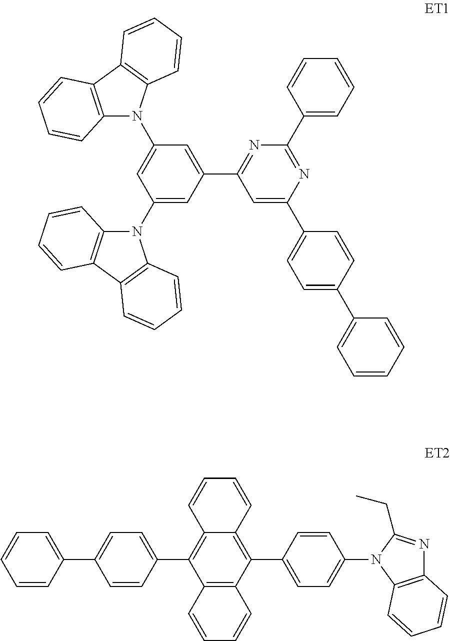 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; CCc1nc2ccccc2n1-c1ccc(-c2c3ccccc3c(-c3ccc(-c4ccccc4)cc3)c3ccccc23)cc1]

### Example 2-2 to 2-7

In the same manner as in Example 2-1 except for using each aromatic amine derivative listed in Table 2 as the first hole transporting material, each organic EL device of Examples 2-2 to 2-7 was produced.

### Comparative Examples 2-1 and 2-2

In the same manner as in Example 2-1 except for using each aromatic amine derivative listed in Table 2 as the first hole transporting material, each organic EL device of Comparative Examples 2-1 and 2-2 was produced.

### Evaluation of Emission Performance of Organic EL Device

Each of the obtained organic EL devices was allowed to emit light by a direct current drive to measure the luminance (L) and the current density. Using the measured results, the current efficiency (L/J) and the driving voltage (V) each at a current density of 10 mA/cm² were determined. In addition, the device lifetime at a current density of 50 mA/cm² was determined. The “80% lifetime” is the time taken until the luminance was reduced to 80% of the initial luminance when driving at a constant current. The results are shown in Table 2.

TABLE 2

| | | Results of Measurement | | |
|---|---|---|---|---|
| | | Emission | | |
| | First hole | efficiency | Driving voltage | 80% |
| | transporting | (cd/A) | (V) | Lifetime |
| | material | @10 mA/cm² | @10 mA/cm² | (h) |
| Examples | | | | |
| 2-1 | HT1 | 8.1 | 3.8 | 350 |
| 2-2 | HT2 | 7.8 | 4.1 | 330 |
| 2-3 | HT3 | 8.0 | 3.8 | 350 |
| 2-4 | HT4 | 7.5 | 4.2 | 300 |
| 2-5 | HT5 | 7.8 | 4.2 | 300 |
| 2-6 | HT6 | 7.7 | 4.1 | 360 |
| 2-7 | HT7 | 7.8 | 4.2 | 300 |
| Comparative | | | | |
| Examples | | | | |
| 2-1 | comparative | 6.6 | 4.3 | 250 |
| | compound 1 | | | |
| 2-2 | comparative | 6.2 | 4.0 | 270 |
| | compound 2 | | | |

As seen from Table 2, it can be found that an organic EL device which exhibits a high efficiency even when driving at a low voltage and exhibits a long lifetime is obtained by using the aromatic amine derivative of the invention.

The present application claims the priority of Japanese Patent Application No. 2013-128789 filed on Jun. 19, 2013 and the disclosures in its specification and drawings are hereby incorporated by reference.

### INDUSTRIAL APPLICABILITY

According to the present invention, an organic EL device which is driven at a reduced driving voltage and has a ling lifetime is provided.

1: An aromatic amine derivative represented by formula (1): 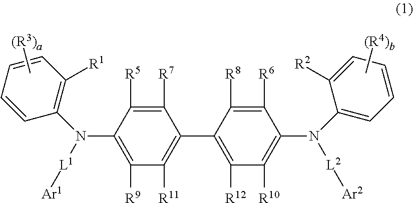
wherein:
Ar¹ and Ar² each independently represent a substituted or unsubstituted aryl group having 6 to 50 ring carbon atoms or a substituted or unsubstituted heteroaryl group having 3 to 50 ring atoms;
L¹ and L² each independently represent a single bond, a substituted or unsubstituted arylene group having 6 to 50 ring carbon atoms, or a substituted or unsubstituted heteroarylene group having 3 to 50 ring atoms;
R¹ to R⁴ each independently represents a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;
a and b each represent an integer of 0 to 4; and
R⁵ to R¹² each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group;
provided that at least one of R⁵ and R⁷ is not a hydrogen atom, wherein when R⁵ is not a hydrogen atom, R⁶ is not a hydrogen atom, and when R⁷ is not a hydrogen atom, R⁸ is not a hydrogen atom.

2: The aromatic amine derivative according to claim 1, wherein the aromatic amine derivative is represented by formula (2): 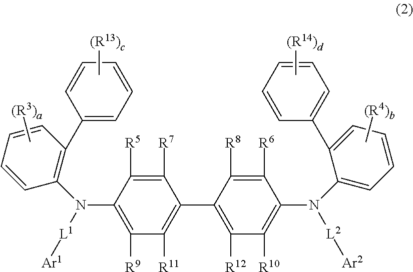
wherein:
R¹³ and R¹⁴ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;
c and d each represent an integer of 0 to 5, and when c and d each represent 2, the groups R¹³ or the groups R¹⁴ may be bonded to each other to form a ring, provided that R³ together with its adjacent aromatic ring and R¹⁴ together with its adjacent aromatic ring do not form a dibenzofuran ring, a dibenzothiophene ring or a fluorene ring; and
Ar¹, Ar², L¹, L², R³ to R¹², a, and b are as defined in formula (1).

3: The aromatic amine derivative according to claim 2, wherein the aromatic amine derivative is represented by formula (3): 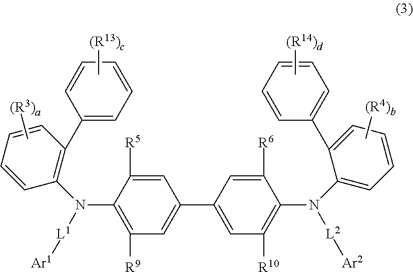
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², L¹, L², R³, R⁴, R⁹, R¹⁰, R¹³, R¹⁴, a, b, c, and d are as defined in formula (2).

4: The aromatic amine derivative according to claim 2, wherein the aromatic amine derivative is represented by formula (4): 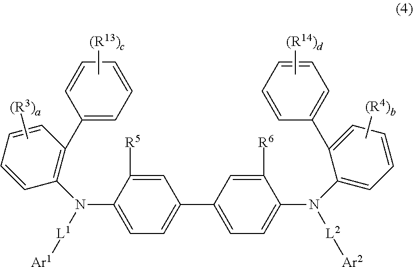
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², L¹, L², R³, R⁴, R¹³, R¹⁴, a, b, c, and d are as defined in formula (2).

5: The aromatic amine derivative according to claim 2, wherein the aromatic amine derivative is represented by formula (5): 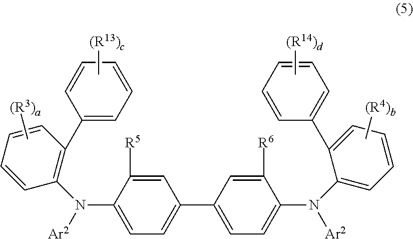
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², R³, R⁴, R¹³, R¹⁴, a, b, c, and d are as defined in formula (2).

6: The aromatic amine derivative according to claim 2, wherein the aromatic amine derivative is represented by formula (6): 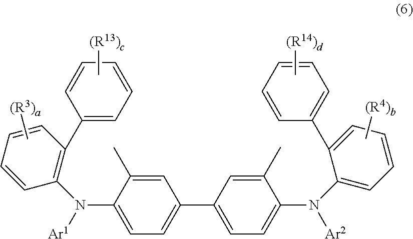
wherein Ar¹, Ar², R³, R⁴, R¹³, R¹⁴, a, b, c, and d are as defined in formula (2).

7: The aromatic amine derivative according to claim 2, wherein the aromatic amine derivative is represented by formula (7): 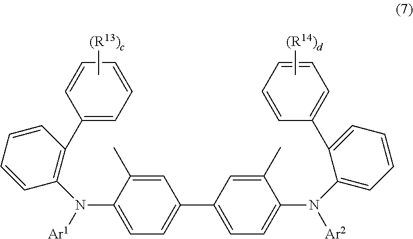
wherein Ar¹, Ar², R¹³, R¹⁴, c, and d are as defined in formula (2).

8: The aromatic amine derivative according to claim 1, wherein the aromatic amine derivative is represented by formula (8): 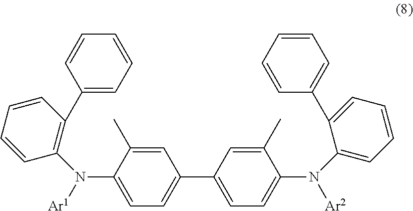
wherein Ar¹ and Ar² are as defined in formula (1).

9: The aromatic amine derivative according to claim 3, wherein Ar¹ and Ar² each represent a substituted or unsubstituted aryl group having 10 to 50 ring carbon atoms.

10: The aromatic amine derivative according to claim 2, wherein the aromatic amine derivative is represented by formula (26): 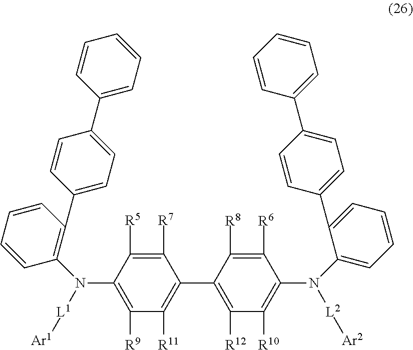
wherein Ar¹, Ar², L¹, L², and R⁵ to R¹² are as defined in formula (2).

11: The aromatic amine derivative according to claim 10, wherein R⁵ is not a hydrogen atom and R⁶ is not a hydrogen atom.

12: The aromatic amine derivative according to claim 1, wherein the aromatic amine derivative is represented by formula (17): 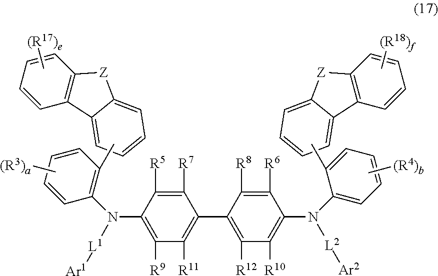
wherein:
Z represents —O—, —S—, or —CR¹⁹R²⁰—, wherein R¹⁹ and R²⁰ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group;
R¹⁷ and R¹⁸ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group;
e and f each independently represent an integer of 0 to 4; and
Ar¹, Ar², L¹, L², R³ to R¹², a, and b are as defined in formula (1).

13: The aromatic amine derivative according to claim 12, wherein the aromatic amine derivative is represented by formula (18): 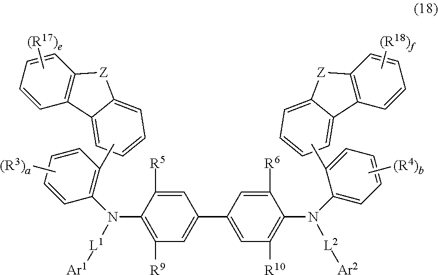
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², L¹, L², R³, R⁴, R⁹, R¹⁰, R¹⁷, R¹⁸, a, b, e, f, and Z are as defined in formula (17).

14: The aromatic amine derivative according to claim 12, wherein the aromatic amine derivative is represented by formula (19): 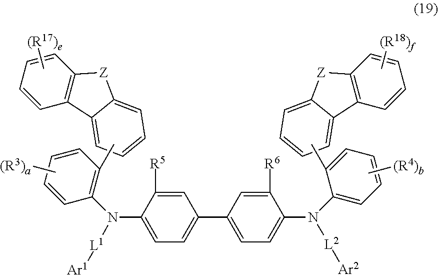
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², L¹, L², R³, R⁴, R¹⁷, R¹⁸, a, b, e, f and Z are as defined in formula (17).

15: The aromatic amine derivative according to claim 12, wherein the aromatic amine derivative is represented by formula (20): 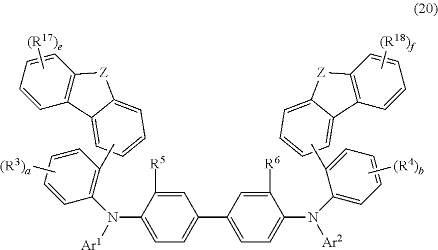
wherein:
R⁵ and R⁶ each independently represent a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group; and
Ar¹, Ar², R³, R⁴, R¹⁷, R¹⁸, a, b, e, f, and Z are as defined in formula (17).

16: The aromatic amine derivative according to claim 12, wherein the aromatic amine derivative is represented by formula (21): 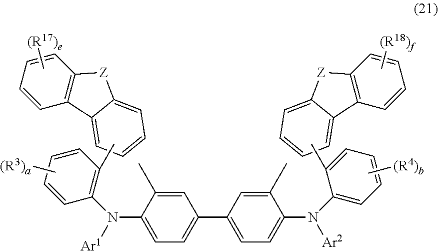
wherein Ar¹, Ar², R³, R⁴, R¹⁷, R¹⁸, a, b, e, f, and Z are as defined in formula (17).

17: The aromatic amine derivative according to claim 12, wherein the aromatic amine derivative is represented by formula (22): 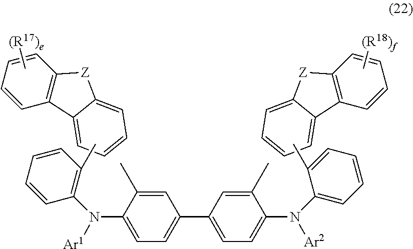
wherein Ar¹, Ar², R¹⁷, R¹⁸, e, f, and Z are as defined in formula (17).

18: The aromatic amine derivative according to claim 1, wherein the aromatic amine derivative is represented by any one of formulae (23) to (25): 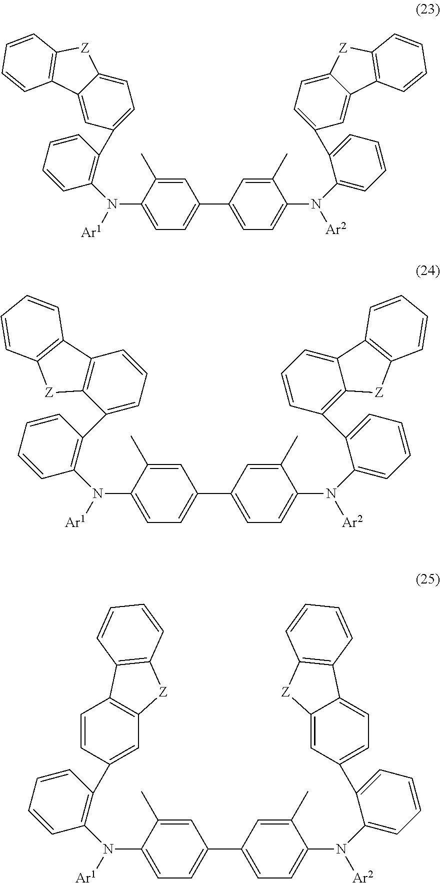
wherein Ar¹, Ar² and Z are as defined in formula (17).

19: The aromatic amine derivative according to claim 1, wherein L¹ and L² in any of formulae (17) to (19) and (26) each independently represent a single bond or a divalent group represented by formula (27): 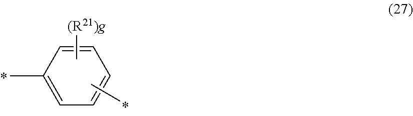
wherein:
each R²¹ independently represents a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 ring carbon atoms, a substituted or unsubstituted arylthio group having 5 to 50 ring carbon atoms, a substituted or unsubstituted alkoxycarbonyl group having 2 to 50 carbon atoms, a halogen atom, a cyano group, a nitro group, a silyl group, a hydroxyl group, or a carboxyl group; and
g represents an integer of 0 to 4.

20: The aromatic amine derivative according to claim 1, wherein Ar¹ and Ar² in any of formulae (1) to (8) and (17) to (26) are each independently represented by any one of formulae (9) to (16): 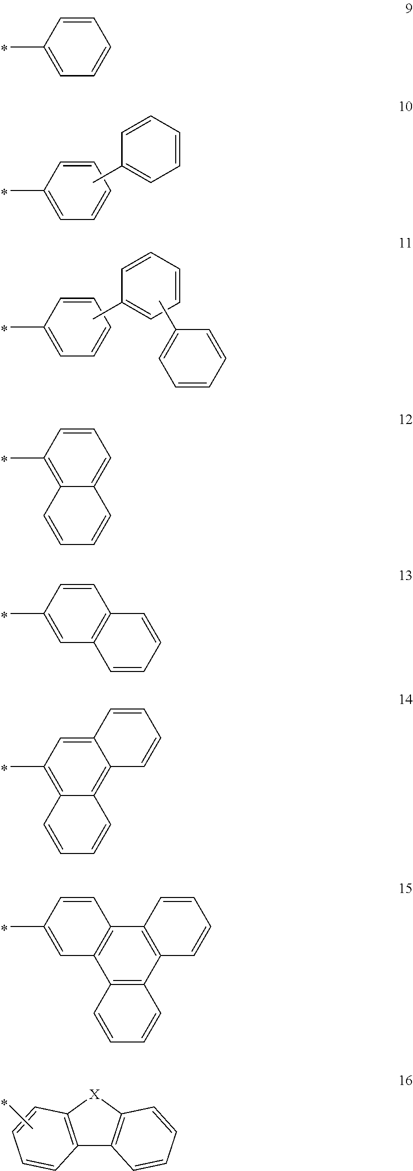
wherein:
X represents —O—, —S—, or —CR¹⁵R¹⁶—; and
R¹⁵ and R¹⁶ each independently represent a hydrogen atom, a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, or a silyl group.

21: A material for organic electroluminescence devices, comprising the aromatic amine derivative according to claim 1.

22: A hole transporting material for organic electroluminescence devices, comprising the aromatic amine derivative according to claim 1.

23: A hole transporting material for organic electroluminescence devices, comprising a hole transporting layer adjacent to an acceptor layer and the hole transporting layer comprises the aromatic amine derivative according to claim 1.

24: An organic electroluminescence device, comprising an organic thin film layer between an anode and a cathode opposite to the anode and at least one layer of the organic thin film layer comprises the aromatic amine derivative according to claim 1.
